SEQUENCE LISTING (89 sequences)

SEQ ID NO: 1 - DNA, 118 bp, synthetic construct
  misc_feature 1..118: note = miR30 CCR5 coding sequence
  source 1..118: mol_type = other DNA
aggtatattgctgttgacagtgagcgactgtaaactgagcttgctctactgtgaagccacagatgggtagagcaagcacagtttaccgctgcctactgcctcggacttcaaggggctt

SEQ ID NO: 2 - DNA, 116 bp, synthetic construct
  misc_feature 1..116: note = miR21 Vif coding sequence
  source 1..116: mol_type = other DNA
catctccatggctgtaccaccttgtcgggggatgtgtacttctgaacttgtgttgaatctcatggagttcagaagaacacatccgcactgacattttggtatctttcatctgacca

SEQ ID NO: 3 - DNA, 114 bp, synthetic construct
  misc_feature 1..114: note = miR185 Tat coding sequence
  source 1..114: mol_type = other DNA
gggcctggctcgagcagggggcgagggattccgcttcttcctgccatagcgtggtcccctcccctatggcaggcagaagcggcaccttccctcccaatgaccgcgtcttcgtcg

SEQ ID NO: 4 - DNA, 1104 bp, synthetic construct
  misc_feature 1..1104: note = Elongation Factor-1 alpha (EF1-alpha) promoter
  source 1..1104: mol_type = other DNA
ccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 5 - DNA, 18 bp, synthetic construct
  misc_feature 1..18: note = CCR5 miRNA target sequence
  source 1..18: mol_type = other DNA
gagcaagctcagtttaca

SEQ ID NO: 6 - DNA, 21 bp, synthetic construct
  misc_feature 1..21: note = Vif miRNA coding sequence
  source 1..21: mol_type = other DNA
gggatgtgtacttctgaactt

SEQ ID NO: 7 - DNA, 20 bp, synthetic construct
  misc_feature 1..20: note = Tat miRNA coding sequence
  source 1..20: mol_type = other DNA
tccgcttcttcctgccatag

SEQ ID NO: 8 - DNA, 126 bp, synthetic construct
  misc_feature 1..126: note = TAR decoy sequence
  source 1..126: mol_type = other DNA
cttgcaatgatgtcgtaatttgcgtcttacctcgttctcgacagcgaccagatctgagcctgggagctctctggctgtcagtaagctggtacagaaggttgacgaaaattcttactgagcaagaaa

SEQ ID NO: 9 - DNA, 21 bp, synthetic construct
  misc_feature 1..21: note = Rev/Tat shRNA target sequence #1
  source 1..21: mol_type = other DNA
gcggagacagcgacgaagagc

SEQ ID NO: 10 - DNA, 56 bp, synthetic construct
  misc_feature 1..56: note = Rev/Tat shRNA coding sequence #1
  source 1..56: mol_type = other DNA
gcggagacagcgacgaagagcttcaagagagctcttcgtcgctgtctccgcttttt

SEQ ID NO: 11 - DNA, 19 bp, synthetic construct
  misc_feature 1..19: note = Gag shRNA target sequence
  source 1..19: mol_type = other DNA
gaagaaatgatgacagcat

SEQ ID NO: 12 - DNA, 52 bp, synthetic construct
  misc_feature 1..52: note = Gag shRNA coding sequence
  source 1..52: mol_type = other DNA
gaagaaatgatgacagcatttcaagagaatgctgtcatcatttcttcttttt

SEQ ID NO: 13 - DNA, 18 bp, synthetic construct
  misc_feature 1..18: note = Pol shRNA target sequence
  source 1..18: mol_type = other DNA
caggagcagatgatacag

SEQ ID NO: 14 - DNA, 47 bp, synthetic construct
  misc_feature 1..47: note = Pol shRNA coding sequence
  source 1..47: mol_type = other DNA
caggagatgatacagttcaagagactgtatcatctgctcctgttttt

SEQ ID NO: 15 - DNA, 19 bp, synthetic construct
  misc_feature 1..19: note = CCR5 shRNA target sequence #1
  source 1..19: mol_type = other DNA
gtgtcaagtccaatctatg

SEQ ID NO: 16 - DNA, 52 bp, synthetic construct
  misc_feature 1..52: note = CCR5 shRNA coding sequence #1
  source 1..52: mol_type = other DNA
gtgtcaagtccaatctatgttcaagagacatagattggacttgacacttttt

SEQ ID NO: 17 - DNA, 19 bp, synthetic construct
  misc_feature 1..19: note = CCR5 shRNA target sequence #2
  source 1..19: mol_type = other DNA
gagcatgactgacatctac

SEQ ID NO: 18 - DNA, 52 bp, synthetic construct
  misc_feature 1..52: note = CCR5 shRNA coding sequence #2
  source 1..52: mol_type = other DNA
gagcatgactgacatctacttcaagagagtagatgtcagtcatgctcttttt

SEQ ID NO: 19 - DNA, 19 bp, synthetic construct
  misc_feature 1..19: note = CCR5 shRNA target sequence #3
  source 1..19: mol_type = other DNA
gtagctctaacaggttgga

SEQ ID NO: 20 - DNA, 52 bp, synthetic construct
  misc_feature 1..52: note = CCR5 shRNA coding sequence #3
  source 1..52: mol_type = other DNA
gtagctctaacaggttggattcaagagatccaacctgttagagctacttttt

SEQ ID NO: 21 - DNA, 19 bp, synthetic construct
  misc_feature 1..19: note = CCR5 shRNA target sequence #4
  source 1..19: mol_type = other DNA
gttcagaaactacctctta

SEQ ID NO: 22 - DNA, 52 bp, synthetic construct
  misc_feature 1..52: note = CCR5 shRNA coding sequence #4
  source 1..52: mol_type = other DNA
gttcagaaactacctcttattcaagagataagaggtagtttctgaacttttt

SEQ ID NO: 23 - DNA, 20 bp, synthetic construct
  misc_feature 1..20: note = CCR5 shRNA target sequence #5
  source 1..20: mol_type = other DNA
gagcaagctcagtttacacc

SEQ ID NO: 24 - DNA, 54 bp, synthetic construct
  misc_feature 1..54: note = CCR5 shRNA coding sequence #5
  source 1..54: mol_type = other DNA
gagcaagctcagtttacaccttcaagagaggtgtaaactgagcttgctcttttt

SEQ ID NO: 25 - DNA, 141 bp, synthetic construct
  misc_feature 1..141: note = Homo sapiens CCR5 mRNA target sequence 1
  source 1..141: mol_type = other DNA
atggattatcaagtgtcaagtccaatctatgacatcaattattatacatcggagccctgccaaaaaatcaatgtgaagcaaatcgcagcccgcctcctgcctccgctctactcactggtgttcatctttggttttgtgggc

SEQ ID NO: 26 - DNA, 633 bp, synthetic construct
  misc_feature 1..633: note = Homo sapiens CCR5 mRNA target sequence 2
  source 1..633: mol_type = other DNA
aacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaac

SEQ ID NO: 27 - DNA, 70 bp, synthetic construct
  misc_feature 1..70: note = Homo sapiens CCR5 mRNA target sequence 3
  source 1..70: mol_type = other DNA
accttccaggaattctttggcctgaataattgcagtagctctaacaggttggaccaagctatgcaggtga

SEQ ID NO: 28 - DNA, 140 bp, synthetic construct
  misc_feature 1..140: note = Homo sapiens CCR5 mRNA target sequence 4
  source 1..140: mol_type = other DNA
cagagactcttgggatgacgcactgctgcatcaaccccatcatctatgcctttgtcggggagaagttcagaaactacctcttagtcttcttccaaaagcacattgccaaacgcttctgcaaatgctgttctattttccag

SEQ ID NO: 29 - DNA, 75 bp, synthetic construct
  misc_feature 1..75: note = Homo sapiens CCR5 mRNA target sequence 5
  source 1..75: mol_type = other DNA
caagaggctcccgagcgagcaagctcagtttacacccgatccactggggagcaggaaatatctgtgggcttgtga

SEQ ID NO: 30 - DNA, 359 bp, synthetic construct
  misc_feature 1..359: note = miR30-CCR5/miR21-Vif/miR185 Tat microRNA cluster coding sequence
  source 1..359: mol_type = other DNA
aggtatattgctgttgacagtgagcgactgtaaactgagcttgctctactgtgaagccacagatgggtagagcaagcacagtttaccgctgcctactgcctcggacttcaaggggcttcccgggcatctccatggctgtaccaccttgtcgggggatgtgtacttctgaacttgtgttgaatctcatggagttcagaagaacacatccgcactgacattttggtatctttcatctgaccagctagcgggcctggctcgagcagggggcgagggattccgcttcttcctgccatagcgtggtcccctcccctatggcaggcagaagcggcaccttccctcccaatgaccgcgtcttcgtc

SEQ ID NO: 31 - DNA, 591 bp, synthetic construct
  misc_feature 1..591: note = Long WPRE sequence
  source 1..591: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcct

SEQ ID NO: 32 - DNA, 1469 bp, synthetic construct
  misc_feature 1..1469: note = Elongation Factor-1 alpha (EF1-alpha) promoter; miR30CCR5; miR21Vif; miR185 Tat cluster coding sequence
  source 1..1469: mol_type = other DNA
ccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgtacaaggtatattgctgttgacagtgagcgactgtaaactgagcttgctctactgtgaagccacagatgggtagagcaagcacagtttaccgctgcctactgcctcggacttcaaggggcttcccgggcatctccatggctgtaccaccttgtcgggggatgtgtacttctgaacttgtgttgaatctcatggagttcagaagaacacatccgcactgacattttggtatctttcatctgaccagctagcgggcctggctcgagcagggggcgagggattccgcttcttcctgccatagcgtggtcccctcccctatggcaggcagaagcggcaccttccctcccaatgaccgcgtcttcgtc

SEQ ID NO: 33 - DNA, 228 bp, synthetic construct
  misc_feature 1..228: note = Rous Sarcoma virus (RSV) promoter
  source 1..228: mol_type = other DNA
gtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacg

SEQ ID NO: 34 - DNA, 180 bp, synthetic construct
  misc_feature 1..180: note = 5 Long terminal repeat (LTR)
  source 1..180: mol_type = other DNA
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 35 - DNA, 41 bp, synthetic construct
  misc_feature 1..41: note = Psi Packaging signal
  source 1..41: mol_type = other DNA
tacgccaaaaattttgactagcggaggctagaaggagagag

SEQ ID NO: 36 - DNA, 233 bp, synthetic construct
  misc_feature 1..233: note = Rev response element (RRE)
  source 1..233: mol_type = other DNA
aggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcc

SEQ ID NO: 37 - DNA, 118 bp, synthetic construct
  misc_feature 1..118: note = Central polypurine tract (cPPT)
  source 1..118: mol_type = other DNA
ttttaaaagaaaaggggggattggggggtacagtgcaggggaaagaatagtagacataatagcaacagacatacaaactaaagaattacaaaaacaaattacaaaattcaaaatttta

SEQ ID NO: 38 - DNA, 250 bp, synthetic construct
  misc_feature 1..250: note = 3 delta LTR
  source 1..250: mol_type = other DNA
tggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtca

SEQ ID NO: 39 - DNA, 352 bp, synthetic construct
  misc_feature 1..352: note = CMV early (CAG) enhancer; Enhance Transcription
  source 1..352: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatc

SEQ ID NO: 40 - DNA, 290 bp, synthetic construct
  misc_feature 1..290: note = Chicken beta actin (CAG) promoter; Transcription
  source 1..290: mol_type = other DNA
gctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 41 - DNA, 960 bp, synthetic construct
  misc_feature 1..960: note = Chicken beta actin intron; Enhance gene expression
  source 1..960: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgg

SEQ ID NO: 42 - DNA, 1503 bp, synthetic construct
  misc_feature 1..1503: note = HIV Gag; Viral capsid
  source 1..1503: mol_type = other DNA
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 43 - DNA, 1872 bp, synthetic construct
  misc_feature 1..1872: note = HIV Pol; Protease and reverse transcriptase
  source 1..1872: mol_type = other DNA
atgaatttgccaggaagatggaaaccaaaaatgatagggggaattggaggttttatcaaagtaggacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaaatatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagatgggttggtcagtgctggaatcaggaaagtacta

SEQ ID NO: 44 - DNA, 867 bp, synthetic construct
  misc_feature 1..867: note = HIV Integrase; Integration of viral RNA
  source 1..867: mol_type = other DNA
tttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaa

SEQ ID NO: 45 - DNA, 234 bp, synthetic construct
  misc_feature 1..234: note = HIV RRE; Binds Rev element
  source 1..234: mol_type = other DNA
aggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcct

SEQ ID NO: 46 - DNA, 351 bp, synthetic construct
  misc_feature 1..351: note = HIV Rev; Nuclear export and stabilize viral mRNA
  source 1..351: mol_type = other DNA
atggcaggaagaagcggagacagcgacgaagaactcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatag

SEQ ID NO: 47 - DNA, 448 bp, synthetic construct
  misc_feature 1..448: note = Rabbit beta globin poly A; RNA stability
  source 1..448: mol_type = other DNA
agatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatc

SEQ ID NO: 48 - DNA, 450 bp, synthetic construct
  misc_feature 1..450: note = Rabbit beta globin poly A; RNA stability
  source 1..450: mol_type = other DNA
agatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 49 - DNA, 577 bp, synthetic construct
  misc_feature 1..577: note = CMV promoter; Transcription
  source 1..577: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagc

SEQ ID NO: 50 - DNA, 573 bp, synthetic construct
  misc_feature 1..573: note = Beta globin intron; Enhance gene expression
  source 1..573: mol_type = other DNA
gtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacag

SEQ ID NO: 51 - DNA, 1519 bp, synthetic construct
  misc_feature 1..1519: note = VSV-G; Glycoprotein envelope-cell entry
  source 1..1519: mol_type = other DNA
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatga

SEQ ID NO: 52 - DNA, 511 bp, synthetic construct
  misc_feature 1..511: note = Promoter; PGK
  source 1..511: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 53 - DNA, 1162 bp, synthetic construct
  misc_feature 1..1162: note = Promoter; UbC
  source 1..1162: mol_type = other DNA
gcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaa

SEQ ID NO: 54 - DNA, 120 bp, synthetic construct
  misc_feature 1..120: note = Poly A; SV40
  source 1..120: mol_type = other DNA
gtttattgcagcttataatggttacaaataaagcaatagcatcacaaatttcacaaataaagcatttttttcactgcattctagttgtggtttgtccaaactcatcaatgtatcttatca

SEQ ID NO: 55 - DNA, 227 bp, synthetic construct
  misc_feature 1..227: note = Poly A; bGH
  source 1..227: mol_type = other DNA
gactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 56 - DNA, 1512 bp, synthetic construct
  misc_feature 1..1512: note = HIV Gag; Bal
  source 1..1512: mol_type = other DNA
atgggtgcgagagcgtcagtattaagcgggggagaattagataggtgggaaaaaattcggttaaggccagggggaaagaaaaaatatagattaaaacatatagtatgggcaagcagggaactagaaagattcgcagtcaatcctggcctgttagaaacatcagaaggctgcagacaaatactgggacagctacaaccatcccttcagacaggatcagaagaacttagatcattatataatacagtagcaaccctctattgtgtacatcaaaagatagaggtaaaagacaccaaggaagctttagacaaaatagaggaagagcaaaacaaatgtaagaaaaaggcacagcaagcagcagctgacacaggaaacagcggtcaggtcagccaaaatttccctatagtgcagaacctccaggggcaaatggtacatcaggccatatcacctagaactttaaatgcatgggtaaaagtaatagaagagaaagctttcagcccagaagtaatacccatgttttcagcattatcagaaggagccaccccacaagatttaaacaccatgctaaacacagtggggggacatcaagcagccatgcaaatgttaaaagaacccatcaatgaggaagctgcaagatgggatagattgcatcccgtgcaggcagggcctgttgcaccaggccagataagagatccaaggggaagtgacatagcaggaactaccagtacccttcaggaacaaataggatggatgacaagtaatccacctatcccagtaggagaaatctataaaagatggataatcctgggattaaataaaatagtaaggatgtatagccctaccagcattttggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcagctacactagaagaaatgatgacagcatgtcagggagtgggaggacccagccataaagcaagaattttggcagaagcaatgagccaagtaacaaattcagctaccataatgatgcagaaaggcaattttaggaaccaaagaaagattgttaaatgtttcaattgtggcaaagaagggcacatagccagaaactgcagggcccctaggaaaaggggctgttggaaatgtggaaaggaaggacaccaaatgaaagactgtactgagagacaggctaattttttagggaaaatctggccttcccacaaaggaaggccagggaatttccttcagagcagaccagagccaacagccccaccagccccaccagaagagagcttcaggtttggggaagagacaacaactccctctcagaagcaggagctgatagacaaggaactgtatcctttagcttccctcagatcactctttggcaacgacccctcgtcacaataa

SEQ ID NO: 57 - DNA, 1872 bp, synthetic construct
  misc_feature 1..1872: note = HIV Pol; Bal
  source 1..1872: mol_type = other DNA
atgaatttgccaggaagatggaaaccaaaaatgatagggggaattggaggttttatcaaagtaagacagtatgatcagatactcatagaaatctgtggacataaagctataggtacagtattaataggacctacacctgtcaacataattggaagaaatctgttgactcagattggttgcactttaaattttcccattagtcctattgaaactgtaccagtaaaattaaaaccaggaatggatggcccaaaagttaaacaatggccactgacagaagaaaaaataaaagcattaatggaaatctgtacagaaatggaaaaggaagggaaaatttcaaaaattgggcctgaaaatccatacaatactccagtatttgccataaagaaaaaagacagtactaaatggagaaaattagtagatttcagagaacttaataagaaaactcaagacttctgggaagtacaattaggaatacacatcccgcaggggttaaaaaagaaaaaatcagtaacagtactggatgtgggtgatgcatatttttcagttcccttagataaagaattcaggaagtatactgcatttaccatacctagtataaacaatgaaacaccagggatcagatatcagtacaatgtacttccacagggatggaaaggatcaccagcaatatttcaaagtagcatgacaagaatcttagagccttttagaaaacaaaatccagaaatagtgatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaagacagctggactgtcaatgacatacagaagttagtgggaaaattgaattgggcaagtcagatttacccaggaattaaagtaaagcaattatgtaggctccttaggggaaccaaggcattaacagaagtaataccactaacaaaagaaacagagctagaactggcagagaacagggaaattctaaaagaaccagtacatggggtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactacccatacaaaaagaaacatgggaaacatggtggacagagtattggcaagccacctggattcctgagtgggagtttgtcaatacccctcccttagtgaaattatggtaccagttagagaaagaacccataataggagcagaaacattctatgtagatggagcagctaaccgggagactaaattaggaaaagcaggatatgttactaacagaggaagacaaaaagttgtctccctaactgacacaacaaatcagaagactgagttacaagcaattcatctagctttacaagattcaggattagaagtaaacatagtaacagactcacaatatgcattaggaatcattcaagcacaaccagataaaagtgaatcagagttagtcagtcaaataatagaacagttaataaaaaaggaaaaggtctacctggcatgggtaccagcgcacaaaggaattggaggaaatgaacaagtagataaattagtcagtactggaatcaggaaagtacta

SEQ ID NO: 58 - DNA, 867 bp, synthetic construct
  misc_feature 1..867: note = HIV Integrase; Bal
  source 1..867: mol_type = other DNA
tttttagatggaatagatatagcccaagaagaacatgagaaatatcacagtaattggagagcaatggctagtgattttaacctgccacctgtggtagcaaaagaaatagtagccagctgtgataaatgtcagctaaaaggagaagccatgcatggacaagtagactgtagtccaggaatatggcaactagattgtacacatttagaaggaaaaattatcctggtagcagttcatgtagccagtggatatatagaagcagaagttattccagcagagacagggcaggaaacagcatactttctcttaaaattagcaggaagatggccagtaaaaacaatacatacagacaatggcagcaatttcactagtactacagtcaaggccgcctgttggtgggcggggatcaagcaggaatttggcattccctacaatccccaaagtcagggagtagtagaatctataaataaagaattaaagaaaattataggacaggtaagagatcaggctgaacatcttaaaacagcagtacaaatggcagtattcatccacaattttaaaagaaaaggggggattggggggtatagtgcaggggaaagaatagtagacataatagcaacagacatacaaactaaagaattacaaaaacaaattacaaaaattcaaaattttcgggtttattacagggacagcagagatccactttggaaaggaccagcaaagcttctctggaaaggtgaaggggcagtagtaatacaagataatagtgacataaaagtagtaccaagaagaaaagcaaagatcattagggattatggaaaacagatggcaggtgatgattgtgtggcaagtagacaggatgaggattag

SEQ ID NO: 59 - DNA, 1695 bp, synthetic construct
  misc_feature 1..1695: note = Envelope; RD114
  source 1..1695: mol_type = other DNA
atgaaactcccaacaggaatggtcattttatgtagcctaataatagttcgggcagggtttgacgacccccgcaaggctatcgcattagtacaaaaacaacatggtaaaccatgcgaatgcagcggagggcaggtatccgaggccccaccgaactccatccaacaggtaacttgcccaggcaagacggcctacttaatgaccaaccaaaaatggaaatgcagagtcactccaaaaaatctcacccctagcgggggagaactccagaactgcccctgtaacactttccaggactcgatgcacagttcttgttatactgaataccggcaatgcagggcgaataataagacatactacacggccaccttgcttaaaatacggtctgggagcctcaacgaggtacagatattacaaaaccccaatcagctcctacagtccccttgtaggggctctataaatcagcccgtttgctggagtgccacagcccccatccatatctccgatggtggaggacccctcgatactaagagagtgtggacagtccaaaaaaggctagaacaaattcataaggctatgcatcctgaacttcaataccaccccttagccctgcccaaagtcagagatgaccttagccttgatgcacggacttttgatatcctgaataccacttttaggttactccagatgtccaattttagccttgcccaagattgttggctctgtttaaaactaggtacccctacccctcttgcgatacccactccctctttaacctactccctagcagactccctagcgaatgcctcctgtcagattatacctcccctcttggttcaaccgatgcagttctccaactcgtcctgtttatcttcccctttcattaacgatacggaacaaatagacttaggtgcagtcacctttactaactgcacctctgtagccaatgtcagtagtcctttatgtgccctaaacgggtcagtcttcctctgtggaaataacatggcatacacctatttaccccaaaactggacaggactttgcgtccaagcctccctcctccccgacattgacatcatcccgggggatgagccagtccccattcctgccattgatcattatatacatagacctaaacgagctgtacagttcatccctttactagctggactgggaatcaccgcagcattcaccaccggagctacaggcctaggtgtctccgtcacccagtatacaaaattatcccatcagttaatatctgatgtccaagtcttatccggtaccatacaagatttacaagaccaggtagactcgttagctgaagtagttctccaaaataggaggggactggacctactaacggcagaacaaggaggaatttgtttagccttacaagaaaaatgctgtttttatgctaacaagtcaggaattgtgagaaacaaaataagaaccctacaagaagaattacaaaaacgcagggaaagcctggcatccaaccctctctggaccgggctgcagggctttcttccgtacctcctacctctcctgggacccctactcaccctcctactcatactaaccattgggccatgcgttttcaatcgattggtccaatttgttaaagacaggatctcagtggtccaggctctggttttgactcagcaatatcaccagctaaaacccatagagtacgagccatga

SEQ ID NO: 60 - DNA, 2013 bp, synthetic construct
  misc_feature 1..2013: note = Envelope; GALV
  source 1..2013: mol_type = other DNA
atgcttctcacctcaagcccgcaccaccttcggcaccagatgagtcctgggagctggaaaagactgatcatcctcttaagctgcgtattcggagacggcaaaacgagtctgcagaataagaacccccaccagcctgtgaccctcacctggcaggtactgtcccaaactggggacgttgtctgggacaaaaaggcagtccagcccctttggacttggtggccctctcttacacctgatgtatgtgccctggcggccggtcttgagtcctgggatatcccgggatccgatgtatcgtcctctaaaagagttagacctcctgattcagactatactgccgcttataagcaaatcacctggggagccatagggtgcagctaccctcgggctaggaccaggatggcaaattcccccttctacgtgtgtccccgagctggccgaacccattcagaagctaggaggtgtggggggctagaatccctatactgtaaagaatggagttgtgagaccacgggtaccgtttattggcaacccaagtcctcatgggacctcataactgtaaaatgggaccaaaatgtgaaatgggagcaaaaatttcaaaagtgtgaacaaaccggctggtgtaaccccctcaagatagacttcacagaaaaaggaaaactctccagagattggataacggaaaaaacctgggaattaaggttctatgtatatggacacccaggcatacagttgactatccgcttagaggtcactaacatgccggttgtggcagtgggcccagaccctgtccttgcggaacagggacctcctagcaagcccctcactctccctctctccccacggaaagcgccgcccacccctctacccccggcggctagtgagcaaacccctgcggtgcatggagaaactgttaccctaaactctccgcctcccaccagtggcgaccgactctttggccttgtgcagggggccttcctaaccttgaatgctaccaacccaggggccactaagtcttgctggctctgtttgggcatgagccccccttattatgaagggatagcctcttcaggagaggtcgcttatacctccaaccatacccgatgccactggggggcccaaggaaagcttaccctcactgaggtctccggactcgggtcatgcatagggaaggtgcctcttacccatcaacatctttgcaaccagaccttacccatcaattcctctaaaaaccatcagtatctgctcccctcaaaccatagctggtgggcctgcagcactggcctcaccccctgcctctccacctcagtttttaatcagtctaaagacttctgtgtccaggtccagctgatcccccgcatctattaccattctgaagaaaccttgttacaagcctatgacaaatcaccccccaggtttaaaagagagcctgcctcacttaccctagctgtcttcctggggttagggattgcggcaggtataggtactggctcaaccgccctaattaaagggcccatagacctccagcaaggcctaaccagcctccaaatcgccattgacgctgacctccgggcccttcaggactcaatcagcaagctagaggactcactgacttccctatctgaggtagtactccaaaataggagaggccttgacttactattccttaaagaaggaggcctctgcgcggccctaaaagaagagtgctgtttttatgtagaccactcaggtgcagtacgagactccatgaaaaaacttaaagaaagactagataaaagacagttagagcgccagaaaaaccaaaactggtatgaagggtggttcaataactccccttggtttactaccctactatcaaccatcgctgggcccctattgctcctccttttgttactcactcttgggccctgcatcatcaataaattaatccaattcatcaatgataggataagtgcagtcaaaattttagtccttagacagaaatatcagaccctagataacgaggaaaacctttaa

SEQ ID NO: 61 - DNA, 1530 bp, synthetic construct
  misc_feature 1..1530: note = Envelope; FUG
  source 1..1530: mol_type = other DNA
atggttccgcaggttcttttgtttgtactccttctgggtttttcgttgtgtttcgggaagttccccatttacacgataccagacgaacttggtccctggagccctattgacatacaccatctcagctgtccaaataacctggttgtggaggatgaaggatgtaccaacctgtccgagttctcctacatggaactcaaagtgggatacatctcagccatcaaagtgaacgggttcacttgcacaggtgttgtgacagaggcagagacctacaccaactttgttggttatgtcacaaccacattcaagagaaagcatttccgccccaccccagacgcatgtagagccgcgtataactggaagatggccggtgaccccagatatgaagagtccctacacaatccataccccgactaccactggcttcgaactgtaagaaccaccaaagagtccctcattatcatatccccaagtgtgacagatttggacccatatgacaaatcccttcactcaagggtcttccctggcggaaagtgctcaggaataacggtgtcctctacctactgctcaactaaccatgattacaccatttggatgcccgagaatccgagaccaaggacaccttgtgacatttttaccaatagcagagggaagagagcatccaacgggaacaagacttgcggctttgtggatgaaagaggcctgtataagtctctaaaaggagcatgcaggctcaagttatgtggagttcttggacttagacttatggatggaacatgggtcgcgatgcaaacatcagatgagaccaaatggtgccctccagatcagttggtgaatttgcacgactttcgctcagacgagatcgagcatctcgttgtggaggagttagttaagaaaagagaggaatgtctggatgcattagagtccatcatgaccaccaagtcagtaagtttcagacgtctcagtcacctgagaaaacttgtcccagggtttggaaaagcatataccatattcaacaaaaccttgatggaggctgatgctcactacaagtcagtccggacctggaatgagatcatcccctcaaaagggtgtttgaaagttggaggaaggtgccatcctcatgtgaacggggtgtttttcaatggtataatattagggcctgacgaccatgtcctaatcccagagatgcaatcatccctcctccagcaacatatggagttgttggaatcttcagttatccccctgatgcaccccctggcagacccttctacagttttcaaagaaggtgatgaggctgaggattttgttgaagttcacctccccgatgtgtacaaacagatctcaggggttgacctgggtctcccgaactggggaaagtatgtattgatgactgcaggggccatgattggcctggtgttgatattttccctaatgacatggtgcagagttggtatccatctttgcattaaattaaagcacaccaagaaaagacagatttatacagacatagagatgaaccgacttggaaagtaa

SEQ ID NO: 62 - DNA, 1497 bp, synthetic construct
  misc_feature 1..1497: note = Envelope; LCMV
  source 1..1497: mol_type = other DNA
atgggtcagattgtgacaatgtttgaggctctgcctcacatcatcgatgaggtgatcaacattgtcattattgtgcttatcgtgatcacgggtatcaaggctgtctacaattttgccacctgtgggatattcgcattgatcagtttcctacttctggctggcaggtcctgtggcatgtacggtcttaagggacccgacatttacaaaggagtttaccaatttaagtcagtggagtttgatatgtcacatctgaacctgaccatgcccaacgcatgttcagccaacaactcccaccattacatcagtatggggacttctggactagaattgaccttcaccaatgattccatcatcagtcacaacttttgcaatctgacctctgccttcaacaaaaagacctttgaccacacactcatgagtatagtttcgagcctacacctcagtatcagagggaactccaactataaggcagtatcctgcgacttcaacaatggcataaccatccaatacaacttgacattctcagatcgacaaagtgctcagagccagtgtagaaccttcagaggtagagtcctagatatgtttagaactgccttcggggggaaatacatgaggagtggctggggctggacaggctcagatggcaagaccacctggtgtagccagacgagttaccaatacctgattatacaaaatagaacctgggaaaaccactgcacatatgcaggtccttttgggatgtccaggattctcctttcccaagagaagactaagttcttcactaggagactagcgggcacattcacctggactttgtcagactcttcaggggtggagaatccaggtggttattgcctgaccaaatggatgattcttgctgcagagcttaagtgtttcgggaacacagcagttgcgaaatgcaatgtaaatcatgatgccgaattctgtgacatgctgcgactaattgactacaacaaggctgctttgagtaagttcaaagaggacgtagaatctgccttgcacttattcaaaacaacagtgaattctttgatttcagatcaactactgatgaggaaccacttgagagatctgatgggggtgccatattgcaattactcaaagttttggtacctagaacatgcaaagaccggcgaaactagtgtccccaagtgctggcttgtcaccaatggttcttacttaaatgagacccacttcagtgatcaaatcgaacaggaagccgataacatgattacagagatgttgaggaaggattacataaagaggcaggggagtacccccctagcattgatggaccttctgatgttttccacatctgcatatctagtcagcatcttcctgcaccttgtcaaaataccaacacacaggcacataaaaggtggctcatgtccaaagccacaccgattaaccaacaaaggaatttgtagttgtggtgcatttaaggtgcctggtgtaaaaaccgtctggaaaagacgctga

SEQ ID NO: 63 - DNA, 1692 bp, synthetic construct
  misc_feature 1..1692: note = Envelope; FPV
  source 1..1692: mol_type = other DNA
atgaacactcaaatcctggttttcgcccttgtggcagtcatccccacaaatgcagacaaaatttgtcttggacatcatgctgtatcaaatggcaccaaagtaaacacactcactgagagaggagtagaagttgtcaatgcaacggaaacagtggagcggacaaacatccccaaaatttgctcaaaagggaaaagaaccactgatcttggccaatgcggactgttagggaccattaccggaccacctcaatgcgaccaatttctagaattttcagctgatctaataatcgagagacgagaaggaaatgatgtttgttacccggggaagtttgttaatgaagaggcattgcgacaaatcctcagaggatcaggtgggattgacaaagaaacaatgggattcacatatagtggaataaggaccaacggaacaactagtgcatgtagaagatcagggtcttcattctatgcagaaatggagtggctcctgtcaaatacagacaatgctgctttcccacaaatgacaaaatcatacaaaaacacaaggagagaatcagctctgatagtctggggaatccaccattcaggatcaaccaccgaacagaccaaactatatgggagtggaaataaactgataacagtcgggagttccaaatatcatcaatcttttgtgccgagtccaggaacacgaccgcagataaatggccagtccggacggattgattttcattggttgatcttggatcccaatgatacagttacttttagtttcaatggggctttcatagctccaaatcgtgccagcttcttgaggggaaagtccatggggatccagagcgatgtgcaggttgatgccaattgcgaaggggaatgctaccacagtggagggactataacaagcagattgccttttcaaaacatcaatagcagagcagttggcaaatgcccaagatatgtaaaacaggaaagtttattattggcaactgggatgaagaacgttcccgaaccttccaaaaaaaggaaaaaaagaggcctgtttggcgctatagcagggtttattgaaaatggttgggaaggtctggtcgacgggtggtacggtttcaggcatcagaatgcacaaggagaaggaactgcagcagactacaaaagcacccaatcggcaattgatcagataaccggaaagttaaatagactcattgagaaaaccaaccagcaatttgagctaatagataatgaattcactgaggtggaaaagcagattggcaatttaattaactggaccaaagactccatcacagaagtatggtcttacaatgctgaacttcttgtggcaatggaaaaccagcacactattgatttggctgattcagagatgaacaagctgtatgagcgagtgaggaaacaattaagggaaaatgctgaagaggatggcactggttgctttgaaatttttcataaatgtgacgatgattgtatggctagtataaggaacaatacttatgatcacagcaaatacagagaagaagcgatgcaaaatagaatacaaattgacccagtcaaattgagtagtggctacaaagatgtgatactttggtttagcttcggggcatcatgctttttgcttcttgccattgcaatgggccttgttttcatatgtgtgaagaacggaaacatgcggtgcactatttgtatataa

SEQ ID NO: 64 - DNA, 1266 bp, synthetic construct
  misc_feature 1..1266: note = Envelope; RRV
  source 1..1266: mol_type = other DNA
agtgtaacagagcactttaatgtgtataaggctactagaccatacctagcacattgcgccgattgcggggacgggtacttctgctatagcccagttgctatcgaggagatccgagatgaggcgtctgatggcatgcttaagatccaagtctccgcccaaataggtctggacaaggcaggcacccacgcccacacgaagctccgatatatggctggtcatgatgttcaggaatctaagagagattccttgagggtgtacacgtccgcagcgtgctccatacatgggacgatgggacacttcatcgtcgcacactgtccaccaggcgactacctcaaggtttcgttcgaggacgcagattcgcacgtgaaggcatgtaaggtccaatacaagcacaatccattgccggtgggtagagagaagttcgtggttagaccacactttggcgtagagctgccatgcacctcataccagctgacaacggctcccaccgacgaggagattgacatgcatacaccgccagatataccggatcgcaccctgctatcacagacggcgggcaacgtcaaaataacagcaggcggcaggactatcaggtacaactgtacctgcggccgtgacaacgtaggcactaccagtactgacaagaccatcaacacatgcaagattgaccaatgccatgctgccgtcaccagccatgacaaatggcaatttacctctccatttgttcccagggctgatcagacagctaggaaaggcaaggtacacgttccgttccctctgactaacgtcacctgccgagtgccgttggctcgagcgccggatgccacctatggtaagaaggaggtgaccctgagattacacccagatcatccgacgctcttctcctataggagtttaggagccgaaccgcacccgtacgaggaatgggttgacaagttctctgagcgcatcatcccagtgacggaagaagggattgagtaccagtggggcaacaacccgccggtctgcctgtgggcgcaactgacgaccgagggcaaaccccatggctggccacatgaaatcattcagtactattatggactataccccgccgccactattgccgcagtatccggggcgagtctgatggccctcctaactctggcggccacatgctgcatgctggccaccgcgaggagaaagtgcctaacaccgtacgccctgacgccaggagcggtggtaccgttgacactggggctgctttgctgcgcaccgagggcgaatgca

SEQ ID NO: 65 - DNA, 1938 bp, synthetic construct
  misc_feature 1..1938: note = Envelope; MLV 10A1
  source 1..1938: mol_type = other DNA
atggaaggtccagcgttctcaaaaccccttaaagataagattaacccgtggaagtccttaatggtcatgggggtctatttaagagtagggatggcagagagcccccatcaggtctttaatgtaacctggagagtcaccaacctgatgactgggcgtaccgccaatgccacctcccttttaggaactgtacaagatgccttcccaagattatattttgatctatgtgatctggtcggagaagagtgggacccttcagaccaggaaccatatgtcgggtatggctgcaaataccccggagggagaaagcggacccggacttttgacttttacgtgtgccctgggcataccgtaaaatcggggtgtggggggccaagagagggctactgtggtgaatggggttgtgaaaccaccggacaggcttactggaagcccacatcatcatgggacctaatctcccttaagcgcggtaacaccccctgggacacgggatgctccaaaatggcttgtggcccctgctacgacctctccaaagtatccaattccttccaaggggctactcgagggggcagatgcaaccctctagtcctagaattcactgatgcaggaaaaaaggctaattgggacgggcccaaatcgtggggactgagactgtaccggacaggaacagatcctattaccatgttctccctgacccgccaggtcctcaatatagggccccgcatccccattgggcctaatcccgtgatcactggtcaactacccccctcccgacccgtgcagatcaggctccccaggcctcctcagcctcctcctacaggcgcagcctctatagtccctgagactgccccaccttctcaacaacctgggacgggagacaggctgctaaacctggtagaaggagcctatcaggcgcttaacctcaccaatcccgacaagacccaagaatgttggctgtgcttagtgtcgggacctccttattacgaaggagtagcggtcgtgggcacttataccaatcattctaccgccccggccagctgtacggccacttcccaacataagcttaccctatctgaagtgacaggacagggcctatgcatgggagcactacctaaaactcaccaggccttatgtaacaccacccaaagtgccggctcaggatcctactaccttgcagcacccgctggaacaatgtgggcttgtagcactggattgactccctgcttgtccaccacgatgctcaatctaaccacagactattgtgtattagttgagctctggcccagaataatttaccactcccccgattatatgtatggtcagcttgaacagcgtaccaaatataagagggagccagtatcgttgaccctggcccttctgctaggaggattaaccatgggagggattgcagctggaatagggacggggaccactgccctaatcaaaacccagcagtttgagcagcttcacgccgctatccagacagacctcaacgaagtcgaaaaatcaattaccaacctagaaaagtcactgacctcgttgtctgaagtagtcctacagaaccgaagaggcctagatttgctcttcctaaaagagggaggtctctgcgcagccctaaaagaagaatgttgtttttatgcagaccacacgggactagtgagagacagcatggccaaactaagggaaaggcttaatcagagacaaaaactatttgagtcaggccaaggttggttcgaagggcagtttaatagatccccctggtttaccaccttaatctccaccatcatgggacctctaatagtactcttactgatcttactctttggaccctgcattctcaatcgattggtccaatttgttaaagacaggatctcagtggtccaggctctggttttgactcaacaatatcaccagctaaaacctatagagtacgagccatga

SEQ ID NO: 66 - DNA, 2030 bp, synthetic construct
  misc_feature 1..2030: note = Envelope; Ebola
  source 1..2030: mol_type = other DNA
atgggtgttacaggaatattgcagttacctcgtgatcgattcaagaggacatcattctttctttgggtaattatccttttccaaagaacattttccatcccacttggagtcatccacaatagcacattacaggttagtgatgtcgacaaactggtttgccgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccacaaagagggtgctttcttcctgtatgaccgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaagctaccggttttggaaccaatgagacagagtatttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaacctcactagaaaaattcgcagtgaagagttgtctttcacagctgtatcaaacagagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctgacaacccttgccacaatctccacgagtcctcaaccccccacaaccaaaccaggtccggacaacagcacccacaatacacccgtgtataaacttgacatctctgaggcaactcaagttgaacaacatcaccgcagaacagacaacgacagcacagcctccgacactccccccgccacgaccgcagccggacccctaaaagcagagaacaccaacacgagcaagggtaccgacctcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggaggagagctcgaagagaagcaattgtcaatgctcaacccaaatgcaaccctaatttacattactggactactcaggatgaaggtgctgcaatcggactggcctggataccatatttcgggccagcagccgagggaatttacatagaggggctgatgcacaatcaagatggtttaatctgtgggttgagacagctggccaacgagacgactcaagctcttcaactgttcctgagagccacaaccgagctacgcaccttttcaatcctcaaccgtaaggcaattgatttcttgctgcagcgatggggcggcacatgccacattttgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttataattgcagttatcgctttattctgtatatgcaaatttgtcttttag

SEQ ID NO: 67 - DNA, 389 bp, synthetic construct
  misc_feature 1..389: note = Short WPRE sequence
  source 1..389: mol_type = other DNA
aatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 68 - DNA, 31 bp, synthetic construct
  misc_feature 1..31: note = Helper Plasmid Forward Primer
  source 1..31: mol_type = other DNA
taagcagaattcatgaatttgccaggaagat

SEQ ID NO: 69 - DNA, 36 bp, synthetic construct
  misc_feature 1..36: note = Helper Plasmid Reverse Primer
  source 1..36: mol_type = other DNA
ccatacaatgaatggacactaggcggccgcacgaat

SEQ ID NO: 70 - DNA, 2745 bp, synthetic construct
  misc_feature 1..2745: note = Gag, Pol, Integrase fragment
  source 1..2745: mol_type = other DNA
gaattcatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaa

SEQ ID NO: 71 - DNA, 1586 bp, synthetic construct
  misc_feature 1..1586: note = DNA Fragment containing Rev, RRE and rabbit beta globin poly A
  source 1..1586: mol_type = other DNA
tctagaatggcaggaagaagcggagacagcgacgaagagctcatcagaacagtcagactcatcaagcttctctatcaaagcaacccacctcccaatcccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcagcggccgccccggg

SEQ ID NO: 72 - DNA, 1614 bp, synthetic construct
  misc_feature 1..1614: note = DNA fragment containing the CAG enhancer/promoter/intron sequence
  source 1..1614: mol_type = other DNA
acgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggaattc

SEQ ID NO: 73 - DNA, 1531 bp, synthetic construct
  misc_feature 1..1531: note = DNA fragment containing VSV-G
  source 1..1531: mol_type = other DNA
gaattcatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgagaattc

SEQ ID NO: 74 - DNA, 1227 bp, synthetic construct
  misc_feature 1..1227: note = Helper plasmid containing RRE and rabbit beta globin poly A
  source 1..1227: mol_type = other DNA
tctagaaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcacccggg

SEQ ID NO: 75 - DNA, 884 bp, synthetic construct
  misc_feature 1..884: note = RSV promoter and HIV Rev
  source 1..884: mol_type = other DNA
caattgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctccaagctcgagctcgtttagtgaaccgtcagatcgcctggagacgccatccacgctgttttgacctccatagaagacaccgggaccgatccagcctcccctcgaagctagcgattaggcatctcctatggcaggaagaagcggagacagcgacgaagaactcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtctaga

SEQ ID NO: 76 - DNA, 19 bp, synthetic construct
  misc_feature 1..19: note = Rev/Tat shRNA target sequence #2
  source 1..19: mol_type = other DNA
atggcaggaagaagcggag

SEQ ID NO: 77 - DNA, 52 bp, synthetic construct
  misc_feature 1..52: note = Rev/Tat shRNA coding sequence #2
  source 1..52: mol_type = other DNA
atggcaggaagaagcggagttcaagagactccgcttcttcctgccatttttt

SEQ ID NO: 78 - DNA, 279 bp, synthetic construct
  misc_feature 1..279: note = H1 promoter and shRT sequence
  source 1..279: mol_type = other DNA
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacttggatccgcggagacagcgacgaagagcttcaagagagctcttcgtcgctgtctccgcttttt

SEQ ID NO: 79 - DNA, 275 bp, synthetic construct
  misc_feature 1..275: note = H1 CCR5 sequence
  source 1..275: mol_type = other DNA
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacttggatccgtgtcaagtccaatctatgttcaagagacatagattggacttgacacttttt

SEQ ID NO: 80 - DNA, 642 bp, synthetic construct
  misc_feature 1..642: note = CAG promoter
  source 1..642: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 81 - DNA, 3992 bp, synthetic construct
  misc_feature 1..3992: note = pRSV Rev
  source 1..3992: mol_type = other DNA
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctccaagctcgagctcgtttagtgaaccgtcagatcgcctggagacgccatccacgctgttttgacctccatagaagacaccgggaccgatccagcctcccctcgaagctagtcgattaggcatctcctatggcaggaagaagcggagacagcgacgaagacctcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagctatagcagtagctgaggggacagatagggttatagaagtagtacaagaagcttggcactggccgtcgttttacatgatctgagcctgggagatctctggctaactagggaacccactgcttaagcctcaataaagcttgccttgagtgcttcaagtagtgtgtgcccgtctgttgtgtgactctggtaactagagatcacaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 82 - DNA, 6363 bp, synthetic construct
  misc_feature 1..6363: note = pCMV-VSV-G
  source 1..6363: mol_type = other DNA
gagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagctcctgggcaacgtgctggttgttgtgctgtctcatcattttggcaaagaattcctcgacggatccgccatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtgataaggatccgtcgaggaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacggatcggccgcaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctaga

SEQ ID NO: 83 - DNA, 9496 bp, synthetic construct
  misc_feature 1..9496: note = PSPAX2 delta Rev
  source 1..9496: mol_type = other DNA
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgggccggccgcgttgacgcgcacggcaagaggcgaggggcggcgactggtgagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaggacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaaatatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagatgggttggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaattctgcaacaactgctgtttatccatttcagaattggaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttgctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 84 - DNA, 21 bp, synthetic construct
  misc_feature 1..21: note = Vif miRNA target sequence
  source 1..21: mol_type = other DNA
aagttcagaagtacacatccc

SEQ ID NO: 85 - DNA, 20 bp, synthetic construct
  misc_feature 1..20: note = Tat miRNA target sequence
  source 1..20: mol_type = other DNA
ctatggcaggaagaagcgga

SEQ ID NO: 86 - DNA, 1110 bp, synthetic construct
  misc_feature 1..1110: note = Elongation Factor-1 alpha (EF1-alpha) promoter with 3 restriction recognition site
  source 1..1110: mol_type = other DNA
ccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgtaca

SEQ ID NO: 87 - DNA, 130 bp, synthetic construct
  misc_feature 1..130: note = miR30 CCR5 coding sequence with 5 and 3 restriction recognition sites
  source 1..130: mol_type = other DNA
tgtacaaggtatattgctgttgacagtgagcgactgtaaactgagcttgctctactgtgaagccacagatgggtagagcaagcacagtttaccgctgcctactgcctcggacttcaaggggcttgctagc

SEQ ID NO: 88 - DNA, 122 bp, synthetic construct
  misc_feature 1..122: note = miR21 Vif coding sequence with 5 restriction recognition site
  source 1..122: mol_type = other DNA
cccgggcatctccatggctgtaccaccttgtcgggggatgtgtacttctgaacttgtgttgaatctcatggagttcagaagaacacatccgcactgacattttggtatctttcatctgacca

SEQ ID NO: 89 - DNA, 125 bp, synthetic construct
  misc_feature 1..125: note = miR185 Tat coding sequence with 5 and 3 restriction recognition sites
  source 1..125: mol_type = other DNA
gctagcgggcctggctcgagcagggggcgagggattccgcttcttcctgccatagcgtggtcccctcccctatggcaggcagaagcggcaccttccctcccaatgaccgcgtcttcgtccccggg

(19) United States
(12) Patent Application Publication
(10) Pub. No.: US 2024/0115604 A1
(43) Pub. Date: Apr. 11, 2024

(54) METHODS OF MANUFACTURING GENETICALLY-MODIFIED LYMPHOCYTES

(71) Applicant: American Gene Technologies International Inc., Rockville, MD (US)

(72) Inventors: Haishan Li, Rockville, MD (US); Charles David Pauza, Rockville, MD (US)

(21) Appl. No.: 18/227,747

(22) Filed: Jul. 28, 2023

Related U.S. Application Data
(63) Continuation of application No. 17/042,043, filed on Sep. 25, 2020.
(60) Provisional application No. 62/648,804, filed on Mar. 27, 2018.

(51) Int. Cl. A61K 35/17; A61K 38/16; A61K 39/21; C12N 5/0783

(52) U.S. Cl. CPC A61K 35/17; A61K 38/162; A61K 39/21; C12N 5/0636; C12N 2310/141

(57) ABSTRACT

The present disclosure relates generally to immunization and immunotherapy for the treatment or prevention of HIV. In particular, the methods include purifying peripheral blood mononuclear cells (PBMC) from a source, stimulating the PBMC with at least one HIV-specific peptide, depleting at least one subset of cells from the PBMC, wherein the at least one subset of cells comprises any one or more of CD8+ T cells, CD4+ T cells, γδ cells, NK cells, B cells, T regulatory cells, and NKT cells, transducing the depleted PBMC with a viral delivery system encoding at least one genetic element, culturing the transduced PBMC for at least one day, and harvesting the cultured PBMC.

### CROSS-REFERENCE TO RELATED APPLICATION

This application is a continuation of U.S. patent application Ser. No. 17/042,043, filed on Sep. 25, 2020 entitled “METHODS OF MANUFACTURING GENETICALLY-MODIFIED LYMPHOCYTES,” which is a US National Stage Application of PCT Application No. PCT/US2019/024410, filed on Mar. 27, 2019 and entitled METHODS OF MANUFACTURING GENETICALLY-MODIFIED LYMPHOCYTES,” which claims priority to U.S. Provisional Patent Application No. 62/648,804, filed on Mar. 27, 2018 entitled “METHODS OF MANUFACTURING GENETICALLY-MODIFIED LYMPHOCYTES,” the disclosures of which are incorporated herein by reference in their entirety.

### FIELD

The present disclosure relates generally to the field of immunization and immunotherapy for the treatment and prevention of HIV. In particular, the disclosed methods relate to obtaining, processing, and proliferating leukocytes, including depletion of non-target cells, from HIV+ individuals seeking a cure in order to prepare a cell product suitable for infusion to such HIV+ individuals.

### BACKGROUND

Combination antiretroviral therapy (cART) (also known as Highly Active Antiretroviral Therapy or HAART) limits HIV-1 replication and slows disease progression, but drug toxicities and the emergence of drug-resistant viruses are challenges for long-term control in HIV-infected persons. Additionally, traditional antiretroviral therapy, while successful at delaying the onset of AIDS or death, has yet to provide a cure. Alternative treatment strategies are needed.

Intense interest in immunotherapy for HIV infection has been precipitated by emerging data indicating that the immune system has a major, albeit usually insufficient, role in limiting HIV replication. Some studies have tested vaccines against HIV, but success has been limited to date. Additionally, there has been interest in augmenting HIV immunotherapy by utilizing gene therapy techniques, but as with other immunotherapy approaches, success has been limited.

Gene therapy is one of the ripest areas of biomedical research with the potential to create new therapeutics that may involve the use of viral vectors. In view of the wide variety of potential genes available for therapy, an efficient means of delivering these genes is needed to fulfill the promise of gene therapy as a means of treating infectious and non-infectious diseases. Several viral systems including murine retrovirus, adenovirus, parvovirus (adeno-associated virus), coxsackie virus, measles virus, picornavirus, flavivirus, vaccinia virus, and herpes virus have been proposed as therapeutic gene transfer vectors. However, in vivo application of viral vectors is often limited by host immune responses against viral structural proteins and/or transduced gene products.

Although lentiviral vectors do not generally induce cytotoxicity and do not elicit strong host immune responses, some lentiviruses such as HIV-1, which encode several immunostimulatory gene products, have the potential to cause cytotoxicity and induce strong immune responses in vivo. Another important issue related to the use of lentiviral vectors is that of possible cytopathogenicity or functional unresponsiveness of T cells upon exposure to certain cytotoxic viral proteins. Likewise, the possibility of generating replication-competent, virulent lentivirus by recombination is often a concern. For HIV immunotherapy using gene therapy techniques, the failure to obtain sufficient numbers of HIV-specific CD4 T cells with protective genetic modifications typically results in a rapid reemergence of HIV upon termination of antiretroviral therapy.

Previous efforts to achieve a cure for HIV have fallen short for these reasons, among others. Accordingly, there remains a need for improved treatments of HIV.

### SUMMARY

In one aspect, a method is provided. The method includes purifying peripheral blood mononuclear cells (PBMC) from a source, stimulating the PBMC with at least one HIV-specific peptide, depleting at least one subset of cells from the PBMC, wherein the at least one subset of cells comprises any one or more of CD8+ T cells, CD4+ T cells, γδ cells, NK cells, B cells, T regulatory cells, and NKT cells, transducing the depleted PBMC with a viral delivery system encoding at least one genetic element, culturing the transduced PBMC for at least one day, and harvesting the cultured PBMC. In embodiments, the at least one subset of cells comprises any one or more of CD8+ T cells, γδ cells, NK cells, B cells, neutrophils, basophils, eosinophils, mast cells, dendritic cells, and NKT cells.

In embodiments, the culturing occurs in a static culture system or a semi-static culture system. In embodiments, the at least one HIV-specific peptide comprises a pool of synthetic, overlapping peptides. In embodiments, the pool of synthetic, overlapping peptides represents the HIV Gag polyprotein. In embodiments, the depleting comprises separating the at least one subset of cells from the depleted PBMC. In embodiments, the separating comprises magnetic bead separation. In embodiments, the at least one genetic element comprises a small RNA capable of inhibiting production of chemokine receptor CCR5 or at least one small RNA capable of targeting an HIV RNA sequence. In embodiments, the HIV RNA sequence comprises a HIV Vif sequence, a HIV Tat sequence, or a variant thereof. In embodiments, the at least one genetic element comprises a microRNA or a shRNA. In embodiments, the at least one genetic element comprises a microRNA having at least 80%, or at least 85%, or at least 90%, or at least 95% identity with at least one of SEQ ID NO: 1, SEQ ID NO: 2, SEQ ID NO: 3, or SEQ ID NO: 30.

In another aspect, a genetically-modified lymphocyte for treatment of a subject infected with HIV is provided. The genetically-modified lymphocyte is prepared by a process including the steps of purifying peripheral blood mononuclear cells (PBMC) comprising at least one lymphocyte from the subject, stimulating the at least one lymphocyte with at least one HIV-specific peptide, depleting at least one subset of cells from the PBMC, wherein the at least one subset of cells comprises any one or more of CD8+ T cells, CD4+ T cells, γδ cells, NK cells, B cells, T regulatory cells, and NKT cells, transducing the at least one lymphocyte with a viral delivery system encoding at least one genetic element, culturing the PBMC for at least one day, and harvesting the cultured PBMC.

In another aspect, a genetically-modified lymphocyte for treatment of a subject immunized with a preventive or therapeutic HIV vaccine is provided. The genetically-modified lymphocyte is prepared by a process including the steps of purifying peripheral blood mononuclear cells (PBMC) comprising at least one lymphocyte from the subject, stimulating the at least one lymphocyte with at least one HIV-specific peptide, depleting at least one subset of cells from the PBMC, wherein the at least one subset of cells comprises any one or more of CD8+ T cells, CD4+ T cells, γδ cells, NK cells, B cells, T regulatory cells, and NKT cells, transducing the at least one lymphocyte with a viral delivery system encoding at least one genetic element, culturing the PBMC for at least one day, and harvesting the cultured PBMC.

In another aspect, a genetically-modified lymphocyte for treatment of a subject exposed to, but not infected with HIV is provided. The genetically-modified lymphocyte is prepared by a process including the steps of purifying peripheral blood mononuclear cells (PBMC) comprising at least one lymphocyte from the subject, stimulating the at least one lymphocyte with at least one HIV-specific peptide, depleting at least one subset of cells from the PBMC, wherein the at least one subset of cells comprises any one or more of CD8+ T cells, CD4+ T cells, γδ cells, NK cells, B cells, T regulatory cells, and NKT cells, transducing the at least one lymphocyte with a viral delivery system encoding at least one genetic element, culturing the PBMC for at least one day, and harvesting the cultured PBMC.

In another aspect, a method is provided that comprises stimulating PBMC with at least one HIV-specific peptide; depleting at least one subset of cells from the PBMC, wherein the at least one subset of cells comprises any one or more of CD8+ T cells, CD4+ T cells, γδ cells, NK cells, B cells, T regulatory cells, and NKT cells; and transducing the depleted PBMC with a viral delivery system encoding at least one genetic element. In embodiments, the at least one HIV-specific peptide comprises a HIV gag peptide. In embodiments, the at least one genetic element comprises a small RNA capable of inhibiting production of chemokine receptor CCR5 or at least one small RNA capable of targeting an HIV RNA sequence. In embodiments, the HIV RNA sequences comprise an HIV Vif sequence, an HIV Tat sequence, or a variant thereof.

### BRIEF DESCRIPTION OF THE DRAWINGS

FIG. 1 depicts a flow diagram of an ex vivo treatment method of the present disclosure.

FIG. 2 depicts CD4+ T cell alteration and prevention of new infection in accordance with the present disclosure.

FIG. 3 depicts an exemplary lentiviral vector system comprised of a therapeutic vector, a helper plasmid, and an envelope plasmid. The therapeutic vector shown here is a preferred therapeutic vector, which is also referred to herein as AGT103, and contains miR30CCR5-miR21Vif-miR185-Tat.

FIG. 4 depicts an exemplary 3-vector lentiviral vector system in a circularized form.

FIG. 5 depicts an exemplary 4-vector lentiviral vector system in a circularized form.

FIG. 6 depicts a further exemplary 3-vector lentiviral vector system in a circularized form.

FIG. 7 depicts exemplary vector sequences. Positive (i.e., genomic) strand sequences of the promoter and miR cluster were developed for inhibiting the spread of CCR5-tropic HIV strains. The upper sequence (SEQ ID NO: 86) is the EF-1alpha promoter, which contains a restriction recognition site on its 3′ end. The bottom three sequences are the miR30 CCR5 (SEQ ID NO: 87), miR21 Vif (SEQ ID NO: 88), and miR185 Tat (SEQ ID NO: 89) sequences that contain restriction recognition sites. The portions of the sequences not underlined represent the restriction recognition sites.

FIG. 8 depicts a flow diagram for an exemplary method of producing CD4+ T cells, including a step of depleting CD8+ T cells.

FIG. 9 depicts data demonstrating a substantial increase of CD4+ T cells when Gag peptide-stimulated cells were depleted of CD8+ T cells.

FIG. 10 depicts data demonstrating an increase in CD4+ cells and an outgrowth of Vδ1 cells when CD8+ T cells were depleted.

FIG. 11 depicts data demonstrating that CD8+ T cell depletion increased the yield of target cells by approximately 6-fold.

FIG. 12 depicts data demonstrating a 3-fold increase of CD4+ T cells when Gag peptide-stimulated cells were depleted of CD8+ T cells.

FIG. 13 depicts data demonstrating that CD8+ T cell depletion resulted in expansion of CD56+ NK cells.

FIG. 14 depicts a flow diagram for an exemplary method of producing CD4+ T cells, including a step of depleting CD8+ T cells, CD56+ cells, CD19+ B cells and γδ cells.

FIG. 15 depicts data demonstrating the effect on CD4+ T cell yield using various cell depletion strategies including (i) no depletion; (ii) CD8+ T cell depletion; (iii) CD8+ T cell depletion and γδ cell depletion; and (iv) CD8+ T cell depletion, γδ cell depletion, and B cell depletion.

FIG. 16 depicts data demonstrating the effect on CD4+ T cell yield using various cell depletion strategies including (i) no depletion; (ii) CD8+ T cell depletion; and (iii) CD8+ T cell depletion, CD56+ cell depletion, γδ cell depletion, and B cell depletion,

FIG. 17 depicts a flow diagram for an exemplary method of producing CD4+ T cells, including a 4-way cell depletion step to deplete CD8+ T cells, CD56+ cells, CD19+ cells, and γδ cells.

FIG. 18 depicts data demonstrating results of CD4+ T cell expression using a 4-way cell depletion process.

### DETAILED DESCRIPTION

### Overview

Disclosed herein are methods and compositions for treating and/or preventing human immunodeficiency virus (HIV) disease to achieve a functional cure. The methods and compositions include integrating lentivirus, non-integrating lentivirus, and related viral vector technology as described below.

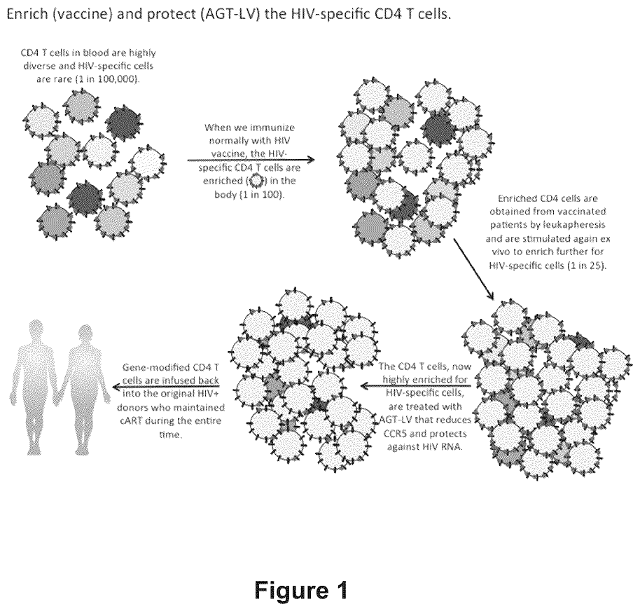

Disclosed herein are therapeutic viral vectors (e.g., lentiviral vectors), immunotherapies, and methods for their use for treating HIV infection. In embodiments, methods and compositions for achieving a functional cure for HIV infection are provided. As depicted in FIG. 1 herein, various aspects and embodiments of the disclosure may include a first stimulation event, for example a first therapeutic immunization with vaccines intended to produce strong immune responses against HIV in HIV-infected patients, for example with stable suppression of viremia due to daily administration of HAART. In embodiments, the leukocyte fraction is purified from an apheresis product. This is followed by (1) stimulation of purified leukocytes with at least one synthetic peptide representing an immunogenic HIV protein, (2) depleting unnecessary cells by a physical separation method, (3) transducing the stimulated and depleted leukocyte population with a therapeutic lentivirus vector, (4) transferring transduced cells to a static culture vessel for at least one day of growth, (5) harvesting cells from the static culture vessel, (6) substituting the growth medium with cryopreservation solution and freezing cells ready for infusion, and (7) re-infusion back into the original patient.

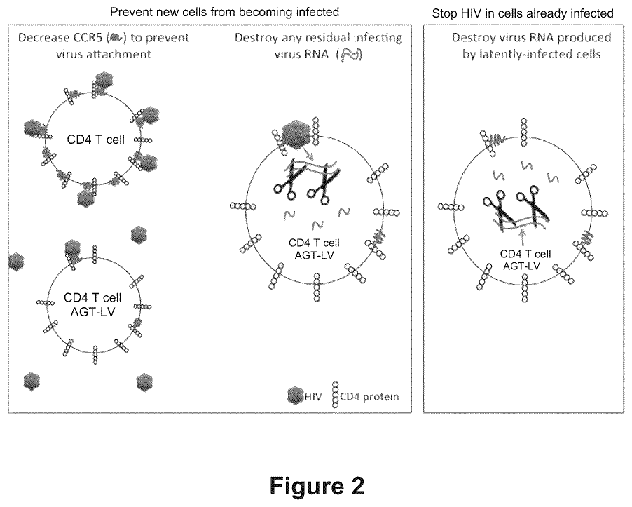

The various methods and compositions can be used to prevent infused cells, such as CD4+ T cells, from becoming infected with HIV once they are again present in the body of a person with HIV infection. For example, as illustrated in FIG. 2, to prevent infused cells from becoming infected, CCR5 expression can be modulated to prevent virus penetration. Further, destruction of any residual infecting viral RNA can also be targeted. In respect of the foregoing, and in reference to FIG. 2 herein, compositions and methods are provided to stop the HIV viral cycle in cells that become infected with HIV even in the absence of CCR5, or cells that were already infected before transduction and the need is to prevent the release of infectious virus. To stop the HIV viral cycle, viral RNA produced by latently-infected cells, such as latently-infected CD4+ T cells, is targeted.

Previous efforts to achieve a cure for HIV have fallen short due to, among others, the failure to obtain sufficient numbers of HIV-specific CD4 T cells with protective genetic modifications. When this number is below a critical threshold, a cure as described herein is not achieved. For example, upon termination of antiretroviral therapy HIV re-emergence generally follows. Thereafter, patients often experience rapid destruction of HIV-specific CD4 T cells, followed by return to progression of disease despite prior genetic therapy. By employing selective enrichment for HIV-specific T cells in accordance with the compositions and methods described herein, a new HIV treatment regimen has been developed including, in various embodiments, a cure.

### Definitions and Interpretation

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g.: Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane, Using Antibodies: A Laboratory Manual; Cold Spring Harbor Laboratory Press, Cold Spring Harbor, N.Y. (1998); and Coligan et al., Short Protocols in Protein Science, Wiley, John & Sons, Inc. (2003). Any enzymatic reactions or purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

As used herein, the terms “administration of” or “administering” an active agent means providing an active agent of the disclosure to the subject in need of treatment in a form that can be introduced into that individual's body in a therapeutically useful form and therapeutically effective amount.

As used herein, the term “AGT103” refers to a particular embodiment of a lentiviral vector that contains a miR30-CCR5/miR21-Vif/miR185-Tat microRNA cluster sequence, as detailed herein.

As used herein, the term “AGT103-T” refers to a cell that has been transduced with a lentivirus that contains the AGT103 lentiviral vector.

As used herein, the term “CCR5” refers to C-C chemokine receptor 5. Reference herein to “CCR5delta32” is reference to a mutant genotype in the CCR5 gene.

As used herein, the term “CD” refers to a particular cluster of differentiation protein. A non-limiting example of this terminology as used herein is CD4 protein expression. Examples of such proteins include but are not limited to CD4.

As used herein, the term “cART” refers to combination antiretroviral therapy. The term “cART” may be used synonymously with HAART (Highly Active Antiretroviral Therapy).

As used herein, the phrase “coding sequence” describes any viral vector sequence capable of being transcribed or reverse transcribed. A “coding sequence” includes, without limitation, exogenous sequences (e.g., sequences on vectors that have been transduced or transfected into cells) capable of being transcribed or reverse transcribed.

As used herein, the transitional term “comprising,” when used to define compositions and methods, means that the compositions and methods include the recited elements, but does not exclude others. As used herein, “consisting of,” when used to define compositions and methods, means that the compositions and methods exclude more than trace elements of other ingredients for compositions and substantial method steps. Embodiments defined by each of these transitional terms are within the scope of this disclosure. For example, it is intended that the methods and compositions can include additional steps and components (comprising) or alternatively including steps and compositions of no significance (consisting essentially of) or alternatively, intending only the stated method steps or compositions (consisting of). Further, as used herein, the term “includes” means includes without limitation.

As used herein, the term “engraftment” refers to the ability for one skilled in the art to determine a quantitative level of sustained engraftment in a subject following infusion of a cellular source (see for e.g.: Rosenberg et al., N. Engl. J. Med. 323:570-578 (1990); Dudley el al., J. Immunother. 24:363-373 (2001); Yee et al., Curr. Opin. Immunol. 13:141-146 (2001); Rooney et al., Blood 92:1549-1555 (1998)).

As used herein, the terms, “expression,” “expressed,” or “encodes” refer to the process by which polynucleotides are transcribed into mRNA and/or the process by which the transcribed mRNA is subsequently being translated into peptides, polypeptides, or proteins. Expression may include splicing of the mRNA in a eukaryotic cell or other forms of post-transcriptional modification or post-translational modification.

As used herein, the term “functional cure”, as referenced above and herein, and as further defined herein, refers to a state or condition wherein HIV+ individuals who previously required ongoing HIV therapies such as cART or HAART, may survive with low or undetectable virus replication using lower doses, intermittent doses, alternate drug combinations or single agents, or discontinued dosing of such HIV therapies. An individual may be said to have been “functionally cured” while still requiring adjunct therapy to maintain low level virus replication and slow or eliminate disease progression. A possible outcome of a functional cure is the eventual eradication of all or virtually all HIV such that no recurrence is detected within a specified time frame, for example, 1 month, 3 months, 6 months, 1 year, 3 years, and 5 years, and all other time frames as may be defined.

As used herein, the phrase “HIV-specific peptide” refers to any peptide that is capable of generating an anti-HIV response in a T cell, including a CD4+ T cell, or is otherwise related to, or expressed or encoded by, HIV, including without limitation any GAG peptide.

As used herein, the term “HIV vaccine” encompasses immunogens plus vehicle plus adjuvant intended to elicit HIV-specific immune responses. The term “HIV vaccine” is within the meaning of the term “stimulatory agent” as described herein. A “HIV vaccine” may include purified or whole inactivated virus particles that may be HIV or recombinant virus vectors capable of expressing HIV proteins, protein fragments or peptides, glycoprotein fragments or glycopeptides, in addition to recombinant bacterial vectors, plasmid DNA or RNA capable of directing cells to producing HIV proteins, glycoproteins or protein fragments able to elicit specific immunity. Alternately, specific methods for immune stimulation including anti-CD3/CD28 beads, T cell receptor-specific antibodies, mitogens, superantigens, cytokines and other chemical or biological stimuli may be used to activate dendritic, T or B cells for the purposes of enriching HIV-specific CD4 T cells prior to transduction or for in vitro assay of lentivirus-transduced CD4 T cells. Activating substances may be soluble, polymeric assemblies, liposome or endosome-based or linked to beads. Cytokines including interleukin-2, 6, 7, 12, 15, 23 or others may be added to improve cellular responses to stimuli and/or improve the survival of CD4 T cells throughout the culture and transduction intervals. Alternately, and without limiting any of the foregoing, the term “HIV vaccine” encompasses the MVA/HIV62B vaccine, and variants and analogs thereof. The MVA/HIV62B vaccine is a known highly attenuated double recombinant MVA vaccine. The MVA/HIV62B vaccine was constructed through the insertion of HIV-1 gag-pol and env sequences into the known MVA vector (see, e.g.: Goepfert et al. (2014) J. Infect. Dis. 210(1): 99-110, and see WO2006026667, both of which are incorporated herein by reference). The term “HIV vaccine” also includes any one or more vaccines provided in Table 1, below and in any similar tables contained in the priority documents (all of which are incorporated herein in their entirety).

TABLE 1

| IAVI Clinical Trial ID* | Prime** |
|---|---|
| HVTN 704 AMP | VRC-HIVMAB060-00-AB |
| VAC89220HPX2004 | Ad26.Mos.HIV Trivalent |
| 01-I-0079 | VRC4302 |
| 04/400-003-04 | APL 400-003 GENEVAX-HIV |
| 10-1074 | 10-1074 |
| 87 I-114 | gp160 Vaccine (Immuno-AG) |
| ACTG 326; PACTG 326 | ALVAC vCP1452 |
| Ad26.ENVA.01 | Ad26.EnvA-01 |
| Ad5HVR48.ENVA.01 | Ad5HVR48.ENVA.01 |
| ANRS VAC 02 | rgp 160 + peptide V3 ANRS |
| | VAC 02 |
| ANRS VAC 04 | LIPO-6 |
| ANRS VAC 05 | ALVAC vCP125 |
| ANRS VAC 07 | ALVAC vCP300 |
| ANRS VAC 08 | ALVAC-HIV MN120TMG |
| | strain (vCP205) |
| ANRS VAC 09 bis | LIPO-6 |
| ANRS VAC 12 | LPHIV1 |
| ANRS VAC 14 | gp160 MN/LAI |
| ANRS VAC 16 | LPHIV1 |
| ANRS VAC 18 | LIPO-5 |
| APL 400-003RX101 | APL 400-003 GENEVAX-HIV |
| AVEG 002 | HIVAC-1e |
| AVEG 003 | VaxSyn gp160 Vaccine |
| | (MicroGeneSys) |
| AVEG 004 | gp160 Vaccine (Immuno-AG) |
| AVEG 005A/B | Env 2-3 |
| AVEG 006X; VEU 006 | MN rgp120 |
| AVEG 007A/B | rgp120/HIV-1 SF-2 |
| AVEG 011 | UBI HIV-1 Peptide |
| | Immunogen, Multivalent |
| AVEG 013A | gp160 Vaccine (Immuno-AG) |
| AVEG 014A/B | TBC-3B |
| AVEG 017 | UBI HIV-1 Peptide Vaccine, |
| | Microparticulate Monovalent |
| AVEG 019 | p17/p24:Ty-VLP |
| AVEG 020 | gp120 C4-V3 |
| AVEG 021 | P3C541b Lipopeptide |
| AVEG 022 | ALVAC-HIV MN120TMG |
| | strain (vCP205) |
| AVEG 028 | Salmonella typhi CVD 908- |
| | HIV-1 LAI gp 120 |
| AVEG 031 | APL 400-047 |
| AVEG 034/034A | ALVAC vCP1433 |
| C060301 | GTU-MultiHIV |
| C86P1 | HIV gp140 ZM96 |
| Cervico-vaginal CN54gp140- | CN54gp140 |
| hsp70 Conjugate Vaccine | |
| (TL01) | |
| CM235 and SF2gp120 | CM235 (ThaiE) gp120 plus |
| | SF2(B) gp120 |
| CombiHIVvac (KombiVIChvak) | CombiHIVvac |
| CRC282 | P2G12 |
| CUTHIVAC002 | DNA-C CN54ENV |
| DCVax-001 | DCVax-001 |
| DNA-4 | DNA-4 |
| DP6?001 | DP6?001 DNA |
| DVP-1 | EnvDNA |
| EN41-UGR7C | EN41-UGR7C |
| EnvPro | EnvPro |
| EuroNeut41 | EN41-FPA2 |
| EV01 | NYVAC-C |
| EV02 (EuroVacc 02) | DNA-C |
| Extention HVTN 073E/SAAVI | Sub C gp140 |
| 102 | |
| F4/AS01 | F4/AS01 |
| FIT Biotech | GTU-Nef |
| Guangxi CDC DNA vaccine | Chinese DNA |
| HGP-30 memory responses | HGP-30 |
| HIV-CORE002 | ChAdV63.HIVconsv |
| HIV-POL-001 | MVA-mBN32 |
| HIVIS 01 | HIVIS-DNA |
| HIVIS 02 | MVA-CMDR |
| HVRF-380-131004 | Vichrepol |
| HVTN 040 | AVX101 |
| HVTN 041 | rgp120w61d |
| HVTN 044 | VRC-HIVDNA009-00-VP |
| HVTN 045 | pGA2/JS7 DNA |
| HVTN 048 | EP HIV-1090 |
| HVTN 049 | Gag and Env DNA/PLG |
| | microparticles |
| HVTN 050/Merck 018 | MRKAd5 HIV-1 gag |
| HVTN 052 | VRC-HIVDNA009-00-VP |
| HVTN 054 | VRC-HIVADV014-00-VP |
| HVTN 055 | TBC-M335 |
| HVTN 056 | MEP |
| HVTN 059 | AVX101 |
| HVTN 060 | HIV-1 gag DNA |
| HVTN 064 | EP HIV-1043 |
| HVTN 065 | pGA2/JS7 DNA |
| HVTN 067 | EP-1233 |
| HVTN 070 | PENNVAX-B |
| HVTN 072 | VRC-HIVDNA044-00-VP |
| HVTN 073 | SAAVI DNA-C2 |
| HVTN 076 | VRC-HIVDNA016-00-VP |
| HVTN 077 | VRC-HIVADV027-00-VP |
| HVTN 078 | NYVAC-B |
| HVTN 082 | VRC-HIVDNA016-00-VP |
| HVTN 084 | VRC-HIVADV054-00-VP |
| HVTN 086, SAAVI 103 | SAAVI MVA-C |
| HVTN 087 | HIV-MAG |
| HVTN 088 | Oligomeric gp140/MF59 |
| HVTN 090 | VSV-Indiana HIV gag vaccine |
| HVTN 092 | DNA-HIV-PT123 |
| HVTN 094 | GEO-D03 |
| HVTN 096 | DNA-HIV-PT123 |
| HVTN 097 | ALVAC-HIV vCP1521 |
| HVTN 100 | ALVAC-HIV-C (vCP2438) |
| HVTN 101 | DNA-HIV-PT123 |
| HVTN 104 | VRC-HIVMAB060-00-AB |
| HVTN 105 | AIDSVAX B/E |
| HVTN 106 | DNA Nat-B env |
| HVTN 110 | Ad4-mgag |
| HVTN 112 | HIV-1 nef/tat/vif, env pDNA |
| | vaccine |
| HVTN 116 | VRC-HIVMAB060-00-AB |
| HVTN 205 | pGA2/JS7 DNA |
| HVTN 702 | ALVAC-HIV-C (vCP2438) |
| HVTN 703 AMP | VRC-HIVMAB060-00-AB |
| HVTN 908 | pGA2/JS7 DNA |
| IAVI 001 | DNA.HIVA |
| IAVI 016 | MVA.HIVA |
| IAVI A001 | tgAAC09 |
| IAVI A003 | AAV1-PG9 |
| IAVI B001 | Ad35-GRIN/ENV |
| IAVI B002 | Adjuvanted GSK |
| | investigational HIV vaccine |
| | formulation 1 |
| IAVI B003 | Ad26.EnvA-01 |
| IAVI B004 | HIV-MAG |
| IAVI C001 | ADVAX |
| IAVI C002 | ADMVA |
| IAVI D001 | TBC-M4 |
| IAVI N004 HIV-CORE 004 | Ad35-GRIN |
| IAVI R001 | rcAd26.MOS1.HIVEnv |
| IAVI S001 | SeV-G |
| IDEA EV06 | DNA-HIV-PT123 |
| IHV01 | Full-Length Single Chain |
| | (FLSC) |
| IMPAACT P1112 | VRC-HIVMAB060-00-AB |
| IPCAVD006 | MVA mosaic |
| IPCAVD008 | Trimeric gp140 |
| IPCAVD009 | Ad26.Mos.HIV Trivalent |
| ISS P-001 | Tat vaccine |
| LFn-p24 vaccine | LFn-p24 |
| MCA-0835 | 3BNC117 |
| Mucovac2 | CN54gp140 |
| MV1-F4 | Measles Vector-GSK |
| MYM-V101 | Virosome-Gp41 |
| NCHECR-AE1 | pHIS-HIV-AE |
| PEACHI-04 | ChAdV63.HIVconsv |
| PedVacc001 & PedVacc002 | MVA.HIVA |
| PolyEnv1 | PolyEnv1 |
| PXVX-HIV-100-001 | Ad4-mgag |
| RISVAC02 | MVA-B |
| RV 151/WRAIR 984 | LFn-p24 |
| RV 158 | MVA-CMDR |
| SG06RS02 | HIV gp140 ZM96 |
| TAB9 | TAB9 |
| TaMoVac II | HIVIS-DNA |
| UBI V106 | UBI HIV-1 Peptide Vaccine, |
| | Microparticulate Monovalent |
| UCLA MIG-001 | TBC-3B |
| UKHVCSpoke003 | DNA-CN54ENV and |
| | ZM96GPN |
| V3-MAPS | V3-MAPS |
| VAX 002 | AIDSVAX B/B |
| VAX 003 | AIDSVAX B/E |
| VRC 602 | VRC-HIVMAB060-00-AB |
| VRC 607 | VRCHIVMAB080-00-AB |

*IAVI is the International AIDS Vaccine Initiative, whose clinical trials database is publicly available at http://www.iavi.org/trials-database/trials.
**As used herein, the term “Prime” refers to the composition initially used as an immunological inoculant in a given clinical trial as referenced in Table 1 herein.

As used herein, the term “in vivo” refers to processes that occur in a living organism. The term “ex vivo” refers to processes that occur outside of a living organism. For example, in vivo treatment refers to treatment that occurs within a patient's body, while ex vivo treatment is one that occurs outside of a patient's body, but still uses or accesses or interacts with tissues from that patient. Thereafter, an ex vivo treatment step may include a subsequent in vivo treatment step.

As used herein, the term “miRNA” refers to a microRNA, and also may be referred to herein as “miR”. The term “microRNA cluster” refers to at least two microRNAs that are situated on a vector in close proximity to each other and are co-expressed.

As used herein, the term “packaging cell line” refers to any cell line that can be used to express a lentiviral particle.

As used herein, the term “PBMC” refers to peripheral blood mononuclear cells.

As used herein, the term “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of ordinary skill in the art) or by visual inspection. Depending on the application, the “percent identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences of the present disclosure can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules of the disclosure. BLAST protein searches can be performed with the XBLAST program, score=50, wordlength=3 to obtain amino acid sequences homologous to the protein molecules of the disclosure. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

As used herein, the term “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, the term “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (see, e.g., Berge et al. (1977) J Pharm Sci 66:1-19).

As used herein, the term “physical method of selection” refers to any physical method that can be used to positively or negatively select for a cell type within a larger mixture of cells (e.g., PBMC). A non-limiting example of a physical method of selection is magnetic bead sorting.

As used herein, the term “SEQ ID NO” is synonymous with the term “Sequence ID No.”

As used herein, the term “small RNA” refers to non-coding RNA that are generally less than about 200 nucleotides or less in length and possess a silencing or interference function. In other embodiments, the small RNA is about 175 nucleotides or less, about 150 nucleotides or less, about 125 nucleotides or less, about 100 nucleotides or less, or about 75 nucleotides or less in length. Such RNAs include microRNA (miRNA), small interfering RNA (siRNA), double stranded RNA (dsRNA), and short hairpin RNA (shRNA). “Small RNA” of the disclosure should be capable of inhibiting or knocking-down gene expression of a target gene, for example through pathways that result in the destruction of the target gene mRNA.

As used herein, the term “static culture” refers to a cell culture environment which is not physically agitated, rocked, or otherwise subjected to intentional movement for the duration of the cell culture interval.

As used herein, the term “semi-static culture” refers to a cell culture environment which is subjected to minimal intentional movement, rocking, or physical agitation for the duration of the cell culture interval.

As used herein, the term “stimulatory agent” refers to any exogenous agent that can stimulate an immune response, and includes, without limitation, a vaccine, a HIV vaccine, and HIV or HIV-related peptides. A stimulatory agent can preferably stimulate a T cell response.

As used herein, the term “subject” includes a human patient but also includes other mammals. The terms “subject,” “individual,” “host,” and “patient” may be used interchangeably herein.

As used herein, the term “T regulatory cells” refers to a subpopulation of immunosuppressive T cells that derive from progenitor cells in the bone marrow and mature in the thymus. T regulatory cells can be identified by the expression of the markers CD4, CD25, and/or FOXP3.

As used herein, the phrase “target sequence” includes any nucleotide sequence capable of being targeted by a regulatory factor. An example of a target sequence includes, but is not limited to, mRNA. Examples of regulatory factors include any regulatory RNA such as, but not limited to, microRNA and shRNA.

As used herein, the term “Tat” refers to the HIV tat gene and its gene product, and variants thereof.

As used herein, the term “therapeutically effective amount” refers to a sufficient quantity of the active agents of the present disclosure, in a suitable composition, and in a suitable dosage form to treat or prevent the symptoms, progression, or onset of the complications seen in patients suffering from a given ailment, injury, disease, or condition. The therapeutically effective amount will vary depending on the state of the patient's condition or its severity, and the age, weight, etc., of the subject to be treated. A therapeutically effective amount can vary, depending on any of a number of factors, including, e.g., the route of administration, the condition of the subject, as well as other factors understood by those in the art.

As used herein, the term “therapeutic vector” is synonymous with a lentiviral vector such as the AGT103 vector.

As used herein, the terms “treatment” or “treating” generally refers to an intervention in an attempt to alter the natural course of the subject being treated, and can be performed either for prophylaxis or during the course of clinical pathology. Desirable effects include, but are not limited to, preventing occurrence or recurrence of disease, alleviating symptoms, suppressing, diminishing or inhibiting any direct or indirect pathological consequences of the disease, ameliorating or palliating the disease state, and causing remission or improved prognosis.

As used herein, the term “vaccine”, which is used interchangeably with the term “therapeutic vaccine” refers to an exogenous agent that can elicit an immune response in an individual and includes, without limitation, purified proteins, inactivated viruses, virally vectored proteins, bacterially vectored proteins, peptides or peptide fragments, or virus-like particles (VLPs).

As used herein, the term “variant” refers to a nucleotide sequence that, when compared to a reference sequence, contains at least one of a single nucleotide polymorphism, a single nucleotide variation, a conversion, an inversion, a duplication, a deletion, or a substitution. A “variant” includes amino acid sequences that derive from “variant” nucleotide sequences, as well as post-transcriptional and post translational modifications related thereto.

The term “Vif” refers to the HIV vif gene and its gene product, and variants thereof.

### Description of Aspects of the Disclosure

As detailed herein, in one aspect, a method of treating cells infected with HIV is provided. The method generally includes contacting peripheral blood mononuclear cells (PBMC) isolated from a subject infected with HIV with a therapeutically effective amount of a stimulatory agent, wherein the contacting step is carried out ex vivo; depleting non-target cell populations; transducing the PBMC ex vivo with a viral delivery system encoding at least one genetic element; and culturing the transduced PBMC for a period of time sufficient to achieve such transduction. In embodiments, the transduced PBMC are cultured from about 1 to about 35 days. In embodiments, the method further includes infusing the transduced PBMC into a subject. In embodiments, the subject is a human. In embodiments, the stimulatory agent is a peptide or mixture of peptides, and in embodiments, includes a gag peptide. In further embodiments, the stimulatory agent includes a vaccine. In embodiments, the vaccine is a HIV vaccine, and in further embodiments, the HIV vaccine is a MVA/HIV62B vaccine, or a variant or analog thereof. In embodiments, the viral delivery system includes a lentiviral particle. In embodiments, the at least one genetic element includes a small RNA capable of inhibiting production of chemokine receptor CCR5. In embodiments, the at least one genetic element includes at least one small RNA capable of targeting an HIV RNA sequence. In other embodiments, the at least one genetic element includes a small RNA capable of inhibiting production of chemokine receptor CCR5 and at least one small RNA capable of targeting an HIV RNA sequence. In embodiments, the HIV RNA sequence includes a HIV Vif sequence, a HIV Tat sequence, variants thereof, or RNA sequences from other HIV genes that prevent HIV infection or reduce virus expression in already-infected cells. In embodiments, the at least one genetic element includes at least one of a microRNA or a shRNA. In further embodiments, the at least one genetic element comprises a microRNA cluster.

In embodiments, the at least one genetic element includes a microRNA having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more percent identity with SEQ ID NO: 1. In embodiments, the microRNA has less than 80% sequence identity with SEQ ID NO: 1. In embodiments, the microRNA has greater than 95% sequence identity with SEQ ID NO: 1. In embodiments, the at least one genetic element includes SEQ ID NO: 1.

In embodiments, the at least one genetic element includes a microRNA having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more percent identity with SEQ ID NO: 2. In embodiments, the microRNA has less than 80% sequence identity with SEQ ID NO: 2. In embodiments, the microRNA has greater than 95% sequence identity with SEQ ID NO: 2 In embodiments, the at least one genetic element includes SEQ ID NO: 2.

In embodiments, the at least one genetic element includes a microRNA having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more percent identity with SEQ ID NO: 3. In embodiments, the microRNA has less than 80% sequence identity with SEQ ID NO: 3. In embodiments, the microRNA has greater than 95% sequence identity with SEQ ID NO: 3. In embodiments, the at least one genetic element includes SEQ ID NO: 3.

In embodiments, the microRNA cluster includes a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more percent identity with SEQ ID NO: 30. In embodiments, the microRNA cluster has less than 80% sequence identity with SEQ ID NO: 30. In embodiments, the microRNA cluster has greater than 95% sequence identity with SEQ ID NO: 30. In embodiments, the microRNA cluster includes SEQ ID NO: 30.

In another aspect, a method is disclosed which includes obtaining peripheral blood from HIV+ individuals; fractionating the blood to obtain a PBMC population; contacting the PBMC population with purified antigen-presenting cells or peptides or proteins representing components of HIV; culturing the contacted PBMC population for about 1 to about 12 days to expand an antigen-specific population; depleting one or more non-target cell populations to produce a fraction enriched with cells that respond to peptide stimulation; transducing the enriched cell fraction ex vivo with a viral delivery system as detailed herein; culturing the transduced cell fraction for a period of time sufficient to ensure adequate cell proliferation; and harvesting the transduced cells.

In embodiments, the PBMC population is further purified to produce a purified fraction of PBMC. In embodiments, further purified fractions of PBMC are contacted with peptides or proteins representing components of HIV.

In another aspect, a method is disclosed that includes contacting lymphocytes with at least one HIV peptide and transducing the lymphocytes with a viral delivery system encoding at least one genetic element.

In embodiments, the lymphocytes that are contacted comprise any one or more of T cells, B cells, and NK cells. In embodiments, the lymphocytes that are contacted comprise only T cells. In embodiments, the T cells comprise any one or more of CD4+ T cells, CD8+ T cells, and γδ cells. In embodiments, the T cells comprise only CD4+ T cells.

In embodiments, the lymphocytes that are contacted are derived from PBMC. In embodiments, the method further includes depleting at least one subset of cells in the PBMC. In embodiments, the at least one subset of cells that are depleted includes any one or more of B cells, NK cells, CD8+ T cells, and γδ cells.

In embodiments, the lymphocytes that are contacted are derived from a population of leukocytes. In embodiments, the method further comprises depleting at least one subset of cells in the leukocytes. In embodiments, the at least one subset of cells that are depleted includes any one or more of neutrophils, eosinophils, basophils, monocytes, and certain lymphocytes including any one or more of NK cells, B cells, CD8+ T cells, and γδ cells.

In embodiments, the lymphocytes that are contacted are derived from a population of cells. In embodiments, the method further comprises depleting at least one subset of cells from the population of cells. In embodiments, the at least one subset of cells that are depleted includes any one or more of CD8+ T cells, γδ cells, NK cells, B cells, neutrophils, basophils, mast cells, monocytes, dendritic cells, T regulatory cells, NKT cells, and erythrocytes. In embodiments, the at least one subset of cells that are depleted includes any one or more of any cell type that is not a CD4+ T cell.

In embodiments, the viral delivery system includes a lentiviral particle. In embodiments, the lentiviral particle is any lentiviral particle described herein.

In another aspect, a method of manufacturing a cell product for treating HIV infection in a subject is disclosed. The method generally includes obtaining blood leukocytes; removing leukocytes from the subject and purifying peripheral blood mononuclear cells (PBMC) or defined fractions of PBMC. In embodiments, the method further includes contacting the PBMC or purified fraction of PBMC ex vivo with a therapeutically effective amount of a stimulatory agent; depleting one or more non-target cell populations in order to increase the proportion of antigen-specific T cells; transducing the PBMC or purified fraction of PBMC ex vivo with a viral delivery system encoding at least one genetic element; culturing the transduced PBMC or a purified fraction of PBMC for a period of time sufficient to achieve transduction and growth of the modified cell population; and harvesting the transduced PBMC or a purified fraction of PBMC. In embodiments, the transduced PBMC or purified fraction of PBMC are cultured from about 1 to about 35 days. In embodiments, the method further involves infusing the transduced PBMC or purified fraction of PBMC into a subject. In embodiments, the subject is a human. In embodiments, the at least one of the first stimulatory agents includes a peptide or mixture of peptides, represents one, two, three or more of proteins encoded by the HIV genome, and includes multiple examples of individual peptides to represent sequence variation among known HIV isolates from the country or region where treatment is intended or to accommodate unusual variation among histocompatibility genes for specific patient populations. In embodiments, at least one of the first stimulatory agents includes a gag peptide. In embodiments, the at least one of the first stimulatory agents includes a vaccine. In embodiments, the vaccine is a HIV vaccine, and in further embodiments, the HIV vaccine is a MVA/HIV62B vaccine, or a variant or analog thereof. In embodiments, the first stimulatory agent is a mixture of gag peptides.

In embodiments, the viral delivery system includes a lentiviral particle. In embodiments, the at least one genetic element includes a small RNA capable of inhibiting production of chemokine receptor CCR5. In embodiments, the at least one genetic element includes at least one small RNA capable of targeting an HIV RNA sequence. In embodiments, the at least one genetic element includes a small RNA capable of inhibiting production of chemokine receptor CCR5 and at least one small RNA capable of targeting an HIV RNA sequence. In embodiments, the HIV RNA sequence includes a HIV Vif sequence, a HIV Tat sequence, or variants thereof. In embodiments, the at least one genetic element includes a microRNA or a shRNA, or a cluster thereof. In embodiments, the at least one genetic element comprises a synthetic microRNA cluster.

In embodiments, the at least one genetic element includes a microRNA having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more percent identity with SEQ ID NO: 1. In embodiments, the microRNA has less than 80% sequence identity with SEQ ID NO: 1. In embodiments, the microRNA has greater than 95% sequence identity with SEQ ID NO: 1. In embodiments, the at least one genetic element includes SEQ ID NO: 1.

In embodiments, the at least one genetic element includes a microRNA having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more percent identity with SEQ ID NO: 2. In embodiments, the microRNA has less than 80% sequence identity with SEQ ID NO: 2. In embodiments, the microRNA has greater than 95% sequence identity with SEQ ID NO: 2 In embodiments, the at least one genetic element includes SEQ ID NO: 2.

In embodiments, the at least one genetic element includes a microRNA having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more percent identity with SEQ ID NO: 3. In embodiments, the microRNA has less than 80% sequence identity with SEQ ID NO: 3. In embodiments, the microRNA has greater than 95% sequence identity with SEQ ID NO: 3. In embodiments, the at least one genetic element includes SEQ ID NO: 3.

In embodiments, the microRNA cluster includes a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more percent identity with SEQ ID NO: 30. In embodiments, the microRNA cluster has less than 80% sequence identity with SEQ ID NO: 30. In embodiments, the microRNA cluster has greater than 95% sequence identity with SEQ ID NO: 30. In embodiments, the microRNA cluster includes SEQ ID NO: 30.

In another aspect, a method of manufacturing a cell product for treating HIV in a subject is disclosed. In embodiments, the method includes contacting PBMC or a purified fraction of PBMC with at least one antigen and depleting one or more non-target cell populations in the PBMC. In embodiments, the one or more non-target cell populations includes any one or more of B cells, NK cells, CD8+ T cells, and γδ cells. In embodiments, the at least one antigen is a peptide or mixture of peptides encoded by the HIV genome. In embodiments, the peptide is a gag peptide or the mixture of peptides comprises a gag peptide.

In another aspect, a method of manufacturing a cell product for treating HIV in a subject is disclosed. In embodiments, the method includes contacting leukocytes or a purified fraction of leukocytes with at least one antigen and depleting one or more non-target cell populations of leukocytes. In embodiments, the one or more non-target cell populations includes any one or more of neutrophils, basophils, eosinophils, and monocytes. In embodiments, the one or more non-target cell populations further includes specific lymphocytes including any one or more of B cells, NK cells and CD8+ T cells. In embodiments, the at least one antigen is a peptide or a mixture of peptides encoded by the HIV genome. In embodiments, the peptide is a gag peptide or the mixture of peptides comprises a gag peptide.

In another aspect, a lentiviral vector is disclosed. The lentiviral vector includes at least one encoded genetic element, wherein the at least one encoded genetic element comprises a small RNA capable of inhibiting production of chemokine receptor CCR5 or at least one small RNA capable of targeting an HIV RNA sequence.

In another aspect a lentiviral vector is disclosed in the at least one encoded genetic element comprises a small RNA capable of inhibiting production of chemokine receptor CCR5 and at least one small RNA capable of targeting an HIV RNA sequence. The HIV RNA sequence may include a HIV Vif sequence, a HIV Tat sequence, a sequence from another portion of the HIV genome, or a variant thereof. The at least one encoded genetic element may include a microRNA or a shRNA. The at least one encoded genetic element may include a microRNA cluster.

In embodiments, the at least one genetic element includes a microRNA having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more percent identity with SEQ ID NO: 1. In embodiments, the microRNA has less than 80% sequence identity with SEQ ID NO: 1. In embodiments, the microRNA has greater than 95% sequence identity with SEQ ID NO: 1. In embodiments, the at least one genetic element includes SEQ ID NO: 1.

In embodiments, the at least one genetic element includes a microRNA having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more percent identity with SEQ ID NO: 2. In embodiments, the microRNA has less than 80% sequence identity with SEQ ID NO: 2. In embodiments, the microRNA has greater than 95% sequence identity with SEQ ID NO: 2. In embodiments, the at least one genetic element includes SEQ ID NO: 2.

In embodiments, the at least one genetic element includes a microRNA having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more percent identity with SEQ ID NO: 3. In embodiments, the microRNA has less than 80% sequence identity with SEQ ID NO: 3. In embodiments, the microRNA has greater than 95% sequence identity with SEQ ID NO: 3. In embodiments, the at least one genetic element includes SEQ ID NO: 3.

In embodiments, the microRNA cluster includes a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more percent identity with SEQ ID NO: 30. In embodiments, the microRNA cluster has less than 80% sequence identity with SEQ ID NO: 30. In embodiments, the microRNA cluster has greater than 95% sequence identity with SEQ ID NO: 30. In embodiments, the microRNA cluster includes SEQ ID NO: 30.

In another aspect, a lentiviral vector system for expressing a lentiviral particle is provided. The system includes a lentiviral vector as described herein; at least one envelope plasmid for expressing an envelope protein preferably optimized for infecting a cell; and at least one helper plasmid for expressing a gene of interest, for example any of gag, pol, and rev genes, wherein when the lentiviral vector, the at least one envelope plasmid, and the at least one helper plasmid are transfected into a packaging cell, wherein a lentiviral particle is produced by the packaging cell, wherein the lentiviral particle is capable of modulating a target sequence of interest, for example inhibiting production of chemokine receptor CCR5 or targeting an HIV RNA sequence.

In embodiments, the envelope protein is optimized for infecting an endocytic compartment. In embodiments, the envelope protein is any of the envelope proteins described herein.

In another aspect, a lentiviral particle capable of infecting a cell is disclosed. The lentiviral particle includes at least one envelope protein preferably optimized for infecting a cell, and a lentiviral vector as described herein. The envelope protein may be optimized for infecting a T cell. In embodiments, the envelope protein is optimized for infecting a CD4+ T cell.

In embodiments, the particle is a pseudotyped particle. In embodiments, the pseudotyped particle is any pseudotyped particle described herein. In embodiments, the pseudotyped particle comprises an envelope protein from a virus that is not a lentivirus. In embodiments, the virus from which envelope protein derives is any virus described herein.

In another aspect, a modified cell is disclosed. In embodiments, the modified cell is a CD4+ T cell. In embodiments, the CD4+ T cell is infected with a lentiviral particle as described herein. In embodiments, the CD4+ T cell also has been selected to recognize an HIV antigen based on the prior immunization with a stimulatory agent. In a further embodiment, the HIV antigen that is recognized by the CD4+ T cell includes a gag antigen. In a further embodiments, the CD4+ T cell expresses a decreased level of CCR5 following infection with the lentiviral particle.

In another aspect, a modified cell is disclosed. In embodiments, the modified cell is a CD4+ T cell that has been infected with a lentiviral particle and an exogenous antigen. In embodiments, the lentiviral particle is any lentiviral particle described herein. In embodiments, the exogenous antigen is an antigen derived from HIV. In embodiments, the antigen derived from HIV is any HIV antigen described herein.

In another aspect, a method of selecting a subject for a therapeutic treatment regimen is disclosed. The method generally includes immunizing the subject with an effective amount of a first stimulatory agent; removing leukocytes from the subject and purifying peripheral blood mononuclear cells (PBMC) and determining a first quantifiable measurement associated with at least one factor associated with the PBMC; contacting the PBMC ex vivo with a therapeutically effective amount of a second stimulatory agent, and determining a second measurement associated with the at least one factor associated with the PBMC, whereby when the second quantifiable measurement is different (e.g., higher) than the first quantifiable measurement, the subject is selected for the treatment regimen. The at least one factor may be T cell proliferation or IFN gamma production.

In another aspect, a method is disclosed. The method includes purifying peripheral blood mononuclear cells (PBMC) from a source, stimulating the PBMC with at least one HIV-specific peptide, depleting at least one subset of cells from the PBMC, wherein the at least one subset of cells comprises any one or more of CD8+ T cells, CD4+ T cells, γδ cells, NK cells, B cells, T regulatory cells, and NKT cells; transducing the depleted PBMC with a viral delivery system encoding at least one genetic element, culturing the transduced PBMC for at least one day, and harvesting the cultured PBMC.

In embodiments, the purifying the PBMC comprises removing any one or more of residual red blood cells, residual platelets, and residual inflammatory cells. In embodiments, the purifying comprises separating any one or more of residual red blood cells, residual platelets, and residual inflammatory cells on the basis of cell density. In embodiments, the separating on the basis of cell density comprises differential centrifugation. However, any suitable method of purifying and/or separating the PBMC is contemplated.

In embodiments, the purifying comprises separating any one or more of residual red blood cells, residual platelets, and residual inflammatory cells using magnetic bead separation. In embodiments, the purifying comprises separating other contaminants from the PBMC using magnetic bead separation. In embodiments, the magnetic bead separation comprises using antibodies to aid in positive selection of leukocytes. In embodiments, the magnetic bead separation comprises using antibodies to aid in negative selection of residual red blood cells, residual platelets residual inflammatory cells, and/or other contaminants. In embodiments, the antibodies are monoclonal antibodies. In embodiments, the purifying the PBMC comprises using an automated system.

In embodiments, the transduced PBMC are cultured for more than one day, for example, more than 24 hours, more than 30 hours, more than 36 hours, more than 42 hours, more than 48 hours, more than 48 hours, more than 54 hours, more than 60 hours, more than 66 hours, more than 72 hours, more than 78 hours, more than 84 hours, more than 90 hours, or more than 96 hours.

In embodiments, the culturing occurs in a static culture system. In embodiments, the culturing occurs in a semi-static culture system. In embodiments, the cultured cells adhere to the culture plate or flask. In embodiments, the cultured cells are in a suspension. In embodiments, some of the cultured cells adhere to the culture plate or flask and some cells are in a suspension.

In embodiments, the at least one HIV-specific peptide comprises a pool of synthetic peptides. In embodiments, synthetic peptides of the pool of synthetic peptides overlap. In embodiments, synthetic peptides of the pool of synthetic peptides do not overlap. In embodiments, the pool of synthetic peptides comprises approximately 150 individual peptides. In embodiments, the pool of peptides may include sequence variants of individual peptides that are included to match the prevailing HIV sequences found in a particular region or sub-population, or to better match the requirements for peptide binding and T cell receptor recognition dictated by particular haplotypes in the major histocompatibility gene clusters. In embodiments, the pool of synthetic peptides comprises less than 150 individual peptides, for example, less than 140 individual peptides, less than 130 individual peptides, less than 120 individual peptides, less than 110 individual peptides, less than 100 individual peptides, less than 90 individual peptides, less than 80 individual peptides, less than 70 individual peptides, less than 60 individual peptides, or less than 50 individual peptides. In embodiments, the pool of synthetic peptides comprises more than 150 individual peptides, for example, more than 160 individual peptides, more than 170 individual peptides, more than 180 individual peptides, more than 190 individual peptides, more than 200 individual peptides, more than 210 individual peptides, more than 220 individual peptides, more than 230 individual peptides, more than 240 individual peptides, or more than 250 individual peptides.

In embodiments, the pool of synthetic peptides represents the HIV Gag polyprotein. However, the pool of synthetic peptides may represent any HIV-specific peptide.

In embodiments, stimulating the PBMC with at least one HIV-specific peptide comprises infecting PBMC with recombinant vaccinia. In embodiments, the vaccinia expresses Gag proteins. In embodiments, the vaccinia comprises any one or more of Gag proteins, Pol proteins, and Env proteins. In embodiments, the vaccinia targets white blood cells. In embodiments, the vaccinia targets any one or more of neutrophils, eosinophils, basophils, lymphocytes and monocytes. In embodiments, the vaccinia targets macrophages. In embodiments, the vaccinia targets any antigen presenting cell. In embodiments, the antigen presenting cell is a B cell. In embodiments, the antigen presenting cell is a dendritic cell.

In embodiments, stimulating the PBMC with at least one HIV-specific peptide comprises adding a purified Gag protein directly to PBMC culture. In embodiments, the purified Gag protein comprises a vaccine. In embodiments, the purified Gag protein comprises Gag p24 protein vaccine. In embodiments, stimulating the PBMC with at least one HIV-specific peptide comprises adding a purified Env protein directly to PBMC culture. In embodiments, stimulating the PBMC with at least one HIV-specific peptide comprises adding a purified Pol protein directly to PBMC culture.

In embodiments, stimulating the PBMC with at least one HIV-specific peptide comprises adding customized peptides that comprise various sequence variations of known HIV sequences. In embodiments the customized peptides comprise an array of peptides.

In embodiments, the depleting comprises separating the at least one subset of cells from the depleted PBMC. In embodiments, the at least one subset of cells comprises CD8+ T cells. In embodiments, the at least one subset of cells comprises CD8+ T cells, CD56+ NK cells, B cells, and γδ cells. In embodiments, the at least one subset of cells comprises CD8+ T cells and γδ cells. In embodiments, the at least one subset of cells comprises CD8+ T cells, γδ cells, and B cells. However, the at least one subset of cells may comprise any subset of cells present in the PBMC including, without limitation, any one or more of CD8+ T cells, CD4+ T cells, γδ cells, NK cells, B cells, T regulatory cells, and NKT cells.

In embodiments, the separating comprises magnetic bead separation. In embodiments, depleting comprises a method of using iron-labeled antibodies against cell surface markers to identify any unwanted subset of cells. In embodiments, the method further comprises removing the unwanted subset of cells by retaining them on a magnetic column.

In embodiments, the depleting comprises positive selection for Gag-responsive cells that are secreting interferon-gamma. In embodiments, the Gag-responsive cells are macrophages, B lymphocytes, and/or T lymphocytes. In embodiments, the Gag-responsive cells secrete other types of interferons such as interferon-alpha, interferon-beta, or interferon-kappa.

In embodiments, the depleting comprises utilizing unlabeled antibodies that identify unwanted cell types. In embodiments, the method further comprises retaining antibody-bound cells on a column. In embodiments, the column is coated with Protein A. In embodiments, the column is coated with Protein G. In embodiments, the column is coated with anti-immunoglobulin or another immunoglobulin binding compound that removes effectively the cells bound to the targeting antibodies.

In embodiments, the depleting comprises purifying target cells based on density. In embodiments, the depleting comprises using density gradient centrifugation.

In embodiments, the depleting comprises labeling unwanted leukocytes with primary antibodies and killing the cell with secondary antibodies that bind to constant regions of the monoclonal antibodies. In embodiments, the secondary antibody is chemically linked to a toxin. In embodiments, the toxin is Diphtheria toxin. In embodiments, the antibodies are monoclonal. In embodiments, the antibodies are polyclonal.

In embodiments, the at least one genetic element comprises a small RNA capable of inhibiting production of chemokine receptor CCR5 or at least one small RNA capable of targeting an HIV RNA sequence. In embodiments, the HIV RNA sequence comprises a HIV Vif sequence, a HIV Tat sequence, or a variant thereof.

In embodiments, the at least one genetic element comprises a non-coding RNA. In embodiments, the non-coding RNA is a microRNA. In embodiments, the non-coding RNA is a siRNA. In embodiments, the non-coding RNA is a piRNA. In embodiments, the non-coding RNA is a snoRNA. In embodiments, the non-coding RNA is a snRNA. In embodiments, the non-coding RNA is a exRNA. In embodiments, the at least one genetic element comprises a microRNA or a shRNA.

In embodiments, the at least one genetic element comprises a microRNA having at least 80%, or at least 85%, or at least 90%, or at least 95% identity with at least one of SEQ ID NO: 1, SEQ ID NO: 2, SEQ ID NO: 3, or SEQ ID NO: 30. In embodiments, the at least one genetic element comprises a microRNA having less than 80% identity with at least one of SEQ ID NO: 1, SEQ ID NO: 2, SEQ ID NO: 3, or SEQ ID NO: 30, for example, 75% identity, 70% identity, 65% identity, 60% identity, 55% identity, or 50% identity.

In another aspect, a genetically-modified lymphocyte for treatment of a subject infected with HIV is disclosed. The genetically-modified lymphocyte is prepared by a process comprising the steps of purifying peripheral blood mononuclear cells (PBMC) from the subject, stimulating the PBMC with at least one HIV-specific peptide, depleting at least one subset of cells from the PBMC, wherein the at least one subset of cells comprises any one or more of CD8+ T cells, CD4+ T cells, γδ cells, NK cells, T regulatory cells, and NKT cells, transducing the depleted PBMC with a viral delivery system encoding at least one genetic element, culturing the transduced PBMC for at least one day, and harvesting the cultured PBMC.

In embodiments, the subject infected with HIV is in the latent period of HIV infection. In embodiments, the subject infected with HIV is experiencing few or no clinical symptoms of the HIV infection. In embodiments, the subject infected with HIV is in the acute phase of HIV infection. In embodiments, the subject infected with HIV is in the chronic phase of the HIV infection. In embodiments, the subject infected with HIV has experienced viremia in which HIV has entered the bloodstream. In embodiments, the subject infected with HIV has a depleted CD4+ T cells as a result of the HIV infection. In embodiments, the subject infected with HIV has a depleted CD4+ T cells in any one or more of the peripheral blood, the gastrointestinal tract, the lymph nodes, and the lymphatic tissue. In embodiments, the CD4+ T cells are preferentially depleted in the gastrointestinal tract. In embodiments, the subject infected with HIV has depleted CCR5+ CD4+ T cells in any one or more the peripheral blood, the gastrointestinal tract, the lymph nodes, and the lymphatic tissue.

In another aspect, a genetically-modified lymphocyte for treatment of a subject immunized with a preventive or therapeutic HIV vaccine is disclosed. The genetically-modified lymphocyte is prepared by a process comprising the steps of purifying peripheral blood mononuclear cells (PBMC) from the subject, stimulating the PBMC with at least one HIV-specific peptide, depleting at least one subset of cells from the PBMC, wherein the at least one subset of cells comprises any one or more of CD8+ T cells, CD4+ T cells, γδ cells, NK cells, B cells, neutrophils, T regulatory cells, and NKT cells, transducing the depleted PBMC with a viral delivery system encoding at least one genetic element, culturing the transduced PBMC for at least one day, and harvesting the cultured PBMC.

In embodiments, the subject immunized with a preventive or therapeutic vaccine is immunized with a vaccine that comprises a whole organism. In embodiments, the subject immunized with a preventive or therapeutic vaccine is immunized with a vaccine that comprises a live-attenuated organism. In embodiments, the subject immunized with a preventive or therapeutic vaccine is immunized with a vaccine that comprises an antiretroviral. In embodiments, the subject immunized with a preventive or therapeutic vaccine is immunized with a vaccine that comprises an antibody. In embodiments, the subject immunized with a preventive or therapeutic vaccine is immunized with a vaccine that causes stimulation of T cell immunity. In embodiments, the subject immunized with a preventive or therapeutic vaccine is immunized with a vaccine that stimulates endogenous antibodies. In embodiments, the subject immunized with a preventive or therapeutic HIV vaccine is immunized with RV 144 prime and boost vaccines.

In another aspect, a genetically-modified lymphocyte for treatment of a subject exposed to, but not infected with HIV, or made immune to HIV through preventive vaccination, is disclosed. These individuals may have been exposed to infectious HIV one or multiple times particularly through contact with genital fluids and subsequently developed an immune response against HIV but are not-infected by diagnostic criteria including assay for plasma viral RNA or isolation of infectious virus from PBMC. These individuals may have received one or more HIV preventive vaccines in a single or multiple doses. Because they have detectable HIV immunity it is possible to manufacture the AGT103-T product and by infusing genetically-modified lymphocytes, increase the level of HIV immunity and make it sufficiently durable to withstand subsequent exposures to infectious HIV. In embodiments, the genetically-modified lymphocyte is prepared by a process comprising the steps of purifying peripheral blood mononuclear cells (PBMC) from the subject; stimulating the PBMC with at least one HIV-specific peptide, depleting at least one subset of cells from the PBMC, wherein the at least one subset of cells comprises any one or more of CD8+ T cells, CD4+ T cells, γδ cells, NK cells, B cells, T regulatory cells, and NKT cells, transducing the depleted PBMC with a viral delivery system encoding at least one genetic element, culturing the transduced PBMC for at least one day, and harvesting the cultured PBMC.

### Human Immunodeficiency Virus (HIV)

Human Immunodeficiency Virus, which is also commonly referred to as “HIV”, is a retrovirus that causes acquired immunodeficiency syndrome (AIDS) in humans. AIDS is a condition in which progressive failure of the immune system allows life-threatening opportunistic infections and cancers to thrive. Without treatment, average survival time after infection with HIV is estimated to be 9 to 11 years, depending upon the HIV subtype and genetics of the host population. Infection with HIV occurs by the transfer of bodily fluids, including but not limited to blood, semen, vaginal fluid, pre-ejaculate, saliva, tears, lymph or cerebro-spinal fluid, or breast milk, or use of contaminated blood or tissue products. HIV may be present in an infected individual as both free virus particles and within infected immune cells.

HIV infects vital cells in the human immune system such as helper T cells, although tropism can vary among HIV subtypes. Immune cells that may be specifically susceptible to HIV infection include but are not limited to CD4+ T cells, macrophages, and dendritic cells. HIV infection leads to low levels of CD4+ T cells through a number of mechanisms, including but not limited to apoptosis of uninfected bystander cells, direct viral killing of infected cells, and killing of infected CD4+ T cells by cytotoxic lymphocytes that recognize infected cells. When CD4+ T cell numbers decline below a critical level, cell-mediated immunity is lost, and the body becomes progressively more susceptible to opportunistic infections and cancer. Structurally, HIV is distinct from many other retroviruses. The RNA genome consists of at least seven structural landmarks (LTR, TAR, RRE, PE, SLIP, CRS, and INS), and at least nine genes (gag, pol, env, tat, rev, nef, vif, vpr, vpu, and sometimes a tenth tev, which is a fusion of tat, env and rev), encoding 19 proteins. Three of these genes, gag, pol, and env, contain information needed to make the structural proteins for new virus particles.

HIV replicates primarily in CD4 T cells and causes cellular destruction or dysregulation to reduce host immunity. Because HIV establishes infection as an integrated provirus and may enter a state of latency wherein virus expression in a particular cell decreases below the level required for cytopathology affecting that cell or detection by the host immune system, HIV is difficult to treat and has not been eradicated even after prolonged intervals of combination antiretroviral therapy (cART). In most cases, HIV infection causes fatal disease although survival may be prolonged by cART.

A major goal in the fight against HIV is to develop strategies for curing disease. Prolonged cART has not accomplished this goal, so investigators have turned to alternative procedures. Early efforts to improve host immunity by therapeutic immunization (using a vaccine after infection has occurred) had marginal or no impact. Likewise, treatment intensification had moderate or no impact.

Some progress has been made using genetic therapy, but positive results are sporadic and found only among rare human beings carrying defects in one or both alleles of the gene encoding CCR5, which plays a critical role in viral penetration of host cells. However, many investigators are optimistic that genetic therapy holds the best promise for eventually achieving an HIV cure.

The methods and compositions of the disclosure are able to achieve a cure that may or may not include complete eradication of all HIV from the body. As mentioned above, a cure is defined as a state or condition wherein HIV+ individuals who previously required cART, may survive with low or undetectable virus replication and using lower or intermittent doses of cART, or are potentially able to discontinue cART altogether. As used herein, a cure may still possibly require adjunct therapy to maintain low level virus replication and slow or eliminate disease progression. A possible outcome of a cure is the eventual eradication of HIV to prevent all possibility of recurrence.

The primary obstacles to achieving a cure lie in the basic biology of HIV itself. Virus infection deletes CD4 T cells that are critical for nearly all immune functions. Most importantly, HIV infection and depletion of CD4 T cells requires activation of individual cells. Activation is a specific mechanism for individual CD4 T cell clones that recognize pathogens or other molecules, using a rearranged T cell receptor.

In the case of HIV, infection activates a population of HIV-specific T cells that become infected and are consequently depleted before other T cells that are less specific for the virus, which effectively cripples the immune system's defense against the virus. The capacity for HIV-specific T cell responses is rebuilt during prolonged cART; however, when cART is interrupted the rebounding virus infection repeats the process and again deletes the virus-specific cells, resetting the clock on disease progression.

A cure is possible if enough HIV-specific CD4 T cells are protected to allow for a host's native immunity to confront and control HIV once cART is interrupted. In one embodiment, the present disclosure provides methods and compositions for improving the effectiveness of genetic therapy to provide a cure of HIV disease. In another embodiment, the present disclosure provides methods and compositions for enhancing host immunity against HIV to provide a cure. In yet another embodiment, the present disclosure provides methods and compositions for enriching HIV-specific CD4 T cells in a patient to achieve a cure.

In embodiments, treatment results in enriching a subject's HIV-specific CD4 T cells by about 100%, about 200%, about 300%, about 400%, about 500%, about 600%, about 700%, about 800%, about 900%, or about 1000%.

### Gene Therapy

Viral vectors are used to deliver genetic constructs to host cells for the purposes of disease therapy or prevention.

Genetic constructs can include, but are not limited to, functional genes or portions of genes to correct or complement existing defects, DNA sequences encoding regulatory proteins, DNA sequences encoding regulatory RNA molecules including antisense, short homology RNA, long non-coding RNA, small interfering RNA or others, and decoy sequences encoding either RNA or proteins designed to compete for critical cellular factors to alter a disease state. Gene therapy involves delivering these therapeutic genetic constructs to target cells to provide treatment or alleviation of a particular disease.

There are multiple ongoing efforts to utilize genetic therapy in the treatment of HIV disease, but thus far, the results have been poor. A small number of treatment successes were obtained in rare HIV patients carrying a spontaneous deletion of the CCR5 gene (an allele known as CCR5delta32).

Lentivirus-delivered nucleases or other mechanisms for gene deletion/modification may be used to lower the overall expression of CCR5 and/or help to lower HIV replication. At least one study has reported having success in treating the disease when lentivirus was administered in patients with a genetic background of CCR5delta32. However, this was only one example of success, and many other patients without the CCR5delta32 genotype have not been treated as successfully. Consequently, there is a substantial need to improve the performance of viral genetic therapy against HIV, both in terms of performance for the individual viral vector construct and for improved use of the vector through a strategy for achieving functional HIV cure.

For example, some existing therapies rely on zinc finger nucleases to delete a portion of CCR5 in an attempt to render cells resistant to HIV infection. However, even after optimal treatment, only 30% of T cells had been modified by the nuclease at all, and of those that were modified, only 10% of the total CD4 T cell population had been modified in a way that would prevent HIV infection. In contrast, the disclosed methods result in virtually every cell carrying a lentivirus transgene having a reduction in CCR5 expression below the level needed to allow HIV infection.

For the purposes of the disclosed methods, gene therapy can include, but is not limited to, affinity-enhanced T cell receptors, chimeric antigen receptors on CD4 T cells (or alternatively on CD8 T cells), modification of signal transduction pathways to avoid cell death cause by viral proteins, increased expression of HIV restriction elements including TREX, SAMHD1, MxA or MxB proteins, APOBEC complexes, TRIMS-alpha complexes, tetherin (BST2), and similar proteins identified as being capable of reducing HIV replication in mammalian cells.

### Immunotherapy

Historically, vaccines have been a go-to weapon against deadly infectious diseases, including smallpox, polio, measles, and yellow fever. Unfortunately, there is no currently approved vaccine for HIV. The HIV virus has unique ways of evading the immune system, and the human body seems incapable of mounting an effective immune response against it. As a result, scientists do not have a clear picture of what is needed to provide protection against HIV.

However, immunotherapy may provide a solution that was previously unaddressed by conventional vaccine approaches. Immunotherapy, also called biologic therapy, is a type of treatment designed to boost the body's natural defenses to fight infections or cancer. It uses materials either made by the body or in a laboratory to improve, target, or restore immune system function.

In embodiments, immunotherapeutic approaches may be used to enrich a population of HIV-specific CD4 T cells for the purpose of increasing the host's anti-HIV immunity. In embodiments, integrating or non-integrating lentivirus vectors may be used to transduce a host's immune cells for the purposes of increasing the host's anti-HIV immunity. In embodiments, a vaccine comprising HIV proteins including but not limited to a killed particle, a virus-like particle, HIV peptides or peptide fragments, a recombinant viral vector, a recombinant bacterial vector, a purified subunit or plasmid DNA combined with a suitable vehicle and/or biological or chemical adjuvants to increase a host's immune responses may be used to enrich the population of virus-specific T cells or antibodies, and these methods may be further enhanced through the use of HIV-targeted genetic therapy using lentivirus or other viral vector.

### Methods

In one aspect, the disclosure provides methods for using viral vectors to achieve a cure for HIV disease. The methods generally include immunotherapy to enrich the proportion of HIV-specific CD4 T cells, followed by lentivirus transduction to deliver inhibitors of HIV and CCR5 and CXCR4 as required.

In one embodiment, the methods include a first stimulation event to enrich a proportion of HIV-specific CD4 T cells. The first stimulation can include administration of one or more of any agent suitable for enriching a patient's HIV-specific CD4+ T cells including but not limited to a vaccine.

Therapeutic vaccines can include one or more HIV protein with protein sequences representing the predominant viral types of the geographic region where treatment is occurring. Therapeutic vaccines will include purified proteins, inactivated viruses, virally vectored proteins, bacterially vectored proteins, peptides or peptide fragments, virus-like particles (VLPs), biological or chemical adjuvants including cytokines and/or chemokines, vehicles, and methods for immunization. Vaccinations may be administered according to standard methods known in the art and HIV patients may continue antiretroviral therapy during the interval of immunization and subsequent ex vivo lymphocyte culture including lentivirus transduction.

In some embodiments, HIV+ patients are immunized with an HIV vaccine, increasing the frequency of HIV-specific CD4 T cells by about 2, about 25, about 250, about 500, about 750, about 1000, about 1250, or about 1500-fold (or any amount in between these values). The vaccine may be any clinically utilized or experimental HIV vaccine, including the disclosed lentiviral, other viral vectors or other bacterial vectors used as vaccine delivery systems. In another embodiment, the vectors encode virus-like particles (VLPs) to induce higher titers of neutralizing antibodies. In another embodiment, the vectors encode peptides or peptide fragments associated with HIV including but not limited to gag, pol, and env, tat, rev, nef, vif, vpr, vpu, and tev, as well as LTR, TAR, RRE, PE, SLIP, CRS, and INS. Alternatively, the HIV vaccine used in the disclosed methods may comprise purified proteins, inactivated viruses, virally vectored proteins including HIV-specific antibodies or antibody-like molecules or CD4-like molecules, bacterially vectored proteins, peptides or peptide fragments, virus-like particles (VLPs), or biological or chemical adjuvants including cytokines and/or chemokines.

In one embodiment, the methods include ex vivo re-stimulation of CD4 T cells from persons or patients previously immunized by therapeutic vaccination, using purified proteins, inactivated viruses, virally vectored proteins, bacterially vectored proteins, biological or chemical adjuvants including cytokines and/or chemokines, vehicles, and methods for re-stimulation. Ex vivo re-stimulation may be performed using the same vaccine or immune stimulating compound used for in vivo immunization, or it may be performed using a different vaccine or immune stimulating compound than those used for in vivo immunization. Moreover, in some embodiments, the patient does not require prior therapeutic vaccination or re-stimulation of CD4 T cells if the individual has sufficiently high antigen-specific CD4 T cell responses to HIV proteins. In these embodiments, such a patient may only require ex vivo stimulation of CD4 T cells with viral antigens, vaccines or peptides followed by selection for HIV-specific T cells based on the response to stimulation. Enriched cell preparations may include 1%, 5%, 10%, 20%, 30%, 40%, 50% or more of the HIV-specific CD4+ T cells and are used for lentivirus transduction of genes able to protection from HIV-mediated depletion. Stimulation with polyclonal mitogen plus cytokines increases the number of enriched and transduced T cells until appropriate levels are reached for infusion back into the original patient.

In embodiments, peripheral blood mononuclear cells (PBMCs) are obtained by leukapheresis and treated ex vivo to obtain about 1×10⁹ CD4 T cells of which about 0.1%, about 1%, about 5% or about 10% or about 30% or about 40% or about 50% are both HIV-specific in terms of antigen responses, and HIV-resistant by virtue of carrying the therapeutic transgene delivered by the disclosed lentivirus vector. Alternatively, about 1×10⁷, about 1×10⁸, about 1×10⁹, about 1×10¹⁰, about 1×10¹¹, or about 1×10 12 CD4 T cells may be isolated for re-stimulation. Any suitable amount of CD4 T cells are isolated for ex vivo re-stimulation.

The isolated CD4 T cells can be cultured in appropriate medium throughout re-stimulation with HIV vaccine antigens, which may include antigens present in the prior therapeutic vaccination. Antiretroviral therapeutic drugs including inhibitors of reverse transcriptase, protease or integrase may be added to prevent virus re-emergence during prolonged ex vivo culture. CD4 T cell re-stimulation may be used to enrich the proportion of HIV-specific CD4 T cells in culture. The same procedure may also be used for analytical objectives wherein smaller blood volumes with peripheral blood mononuclear cells obtained by purification, are used to identify HIV-specific T cells and measure the frequency of this sub-population. The proportion of HIV-specific CD4 T cells in culture may be enriched or increased through depletion of one or more other cell subsets.

The PBMC fraction may be enriched for HIV-specific CD4 T cells by contacting the cells with HIV proteins matching or complementary to the components of the vaccine previously used for in vivo immunization. Ex vivo re-stimulation can increase the relative frequency of HIV-specific CD4 T cells by about 2, about 5, about 10, about 25, about 50, about 75, about 100, about 125, about 150, about 175, or about 200-fold. The methods may additionally include combining in vivo therapeutic immunization and ex vivo re-stimulation of CD4 T cells with ex vivo lentiviral transduction and culturing.

Thus, in one embodiment, the re-stimulated PBMC or fraction of PBMC that has been enriched for HIV-specific CD4 T cells can be cultured for 1, 2, 3, 4, 5 or up to 12, 20 or 30 days before activating again with a polyclonal mitogen or other plant- or fungal based agglutinins or other reagents capable of recognizing cell surface CD3 and CD28 to cross link these molecules and cause polyclonal T cell activation. After polyclonal stimulation, cells may be transduced with therapeutic anti-HIV lentivirus or other vectors and maintained in culture for a sufficient period of time for such transduction, for example from about 1 to about 21 days, including up to about 35 days. Alternatively, the cells may be cultured for about 1-about 18 days, about 1-about 15 days, about 1-about 12 days, about 1-about 9 days, or about 3-about 7 days. Thus, the transduced cells may be cultured for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, or about 35 days. Activation with a polyclonal mitogen may or may not be included in the cell product manufacturing process.

In further embodiments, once the transduced cells have been cultured for a sufficient period of time, transduced CD4 T cells are infused back into the original patient. Infusion can be performed using various devices and methods known in the art. In some embodiments, infusion may be accompanied by pre-treatment with cyclophosphamide or similar compounds to increase the efficiency of re-engraftment.

In some embodiments, a CCR5-targeted therapy may be added to a subject's antiretroviral therapy regimen, which was continued throughout the treatment process. Examples of CCR5-targeted therapies include but are not limited to Maraviroc (a CCR5 antagonist) or Rapamycin (immunosuppressive agent that lowers CCR5). In some embodiments, the antiretroviral therapy may be ceased and the subject can be tested for virus rebound. If no rebound occurs, adjuvant therapy can also be removed and the subject can be tested again for virus rebound.

In various embodiments, continued virus suppression with reduced or no antiretroviral therapy including cART or HAART, and reduced or no adjuvant therapy for about 26 weeks can be considered a functional cure for HIV. Other definitions of a cure are described herein.

The lentiviral and other vectors used in the disclosed methods may encode at least one, at least two, at least three, at least four, or at least five genes, or at least six genes, or at least seven genes, or at least eight genes, or at least nine genes, or at least ten genes, or at least eleven genes, or at least twelve genes of interest. Given the versatility and therapeutic potential of HIV-targeted gene therapy, a viral vector of the disclosure may encode genes or nucleic acid sequences that include but are not limited to (i) an antibody directed to an antigen associated with an infectious disease or a toxin produced by the infectious pathogen, (ii) cytokines including interleukins that are required for immune cell growth or function and may be therapeutic for immune dysregulation encountered in HIV and other chronic or acute human viral or bacterial pathogens, (iii) factors that suppress the growth of HIV in vivo including CD8 suppressor factors, (iv) mutations or deletions of chemokine receptor CCR5, mutations or deletions of chemokine receptor CXCR4, or mutations or deletions of chemokine receptor CXCR5, (v) antisense DNA or RNA against specific receptors or peptides associated with HIV or host protein associated with HIV, (vi) small interfering RNA against specific receptors or peptides associated with HIV or host protein associated with HIV, or (vii) a variety of other therapeutically useful sequences that may be used to treat HIV or AIDS.

Additional examples of HIV-targeted gene therapy that can be used in the disclosed methods include, but are not limited to, affinity-enhanced T cell receptors, chimeric antigen receptors on CD4 T cells (or alternatively on CD8 T cells), modification of signal transduction pathways to avoid cell death cause by viral proteins, increased expression of HIV restriction elements including TREX, SAMHD1, MxA or MxB proteins, APOBEC complexes, TRIMS-alpha complexes, tetherin (BST2), and similar proteins identified as being capable of reducing HIV replication in mammalian cells.

In some embodiments, a patient may be undergoing cART or HAART concurrently while being treated according to the methods of the disclosure. In other embodiments, a patient may undergo cART or HAART before or after being treated according to the methods of the disclosure. In some embodiments, cART or HAART is maintained throughout treatment according to the methods of the disclosure and the patient may be monitored for HIV viral burden in blood and frequency of lentivirus-transduced CD4 T cells in blood. Preferably, a patient receiving cART or HAART prior to being treated according to the methods of the disclosure is able to discontinue or reduce cART or HAART following treatment according to the methods of the disclosure.

For efficacy purposes, the frequency of transduced, HIV-specific CD4 T cells, which is a novel surrogate marker for gene therapy effects, may be determined, as discussed in more detail herein.

### Compositions

In various aspects, the disclosure provides lentiviral vectors capable of delivering genetic constructs to inhibit HIV penetration of susceptible cells. For instance, one mechanism of action in accordance herein is to reduce mRNA levels for CCR5 and/or CXCR4 chemokine receptors for reducing the rates for viral entry into susceptible cells.

Alternatively, the disclosed lentiviral vectors are capable of inhibiting the formation of HIV-infected cells by reducing the stability of incoming HIV genomic RNA. And in yet another embodiment, the disclosed lentivirus vectors are capable of preventing HIV production from a latently infected cell, wherein the mechanism of action is to cause instability of viral RNA sequences through the action of inhibitory RNA including short-homology, small-interfering or other regulatory RNA species.

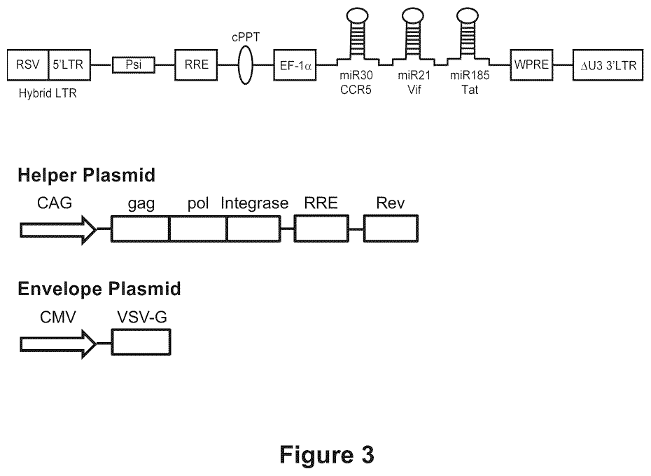

The therapeutic lentiviruses disclosed generally comprise at least one of two types of genetic cargo. First, the lentiviruses may encode genetic elements that direct expression of small RNA capable of inhibiting the production of chemokine receptors CCR5 and/or CXCR4 that are important for HIV penetration of susceptible cells. The second type of genetic cargo includes constructs capable of expressing small RNA molecules targeting HIV RNA sequences for the purpose of preventing reverse transcription, RNA splicing, RNA translation to produce proteins, or packaging of viral genomic RNA for particle production and spreading infection. An exemplary structure is diagrammed in FIG. 3.

As shown in FIG. 3 (top panel), an exemplary construct may comprise numerous sections or components. For example, in one embodiment, an exemplary LV construct may comprise the following sections or components:
RSV—a Rous Sarcoma virus long terminal repeat;
5′LTR—a portion of an HIV long terminal repeat that can be truncated to prevent replication of the vector after chromosomal integration;
Psi—a packaging signal that allows for incorporation of the vector RNA genome into viral particles during packaging;
RRE—a Rev Responsive element can be added to improve expression from the transgene by mobilizing RNA out of the nucleus and into the cytoplasm of cells;
cPPT—a Poly purine tract that facilitates second strand DNA synthesis prior to integration of the transgene into the host cell chromosome;
Promoter—a promoter initiates RNA transcription from the integrated transgene to express micro-RNA clusters (or other genetic elements of the construct), and in some embodiments, the vectors may use an EF-1 promoter;
Anti-CCR5—a micro RNA targeting messenger RNA for the host cell factor CCR5 to reduce its expression on the cell surface;
Anti-Rev/Tat—a micro RNA targeting HIV genomic or messenger RNA at the junction between HIV Rev and Tat coding regions, which is sometimes designated miRNA Tat or given a similar description in this application;
Anti-Vif—a micro RNA targeting HIV genomic or messenger RNA within the Vif coding region;
WPRE—a woodchuck hepatitis virus post-transcriptional regulatory element is an additional vector component that can be used to facilitate RNA transport of the nucleus; and
deltaU3 3′LTR—a modified version of a HIV 3′ long terminal repeat where a portion of the U3 region has been deleted to improve safety of the vector.

One of ordinary skill in the art will recognize that the above components are merely examples, and that such components may be reorganized, substituted with other elements, or otherwise changed, so long as the construct is able to prevent expression of HIV genes and decrease the spread of infection.

Vectors of the disclosure may include either or both of the types of genetic cargo discussed above (i.e., genetic elements that direct expression of a gene or small RNAs, such as siRNA, shRNA, or miRNA that can prevent translation or transcription), and the vectors of the disclosure may also encode additionally useful products for the purpose of treatment or diagnosis of HIV. For instance, in some embodiments, these vectors may also encode green fluorescent protein (GFP) or truncated and biologically inactive cell surface molecules for the purpose of tracking the vectors or antibiotic resistance genes for the purposes of selectively maintaining genetically-modified cells in vivo.

The combination of genetic elements incorporated into the disclosed vectors is not particularly limited. For example, a vector herein may encode a single small RNA, two small RNAs, three small RNA, four small RNAs, five small RNAs, six small RNAs, seven small RNAs, eight small RNAs, nine small RNAs, or ten small RNAs, or eleven small RNAs, or twelve small RNAs. Such vectors may additionally encode other genetic elements to function in concert with the small RNAs to prevent expression and infection of HIV.

Those of ordinary skill in the art will understand that the therapeutic lentivirus may substitute alternate sequences for the promoter region, targeting of regulatory RNA, and types of regulatory RNA. Further, the therapeutic lentivirus of the disclosure may comprise changes in the plasmids used for packaging the lentivirus particles; these changes are required to increase levels of production in vitro.

### Lentiviral Vector System

A lentiviral virion (particle) in accordance with various aspects and embodiments herein is expressed by a vector system encoding the necessary viral proteins to produce a virion (viral particle). In various embodiments, one vector containing a nucleic acid sequence encoding the lentiviral pol proteins is provided for reverse transcription and integration, operably linked to a promoter. In another embodiment, the pol proteins are expressed by multiple vectors. In other embodiments, vectors containing a nucleic acid sequence encoding the lentiviral Gag proteins for forming a viral capsid, operably linked to a promoter, are provided. In embodiments, this gag nucleic acid sequence is on a separate vector than at least some of the pol nucleic acid sequence. In other embodiments, the gag nucleic acid is on a separate vector from all the pol nucleic acid sequences that encode pol proteins.

Numerous modifications can be made to the vectors herein, which are used to create the particles to further minimize the chance of obtaining wild type revertants. These include, but are not limited to deletions of the U3 region of the LTR, tat deletions and matrix (MA) deletions. In embodiments, the gag, pol and env vector(s) do not contain nucleotides from the lentiviral genome that package lentiviral RNA, referred to as the lentiviral packaging sequence.

The vector(s) forming the particle preferably do not contain a nucleic acid sequence from the lentiviral genome that expresses an envelope protein. Preferably, a separate vector that contains a nucleic acid sequence encoding an envelope protein operably linked to a promoter is used. This env vector also does not contain a lentiviral packaging sequence. In one embodiment the env nucleic acid sequence encodes a lentiviral envelope protein.

In another embodiment the envelope protein is not from the lentivirus, but from a different virus. The resultant particle is referred to as a pseudotyped particle. By appropriate selection of envelopes one can “infect” virtually any cell. For example, one can use an env gene that encodes an envelope protein that targets an endocytic compartment. Examples of viruses from which such env genes and envelope proteins can derive include the influenza virus (e.g., the Influenza A virus, Influenza B virus, Influenza C virus, Influenza D virus, Isavirus, Quaranjavirus, and Thogotovirus), the Vesiculovirus (e.g., Indiana vesiculovirus), alpha viruses (e.g., the Semliki forest virus, Sindbis virus, Aura virus, Barmah Forest virus, Bebaru virus, Cabassou virus, Getah virus, Highlands J virus, Trocara virus, Una Virus, Ndumu virus, and Middleburg virus, among others), arenaviruses (e.g., the lymphocytic choriomeningitis virus, Machupo virus, Junin virus and Lassa Fever virus), flaviviruses (e.g., the tick-borne encephalitis virus, Dengue virus, hepatitis C virus, GB virus, Apoi virus, Bagaza virus, Edge Hill virus, Jugra virus, Kadam virus, Dakar bat virus, Modoc virus, Powassan virus, Usutu virus, and Sal Viej a virus, among others), rhabdoviruses (e.g., vesicular stomatitis virus, rabies virus), paramyxoviruses (e.g., mumps or measles) and orthomyxoviruses (e.g., influenza virus).

Other env gene envelope proteins that can preferably be used include those derived from endogenous retroviruses (e.g., feline endogenous retroviruses and baboon endogenous retroviruses) and closely related gammaretroviruses (e.g., the Moloney Leukemia Virus, MLV-E, MLV-A, Gibbon Ape Leukemia Virus, GALV, Feline leukemia virus, Koala retrovirus, Trager duck spleen necrosis virus, Viper retrovirus, Chick syncytial virus, Gardner-Arnstein feline sarcoma virus, and Porcine type-C oncovirus, among others). These gammaretroviruses can be used as sources of env genes and envelope proteins for targeting primary cells. The gammaretroviruses are particularly preferred where the host cell is a primary cell.

Envelope proteins can be selected to target a specific desired host cell. For example, targeting specific receptors such as a dopamine receptor can be used for brain delivery. Another target can be vascular endothelium. These cells can be targeted using an envelope protein derived from any virus in the Filoviridae family (e.g., Cuevaviruses, Dianloviruses, Ebolaviruses, and Marburgviruses). Species of Ebolaviruses include Tai Forest ebolavirus, Zaire ebolavirus, Sudan ebolavirus, Bundibugyo ebolavirus, and Reston ebolavirus.

In addition, in embodiments, glycoproteins can undergo post-transcriptional modifications. For example, in an embodiment, the GP of Ebola, can be modified after translation to become the GP1 and GP2 glycoproteins. In another embodiment, one can use different lentiviral capsids with a pseudotyped envelope (e.g., FIV or SHIV [U.S. Pat. No. 5,654,195]). A SHIV pseudotyped vector can readily be used in animal models such as monkeys.

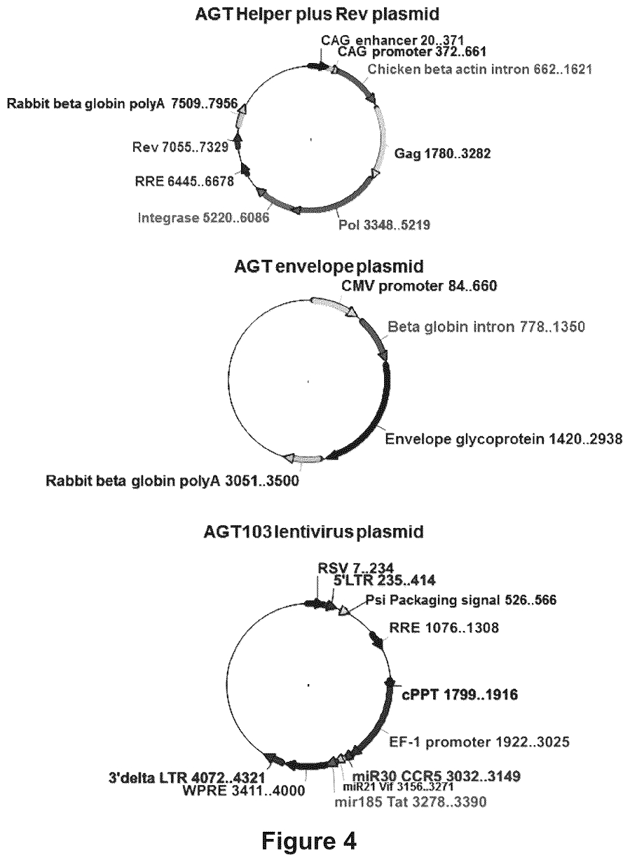

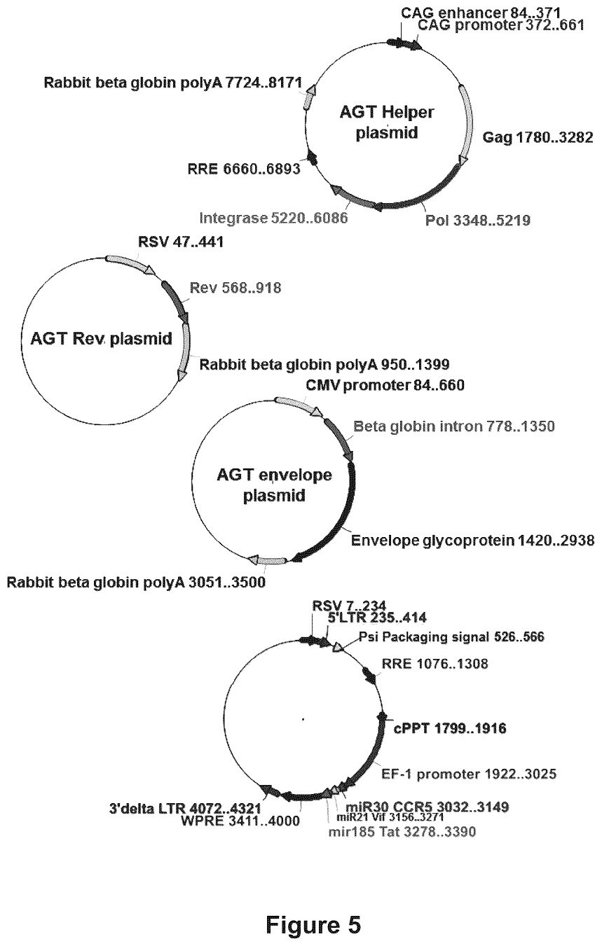

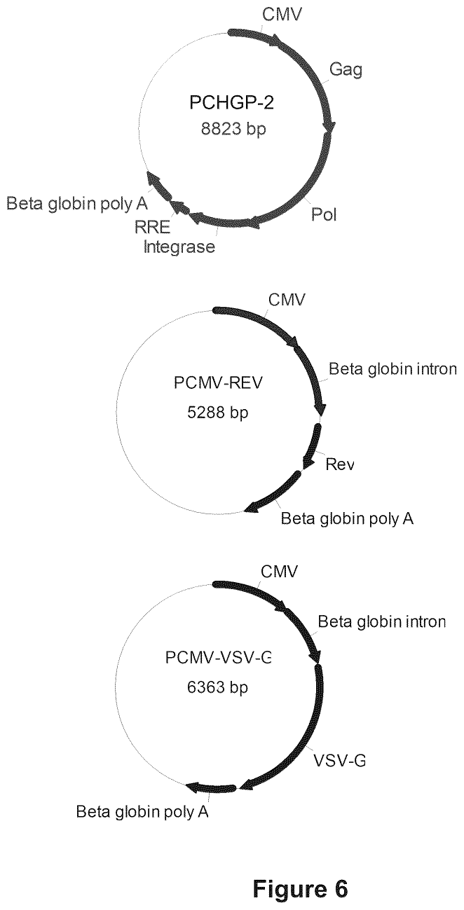

Lentiviral vector systems as provided herein typically include at least one helper plasmid comprising at least one of a gag, pol, or rev gene. Each of the gag, pol and rev genes may be provided on individual plasmids, or one or more genes may be provided together on the same plasmid. In one embodiment, the gag, pol, and rev genes are provided on the same plasmid (e.g., FIG. 4). In another embodiment, the gag and pol genes are provided on a first plasmid and the rev gene is provided on a second plasmid (e.g., FIG. 5). Accordingly, both 3-vector (e.g., FIGS. 4 and 6) and 4-vector (e.g., FIG. 5) systems can be used to produce a lentivirus as described herein. In embodiments, the therapeutic vector, at least one envelope plasmid and at least one helper plasmid are transfected into a packaging cell, for example a packaging cell line. A non-limiting example of a packaging cell line is the 293T/17 HEK cell line. When the therapeutic vector, the envelope plasmid, and at least one helper plasmid are transfected into the packaging cell line, a lentiviral particle is ultimately produced.

In another aspect, a lentiviral vector system for expressing a lentiviral particle is disclosed. The system includes a lentiviral vector as described herein; an envelope plasmid for expressing an envelope protein optimized for infecting a cell; and at least one helper plasmid for expressing gag, pol, and rev genes, wherein when the lentiviral vector, the envelope plasmid, and the at least one helper plasmid are transfected into a packaging cell line, a lentiviral particle is produced by the packaging cell line, wherein the lentiviral particle is capable of inhibiting production of chemokine receptor CCR5 or targeting an HIV RNA sequence.

In another aspect, the lentiviral vector, which is also referred to herein as a therapeutic vector, includes the following elements: hybrid 5′ long terminal repeat (RSV/5′ LTR) (SEQ ID NOS: 33-34), Psi sequence (RNA packaging site) (SEQ ID NO: 35), RRE (Rev-response element) (SEQ ID NO: 36), cPPT (polypurine tract) (SEQ ID NO: 37), EF-la promoter (SEQ ID NO: 4), miR30CCR5 (SEQ ID NO: 1), miR21Vif (SEQ ID NO: 2), miR185Tat (SEQ ID NO: 3), Woodchuck Post-Transcriptional Regulatory Element (WPRE) (SEQ ID NOS: 31 or 67), and AU3 3′ LTR (SEQ ID NO: 38). In another aspect, sequence variation, by way of substitution, deletion, addition, or mutation can be used to modify the sequences references herein.

In another aspect, a helper plasmid includes the following elements: CAG promoter (SEQ ID NO: 40); HIV component gag (SEQ ID NO: 42); HIV component pol (SEQ ID NO: 43); HIV Int (SEQ ID NO: 44); HIV RRE (SEQ ID NO: 45); and HIV Rev (SEQ ID NO: 46). In another aspect, the helper plasmid may be modified to include a first helper plasmid for expressing the gag and pol genes, and a second and separate plasmid for expressing the rev gene. In another aspect, sequence variation, by way of substitution, deletion, addition, or mutation can be used to modify the sequences references herein.

In another aspect, an envelope plasmid includes the following elements: RNA polymerase II promoter (CMV) (SEQ ID NO: 49) and vesicular stomatitis virus G glycoprotein (VSV-G) (SEQ ID NO: 51). In another aspect, sequence variation, by way of substitution, deletion, addition, or mutation can be used to modify the sequences references herein.

In various aspects, the plasmids used for lentiviral packaging are modified by substitution, addition, subtraction or mutation of various elements without loss of vector function. For example, and without limitation, the following elements can replace similar elements in the plasmids that comprise the packaging system: Elongation Factor-1 (EF-1), phosphoglycerate kinase (PGK), and ubiquitin C (UbC) promoters can replace the CMV or CAG promoter. SV40 poly A and bGH poly A can replace the rabbit beta globin poly A.

In another aspect, the HIV sequences in the helper plasmid can be constructed from different HIV strains or clades. For example, the VSV-G glycoprotein can be substituted with membrane glycoproteins derived from gammaretroviruses (e.g., gibbon ape leukemia virus, GALV, murine leukemia virus 10A1, MLV, Koala retrovirus, Trager duck spleen necrosis virus, Viper retrovirus, Chick syncytial virus, Gardner-Arnstein feline sarcoma virus, and Porcine type-C oncovirus, among others), endogenous retroviruses (e.g., feline endogenous virus (RD114), human endogenous retrovirus such as HERV-W, and baboon endogenous retrovirus, BaEV, among others), Lyssavirus (e.g., Rabies virus, FUG), mammarenavirus (e.g., lymphocytic choriomeningitis virus, LCMV, Influenza viruses such as the Influenza A virus, Influenza A fowl plague virus, FPV, Influenza B virus, Influenza C virus, Influenza D virus, Isavirus, Quaranjavirus, and Thogotovirus), Alphavirus (e.g., Ross River alphavirus, RRV, or Ebola viruses, EboV, such as Sudan ebolavirus, Tai Forest ebolavirus, Zaire ebolavirus, Bundibugyo ebolavirus, and Reston ebolavirus).

Various lentiviral packaging systems can be acquired commercially (e.g., Lenti-vpak packaging kit from OriGene Technologies, Inc., Rockville, MD), and can also be designed as described herein. Moreover, it is within the skill of a person ordinarily skilled in the art to substitute or modify aspects of a lentiviral packaging system to improve any number of relevant factors, including the production efficiency of a lentiviral particle.

### Bioassays

In various aspects, the present disclosure includes bioassays for determining the success of HIV treatment for achieving a functional cure. These assays provide a method for measuring the efficacy of the disclosed methods of immunization and treatment by measuring the frequency of transduced, HIV specific CD4 T cells in a patient. HIV-specific CD4 T cells are recognizable because, among others, they proliferate, change the composition of cell surface markers, induce signaling pathways including phosphorylation, and/or express specific marker proteins that may be cytokines, chemokines, caspases, phosphorylated signaling molecules or other cytoplasmic and/or nuclear components. Specific responding CD4 T cells are recognized for example, using labeled monoclonal antibodies or specific in situ amplification of mRNA sequences, that allow sorting of HIV-specific cells using flow cytometry sorting, magnetic bead separation or other recognized methods for antigen-specific CD4 T cell isolation. The isolated CD4 T cells are tested to determine the frequency of cells carrying integrated therapeutic lentivirus. Single cell testing methods may also be used including microfluidic separation of individual cells that are coupled with mass spectrometry, PCR, ELISA or antibody staining to confirm responsiveness to HIV and presence of integrated therapeutic lentivirus.

Thus, in various embodiments, following application of a treatment according to the disclosure (e.g., (a) immunization, (b) ex vivo leukocyte/lymphocyte culture and/or depletion of non-target cells; (c) re-stimulation with purified proteins, inactivated viruses, virally vectored proteins, bacterially vectored proteins, biological or chemical adjuvants including cytokines and/or chemokines, vehicles; (d) transduction of the enriched T cells with a viral delivery system; and (e) infusion of the enriched, transduced T cells), a patient may be subsequently assayed to determine the efficacy of the treatment. A threshold value of target T cells in the body may be established to measure a functional cure at a determined value, for example, at about 1×10⁸ HIV-specific CD4 T cells bearing genetic modification from therapeutic lentivirus. Alternatively, the threshold value may be about 1×10⁵, about 1×10⁶, about 1×10⁷, about 1×10⁸, about 1×10⁹, or about 1×10¹⁰ CD4 T cells in the body of the patient.

HIV-specific CD4 T cells bearing genetic modification from therapeutic lentivirus can be determined using any suitable method, such as but not limited to flow cytometry, cell sorting, FACS analysis, DNA cloning, PCR, RT-PCR or Q-PCR, ELISA, FISH, western blotting, southern blotting, high throughput sequencing, RNA sequencing, oligonucleotide primer extension, or other methods known in the art.

While methods for defining antigen specific T cells with genetic modifications are known in the art, utilizing such methods to combine identifying HIV-specific T cells with integrated or non-integrated gene therapy constructs as a standard measure for efficacy is a novel concept in the field of HIV treatment, as described variously herein.

### Doses and Dosage Forms

The disclosed methods and compositions can be used for treating HIV+ patients during various stages of their disease. Accordingly, dosing regimens may vary based upon the condition of the patient and the method of administration.

In various embodiments, HIV-specific vaccines for the initial in vivo immunization are administered to a subject in need in varying doses. In general, vaccines delivered by intramuscular injection include about 10 μg to about 300 μg, about 25 μg to about 275 μg, about 50 μg to about 250 μg, about 75 μg to about 225 μg, or about 100 μg to about 200 μg of HIV protein, either total virus protein prepared from inactivated virus particles, virus-like particles or purified virus protein from recombinant systems or purified from virus preparations. Recombinant viral or bacterial vectors may be administered by any and all of the routes described. Intramuscular vaccines will include about 1 μg to about 100 μg, about 10 μg to about 90 μg, about 20 μg to about 80 μg, about 30 μg to about 70 μg, about 40 μg to about 60 μg, or about 50 μg of suitable adjuvant molecules and be suspended in oil, saline, buffer or water in volumes of 0.1 to 5 ml per injection dose, and may be soluble or emulsion preparations. Vaccines delivered orally, rectally, buccally, at genital mucosal or intranasally, including some virally-vectored or bacterially-vectored vaccines, fusion proteins, liposome formulations or similar preparations, may contain higher amounts of virus protein and adjuvant. Dermal, sub-dermal or subcutaneous vaccines utilize protein and adjuvant amounts more similar to oral, rectal or intranasal-delivered vaccines. Depending on responses to the initial immunization, vaccination may be repeated 1-5 times using the same or alternate routes for delivery. Intervals may be of 2-24 weeks between immunizations. Immune responses to vaccination are measured by testing HIV-specific antibodies in serum, plasma, vaginal secretions, rectal secretions, saliva or bronchoalveolar lavage fluids, using ELISA or similar methodology. Cellular immune responses are tested by in vitro stimulation with vaccine antigens followed by staining for intracellular cytokine accumulation followed by flow cytometry or similar methods including lymphoproliferation, expression of phosphorylated signaling proteins or changes in cell surface activation markers. Upper limits of dosing may be determined based on the individual patient and will depend on toxicity/safety profiles for each individual product or product lot.

Immunization may occur once, twice, three times, or repeatedly. For instance, an agent for HIV immunization may be administered to a subject in need once a week, once every other week, once every three weeks, once a month, every other month, every three months, every six months, every nine months, once a year, every eighteen months, every two years, every 36 months, or every three years.

Immunization will generally occur at least once before ex vivo expansion and enrichment of CD4 T cells, and immunization may occur once, twice, three times, or more after ex vivo leukocyte/lymphocyte culture/re-stimulation and infusion.

In one embodiment, HIV vaccines for immunization are administered as a pharmaceutical composition. In one embodiment, the pharmaceutical composition comprising an HIV vaccine is formulated in a wide variety of nasal, pulmonary, oral, topical, or parenteral dosage forms for clinical application. Each of the dosage forms can comprise various disintegrating agents, surfactants, fillers, thickeners, binders, diluents such as wetting agents or other pharmaceutically acceptable excipients. The pharmaceutical composition comprising an HIV vaccine can also be formulated for injection.

HIV vaccine compositions for the purpose of immunization can be administered using any pharmaceutically acceptable method, such as intranasal, buccal, sublingual, oral, rectal, ocular, parenteral (intravenously, intradermally, intramuscularly, subcutaneously, intracisternally, intraperitoneally), pulmonary, intravaginal, locally administered, topically administered, topically administered after scarification, mucosally administered, via an aerosol, or via a buccal or nasal spray formulation.

Further, the HIV vaccine compositions can be formulated into any pharmaceutically acceptable dosage form, such as a solid dosage form, tablet, pill, lozenge, capsule, liquid dispersion, gel, aerosol, pulmonary aerosol, nasal aerosol, ointment, cream, semi-solid dosage form, and a suspension. Further, the composition may be a controlled release formulation, sustained release formulation, immediate release formulation, or any combination thereof. Further, the composition may be a transdermal delivery system.

In another embodiment, the pharmaceutical composition comprising an HIV vaccine is formulated in a solid dosage form for oral administration, and the solid dosage form can be powders, granules, capsules, tablets or pills. In yet another embodiment, the solid dosage form includes one or more excipients such as calcium carbonate, starch, sucrose, lactose, microcrystalline cellulose or gelatin. In addition, the solid dosage form can include, in addition to the excipients, a lubricant such as talc or magnesium stearate. In some embodiments, the oral dosage form is in immediate release or a modified release form. Modified release dosage forms include controlled or extended release, enteric release, and the like. The excipients used in the modified release dosage forms are commonly known to a person of ordinary skill in the art.

In a further embodiments, the pharmaceutical composition comprising a HIV vaccine is formulated as a sublingual or buccal dosage form. Such dosage forms comprise sublingual tablets or solution compositions that are administered under the tongue and buccal tablets that are placed between the cheek and gum.

In yet a further embodiment, the pharmaceutical composition comprising an HIV vaccine is formulated as a nasal dosage form. Such dosage forms of the present disclosure comprise solution, suspension, and gel compositions for nasal delivery.

In one embodiment, the pharmaceutical composition is formulated in a liquid dosage form for oral administration, such as suspensions, emulsions or syrups. In other embodiments, the liquid dosage form can include, in addition to commonly used simple diluents such as water and liquid paraffin, various excipients such as humectants, sweeteners, aromatics or preservatives. In particular embodiments, the composition comprising HIV vaccine or a pharmaceutically acceptable salt thereof is formulated to be suitable for administration to a pediatric patient.

In one embodiment, the pharmaceutical composition is formulated in a dosage form for parenteral administration, such as sterile aqueous solutions, suspensions, emulsions, non-aqueous solutions or suppositories. In other embodiments, the non-aqueous solutions or suspensions includes propylene glycol, polyethylene glycol, vegetable oils such as olive oil or injectable esters such as ethyl oleate. As a base for suppositories, witepsol, macrogol, tween 61, cacao oil, laurin oil or glycerinated gelatin can be used.

The dosage of the pharmaceutical composition can vary depending on the patient's weight, age, gender, administration time and mode, excretion rate, and the severity of disease.

For the purposes of re-stimulation, lymphocytes, PBMCs, and/or CD4 T cells are generally removed from a patient and isolated for re-stimulation and culturing. The isolated cells may be contacted with the same HIV vaccine or activating agent used for immunization or a different HIV vaccine or activating agent. In one embodiment, the isolated cells are contacted with about 10 ng to 5 μg of an HIV vaccine or activating agent per about 106 cells in culture (or any other suitable amount). More specifically, the isolated cells may be contacted with about 50 ng, about 100 ng, about 200 ng, about 300 ng, about 400 ng, about 500 ng, about 600 ng, about 700 ng, about 800 ng, about 900 ng, about 1 μg, about 1.5 μg, about 2 μg, about 2.5 μg, about 3 μg, about 3.5 μg, about 4 μg, about 4.5 μg, or about 5 μg of an HIV vaccine or activating agent per about 106 cells in culture.

Activating agents or vaccines are generally used once for each in vitro cell culture but may be repeated after intervals of about 15 to about 35 days. For example, a repeat dosing could occur at about 15, about 16, about 17, about 18, about 19, about 20, about 21, about 22, about 23, about 24, about 25, about 26, about 27, about 28, about 29, about 30, about 31, about 32, about 33, about 34, or about 35 days.

For transduction of the enriched, re-stimulated cells, the cells may be transduced with lentiviral vectors or with other known vector systems as disclosed, for example, in FIG. 4 or FIG. 6. The cells being transduced may be contacted with about 1-1,000 viral genomes (measured by RT-PCR assay of culture fluids containing lentivirus vector) per target cell in culture (or any other suitable amount). Lentivirus transduction may be repeated 1-5 times using the same range of 1-1,000 viral genomes per target cell in culture.

### Cellular Enrichment

In various embodiments, cells such as T cells are obtained from an HIV infected patient and cultured. Culturing can occur in multiwell plates in a culture medium comprising conditioned media (“CM”). The levels of supernatant p24gag (“p24”) and viral RNA levels may be assessed by standard means. Those patients whose CM-cultured cells have peak p24 supernatant levels of less than 1 ng/ml may be suitable patients for large-scale T-cell expansion in CM with or without the use of additional anti-viral agents. Additionally, different drugs or drug combinations of interest may be added to different wells and the impact on virus levels in the sample may be assessed by standard means. Those drug combinations providing adequate viral suppression are therapeutically useful combinations. It is within the capacity of a competent technician to determine what constitutes adequate viral suppression in relation to a particular subject. In order to test the effectiveness of drugs of interest in limiting viral expansion, additional factors such as anti-CD3 antibodies may be added to the culture to stimulate viral production. Unlike culture methods for HIV infected cell samples known in the art, CM allows the culture of T cells for periods of over two months, thereby providing an effective system in which to assay long term drug effectiveness.

In a preferred embodiment, the HIV infected cells are obtained from a subject with susceptible transcription mediating protein sequences and susceptible HIV regulatory element sequences. In a more preferred embodiment, the HIV infected cells are obtained from a subject having wild-type transcription-mediating protein sequences and wild-type HIV regulatory sequences.

Stable transduction of primary cells of the hematopoietic system and/or hematopoietic stem cells may be obtained by contacting, in vitro or ex vivo, the surface of the cells with both a lentiviral vector and at least one molecule which binds the cell surface. The cells may be cultured in a ventilated vessel comprising two or more layers under conditions conducive to growth and/or proliferation. In some embodiments, this approach may be used in conjunction with non-CD4+ T cell depletion and/or broad polyclonal expansion.

In another approach to T cell enrichment, PBMCs are stimulated with a peptide and enriched for cells secreting a cytokine, such as interferon-gamma. This approach generally involves stimulating a mixture of cells containing T cells with antigen, and effecting a separation of antigen-stimulated cells according to the degree to which they are labeled with the product. Antigen stimulation is achieved by exposing the cells to at least one antigen under conditions effective to elicit antigen-specific stimulation of at least one T cell. Labeling with the product is achieved by modifying the surface of the cells to contain at least one capture moiety, culturing the cells under conditions in which the product is secreted, released and specifically bound (“captured” or “entrapped”) to said capture moiety; and labeling the captured product with a label moiety, where the labeled cells are not lysed as part of the labeling procedure or as part of the separation procedure. The capture moiety may incorporate detection of cell surface glycoproteins CD3 or CD4 to refine the enrichment step and increase the proportion of antigen-specific T cells in general, of CD4+ T cells in specific.

The following examples are given to illustrate aspects of the present disclosure. It should be understood, however, that the disclosure is not to be limited to the specific conditions or details described in these examples. All printed publications referenced herein are specifically incorporated by reference.

### EXAMPLES

### Example 1: Development of a Lentiviral Vector System

A lentiviral vector system was developed as summarized in FIG. 3 (linear form) and FIG. 4 (circularized form). Referring first to the top portion of FIG. 3, a representative therapeutic vector has been designed and produced with the following elements being from left to right: hybrid 5′ long terminal repeat (RSV/5′ LTR) (SEQ ID NOS: 33-34), Psi sequence (RNA packaging site) (SEQ ID NO: 35), RRE (Rev-response element) (SEQ ID NO: 36), cPPT (polypurine tract) (SEQ ID NO: 37), EF-1α promoter (SEQ ID NO: 4), miR30CCR5 (SEQ ID NO: 1), miR21Vif (SEQ ID NO: 2), miR185Tat (SEQ ID NO: 3), Woodchuck Post-Transcriptional Regulatory Element (WPRE) (SEQ ID NOS: 31 or 67), and AU3 3′ LTR (SEQ ID NO: 38). The therapeutic vector detailed in FIG. 3 is also referred to herein as AGT103.

Referring next to the middle portion of FIG. 3, a helper plasmid has been designed and produced with the following elements being from left to right: CAG promoter (SEQ ID NO: 40); HIV component gag (SEQ ID NO: 42); HIV component pol (SEQ ID NO: 43); HIV Int (SEQ ID NO: 44); HIV RRE (SEQ ID NO: 45); and HIV Rev (SEQ ID NO: 46).

Referring next to the lower portion of FIG. 3, an envelope plasmid has been designed and produced with the following elements being from left to right: RNA polymerase II promoter (CMV) (SEQ ID NO: 49) and vesicular stomatitis virus G glycoprotein (VSV-G) (SEQ ID NO: 51).

Lentiviral particles were produced in 293T/17 HEK cells (purchased from American Type Culture Collection, Manassas, VA) following transfection with the therapeutic vector, the envelope plasmid, and the helper plasmid (as shown in FIG. 3). The transfection of 293T/17 HEK cells, which produced functional viral particles, employed the reagent Poly(ethylenimine) (PEI) to increase the efficiency of plasmid DNA uptake. The plasmids and DNA were initially added separately in culture medium without serum in a ratio of 3:1 (mass ratio of PEI to DNA). After 2-3 days, cell medium was collected and lentiviral particles were purified by high-speed centrifugation and/or filtration followed by anion-exchange chromatography. The concentration of lentiviral particles can be expressed in terms of transducing units/ml (TU/ml). The determination of TU was accomplished by measuring HIV p24 levels in culture fluids (p24 protein is incorporated into lentiviral particles), measuring the number of viral DNA copies per cell by quantitative PCR, or by infecting cells and using light (if the vectors encode luciferase or fluorescent protein markers).

As mentioned above, a 3-vector system (i.e., a 2-vector lentiviral packaging system) was designed for the production of lentiviral particles. A schematic of the 3-vector system is shown in FIG. 4. The schematic of FIG. 4 is a circularized version of the linear system previously described in FIG. 3. Briefly, and with reference to FIG. 4, the top-most vector is a helper plasmid, which, in this case, includes Rev. The vector appearing in the middle of FIG. 4 is the envelope plasmid. The bottom-most vector is the previously described therapeutic vector.

Referring more specifically to FIG. 4, the Helper plus Rev plasmid includes a CAG enhancer (SEQ ID NO: 39); a CAG promoter (SEQ ID NO: 40); a chicken beta actin intron (SEQ ID NO: 41); a HIV gag (SEQ ID NO: 42); a HIV Pol (SEQ ID NO: 43); a HIV Int (SEQ ID NO: 44); a HIV RRE (SEQ ID NO: 45); a HIV Rev (SEQ ID NO: 46); and a rabbit beta globin poly A (SEQ ID NO: 47).

The Envelope plasmid includes a CMV promoter (SEQ ID NO: 49); a beta globin intron (SEQ ID NO: 50); a VSV-G (SEQ ID NO: 51); and a rabbit beta globin poly A (SEQ ID NO: 56).

In an alternate vector system, and with respect to FIG. 6, the vector sequences are provided herein as SEQ ID NOs: 81-83.

Synthesis of a 2-vector lentiviral packaging system including Helper (plus Rev) and Envelope plasmids.

Materials and Methods:

Construction of the helper plasmid: The helper plasmid was constructed by initial PCR amplification of a DNA fragment from the pNL4-3 HIV plasmid (NIH Aids Reagent Program) containing Gag, Pol, and Integrase genes. Primers were designed to amplify the fragment with EcoRI and NotI restriction sites which could be used to insert at the same sites in the pCDNA3 plasmid (Invitrogen). The forward primer was (5′-TAAGCAGAATTCATGAATTTGCCAGGAAGAT-3′) (SEQ ID NO: 68) and reverse primer was (5′-CCATACAATGAATGGACACTAGGCGGCCGCACGAAT-3′) (SEQ ID NO: 69). The sequence for the Gag, Pol, Integrase fragment was as follows:

(SEQ ID NO: 70)
GAATTCATGAATTTGCCAGGAAGATGGAAACCAAAAATGATAGGGGG
AATTGGAGGTTTTATCAAAGTAAGACAGTATGATCAGATACTCATAGAA
ATCTGCGGACATAAAGCTATAGGTACAGTATTAGTAGGACCTACACCTG
TCAACATAATTGGAAGAAATCTGTTGACTCAGATTGGCTGCACTTTAAA
TTTTCCCATTAGTCCTATTGAGACTGTACCAGTAAAATTAAAGCCAGGA
ATGGATGGCCCAAAAGTTAAACAATGGCCATTGACAGAAGAAAAAATAA
AAGCATTAGTAGAAATTTGTACAGAAATGGAAAAGGAAGGAAAAATTTC
AAAAATTGGGCCTGAAAATCCATACAATACTCCAGTATTTGCCATAAAG
AAAAAAGACAGTACTAAATGGAGAAAATTAGTAGATTTCAGAGAACTTA
ATAAGAGAACTCAAGATTTCTGGGAAGTTCAATTAGGAATACCACATCC
TGCAGGGTTAAAACAGAAAAAATCAGTAACAGTACTGGATGTGGGCGAT
GCATATTTTTCAGTTCCCTTAGATAAAGACTTCAGGAAGTATACTGCAT
TTACCATACCTAGTATAAACAATGAGACACCAGGGATTAGATATCAGTA
CAATGTGCTTCCACAGGGATGGAAAGGATCACCAGCAATATTCCAGTGT
AGCATGACAAAAATCTTAGAGCCTTTTAGAAAACAAAATCCAGACATAG
TCATCTATCAATACATGGATGATTTGTATGTAGGATCTGACTTAGAAAT
AGGGCAGCATAGAACAAAAATAGAGGAACTGAGACAACATCTGTTGAGG
TGGGGATTTACCACACCAGACAAAAAACATCAGAAAGAACCTCCATTCC
TTTGGATGGGTTATGAACTCCATCCTGATAAATGGACAGTACAGCCTAT
AGTGCTGCCAGAAAAGGACAGCTGGACTGTCAATGACATACAGAAATTA
GTGGGAAAATTGAATTGGGCAAGTCAGATTTATGCAGGGATTAAAGTAA
GGCAATTATGTAAACTTCTTAGGGGAACCAAAGCACTAACAGAAGTAGT
ACCACTAACAGAAGAAGCAGAGCTAGAACTGGCAGAAAACAGGGAGATT
CTAAAAGAACCGGTACATGGAGTGTATTATGACCCATCAAAAGACTTAA
TAGCAGAAATACAGAAGCAGGGGCAAGGCCAATGGACATATCAAATTTA
TCAAGAGCCATTTAAAAATCTGAAAACAGGAAAGTATGCAAGAATGAAG
GGTGCCCACACTAATGATGTGAAACAATTAACAGAGGCAGTACAAAAAA
TAGCCACAGAAAGCATAGTAATATGGGGAAAGACTCCTAAATTTAAATT
ACCCATACAAAAGGAAACATGGGAAGCATGGTGGACAGAGTATTGGCAA
GCCACCTGGATTCCTGAGTGGGAGTTTGTCAATACCCCTCCCTTAGTGA
AGTTATGGTACCAGTTAGAGAAAGAACCCATAATAGGAGCAGAAACTTT
CTATGTAGATGGGGCAGCCAATAGGGAAACTAAATTAGGAAAAGCAGGA
TATGTAACTGACAGAGGAAGACAAAAAGTTGTCCCCCTAACGGACACAA
CAAATCAGAAGACTGAGTTACAAGCAATTCATCTAGCTTTGCAGGATTC
GGGATTAGAAGTAAACATAGTGACAGACTCACAATATGCATTGGGAATC
ATTCAAGCACAACCAGATAAGAGTGAATCAGAGTTAGTCAGTCAAATAA
TAGAGCAGTTAATAAAAAAGGAAAAAGTCTACCTGGCATGGGTACCAGC
ACACAAAGGAATTGGAGGAAATGAACAAGTAGATAAATTGGTCAGTGCT
GGAATCAGGAAAGTACTATTTTTAGATGGAATAGATAAGGCCCAAGAAG
AACATGAGAAATATCACAGTAATTGGAGAGCAATGGCTAGTGATTTTAA
CCTACCACCTGTAGTAGCAAAAGAAATAGTAGCCAGCTGTGATAAATGT
CAGCTAAAAGGGGAAGCCATGCATGGACAAGTAGACTGTAGCCCAGGAA
TATGGCAGCTAGATTGTACACATTTAGAAGGAAAAGTTATCTTGGTAGC
AGTTCATGTAGCCAGTGGATATATAGAAGCAGAAGTAATTCCAGCAGAG
ACAGGGCAAGAAACAGCATACTTCCTCTTAAAATTAGCAGGAAGATGGC
CAGTAAAAACAGTACATACAGACAATGGCAGCAATTTCACCAGTACTAC
AGTTAAGGCCGCCTGTTGGTGGGCGGGGATCAAGCAGGAATTTGGCATT
CCCTACAATCCCCAAAGTCAAGGAGTAATAGAATCTATGAATAAAGAAT
TAAAGAAAATTATAGGACAGGTAAGAGATCAGGCTGAACATCTTAAGAC
AGCAGTACAAATGGCAGTATTCATCCACAATTTTAAAAGAAAAGGGGGG
ATTGGGGGGTACAGTGCAGGGGAAAGAATAGTAGACATAATAGCAACAG
ACATACAAACTAAAGAATTACAAAAACAAATTACAAAAATTCAAAATTT
TCGGGTTTATTACAGGGACAGCAGAGATCCAGTTTGGAAAGGACCAGCA
AAGCTCCTCTGGAAAGGTGAAGGGGCAGTAGTAATACAAGATAATAGTG
ACATAAAAGTAGTGCCAAGAAGAAAAGCAAAGATCATCAGGGATTATGG
AAAACAGATGGCAGGTGATGATTGTGTGGCAAGTAGACAGGATGAGGAT
TAA

Next, a DNA fragment containing the Rev, RRE, and rabbit beta globin poly A sequence with Xbat and Xmat flanking restriction sites was synthesized by MWG Operon. The DNA fragment was then inserted into the plasmid at the Xbat and Xmat restriction sites The DNA sequence was as follows:

(SEQ ID NO: 71)
TCTAGAATGGCAGGAAGAAGCGGAGACAGCGACGAAGAGCTCATCAG
AACAGTCAGACTCATCAAGCTTCTCTATCAAAGCAACCCACCTCCCAATC
CCGAGGGGACCCGACAGGCCCGAAGGAATAGAAGAAGAAGGTGGAGAGAG
AGACAGAGACAGATCCATTCGATTAGTGAACGGATCCTTGGCACTTATCT
GGGACGATCTGCGGAGCCTGTGCCTCTTCAGCTACCACCGCTTGAGAGAC
TTACTCTTGATTGTAACGAGGATTGTGGAACTTCTGGGACGCAGGGGGTG
GGAAGCCCTCAAATATTGGTGGAATCTCCTACAATATTGGAGTCAGGAGC
TAAAGAATAGAGGAGCTTTGTTCCTTGGGTTCTTGGGAGCAGCAGGAAGC
ACTATGGGCGCAGCGTCAATGACGCTGACGGTACAGGCCAGACAATTATT
GTCTGGTATAGTGCAGCAGCAGAACAATTTGCTGAGGGCTATTGAGGCGC
AACAGCATCTGTTGCAACTCACAGTCTGGGGCATCAAGCAGCTCCAGGCA
AGAATCCTGGCTGTGGAAAGATACCTAAAGGATCAACAGCTCCTAGATCT
TTTTCCCTCTGCCAAAAATTATGGGGACATCATGAAGCCCCTTGAGCATC
TGACTTCTGGCTAATAAAGGAAATTTATTTTCATTGCAATAGTGTGTTGG
AATTTTTTGTGTCTCTCACTCGGAAGGACATATGGGAGGGCAAATCATTT
AAAACATCAGAATGAGTATTTGGTTTAGAGTTTGGCAACATATGCCATAT
GCTGGCTGCCATGAACAAAGGTGGCTATAAAGAGGTCATCAGTATATGAA
ACAGCCCCCTGCTGTCCATTCCTTATTCCATAGAAAAGCCTTGACTTGAG
GTTAGATTTTTTTTATATTTTGTTTTGTGTTATTTTTTTCTTTAACATCC
CTAAAATTTTCCTTACATGTTTTACTAGCCAGATTTTTCCTCCTCTCCTG
ACTACTCCCAGTCATAGCTGTCCCTCTTCTCTTATGAAGATCCCTCGACC
TGCAGCCCAAGCTTGGCGTAATCATGGTCATAGCTGTTTCCTGTGTGAAA
TTGTTATCCGCTCACAATTCCACACAACATACGAGCCGGAAGCATAAAGT
GTAAAGCCTGGGGTGCCTAATGAGTGAGCTAACTCACATTAATTGCGTTG
CGCTCACTGCCCGCTTTCCAGTCGGGAAACCTGTCGTGCCAGCGGATCCG
CATCTCAATTAGTCAGCAACCATAGTCCCGCCCCTAACTCCGCCCATCCC
GCCCCTAACTCCGCCCAGTTCCGCCCATTCTCCGCCCCATGGCTGACTAA
TTTTTTTTATTTATGCAGAGGCCGAGGCCGCCTCGGCCTCTGAGCTATTC
CAGAAGTAGTGAGGAGGCTTTTTTGGAGGCCTAGGCTTTTGCAAAAAGCT
AACTTGTTTATTGCAGCTTATAATGGTTACAAATAAAGCAATAGCATCAC
AAATTTCACAAATAAAGCATTTTTTTCACTGCATTCTAGTTGTGGTTTGT
CCAAACTCATCAATGTATCTTATCAGCGGCCGCCCCGGG

Finally, the CMV promoter of pCDNA3.1 was replaced with the CAG enhancer/promoter plus a chicken beta actin intron sequence. A DNA fragment containing the CAG enhancer/promoter/intron sequence with MluI and EcoRI flanking restriction sites was synthesized by MWG Operon. The DNA fragment was then inserted into the plasmid at the MluI and EcoRI restriction sites. The DNA sequence was as follows:

(SEQ ID NO: 72)
ACGCGTTAGTTATTAATAGTAATCAATTACGGGGTCATTAGTTCATAGC
CCATATATGGAGTTCCGCGTTACATAACTTACGGTAAATGGCCCGCCTGG
CTGACCGCCCAACGACCCCCGCCCATTGACGTCAATAATGACGTATGTTC
CCATAGTAACGCCAATAGGGACTTTCCATTGACGTCAATGGGTGGACTAT
TTACGGTAAACTGCCCACTTGGCAGTACATCAAGTGTATCATATGCCAAG
TACGCCCCCTATTGACGTCAATGACGGTAAATGGCCCGCCTGGCATTATG
CCCAGTACATGACCTTATGGGACTTTCCTACTTGGCAGTACATCTACGTA
TTAGTCATCGCTATTACCATGGGTCGAGGTGAGCCCCACGTTCTGCTTCA
CTCTCCCCATCTCCCCCCCCTCCCCACCCCCAATTTTGTATTTATTTATT
TTTTAATTATTTTGTGCAGCGATGGGGGCGGGGGGGGGGGGGGCGCGCGC
CAGGCGGGGCGGGGCGGGGCGAGGGGCGGGGCGGGGCGAGGCGGAGAGGT
GCGGCGGCAGCCAATCAGAGCGGCGCGCTCCGAAAGTTTCCTTTTATGGC
GAGGCGGCGGCGGCGGCGGCCCTATAAAAAGCGAAGCGCGCGGCGGGCGG
GAGTCGCTGCGTTGCCTTCGCCCCGTGCCCCGCTCCGCGCCGCCTCGCGC
CGCCCGCCCCGGCTCTGACTGACCGCGTTACTCCCACAGGTGAGCGGGCG
GGACGGCCCTTCTCCTCCGGGCTGTAATTAGCGCTTGGTTTAATGACGGC
TCGTTTCTTTTCTGTGGCTGCGTGAAAGCCTTAAAGGGCTCCGGGAGGGC
CCTTTGTGCGGGGGGGAGCGGCTCGGGGGGTGCGTGCGTGTGTGTGTGCG
TGGGGAGCGCCGCGTGCGGCCCGCGCTGCCCGGCGGCTGTGAGCGCTGCG
GGCGCGGCGCGGGGCTTTGTGCGCTCCGCGTGTGCGCGAGGGGAGCGCGG
CCGGGGGCGGTGCCCCGCGGTGCGGGGGGGCTGCGAGGGGAACAAAGGCT
GCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACG
GCCCGGCTTCGGGTGCGGGGCTCCGTGCGGGGCGTGGCGCGGGGCTCGCC
GTGCCGGGCGGGGGGTGGCGGCAGGTGGGGGTGCCGGGCGGGGCGGGGCC
GCCTCGGGCCGGGGAGGGCTCGGGGGAGGGGCGCGGCGGCCCCGGAGCGC
CGGCGGCTGTCGAGGCGCGGCGAGCCGCAGCCATTGCCTTTTATGGTAAT
CGTGCGAGAGGGCGCAGGGACTTCCTTTGTCCCAAATCTGGCGGAGCCGA
AATCTGGGAGGCGCCGCCGCACCCCCTCTAGCGGGCGCGGGCGAAGCGGT
GCGGCGCCGGCAGGAAGGAAATGGGCGGGGAGGGCCTTCGTGCGTCGCCG
CGCCGCCGTCCCCTTCTCCATCTCCAGCCTCGGGGCTGCCGCAGGGGGAC
GGCTGCCTTCGGGGGGGACGGGGCAGGGCGGGGTTCGGCTTCTGGCGTGT
GACCGGCGGGAATTC

Construction of the VSV-G Envelope plasmid:

The vesicular stomatitis Indiana virus glycoprotein (VSV-G) sequence was synthesized by MWG Operon with flanking EcoRI restriction sites. The DNA fragment was then inserted into the pCDNA3.1 plasmid (Invitrogen) at the EcoRI restriction site and the correct orientation was determined by sequencing using a CMV specific primer. The DNA sequence was as follows:

(SEQ ID NO: 73)
GAATTCATGAAGTGCCTTTTGTACTTAGCCTTTTTATTCATTGGGGTGA
ATTGCAAGTTCACCATAGTTTTTCCACACAACCAAAAAGGAAACTGGAAA
AATGTTCCTTCTAATTACCATTATTGCCCGTCAAGCTCAGATTTAAATTG
GCATAATGACTTAATAGGCACAGCCTTACAAGTCAAAATGCCCAAGAGTC
ACAAGGCTATTCAAGCAGACGGTTGGATGTGTCATGCTTCCAAATGGGTC
ACTACTTGTGATTTCCGCTGGTATGGACCGAAGTATATAACACATTCCAT
CCGATCCTTCACTCCATCTGTAGAACAATGCAAGGAAAGCATTGAACAAA
CGAAACAAGGAACTTGGCTGAATCCAGGCTTCCCTCCTCAAAGTTGTGGA
TATGCAACTGTGACGGATGCCGAAGCAGTGATTGTCCAGGTGACTCCTCA
CCATGTGCTGGTTGATGAATACACAGGAGAATGGGTTGATTCACAGTTCA
TCAACGGAAAATGCAGCAATTACATATGCCCCACTGTCCATAACTCTACA
ACCTGGCATTCTGACTATAAGGTCAAAGGGCTATGTGATTCTAACCTCAT
TTCCATGGACATCACCTTCTTCTCAGAGGACGGAGAGCTATCATCCCTGG
GAAAGGAGGGCACAGGGTTCAGAAGTAACTACTTTGCTTATGAAACTGGA
GGCAAGGCCTGCAAAATGCAATACTGCAAGCATTGGGGAGTCAGACTCCC
ATCAGGTGTCTGGTTCGAGATGGCTGATAAGGATCTCTTTGCTGCAGCCA
GATTCCCTGAATGCCCAGAAGGGTCAAGTATCTCTGCTCCATCTCAGACC
TCAGTGGATGTAAGTCTAATTCAGGACGTTGAGAGGATCTTGGATTATTC
CCTCTGCCAAGAAACCTGGAGCAAAATCAGAGCGGGTCTTCCAATCTCTC
CAGTGGATCTCAGCTATCTTGCTCCTAAAAACCCAGGAACCGGTCCTGCT
TTCACCATAATCAATGGTACCCTAAAATACTTTGAGACCAGATACATCAG
AGTCGATATTGCTGCTCCAATCCTCTCAAGAATGGTCGGAATGATCAGTG
GAACTACCACAGAAAGGGAACTGTGGGATGACTGGGCACCATATGAAGAC
GTGGAAATTGGACCCAATGGAGTTCTGAGGACCAGTTCAGGATATAAGTT
TCCTTTATACATGATTGGACATGGTATGTTGGACTCCGATCTTCATCTTA
GCTCAAAGGCTCAGGTGTTCGAACATCCTCACATTCAAGACGCTGCTTCG
CAACTTCCTGATGATGAGAGTTTATTTTTTGGTGATACTGGGCTATCCAA
AAATCCAATCGAGCTTGTAGAAGGTTGGTTCAGTAGTTGGAAAAGCTCTA
TTGCCTCTTTTTTCTTTATCATAGGGTTAATCATTGGACTATTCTTGGTT
CTCCGAGTTGGTATCCATCTTTGCATTAAATTAAAGCACACCAAGAAAAG
ACAGATTTATACAGACATAGAGATGAGAATTC

A 4-vector system (i.e., a 3-vector lentiviral packaging system) has also been designed and produced using the methods and materials described herein. A schematic of the 4-vector system is shown in FIG. 5. Briefly, and with reference to FIG. 5, the top-most vector is a helper plasmid, which, in this case, does not include Rev. The vector second from the top is a separate Rev plasmid. The vector second from the bottom is the envelope plasmid. The bottom-most vector is the previously described therapeutic vector.

Referring, in part, to FIG. 5, the Helper plasmid includes a CAG enhancer (SEQ ID NO: 39); a CAG promoter (SEQ ID NO: 40); a chicken beta actin intron (SEQ ID NO: 41); a HIV gag (SEQ ID NO: 42); a HIV Pol (SEQ ID NO: 43); a HIV Int (SEQ ID NO: 44); a HIV RRE (SEQ ID NO: 45); and a rabbit beta globin poly A (SEQ ID NO: 47).

The Rev plasmid includes a RSV promoter and a HIV Rev (SEQ ID NO: 75); and a rabbit beta globin poly A (SEQ ID NO: 60).

The Envelope plasmid includes a CMV promoter (SEQ ID NO: 49); a beta globin intron (SEQ ID NO: 50); a VSV-G (SEQ ID NO: 51); and a rabbit beta globin poly A (SEQ ID NO: 56).

Synthesis of a 3-Vector Lentiviral Packaging System Including Helper, Rev, and Envelope Plasmids.

Materials and Methods:

Construction of the Helper plasmid without Rev:

The Helper plasmid without Rev was constructed by inserting a DNA fragment containing the RRE and rabbit beta globin poly A sequence. This sequence was synthesized by MWG Operon with flanking XbaI and XmaI restriction sites. The RRE/rabbit poly A beta globin sequence was then inserted into the Helper plasmid at the XbaI and XmaI restriction sites. The DNA sequence is as follows:

(SEQ ID NO: 74)
TCTAGAAGGAGCTTTGTTCCTTGGGTTCTTGGGAGCAGCAGGAAGCAC
TATGGGCGCAGCGTCAATGACGCTGACGGTACAGGCCAGACAATTATTGT
CTGGTATAGTGCAGCAGCAGAACAATTTGCTGAGGGCTATTGAGGCGCAA
CAGCATCTGTTGCAACTCACAGTCTGGGGCATCAAGCAGCTCCAGGCAAG
AATCCTGGCTGTGGAAAGATACCTAAAGGATCAACAGCTCCTAGATCTTT
TTCCCTCTGCCAAAAATTATGGGGACATCATGAAGCCCCTTGAGCATCTG
ACTTCTGGCTAATAAAGGAAATTTATTTTCATTGCAATAGTGTGTTGGAA
TTTTTTGTGTCTCTCACTCGGAAGGACATATGGGAGGGCAAATCATTTAA
AACATCAGAATGAGTATTTGGTTTAGAGTTTGGCAACATATGCCATATGC
TGGCTGCCATGAACAAAGGTGGCTATAAAGAGGTCATCAGTATATGAAAC
AGCCCCCTGCTGTCCATTCCTTATTCCATAGAAAAGCCTTGACTTGAGGT
TAGATTTTTTTTATATTTTGTTTTGTGTTATTTTTTTCTTTAACATCCCT
AAAATTTTCCTTACATGTTTTACTAGCCAGATTTTTCCTCCTCTCCTGAC
TACTCCCAGTCATAGCTGTCCCTCTTCTCTTATGAAGATCCCTCGACCTG
CAGCCCAAGCTTGGCGTAATCATGGTCATAGCTGTTTCCTGTGTGAAATT
GTTATCCGCTCACAATTCCACACAACATACGAGCCGGAAGCATAAAGTGT
AAAGCCTGGGGTGCCTAATGAGTGAGCTAACTCACATTAATTGCGTTGCG
CTCACTGCCCGCTTTCCAGTCGGGAAACCTGTCGTGCCAGCGGATCCGCA
TCTCAATTAGTCAGCAACCATAGTCCCGCCCCTAACTCCGCCCATCCCGC
CCCTAACTCCGCCCAGTTCCGCCCATTCTCCGCCCCATGGCTGACTAATT
TTTTTTATTTATGCAGAGGCCGAGGCCGCCTCGGCCTCTGAGCTATTCCA
GAAGTAGTGAGGAGGCTTTTTTGGAGGCCTAGGCTTTTGCAAAAAGCTAA
CTTGTTTATTGCAGCTTATAATGGTTACAAATAAAGCAATAGCATCACAA
ATTTCACAAATAAAGCATTTTTTTCACTGCATTCTAGTTGTGGTTTGTCC
AAACTCATCAATGTATCTTATCACCCGGG

Construction of the Rev Plasmid:

The RSV promoter and HIV Rev sequence was synthesized as a single DNA fragment by MWG Operon with flanking MfeI and XbaI restriction sites. The DNA fragment was then inserted into the pCDNA3.1 plasmid (Invitrogen) at the MfeI and XbaI restriction sites in which the CMV promoter is replaced with the RSV promoter. The DNA sequence was as follows:

(SEQ ID NO: 75)
CAATTGCGATGTACGGGCCAGATATACGCGTATCTGAGGGGACTAGGG
TGTGTTTAGGCGAAAAGCGGGGCTTCGGTTGTACGCGGTTAGGAGTCCCC
TCAGGATATAGTAGTTTCGCTTTTGCATAGGGAGGGGGAAATGTAGTCTT
ATGCAATACACTTGTAGTCTTGCAACATGGTAACGATGAGTTAGCAACAT
GCCTTACAAGGAGAGAAAAAGCACCGTGCATGCCGATTGGTGGAAGTAAG
GTGGTACGATCGTGCCTTATTAGGAAGGCAACAGACAGGTCTGACATGGA
TTGGACGAACCACTGAATTCCGCATTGCAGAGATAATTGTATTTAAGTGC
CTAGCTCGATACAATAAACGCCATTTGACCATTCACCACATTGGTGTGCA
CCTCCAAGCTCGAGCTCGTTTAGTGAACCGTCAGATCGCCTGGAGACGCC
ATCCACGCTGTTTTGACCTCCATAGAAGACACCGGGACCGATCCAGCCTC
CCCTCGAAGCTAGCGATTAGGCATCTCCTATGGCAGGAAGAAGCGGAGAC
AGCGACGAAGAACTCCTCAAGGCAGTCAGACTCATCAAGTTTCTCTATCA
AAGCAACCCACCTCCCAATCCCGAGGGGACCCGACAGGCCCGAAGGAATA
GAAGAAGAAGGTGGAGAGAGAGACAGAGACAGATCCATTCGATTAGTGAA
CGGATCCTTAGCACTTATCTGGGACGATCTGCGGAGCCTGTGCCTCTTCA
GCTACCACCGCTTGAGAGACTTACTCTTGATTGTAACGAGGATTGTGGAA
CTTCTGGGACGCAGGGGGTGGGAAGCCCTCAAATATTGGTGGAATCTCCT
ACAATATTGGAGTCAGGAGCTAAAGAATAGTCTAGA

The plasmids for the 2-vector and 3-vector packaging systems could be modified with similar elements and the intron sequences could potentially be removed without loss of vector function. For example, the following elements could replace similar elements in the 2-vector and 3-vector packaging system:

Promoters: Elongation Factor-1 (EF-1) (SEQ ID NO: 4), phosphoglycerate kinase (PGK) (SEQ ID NO: 52), and ubiquitin C (UbC) (SEQ ID NO: 53) can replace the CMV (SEQ ID NO: 49) or CAG promoter (SEQ ID NO: 80). These sequences can also be further varied by addition, substitution, deletion or mutation.

Poly A sequences: SV40 poly A (SEQ ID NO: 54) and bGH poly A (SEQ ID NO: 55) can replace the rabbit beta globin poly A (SEQ ID NO: 47). These sequences can also be further varied by addition, substitution, deletion or mutation.

HIV Gag, Pol, and Integrase sequences: The HIV sequences in the Helper plasmid can be constructed from different HIV strains or clades. For example, HIV Gag (SEQ ID NO: 56); HIV Pol (SEQ ID NO: 57); and HIV Int (SEQ ID NO: 58) from the Bal strain can be interchanged with the gag, pol, and int sequences contained in the helper/helper plus Rev plasmids as outlined herein. These sequences can also be further varied by addition, substitution, deletion or mutation.

Envelope: The VSV-G glycoprotein can be substituted with membrane glycoproteins from feline endogenous virus (RD114) (SEQ ID NO: 59), gibbon ape leukemia virus (GALV) (SEQ ID NO: 60), Rabies (FUG) (SEQ ID NO: 61), lymphocytic choriomeningitis virus (LCMV) (SEQ ID NO: 62), influenza A fowl plague virus (FPV) (SEQ ID NO: 63), Ross River alphavirus (RRV) (SEQ ID NO: 64), murine leukemia virus 10A1 (MLV) (SEQ ID NO: 65), or Ebola virus (EboV) (SEQ ID NO: 66). Sequences for these envelopes are identified in the sequence portion herein. Further, these sequences can also be further varied by addition, substitution, deletion or mutation.

In summary, the 3-vector versus 4-vector systems can be compared and contrasted, in part, as follows. The 3-vector lentiviral vector system contains: 1. Helper plasmid: HIV Gag, Pol, Integrase, and Rev/Tat; 2. Envelope plasmid: VSV-G/FUG envelope; and 3. Therapeutic vector: RSV 5′LTR, Psi Packaging Signal, Gag fragment, RRE, Env fragment, cPPT, WPRE, and 3′delta LTR. The 4-vector lentiviral vector system contains: 1. Helper plasmid: HIV Gag, Pol, and Integrase; 2. Rev plasmid: Rev; 3. Envelope plasmid: VSV-G/FUG envelope; and 4. Therapeutic vector: RSV 5′LTR, Psi Packaging Signal, Gag fragment, RRE, Env fragment, cPPT, WPRE, and 3′ delta LTR. Sequences corresponding with the above elements are identified in the sequence listings portion herein.

### Example 2: Development of an Anti-HIV Lentivirus Vector

The purpose of this example was to develop an anti-HIV lentivirus vector.

Inhibitory RNA Designs. The sequence of Homo sapiens chemokine C-C motif receptor 5 (CCR5) (GC03P046377) mRNA was used to search for potential siRNA or shRNA candidates to knockdown CCR5 levels in human cells. Potential RNA interference sequences were chosen from candidates selected by siRNA or shRNA design programs such as from the Broad Institute or the BLOCK-iT RNAi Designer from Thermo Scientific. Individual selected shRNA sequences were inserted into lentiviral vectors immediately 3′ to a RNA polymerase III promoter such as H1, U6, or 7SK to regulate shRNA expression. These lentivirus-shRNA constructs were used to transduce cells and measure the change in specific mRNA levels. The shRNA most potent for reducing mRNA levels were embedded individually within a microRNA backbone to allow for expression by either the CMV or EF-1 alpha RNA polymerase II promoters. The microRNA backbone was selected from mirbase.org. RNA sequences were also synthesized as synthetic siRNA oligonucleotides and introduced directly into cells without using a lentiviral vector.

The genomic sequence of Bal strain of human immunodeficiency virus type 1 (HIV-1 85US_BaL, accession number AY713409) was used to search for potential siRNA or shRNA candidates to knockdown HIV replication levels in human cells. Based on sequence homology and experience, the search focused on regions of the Tat and Vif genes of HIV although an individual of skill in the art will understand that use of these regions is non-limiting and other potential targets might be selected. Importantly, highly conserved regions of gag or pol genes could not be targeted by shRNA because these same sequences were present in the packaging system complementation plasmids needed for vector manufacturing. As with the CCR5 (NM 000579.3, NM 001100168.1-specific) RNAs, potential HIV-specific RNA interference sequences were chosen from candidates selected by siRNA or shRNA design programs such as from the Gene-E Software Suite hosted by the Broad Institute (broadinstitute.org/mai/public) or the BLOCK-iT RNAi Designer from Thermo Scientific (rnadesigner.thermofisher, com/rnai expres s/s etOpti on, do? designOption=shrna&pid=6712627360706061801). Individual selected shRNA sequences were inserted into lentiviral vectors immediately 3′ to a RNA polymerase III promoter such as H1, U6, or 7SK to regulate shRNA expression. These lentivirus-shRNA constructs were used to transduce cells and measure the change in specific mRNA levels. The shRNA most potent for reducing mRNA levels were embedded individually within a microRNA backbone to allow for expression by either the CMV or EF-1alpha RNA polymerase II promoters.

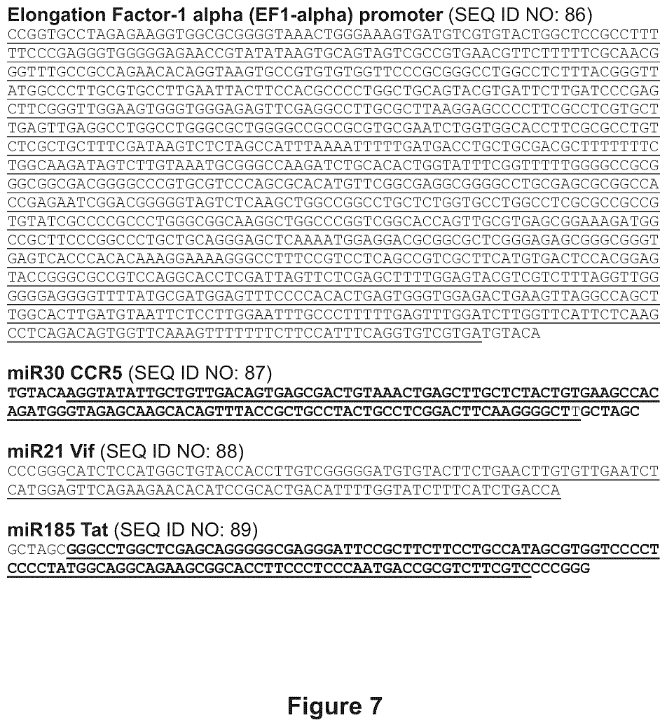

Vector Constructions. For CCR5, Tat or Vif shRNA, oligonucleotide sequences containing BamHI and EcoRI restriction sites were synthesized by Eurofins MWG Operon, LLC. Overlapping sense and antisense oligonucleotide sequences were mixed and annealed during cooling from 70 degrees Celsius to room temperature. The lentiviral vector was digested with the restriction enzymes BamHI and EcoRI for one hour at 37 degrees Celsius. The digested lentiviral vector was purified by agarose gel electrophoresis and extracted from the gel using a DNA gel extraction kit from Invitrogen. The DNA concentrations were determined and vector to oligo (3:1 ratio) were mixed, allowed to anneal, and ligated. The ligation reaction was performed with T4 DNA ligase for 30 minutes at room temperature. 2.5 microliters of the ligation mix were added to 25 microliters of STBL3 competent bacterial cells. Transformation was achieved after heat-shock at 42 degrees Celsius. Bacterial cells were spread on agar plates containing ampicillin and drug-resistant colonies (indicating the presence of ampicillin-resistance plasmids) were recovered, purified and expanded in LB broth. To check for insertion of the oligo sequences, plasmid DNA were extracted from harvested bacteria cultures with the Invitrogen DNA mini prep kit. Insertion of the shRNA sequence in the lentiviral vector was verified by DNA sequencing using a specific primer for the promoter used to regulate shRNA expression. Exemplary vector sequences that were determined to restrict HIV replication can be found in FIG. 7. The shRNA sequences with the highest activity against CCR5, Tat, and Vif gene expression were assembled into the miR30, miR21, and miR185 backbones. This resulted in miR30 CCR5 (SEQ ID NO: 87), miR21 Vif (SEQ ID NO: 88), and miR185 Tat (SEQ ID NO: 89) sequences. The underlined sequences of SEQ ID NOS: 87, 88, and 89 represent the backbones with the shRNA sequences. The sequences that are not underlined of SEQ ID NOS: 87, 88, 89 represent the restriction recognition sequences.

Development of Vectors

It should be noted that not all vectors developed for these experiments necessarily worked as might be predicted. More specifically, a lentivirus vector against HIV might include three main components: 1) inhibitory RNA to reduce the level of HIV binding proteins (receptors) on the target cell surface to block initial virus attachment and penetration; 2) overexpression of the HIV TAR sequence that will sequester viral Tat protein and decrease its ability to transactivate viral gene expression; and 3) inhibitory RNA that attack important and conserved sequences within the HIV genome.

With respect to the first point above, a key cell surface HIV binding protein is the chemokine receptor CCR5. HIV particles attach to susceptible T cells by binding to the CD4 and CCR5 cell surface proteins. Because CD4 is an essential glycoprotein on the cell surface that is important for the immunological function of T cells, this was not chosen as a target to manipulate its expression levels. However, people born homozygous for null mutations in the CCR5 gene and completely lacking receptor expression, live normal lives save for enhanced susceptibility to a few infectious diseases and the possibility of developing rare autoimmunity. Thus, modulating CCR5 was determined to be a relatively safe approach and was a primary target in the development of anti-HIV lentivirus vectors.

With respect to the second point above, the viral TAR sequence is a highly structured region of HIV genomic RNA that binds tightly to viral Tat protein. The Tat:TAR complex is important for efficient generation of viral RNA. Over-expression of the TAR region was envisioned as a decoy molecule that would sequester Tat protein and decrease the levels of viral RNA. However, TAR proved toxic to most mammalian cells including cells used for manufacturing lentivirus particles. Further, TAR was inefficient for inhibiting viral gene expression in other laboratories and has been discarded as a viable component in HIV gene therapy.

In various embodiments, viral gene sequences have been identified that meet 3 criteria: i) Sequences that are reasonably conserved across a range of HIV isolates representative of the epidemic in a geographic region of interest; ii) reduction in RNA levels due to the activity of an inhibitory RNA in a viral vector will reduce the corresponding protein levels by an amount sufficient to meaningfully reduce HIV replication; and iii) the viral gene sequence(s) targeted by inhibitory RNA are not present in the genes required for packaging and assembling viral vector particles during manufacturing. In various embodiments, a sequence at the junction of HIV Tat and Rev genes and a second sequence within the HIV Vif gene have been targeted by inhibitory RNA. The Tat/Rev targeting has an additional benefit of reducing HIV envelope glycoprotein expression because this region overlaps with the envelope gene in the HIV genome.

Various methods for vector development and testing relies first on identifying suitable targets (as described herein) followed by constructing plasmid DNAs expressing individual or multiple inhibitory RNA species for testing in cell models, and finally constructing lentivirus vectors containing inhibitory RNA with proven anti-HIV function. The lentivirus vectors are tested for toxicity, yield during in vitro production, and effectiveness against HIV in terms of reducing CCR5 expression levels or lowering viral gene products to inhibit virus replication.

Table 2 below demonstrates progression through multiple versions of inhibitory constructs until arriving at a clinical candidate. Initially, shRNA (short homology RNA) molecules were designed and expressed from plasmid DNA constructs.

Plasmids 1-4, as detailed in Table 2 below, tested shRNA sequences against Gag, Pol and RT genes of HIV. While each shRNA was active for suppressing viral protein expression in a cell model, there were two important problems that prevented further development. First, the sequences were targeted to a laboratory isolate of HIV that was not representative of Clade B HIV strains currently circulating in North America and Europe. Second, these shRNA targeted critical components in the lentivirus vector packaging system and would severely reduce vector yield during manufacturing. Plasmid 5, as detailed in Table 2, was selected to target CCR5 and provided a lead candidate sequence. Plasmids 6, 7, 8, 9, 10, and 11, as detailed in Table 2, incorporated the TAR sequence and it was found they produced unacceptable toxicity for mammalian cells including cells used for lentivirus vector manufacturing. Plasmid 2, as detailed in Table 2, identified a lead shRNA sequence capable of reducing Tat RNA expression. Plasmid 12, as detailed in Table 2, demonstrated the effectiveness of shCCR5 expressed as a microRNA (miR) in a lentiviral vector and confirmed it should be in the final product. Plasmid 13, as detailed in Table 2, demonstrated the effectiveness of a shVif expressed as a microRNA (miR) in a lentiviral vector and confirmed it should be in the final product. Plasmid 14, as detailed in Table 2, demonstrated the effectiveness of shTat expressed as a microRNA (miR) in a lentiviral vector and confirmed it should be in the final product. Plasmid 15, as detailed in Table 2, contained the miR CCR5, miR Tat and miR Vif in the form of a miR cluster expressed from a single promoter. These miR do not target critical components in the lentivirus vector packaging system and proved to have negligible toxicity for mammalian cells. The miRs within the cluster were equally effective to individual miR that were tested previously, and the overall impact was a substantial reduction in replication of a CCR5-tropic HIV BaL strain.

TABLE 2
Development of HIV Vectors

| Plasmid | Sequence | Material | Description | Remarks | Decision |
|---|---|---|---|---|---|
| 1 | SEQ ID NO: | Lentiviral | shRNA | Wrong target, lab | Abandon |
| | 9 | vector | construct for | virus, no virus test | |
| | | | RT of LAI | | |
| | | | strain | | |
| 2 | SEQ ID NO: | Lentiviral | H1 promoter | Tat protein knock- | Lead |
| | 76 | vector | shRNA | down >90% | |
| | | | Tat/Rev | | |
| | | | overlap | | |

Vector Construction: For Rev/Tat shRNA, oligonucleotide sequences containing BamHI and
EcoRI restriction sites were synthesized by MWG Operon. The two Rev/Tat shRNA
sequences that were synthesized were the Rev/Tat shRNA coding sequence #1 (5′-
GCGGAGACAGCGACGAAGAGCTTCAAGAGAGCTCTTCGTCGCTGTCTCCGCTTTT
T-3′) (SEQ ID NO: 10) and the Rev/Tat shRNA coding sequence #2 (5′-
ATGGCAGGAAGAAGCGGAGTTCAAGAGACTCCGCTTCTTCCTGCCATTTTTT-3′)
(SEQ ID NO: 77). Two different Rev/Tat target sequences were tested for their ability to
decrease Tat mRNA expression: The Rev/Tat shRNA target sequence #1 (5′-
GCGGAGACAGCGACGAAGAGC-3′) (SEQ ID NO: 9) and the Rev/Tat shRNA target
sequence #2 (5′-ATGGCAGGAAGAAGCGGAG-3′) (SEQ ID NO: 76).
Functional test for shRNA against Rev/Tat: Reduction in Tat expression was tested using a
luciferase reporter plasmid which contained a Rev/Tat target sequence inserted into the 3′-
UTR (untranslated region of the mRNA). Separate luciferase reporter plasmids containing
the Rev/Tat shRNA target sequence #1 (SEQ ID NO: 9) and the Rev/Tat shRNA target
sequence #2 (SEQ ID NO: 76) were tested. The Rev/Tat shRNA coding sequence #1 (SEQ
ID NO: 10) was co-transfected with a luciferase reporter plasmid containing the Rev/Tat
shRNA target sequence #1 (SEQ ID NO: 9). The Rev/Tat shRNA coding sequence #2 (SEQ
ID NO: 77) was co-transfected with a luciferase reporter plasmid containing the Rev/Tat
shRNA target sequence #2 (SEQ ID NO: 76).
There was a 90% reduction in light emission in the luciferase reporter plasmid containing the
Rev/Tat shRNA target sequence #1 (SEQ ID NO: 9). This is in contrast to the less than 10%
reduction in light emission in the luciferase reporter plasmid containing the Rev/Tat target
sequence #2 (SEQ ID NO: 76).
Conclusion: The Rev/Tat shRNA coding sequence #1 (SEQ ID NO: 10) was superior to the
RevTat shRNA coding sequence #2 (SEQ ID NO: 77) in terms of reducing mRNA levels in
the luciferase assay system. This indicates potent inhibitory activity of the Rev/Tat shRNA
coding sequence #1 (SEQ ID NO: 10) and it was selected as a lead candidate for further
development.

| | | | | | |
|---|---|---|---|---|---|
| 3 | SEQ ID NO: | Lentiviral | shRNA | Inhibits Gag | Abandon |
| | 12 | vector | construct for | expression but will | |
| | | | LAI Gag | inhibit packaging | |

Vector Construction: For Gag shRNA, oligonucleotide sequences containing BamHI and
EcoRI restriction sites were synthesized by MWG Operon, and tested for its ability to
decrease Gag mRNA expression. The Gag shRNA sequence that was synthesized was the
Gag shRNA coding sequence (5′-
GAAGAAATGATGACAGCATTTCAAGAGAATGCTGTCATCATTTCTTCTTTTT-3′)
(SEQ ID NO: 12). The Gag shRNA target sequence (5′-GAAGAAATGATGACAGCAT-
3′) (SEQ ID NO: 11) was tested for its ability to reduce Gag mRNA expression.
Oligonucleotide sequences were inserted into the pSIH lentiviral vector (System
Biosciences).
Functional test for shRNA against Gag: Reduction in Gag expression was tested using a
luciferase reporter plasmid which contained the Gag shRNA target sequence (SEQ ID NO:
11) inserted into the 3′-UTR (untranslated region of the mRNA). The Gag shRNA coding
sequence (SEQ ID NO: 12) was co-transfected with the luciferase reporter plasmid
containing luciferase and the Gag shRNA target sequence (SEQ ID NO: 11). Co-transfection
of these sequences resulted in nearly a 90% reduction in light emission from the luciferase
reporter plasmid compared to control treatments.
Conclusion: This Gag shRNA coding sequence (SEQ ID NO: 12) is potent against HIV Gag
expression, but was abandoned. The lentivirus packaging system requires production of Gag
from the helper plasmid and shRNA inhibition of Gag will reduce lentivirus vector yield.
This shRNA coding sequence could be used as an oligonucleotide inhibitor of HIV or
incorporated into an alternate viral vector packaging system that uses a different vector
genome or is modified to resist inhibition by this shRNA.

| | | | | | |
|---|---|---|---|---|---|
| 4 | SEQ ID NO: | Lentiviral | shRNA | Inhibits Pol | Abandon |
| | 14 | vector | construct for | expression but will | |
| | | | Pol | inhibit packaging | |

Vector Construction: A Pol shRNA was constructed with oligonucleotide sequences
containing BamHI and EcoRI restriction sites that were synthesized by MWG Operon, and
tested for its ability to decrease Pol mRNA expression. The Pol shRNA sequence that was
synthesized was the Pol shRNA coding sequence (5′-
CAGGAGATGATACAGTTCAAGAGACTGTATCATCTGCTCCTGTTTTT-3′) (SEQ ID
NO: 14). The Pol shRNA target sequence (5′-CAGGAGCAGATGATACAG-3′) (SEQ ID
NO: 13) was tested for its ability to reduce Pol mRNA expression. Oligonucleotide
sequences were inserted into the pSIH lentiviral vector (System Biosciences).
Functional tests for shRNA against HIV Pot: Reduction in Pol expression was tested using a
luciferase reporter plasmid which contained the Pol shRNA target sequence (SEQ ID NO:
13) inserted into the 3′-UTR (untranslated region of the mRNA). The Pol shRNA coding
sequence (SEQ ID NO: 14) was co-transfected with the luciferase reporter plasmid
containing luciferase and the Pol shRNA target sequence (SEQ ID NO: 13). Co-transfection
of these sequences resulted in a 60% reduction in light emission from the luciferase reporter
plasmid compared to control treatments.
Conclusion: This Pol shRNA coding sequence (SEQ ID NO: 14) is potent against HIV Pol
expression, but was abandoned. The lentivirus packaging system requires production of Pol
from the helper plasmid and shRNA inhibition of Pol will reduce lentivirus vector yield. This
shRNA sequence could be used as an oligonucleotide inhibitor of HIV or incorporated into an
alternate viral vector packaging system that uses a different vector genome or is modified to
resist inhibition by this shRNA.

| | | | | | |
|---|---|---|---|---|---|
| 5 | SEQ ID NO: | Lentiviral | shRNA | Best of 5 | Lead |
| | 16 | vector | construct for | candidates, | |
| | | | CCR5 | Extracellular | |
| | | | | CCR5 protein | |
| | | | | reduction >90% | |

Vector Construction: Various CCR5 shRNA coding sequences were constructed with
oligonucleotide sequences containing BamHI and EcoRI restriction sites that were
synthesized by MWG Operon. Oligonucleotide sequences were inserted into the pSIH
lentiviral vector (System Biosciences).
CCR5 shRNA target sequence #1 is (5′-GTGTCAAGTCCAATCTATG-3′) (SEQ ID NO:
15), which corresponds toCCR5 shRNA coding sequence #1 (5′-
GTGTCAAGTCCAATCTATGTTCAAGAGACATAGATTGGACTTGACACTTTTT-3′)
(SEQ ID NO: 16). CCR5 target sequence #1 (SEQ ID NO: 15) is located within the Homo
Sapiens CCR5 mRNA target sequence #1 (SEQ ID NO: 25).
CCR5 shRNA target sequence #2 is (5′-GAGCATGACTGACATCTAC-3′) (SEQ ID NO:
17), which corresponds to CCR5 shRNA coding sequence #2 (5′-
GAGCATGACTGACATCTACTTCAAGAGAGTAGATGTCAGTCATGCTCTTTTT-3′)
(SEQ ID NO: 18). CCR5 target sequence #2 (SEQ ID NO: 17) is located within the Homo
Sapiens CCR5 mRNA target sequence #2 (SEQ ID NO: 26).
CCR5 shRNA target sequence #3 is (5′-GTAGCTCTAACAGGTTGGA-3′) (SEQ ID NO:
19), which corresponds to CCR5 shRNA coding sequence #3 (5′-
GTAGCTCTAACAGGTTGGATTCAAGAGATCCAACCTGTTAGAGCTACTTTTT-3′)
(SEQ ID NO: 20). CCR5 target sequence #3 (SEQ ID NO: 19) is located within the Homo
Sapiens CCR5 mRNA target sequence #3 (SEQ ID NO: 27).
CCR5 shRNA target sequence #4 is (5′-GTTCAGAAACTACCTCTTA-3′) (SEQ ID NO:
21), which corresponds to CCR5 shRNA coding sequence #4 (5′-
GTTCAGAAACTACCTCTTATTCAAGAGATAAGAGGTAGTTTCTGAACTTTTT-3′)
(SEQ ID NO: 22). CCR5 target sequence #4 (SEQ ID NO: 21) is located within the Homo
Sapiens CCR5 mRNA target sequence #4 (SEQ ID NO: 28).
CCR5 shRNA target sequence #5 is (5′-GAGCAAGCTCAGTTTACACC-3′) (SEQ ID NO:
23), which corresponds to CCR5 shRNA coding sequence #5 (5′-
GAGCAAGCTCAGTTTACACCTTCAAGAGAGGTGTAAACTGAGCTTGCTCTTTTT-
3′) (SEQ ID NO: 24). CCR5 target sequence #5 (SEQ ID NO: 23) is located within the
Homo Sapiens CCR5 mRNA target sequence #5 (SEQ ID NO: 29).
Functional test for shRNA against CCR5: The ability of the CCR5 shRNA sequences to
knock-down CCR5 RNA expression was initially tested by separately co-transfecting each of
the CCR5 shRNA target sequences (SEQ ID NOS: 15, 17, 19, 21, and 23) with its
corresponding CCR5 shRNA coding sequence (SEQ ID NOS: 16, 18, 20, 22, 24, and 26).
CCR5 mRNA expression was then assessed by qPCR analysis using CCR5-specific primers.
Conclusion: Based on the reduction in CCR5 mRNA levels CCR5 shRNA coding sequence
#51 (SEQ ID NO: 16) was the most potent in reducing CCR5 gene expression. This shRNA
was selected as a lead candidate.

| | | | | | |
|---|---|---|---|---|---|
| 6 | SIH-U6- | Lentiviral | U6 promoter- | Toxic to cells | Abandon |
| | TAR | vector | TAR | | |
| 7 | SIH-U6- | Lentiviral | U6 promoter- | Toxic to cells | Abandon |
| | TAR-H1- | vector | TAR-H1- | | |
| | shCCR5 | | shCCR5 | | |
| 8 | U6-TAR- | Lentiviral | U6 promoter- | Suppress HIV, | Abandon |
| | H1-shRT | vector | TAR-H1-RT | toxic to cells, poor | |
| | | | | packaging | |
| 9 | U6-TAR- | Lentiviral | Change | Toxic, poor | Abandon |
| | 7SK-shRT | vector | shRNA | packaging | |
| | | | promoter to | | |
| | | | 7SK | | |
| 10 | U6-TAR- | Lentiviral | U6 promoter- | Toxic, poor | Abandon |
| | H1-shRT- | vector | TAR-Hl-RT- | packaging, H1 | |
| | H1-shCCR5 | | H1-shCCR5 | repeats | |
| 11 | U6-TAR- | Lentiviral | Change | Toxic, poor | Abandon |
| | 7SK-shRT- | vector | shRNA | packaging | |
| | H1-CCR5 | | promoter to | | |
| | | | 7SK | | |

Vector Construction: A TAR decoy sequence containing flanking KpnI restriction sites was
synthesized by MWG operon and inserted into the pSIH lentiviral vector (System
Biosciences) at the KpnI site. In this vector, TAR expression is regulated by the U6
promoter. The TAR decoy sequence is (5′-
CTTGCAATGATGTCGTAATTTGCGTCTTACCTCGTTCTCGACAGCGACCAGATCTG
AGCCTGGGAGCTCTCTGGCTGTCAGTAAGCTGGTACAGAAGGTTGACGAAAATT
CTTACTGAGCAAGAAA-3′) (SEQ ID NO: 8). Expression of the TAR decoy sequence was
determined by qPCR analysis using specific primers for the TAR sequence. Additional
vectors were constructed also containing the TAR sequence. The H1 promoter and shRT
sequence was inserted in this vector in the XhoI site. The H1 promoter and shRT sequence is
(5′-
GAACGCTGACGTCATCAACCCGCTCCAAGGAATCGCGGGCCCAGTGTCACTAGG
CGGGAACACCCAGCGCGCGTGCGCCCTGGCAGGAAGATGGCTGTGAGGGACAG
GGGAGTGGCGCCCTGCAATATTTGCATGTCGCTATGTGTTCTGGGAAATCACCAT
AAACGTGAAATGTCTTTGGATTTGGGAATCTTATAAGTTCTGTATGAGACCACTT
GGATCCGCGGAGACAGCGACGAAGAGCTTCAAGAGAGCTCTTCGTCGCTGTCTC
CGCTTTTT-3′) (SEQ ID NO: 78). This vector could express TAR and knockdown RT.
The 7SK promoter was also substituted for the H1 promoter to regulate shRT expression.
Another vector was constructed containing U6 TAR, H1 shRT, and H1 shCCR5. The H1
shCCR5 sequence was inserted into the SpeI site of the plasmid containing U6 TAR and H1
shRT. The H1 CCR5 sequence is (5′-
GAACGCTGACGTCATCAACCCGCTCCAAGGAATCGCGGGCCCAGTGTCACTAGG
CGGGAACACCCAGCGCGCGTGCGCCCTGGCAGGAAGATGGCTGTGAGGGACAG
GGGAGTGGCGCCCTGCAATATTTGCATGTCGCTATGTGTTCTGGGAAATCACCAT
AAACGTGAAATGTCTTTGGATTTGGGAATCTTATAAGTTCTGTATGAGACCACTT
GGATCCGTGTCAAGTCCAATCTATGTTCAAGAGACATAGATTGGACTTGACACTT
TTT-3′) (SEQ ID NO: 79). The 7SK promoter was also substituted for the H1 promoter to
regulate shRT expression.
Functional test for TAR decoy activity: We tested the effect of SIH-U6-TAR on packaging
efficiency. When TAR sequence was included, the yield of vector in the SIH packaging
system was reduced substantially.
Conclusion: Lentivirus vectors expressing the TAR decoy sequence are unsuitable for
commercial development due to low vector yields. These constructs were abandoned.

| | | | | | |
|---|---|---|---|---|---|
| 12 | SEQ ID NO: | Lentiviral | microRNA | Extracellular | Lead |
| | 1 | vector | sequence | CCR5 protein | |
| | | | | reduction >90% | |

Vector Construction: A CCR5 microRNA was constructed with oligonucleotide sequences
containing BsrGI and NotI restriction sites that were synthesized by MWG Operon.
Oligonucleotide sequences were inserted into the pCDH lentiviral vector (System
Biosciences). The EF-1 promoter was substituted for a CMV promoter that was used in the
plasmid construct Test Material 5. The EF-1 promoter was synthesized by MWG Operon
containing flanking ClaI and BsrGI restriction sites and inserted into the pCDH vector
containing shCCR5-1. The EF-1 promoter sequence is (5′-
CCGGTGCCTAGAGAAGGTGGCGCGGGGTAAACTGGGAAAGTGATGTCGTGTACT
GGCTCCGCCTTTTTCCCGAGGGTGGGGGAGAACCGTATATAAGTGCAGTAGTCGC
CGTGAACGTTCTTTTTCGCAACGGGTTTGCCGCCAGAACACAGGTAAGTGCCGTG
TGTGGTTCCCGCGGGCCTGGCCTCTTTACGGGTTATGGCCCTTGCGTGCCTTGAAT
TACTTCCACGCCCCTGGCTGCAGTACGTGATTCTTGATCCCGAGCTTCGGGTTGG
AAGTGGGTGGGAGAGTTCGAGGCCTTGCGCTTAAGGAGCCCCTTCGCCTCGTGCT
TGAGTTGAGGCCTGGCCTGGGCGCTGGGGCCGCCGCGTGCGAATCTGGTGGCAC
CTTCGCGCCTGTCTCGCTGCTTTCGATAAGTCTCTAGCCATTTAAAATTTTTGATG
ACCTGCTGCGACGCTTTTTTTCTGGCAAGATAGTCTTGTAAATGCGGGCCAAGAT
CTGCACACTGGTATTTCGGTTTTTGGGGCCGCGGGCGGCGACGGGGCCCGTGCGT
CCCAGCGCACATGTTCGGCGAGGCGGGGCCTGCGAGCGCGGCCACCGAGAATCG
GACGGGGGTAGTCTCAAGCTGGCCGGCCTGCTCTGGTGCCTGGCCTCGCGCCGCC
GTGTATCGCCCCGCCCTGGGCGGCAAGGCTGGCCCGGTCGGCACCAGTTGCGTG
AGCGGAAAGATGGCCGCTTCCCGGCCCTGCTGCAGGGAGCTCAAAATGGAGGAC
GCGGCGCTCGGGAGAGCGGGCGGGTGAGTCACCCACACAAAGGAAAAGGGCCT
TTCCGTCCTCAGCCGTCGCTTCATGTGACTCCACGGAGTACCGGGCGCCGTCCAG
GCACCTCGATTAGTTCTCGAGCTTTTGGAGTACGTCGTCTTTAGGTTGGGGGGAG
GGGTTTTATGCGATGGAGTTTCCCCACACTGAGTGGGTGGAGACTGAAGTTAGGC
CAGCTTGGCACTTGATGTAATTCTCCTTGGAATTTGCCCTTTTTGAGTTTGGATCT
TGGTTCATTCTCAAGCCTCAGACAGTGGTTCAAAGTTTTTTTCTTCCATTTCAGGT
GTCGTGA-3′) (SEQ ID NO: 4).
Functional test for lentivirus CDH-shCCR5-1: The ability of CCR5 miR and CCR5 shRNA
sequences to knock-down CCR5 expression was determined by transducing CEM-CCR5 T
cells with CCR5 miR or shRNA sequences. Cell surface CCR5 expression was measured by
staining with a fluorescently-labeled monoclonal antibody against CCR5 and measuring the
intensity of the staining by analytical flow cytometry. The most effective shRNA sequence
for targeting CCR5 was CCR5 shRNA coding sequence #1 (SEQ ID NO: 16). However, the
most effective CCR5 target sequence for constructing the synthetic microRNA sequence was
overlapping with CCR5 shRNA coding sequence #5 (SEQ ID NO: 24); this conclusion was
based on sequence alignments and experience with miRNA construction. Finally, the miR30
hairpin sequence was used to construct the synthetic miR30 CCR5 coding sequence which is
(5′-
AGGTATATTGCTGTTGACAGTGAGCGACTGTAAACTGAGCTTGCTCTACTGTGAA
GCCACAGATGGGTAGAGCAAGCACAGTTTACCGCTGCCTACTGCCTCGGACTTCA
AGGGGCTT-3′) (SEQ ID NO: 1). The CCR5 miRNA target sequence is (5′-
GAGCAAGCTCAGTTTACA-3′) (SEQ ID NO: 5).
At multiplicity of infection equal to 5, generating on average 1.25 genome copies of
integrated lentivirus per cell, CCR5 expression levels were reduce by >90% indicating potent
inhibition of CCR5 mRNA by the miR30 CCR5 coding sequence (SEQ ID NO: 1) when it
was transduced into cells as part of a lentivirus vector.
Conclusion: The miR30 CCR5 coding sequence (SEQ ID NO: 1) is potent for reducing
CCR5 cell surface expression and is a lead candidate for a therapeutic lentivirus for HIV.

| | | | | | |
|---|---|---|---|---|---|
| 13 | SEQ ID NO: | Lentiviral | microRNA | Vif protein | Lead |
| | 2 | vector | sequence | reduction >80% | |

Vector Construction: A Vif microRNA was constructed with oligonucleotide sequences
containing BsrGI and NotI restriction sites that were synthesized by MWG Operon.
Oligonucleotide sequences were inserted into the pCDH lentiviral vector (System
Biosciences) containing an EF-1 promoter. Based on sequence alignments and experience
with constructing synthetic miRNA, the miR21 hairpin sequence was used to construct the
synthetic miR21 Vif coding sequence which is (5′-
CATCTCCATGGCTGTACCACCTTGTCGGGGGATGTGTACTTCTGAACTTGTGTTGA
ATCTCATGGAGTTCAGAAGAACACATCCGCACTGACATTTTGGTATCTTTCATCT
GACCA-3′) (SEQ ID NO: 2). The Vif miRNA coding sequence is (5′-
GGGATGTGTACTTCTGAACTT-3′) (SEQ ID NO: 6). The Vif miRNA target sequence is
(5′-AAGTTCAGAAGTACACATCCC) (SEQ ID NO: 84).
Functional test for potency of miR21Vif After transfection of the miR21 Vif coding
sequence (SEQ ID NO: 2), Vif expression was determined by measuring Vif protein
expression by immunoblot analysis using an anti-Vif monoclonal antibody to identify the Vif
protein.
Conclusion: the miR21 Vif coding sequence (SEQ ID NO: 2) reduced Vif protein expression
by >10-fold compared to control treatments as determined by quantitative image analysis of
immunoblot data. This was sufficient to justify the miR21 Vif coding sequence (SEQ ID
NO: 2) as a lead candidate for our therapeutic lentivirus.

| | | | | | |
|---|---|---|---|---|---|
| 14 | SEQ ID NO: | Lentiviral | microRNA | Tat RNA | Lead |
| | 3 | vector | sequence | reduction >80% | |

Vector Construction: A Tat microRNA was constructed with oligonucleotide sequences
containing BsrGI and NotI restriction sites that were synthesized by MWG Operon. The
microRNA cluster (SEQ ID NO: 30-miR30CCR5 miR21Vif miR185 Tat cluster coding
sequence) was inserted into the pCDH lentiviral vector (System Biosciences) containing an
EF-1 promoter. Based on sequence alignments and experience in the construction of
synthetic miRNA, the miR185 hairpin sequence was selected for constructing a synthetic
miR185 Tat coding sequence which is (5′-
GGGCCTGGCTCGAGCAGGGGGCGAGGGATTCCGCTTCTTCCTGCCATAGCGTGGT
CCCCTCCCCTATGGCAGGCAGAAGCGGCACCTTCCCTCCCAATGACCGCGTCTTC
GTCG-3′) (SEQ ID NO: 3). The Tat miRNA coding sequence is (5′-
TCCGCTTCTTCCTGCCATAG-3′) (SEQ ID NO: 7). The Tat miRNA target sequence is
(5′-CTATGGCAGGAAGAAGCGGA-3′) (SEQ ID NO: 85).
Functional test for potency of miR185 Tat: After transfection of the pCDH lentiviral vector
containing the miR30CCR5 miR21Vif miR185Tat cluster coding sequence (SEQ ID NO:
30), Tat expression was determined by measuring Tat mRNA expression with RT-PCR
analysis using Tat specific primers. The ability of the miR185 Tat coding sequence (SEQ ID
NO: 3) to knock down Tat mRNA expression was analyzed against a similar microRNA in
which the Tat coding sequence was inserted into the miR155 backbone.
Conclusion: The miR185 Tat coding sequence (SEQ ID NO: 3) was approximately twice as
potent for reducing Tat mRNA compared to using the miR155 backbone containing the Tat
miRNA coding sequence. Accordingly, the miR185 Tat coding sequence (SEQ ID NO: 3)
was selected as the lead candidate for our therapeutic lentivirus.

| | | | | | |
|---|---|---|---|---|---|
| 15 | SEQ ID NO: | Lentiviral | microRNA | CCR5 | Candidate |
| | 30 | vector | cluster | reduction >90%, | |
| | | | sequence | Vif protein | |
| | | | | reduction >80%, | |
| | | | | Tat RNA | |
| | | | | reduction >80%, | |
| | | | | >95% inhibition of | |
| | | | | HIV replication | |

Vector Construction: A miR30CCR5 miR21Vif miR185Tat microRNA cluster coding
sequence (SEQ ID NO: 30) was constructed with a synthetic DNA fragment containing
BsrGI and NotI restriction sites that was synthesized by MWG Operon. The DNA fragment
was inserted into the pCDH lentiviral vector (System Biosciences) containing the EF-1
promoter. The miR cluster sequence is (5′-
AGGTATATTGCTGTTGACAGTGAGCGACTGTAAACTGAGCTTGCTCTACTGTGAA
GCCACAGATGGGTAGAGCAAGCACAGTTTACCGCTGCCTACTGCCTCGGACTTCA
AGGGGCTTCCCGGGCATCTCCATGGCTGTACCACCTTGTCGGGGGATGTGTACTT
CTGAACTTGTGTTGAATCTCATGGAGTTCAGAAGAACACATCCGCACTGACATTT
TGGTATCTTTCATCTGACCAGCTAGCGGGCCTGGCTCGAGCAGGGGGCGAGGGA
TTCCGCTTCTTCCTGCCATAGCGTGGTCCCCTCCCCTATGGCAGGCAGAAGCGGC
ACCTTCCCTCCCAATGACCGCGTCTTCGTC-3′) (SEQ ID NO: 30) and incorporates
Test Material 12, Test Material 13 and Test Material 14 into a single cluster that can be
expressed under control of the EF-1 promoter.
Functional test for potency of the Lentivirus Vector AGT 103 containing the miR30CCR5
miR21Vif miR185Tat microRNA cluster coding sequence (SEQ ID NO: 30): The AGT103
vector was tested for potency against CCR5 using the assay for reduction in cell surface
CCR5 expression (Test Material 12). The AGT103 vector was tested for potency against Vif
using the assay for reduction in cell surface Vif expression (Test Material 13). The AGT103
vector was tested for potency against Tat using the assay for reduction in cell surface Tat
expression (Test Material 14).
Conclusion: Potency for reducing CCR5 expression by the miRNA cluster was similar to
potency observed for the miR30CCR5 alone. Potency for reducing Vif expression by the
miRNA cluster was similar to potency observed for the miR21Vif alone. Potency for
reducing Tat expression by the miRNA cluster was similar to potency observed for the
miR185Tat alone. The miR30CCR5 miR21Vif miR Vif miR185 Tat microRNA cluster
coding sequence (SEQ ID NO: 30) is potent for reducing cell surface CCR5 levels and for
inhibiting two HIV genes. Thus, AGT103 containing this miRNA cluster was selected as the
therapeutic vector construct for our HIV functional cure program.

Functional Assays. Individual lentivirus vectors containing CCR5, Tat or Vif shRNA coding sequences (e.g., SEQ ID NOS: 1, 2, and 3) and, for experimental purposes, expressing green fluorescent protein (GFP) under control of the CMV Immediate Early Promoter, and designated AGT103/CMV-GFP were tested for their ability to knockdown CCR5, Tat or Vif expression. Mammalian cells were transduced with lentiviral particles either in the presence or absence of polybrene. Cells were collected after 2-4 days; protein and RNA were analyzed for CCR5, Tat or Vif expression. Protein levels were tested by Western blot assay or by labeling cells with specific fluorescent antibodies (CCR5 assay), followed by analytical flow cytometry comparing modified and unmodified cell fluorescence using either the CCR5-specific or isotype control antibodies.

Starting Testing of Lentivirus. T cell culture medium was made using RPMI 1640 supplemented with 10% FBS and 1% penicillin—streptomycin. Cytokine stocks of IL-2 10,000 units/ml, IL-12 1 μg/ml, IL-7 1 μg/ml, IL-15 1 μg/ml were also prepared in advance.

Prior to transduction with the lentivirus, an infectious viral titer was determined and used to calculate the amount of virus to add for the proper multiplicity of infection (MOI).

Day 0-12: Antigen-specific enrichment. On day 0, cryopreserved PBMC were thawed, washed with 10 ml 37° C. medium at 1200 rpm for 10 minutes and resuspended at a concentration of 2×10⁶/ml in 37° C. medium. The cells were cultured at 0.5 ml/well in a 24-well plate at 37° C. in 5% CO2. To define the optimal stimulation conditions, cells were stimulated with combinations of reagents as listed in Table 3 below:

TABLE 3

| 1 | 2 | 3 | 4 | 5 | 6 |
|---|---|---|---|---|---|
| IL-2 + | IL-7 + | Peptides + | Peptides + | MVA + | MVA + |
| IL-12 | IL-15 | IL-2 + | IL-7 + | IL-2 + | IL-7 + |
| | | IL-12 | IL-15 | IL-12 | IL-15 |

Final concentrations: IL-2=20 units/ml, IL-12=10 ng/ml, IL-7=10 ng/ml, IL-15=10 ng/ml, peptides=5 μg/ml individual peptide, MVA MOI=1.

On days 4 and 8, 0.5 ml fresh medium and cytokine at listed concentrations (all concentrations indicate the final concentration in the culture) were added to the stimulated cells.

Day 12-24: non-specific expansion and lentivirus transduction. On day 12, the stimulated cells were removed from the plate by pipetting and resuspended in fresh T cell culture medium at a concentration of 1×10⁶/ml. The resuspended cells were transferred to T25 culture flasks and stimulated with DYNABEADS® Human T-Activator CD3/CD28 following the manufacturer's instruction plus cytokine as listed above; flasks were incubated in the vertical position.

On day 14, AGT103/CMV-GFP was added at MOI 20 and cultures were returned to the incubator for 2 days. At this time, cells were recovered by pipetting, collected by centrifugation at 1300 rpm for 10 minutes, resuspended in the same volume of fresh medium, and centrifuged again to form a loose cell pellet. That cell pellet was resuspended in fresh medium with the same cytokines used in previous steps, with cells at 0.5×10⁶ viable cells per ml.

From days 14 to 23, the number of the cells was evaluated every 2 days and the cells were diluted to 0.5×10⁶/ml with fresh media. Cytokines were added every time.

On day 24, the cells were collected and the beads were removed from the cells. To remove the beads, cells were transferred to a suitable tube that was placed in the sorting magnet for 2 minutes. Supernatant containing the cells was transferred to a new tube. Cells were then cultured for 1 day in fresh medium at 1×10⁶/ml. Assays were performed to determine the frequencies of antigen-specific T cells and lentivirus transduced cells.

To prevent possible viral outgrowth, amprenavir (0.5 ng/ml) was added to the cultures on the first day of stimulation and every other day during the culture.

Examine antigen-specific T cells by intracellular cytokine staining for IFN-gamma. Cultured cells after peptide stimulation or after lentivirus transduction at 1×10 6 cells/ml were stimulated with medium alone (negative control), Gag peptides (5 μg/ml individual peptide), or PHA (5 μg/ml, positive control). After 4 hours, BD GolgiPlug™ (1:1000, BD Biosciences) was added to block Golgi transport. After 8 hours, cells were washed and stained with extracellular (CD3, CD4 or CD8; BD Biosciences) and intracellular (IFN-gamma; BD Biosciences) antibodies with BD Cytofix/Cytoperm™ kit following the manufacturer's instruction. Samples were analyzed on a BD FACSCalibur™ Flow Cytometer. Control samples labeled with appropriate isotype-matched antibodies were included in each experiment. Data were analyzed using Flowj o software.

Lentivirus transduction rate was determined by the frequency of GFP+ cells. The transduced antigen-specific T cells are determined by the frequency of CD3+CD4+GFP+IFN gamma+ cells; tests for CD3+CD8+GFP+IFN gamma+ cells are included as a control.

These results indicate that CD4 T cells, the target T cell population, can be transduced with lentiviruses that are designed to specifically knock down the expression of HIV-specific proteins, thus producing an expandable population of T cells that are immune to the virus. This example serves as a proof of concept indicating that the disclosed lentiviral constructs can be used in combination with vaccination to produce a functional cure in HIV patients.

### Example 3: Clinical Study for Treatment of HIV

AGT103-T is a genetically modified autologous PBMC containing >1×10 7 HIV-specific CD4 T cells that are also transduced with AGT103 lentivirus vector.

A Phase I clinical trial will test the safety and feasibility of infusing ex vivo modified autologous CD4 T cells (AGT103-T) in adult research participants with confirmed HIV infection, CD4+ T-cell counts >500 cells per mm 3 of blood and stable virus suppression below 200 copies per ml of plasma while on cART. All study participants will continue receiving their standard antiretroviral medications through the Phase I clinical trial. Up to 40 study participants receive two doses by intramuscular injection 8 weeks apart, of recombinant modified vaccinia Ankara (rMVA) expressing HIV Gag, Pol and Env proteins. Seven to 10 days after the second immunization a blood sample is collected for in vitro testing to measure the frequency of CD4+ T-cells that respond to stimulation with a pool of overlapping, synthetic peptides representing the HIV-1 Gag polyprotein. Subjects in the upper half of vaccine responders, based on measuring the frequency of Gag-specific CD4 T cells are enrolled in the gene therapy arm and subjects in the lower half of responders do not continue in the study. We anticipate that the cut-off for higher responders is a HIV-specific CD4+ T cell frequency ≥0.065% of total CD4 T cells. Subjects enrolled into the gene therapy arm of our trial undergo leukapheresis followed by purification of PBMC (using Ficoll density gradient centrifugation or negative selection with antibodies) that are cultured ex vivo and stimulated with HIV Gag peptides plus interleukin-2 and interleukin-12 for 12 days, then stimulated again with beads decorated with CD3/CD28 bispecific antibody. The antiretroviral drug Saquinavir is included at 100 nM to prevent emergence of autologous HIV during ex vivo culture. One day after CD3/CD28 stimulation cells are transduced with AGT103 at multiplicity of infection between 1 and 10. The transduced cells are cultured for an additional 7-14 days during which time they expand by polyclonal proliferation. The culture period is ended by harvesting and washing cells, setting aside aliquots for potency and safety release assays, and resuspending the remaining cells in cryopreservation medium. A single dose is ≤1×10¹⁰ autologous PBMC. The potency assay measures the frequency of CD4 T cells that respond to peptide stimulation by expressing interferon-gamma. Other release criteria include the product must include ≥0.5×10⁷ HIV-specific CD4 T cells that are also transduced with AGT103. Another release criterion is that the number of AGT103 genome copies per cell must not exceed 3. Five days before infusion with AGT103-T subjects receive one dose of busulfuram (or Cytoxan) conditioning regimen followed by infusion of ≤1×10¹⁰ PBMC containing genetically modified CD4 T cells.

A Phase II study will evaluate efficacy of AGT103-T cell therapy. Phase II study participants include individuals enrolled previously in our Phase I study who were judged to have successful and stable engraftment of genetically modified, autologous, HIV-specific CD4 T cells and clinical responses defined as positive changes in parameters monitored as described in efficacy assessments (1.3.). Study participants will be asked to add Maraviroc to their existing regimen of antiretroviral medication. Maraviroc is a CCR5 antagonist that will enhance the effectiveness of genetic therapy directed at reducing CCR5 levels. Once the Maraviroc regimen is in place subjects will be asked to discontinue the previous antiretroviral drug regimen and only maintain Maraviroc monotherapy for 28 days or until plasma viral RNA levels exceed 10,000 per ml on 2 sequential weekly blood draws. Persistently high viremia requires participants to return to their original antiretroviral drug regimen with or without Maraviroc according to recommendations of their HIV care physician.

If participants remain HIV suppressed (below 2,000 vRNA copies per ml of plasma) for >28 days on Maraviroc monotherapy, they will be asked to gradually reduce Maraviroc dosing over a period of 4 weeks followed by intensive monitoring for an additional 28 days. Subjects who maintained HIV suppression with Maraviroc monotherapy are considered to have a functional cure. Subjects who maintain HIV suppression even after Maraviroc withdrawal also have a functional cure. Monthly monitoring for 6 months followed by less intensive monitoring will establish the durability of functional cure.

Patient Selection Inclusion Criteria:
Aged between 18 and 60 years.
Documented HIV infection prior to study entry.
Must be willing to comply with study-mandated evaluations; including not changing their antiretroviral regimen (unless medically indicated) during the study period.
CD4+ T-cell count >500 cell per millimeter cubed (cells/mm3)
CD4+ T-cell nadir of >400 cells/mm3
HIV viral load <1,000 copies per milliliter (mL)

Exclusion Criteria:
Any viral hepatitis
Acute HIV infection
HIV viral load >1,000 copies/mL
Active or recent (prior 6 months) AIDS defining complication
Any change in HIV medications within 12 weeks of entering the study
Cancer or malignancy that has not been in remission for at least 5 years with the exception of successfully treated basal cell carcinoma of the skin
Current diagnosis of NYHA grade 3 or 4 congestive heart failure or
uncontrolled angina or arrhythmias
History of bleeding problems
Use of chronic steroids in past 30 days
Pregnant or breast feeding
Active drug or alcohol abuse
Serious illness in past 30 days
Currently participating in another clinical trial or any prior gene therapy

Safety assessments
Acute infusion reaction
Post-infusion safety follow-up

Efficacy assessments—Phase I
Number and frequency of modified CD4 T cells.
Durability of modified CD4 T cells.
In vitro response to Gag peptide re-stimulation (ICS assay) as a measure of memory T cell function.
Polyfunctional anti-HIV CD8 T cell responses compare to pre- and post-vaccination time points.
Frequency of CD4 T cells making doubly spliced HIV mRNA after in vitro stimulation.

Efficacy assessments—Phase II
Number and frequency of genetically modified CD4 T cells.
Maintenance of viral suppression (<2,000 vRNA copies per ml but 2 consecutive weekly draws not exceeding 5×10⁴ vRNA copies per ml are permitted) with Maraviroc monotherapy.
Continued virus suppression during and after Maraviroc withdrawal.
Stable CD4 T cell count.

AGT103-T consists of up to 1×10¹⁰ genetically modified, autologous CD4+ T cells containing ≥1×10⁷ HIV-specific CD4 T cells that are also transduced with AGT103 lentivirus vector. A Phase I clinical trial will test the safety and feasibility of infusing ex vivo modified autologous CD4 T cells (AGT103-T) in adult research participants with confirmed HIV infection, CD4+ T-cell counts >500 cells per mm 3 of blood and stable virus suppression below 200 copies per ml of plasma while on cART. Up to 40 study participants receive two doses by intramuscular injection 8 weeks apart, of recombinant modified vaccinia Ankara (rMVA) expressing HIV Gag, Pol and Env proteins. Seven to 10 days after the second immunization a blood sample is collected for in vitro testing to measure the frequency of CD4+ T-cells that respond to stimulation with a pool of overlapping, synthetic peptides representing the HIV-1 Gag polyprotein. Subjects in the upper half of vaccine responders, based on measuring the frequency of Gag-specific CD4 T cells are enrolled in the gene therapy arm and subjects in the lower half of responders do not continue in the study. We anticipate that the cut-off for higher responders is a HIV-specific CD4+ T cell frequency ≥0.065% of total CD4 T cells. Subjects enrolled into the gene therapy arm of our trial undergo leukapheresis and the CD4+ T cells are enriched by negative selection. The enriched CD4 subset is admixed with 10% the number of cells from the CD4-negative subset to provide a source and antigen-presenting cells. The enriched CD4 T cells are stimulated with HIV Gag peptides plus interleukin-2 and interleukin-12 for 12 days, then stimulated again with beads decorated with CD3/CD28 bispecific antibody. The antiretroviral drug Saquinavir is included at 100 nM to prevent emergence of autologous HIV during ex vivo culture. One day after CD3/CD28 stimulation cells are transduced with AGT103 at multiplicity of infection between 1 and 10. The transduced cells are cultured for an additional 7-14 days during which time they expand by polyclonal proliferation. The culture period is ended by harvesting and washing cells, setting aside aliquots for potency and safety release assays, and resuspending the remaining cells in cryopreservation medium. A single dose is ≤1×10¹⁰ autologous cells enriched for the CD4+ T cell subset. The potency assay measures the frequency of CD4 T cells that respond to peptide stimulation by expressing interferon-gamma. Other release criteria include that the product must include ≥0.5×10⁷ HIV-specific CD4 T cells that are also transduced with AGT103. Another release criterion is that the number of AGT103 genome copies per cell must not exceed 3. Five days before infusion with AGT103-T subjects receive one dose of busulfuram (or Cytoxan) conditioning regimen followed by infusion of ≤1×10¹⁰ enriched and genetically modified CD4 T cell.

A Phase II study will evaluate efficacy of AGT103-T cell therapy. Phase II study participants include individuals enrolled previously in our Phase I study who were judged to have successful and stable engraftment of genetically modified, autologous, HIV-specific CD4 T cells and clinical responses defined as positive changes in parameters monitored as described in efficacy assessments (1.3.). Study participants will be asked to add Maraviroc to their existing regimen of antiretroviral medication. Maraviroc is a CCR5 antagonist that will enhance the effectiveness of genetic therapy directed at reducing CCR5 levels. Once the Maraviroc regimen is in place subjects will be asked to discontinue the previous antiretroviral drug regimen and only maintain Maraviroc monotherapy for 28 days or until plasma viral RNA levels exceed 10,000 per ml on 2 sequential weekly blood draws. Persistently high viremia requires participants to return to their original antiretroviral drug regimen with or without Maraviroc according to recommendations of their HIV care physician.

If participants remain HIV suppressed (below 2,000 vRNA copies per ml of plasma) for >28 days on Maraviroc monotherapy, they will be asked to gradually reduce Maraviroc dosing over a period of 4 weeks followed by intensive monitoring for an additional 28 days. Subjects who maintained HIV suppression with Maraviroc monotherapy are considered to have a functional cure. Subjects who maintain HIV suppression even after Maraviroc withdrawal also have a functional cure. Monthly monitoring for 6 months followed by less intensive monitoring will establish the durability of functional cure.

### Example 4: Generating a Population of CD4+ T Cells Through Depletion of CD8+ T Cells Prior to Peptide Stimulation

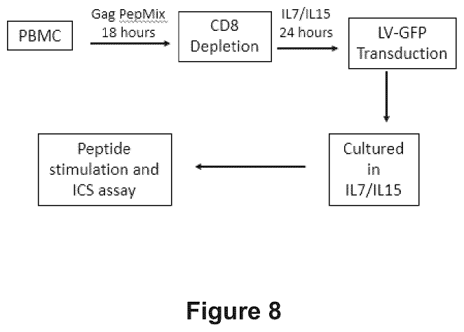

Because CD8+ T cell overgrowth significantly impacted the expansion of target CD4+ T cells, CD8+ T cells were depleted at the beginning of cell expansion to determine whether it would improve CD4+ T cell expansion. Current CD8+ T cell depletion methods require that cells are passed through a magnetic column. To avoid possible impacts of that procedure on antigen presenting cells and CD4+ T cells, the cell depletion was performed after peptide stimulation and before lentivirus transduction when cells were better able to withstand the mechanical stresses. The objective was to increase the final yield of CD4+ T cells and also increase the yield of target cells (HIV gag-protein CD4+ T cells that are also transduced with the AGT103 lentivirus vector). FIG. 8 depicts a schematic of a CD8+ T cell depletion protocol. The method used to deplete CD8+ T cells is described in Example 8.

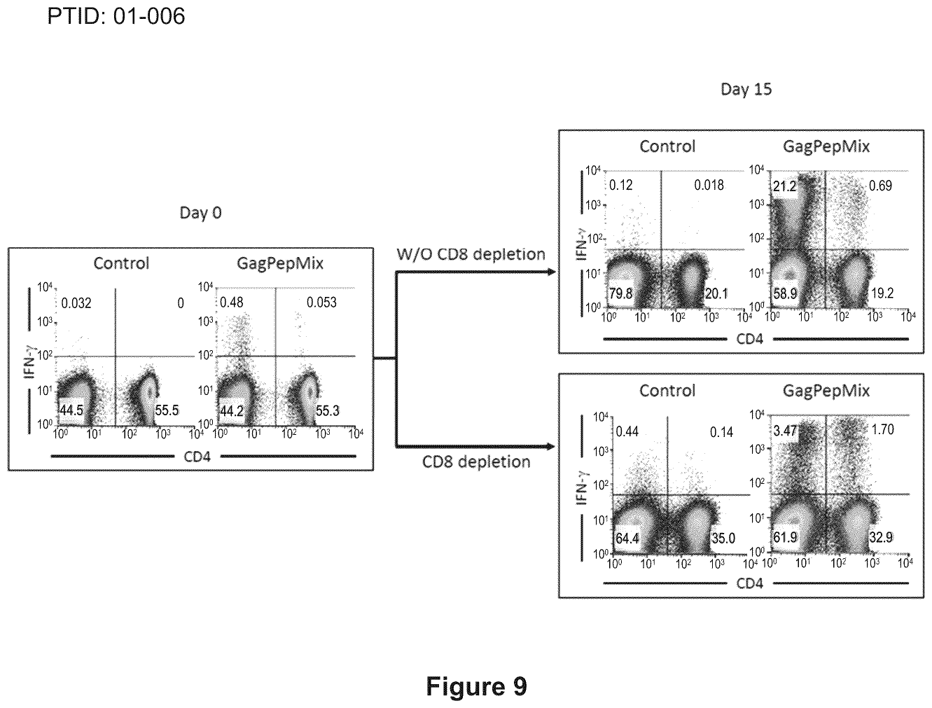

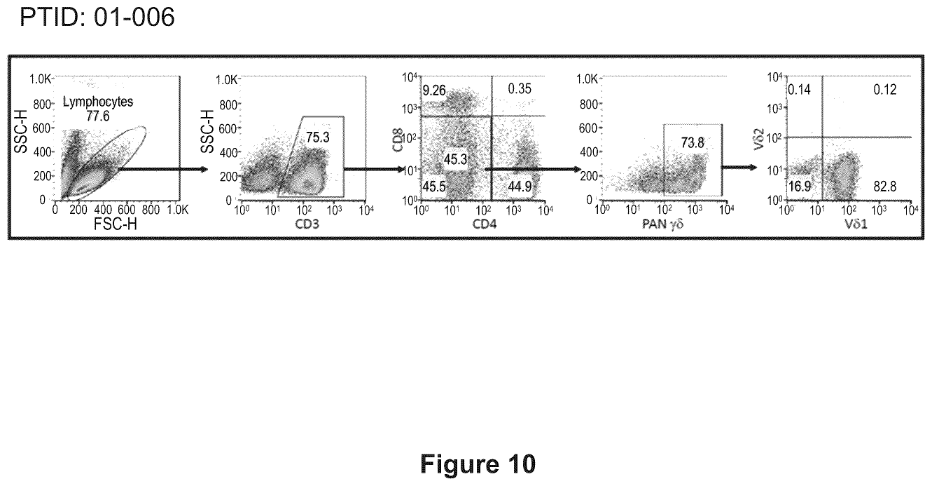

As shown in FIGS. 9, 11 and 12, CD8+ T cell depletion resulted in a substantial increase in the percentage of Gag-specific CD4+ T cells relative to control treatments in which CD8+ T cells were not depleted; Gag-specific cells express interferon-γ and the target cells are interferon-γ+ and CD4+(upper right quadrants). For PTID: 01-006, after CD8+ depletion, the value was 1.7% of total T cells compared to 0.69% in the control treatments in which CD8+ T cells were not depleted (FIG. 9, upper right quadrants of Day 15 GagPepMix). Referring to FIG. 9, on day 0, the lower left quadrant, the lower right quadrant, the upper left quadrant, and the upper right quadrant of the control, had a fluorescence intensity of 44.5%, 55.5%, 0.032%, and 0%, respectively compared to 44.2%, 55.3%, 0.48% and 0.053% for the peptide-stimulated culture (1 day). On day 15, without CD8 depletion, the lower left quadrant, the lower right quadrant, the upper left quadrant, and the upper right quadrant of the control, had a fluorescence intensity of 79.8%, 20.1%, 0.12%, and 0.018%, respectively. On day 15, without CD8 depletion, the lower left quadrant, the lower right quadrant, the upper left quadrant, and the upper right quadrant of the GagPepMix had a fluorescence intensity of 58.9%, 19.2%, 21.2%, and 0.69%, respectively. On day 15, with CD8 depletion, the lower left quadrant, the lower right quadrant, the upper left quadrant, and the upper right quadrant of the control, had a fluorescence intensity of 64.4%, 35.0%, 0.44%, and 0.14%, respectively. On day 15, with CD8 depletion, the lower left quadrant, the lower right quadrant, the upper left quadrant, and the upper right quadrant of the GagPepMix, had a fluorescence intensity of 61.9%, 32.9%, 3.47%, and 1.70%, respectively. The proportion of CD4+ T cells without CD8 depletion was 20.1% (Day 15 Control, sum of upper right and lower right quadrants) compared to 35.1% with CD8 T cell depletion (Day 15 Control, sum of upper right and lower right quadrants). We identified the large population of CD3+/CD4-negative/CD8-negative cells present in the final product as γδ T cells of the subclass Vol (FIG. 10). Thus, in addition to depleting CD8+ T cells it may be necessary to also delete γδ T cells.

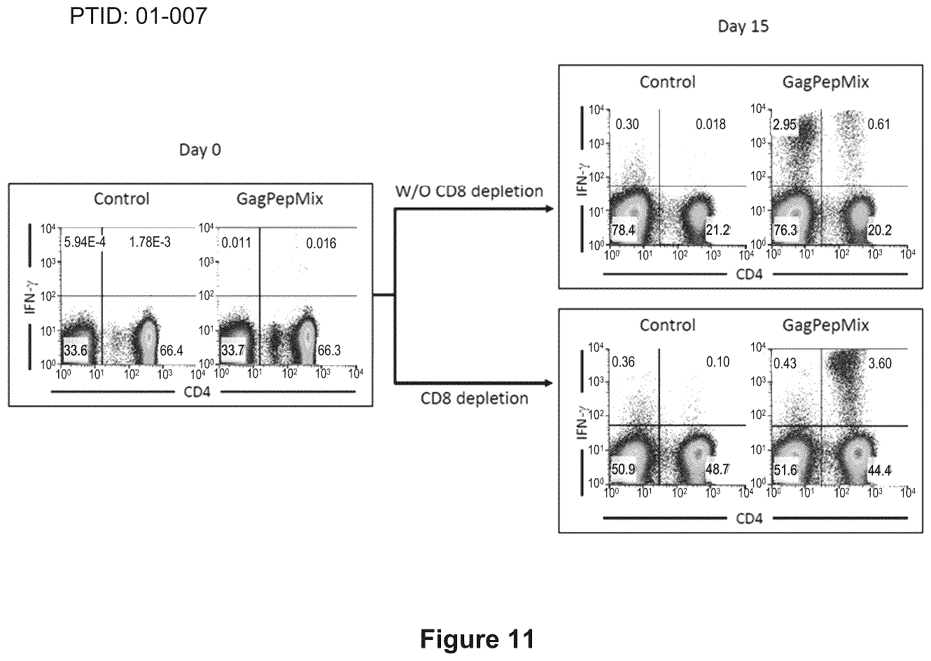

For PTID: 01-007, the CD8+ T cell depletion increased the yield of target cells by approximately 6-fold relative to control treated cells in which CD8+ T cells were not depleted (FIG. 11). Referring to FIG. 11, on day 0, the lower left quadrant, the lower right quadrant, the upper left quadrant, and the upper right quadrant of the control, had a fluorescence intensity of 33.6%, 66.4%, 5.9E−4%, and 1.78E−3%, respectively. On day 0 (following 16 hours exposure to Gag peptides), the lower left quadrant, the lower right quadrant, the upper left quadrant, and the upper right quadrant of the GagPepMix, had a fluorescence intensity of 33.7%, 66.3%, 0.011%, and 0.016%, respectively. On day 15, without CD8 depletion, the lower left quadrant, the lower right quadrant, the upper left quadrant, and the upper right quadrant of the control, had a fluorescence intensity of 78.4%, 21.2%, 0.30%, and 0.018%, respectively. On day 15, without CD8 depletion, the lower left quadrant, the lower right quadrant, the upper left quadrant, and the upper right quadrant of the GagPepMix had a fluorescence intensity of 76.3%, 20.2%, 2.95%, and 0.61%, respectively. On day 15, with CD8 depletion, the lower left quadrant, the lower right quadrant, the upper left quadrant, and the upper right quadrant of the control, had a fluorescence intensity of 50.9%, 48.7%, 0.36%, and 0.10%, respectively. On day 15, with CD8 depletion, the lower left quadrant, the lower right quadrant, the upper left quadrant, and the upper right quadrant of the GagPepMix, had a fluorescence intensity of 51.6%, 44.4%, 0.43%, and 3.60%, respectively.

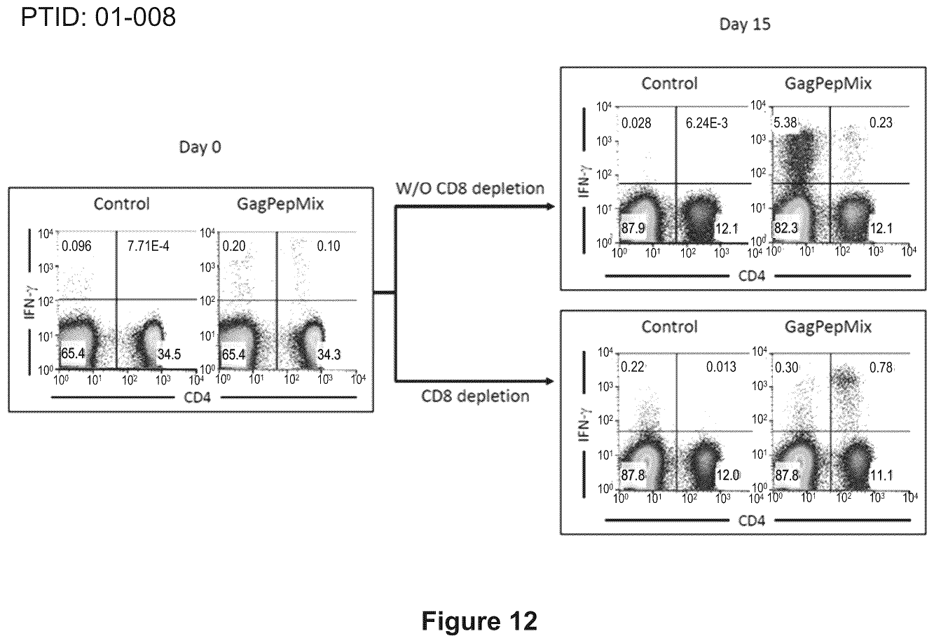

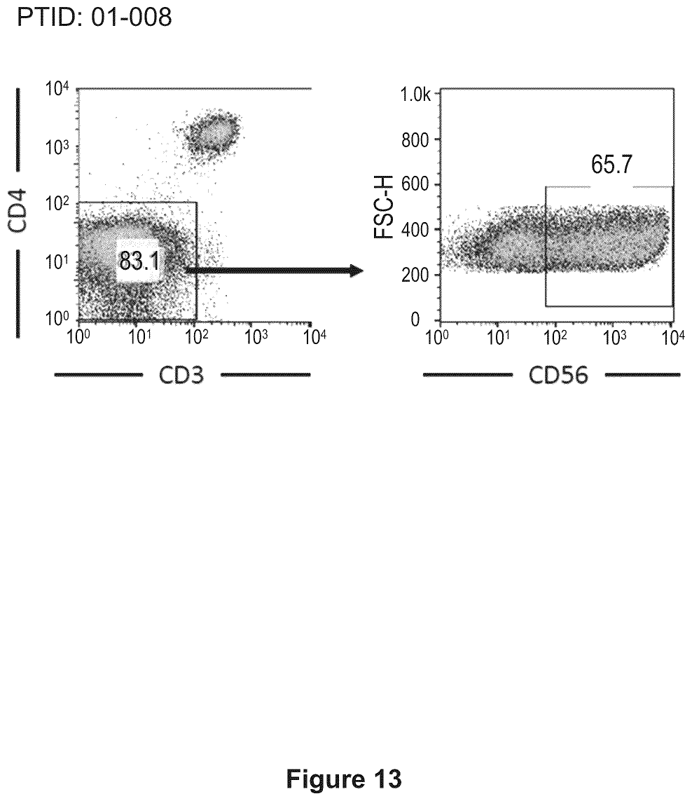

For PTID: 01-008, depletion of CD8+ T cells resulted in increased target cell yield of approximately 3-fold compared to control treated cells in which CD8+ T cells were not depleted (FIG. 12). Referring to FIG. 12, on day 0, the lower left quadrant, the lower right quadrant, the upper left quadrant, and the upper right quadrant of the control, had a fluorescence intensity of 65.4%, 34.5%, 0.096%, and 7.71E−4%, respectively. On day 0, the lower left quadrant, the lower right quadrant, the upper left quadrant, and the upper right quadrant of the GagPepMix, had a fluorescence intensity of 65.4%, 34.3%, 0.20%, and 0.10%, respectively. On day 15, without CD8 depletion, the lower left quadrant, the lower right quadrant, the upper left quadrant, and the upper right quadrant of the control, had a fluorescence intensity of 87.9%, 12.1%, 0.028%, and 6.24E−3%, respectively. On day 15, without CD8 depletion, the lower left quadrant, the lower right quadrant, the upper left quadrant, and the upper right quadrant of the GagPepMix had a fluorescence intensity of 82.3%, 12.1%, 5.38%, and 0.23%, respectively. On day 16, with CD8 depletion, the lower left quadrant, the lower right quadrant, the upper left quadrant, and the upper right quadrant of the control, had a fluorescence intensity of 87.8%, 12.0%, 0.22%, and 0.013%, respectively. On day 16, with CD8 depletion, the lower left quadrant, the lower right quadrant, the upper left quadrant, and the upper right quadrant of the GagPepMix, had a fluorescence intensity of 87.8%, 11.1%, 0.30%, and 0.78%, respectively. For PTID: 01-008, the contaminating cells present at the end of process were identified as CD3+/CD4-negative/CD56+, meaning they are most likely NK cells (FIG. 13).

Together, these data show that CD8+ T cell depletion had a consistent effect of increasing the Gag-responding CD4+ T cells for HIV+ patient PBMC samples.

### Example 5: CD8 Cell Depletion Increased the Percentage of Gag-Specific CD4+ T Cells but Outgrowth of Vol Cells and CD56+ NK Cells Impeded Growth of the Target Cell Population

For PTID: 01-006, CD8+ T cell depletion using the protocol described in Example 8 resulted in a final product that was less than 50% CD4+ T cells and that contained an outgrowth of Vδ1+γδ T cells (FIG. 10). This result suggests that depleting both CD8+ T cells and Vδ1+ T cells may increase the yield of CD4+ T cells and may also increase the yield of target cells. Gating data for lymphocytes showed 77.6% fluorescence intensity (far left graph of FIG. 10). Using the parameter of CD3 expression, the fluorescence intensity was 75.3% (middle, left graph of FIG. 10). Using the parameter of CD4 expression, fluorescence was measured in 4 separate quadrants (middle graph of FIG. 10). The lower left quadrant showed a fluorescence intensity of 45.3% (upper value) and 45.5% (lower value). The lower right quadrant showed a fluorescence intensity of 44.9%. The upper left quadrant showed a fluorescence intensity of 9.26%. The upper left quadrant showed a fluorescence intensity of 0.35%. Using the parameter of PAN γδ, the fluorescence intensity was 73.8% (middle, right graph of FIG. 10). Using the parameter of Vδ1, fluorescence was measured in 4 separate quadrants. The lower left quadrant showed a fluorescence intensity of 16.9%. The lower right quadrant showed a fluorescence intensity of 82.8%. The upper left quadrant showed a fluorescence intensity of 0.14%. The upper right quadrant showed a fluorescence intensity of 0.12%.

For PTID: 01-008, depletion of CD8+ T cells resulted in low yield of CD4+ T cells, which was likely due to expansion of CD56+ NK cells (FIG. 13). The fluorescence intensity in the left graph using the variables of CD3 and CD4 defined 83.1% of cells as negative for both CD3 and CD4 expression, most likely NK cells. The fluorescence intensity in the right graph using the variable of CD56 was 65.7%. Due to their expansion, the CD56+ NK cells competed with the CD4+ T cells in the culture medium. This result suggests that routine depletion of CD56+ NK cells may be another important step in manufacturing of CD4+ T cells.

### Example 6: Generating a Population of CD4+ T Cells Using Various Depletion Protocols

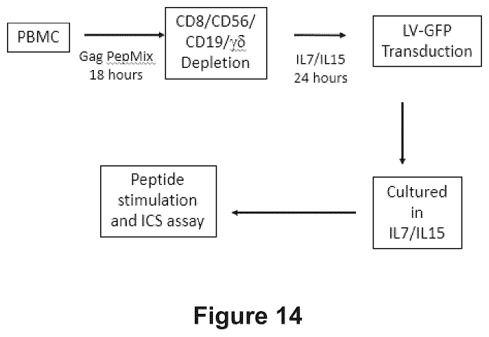

Because CD8+ T cell depletion alone resulted in outgrowth of Vδ1 and CD56+ NK cells, various depletion protocols were developed. A depletion protocol was developed in which CD8+ T cells, CD56 NK cells, CD19 B cells, and γδ cells were all depleted. A flow diagram of this scheme is depicted in FIG. 14.

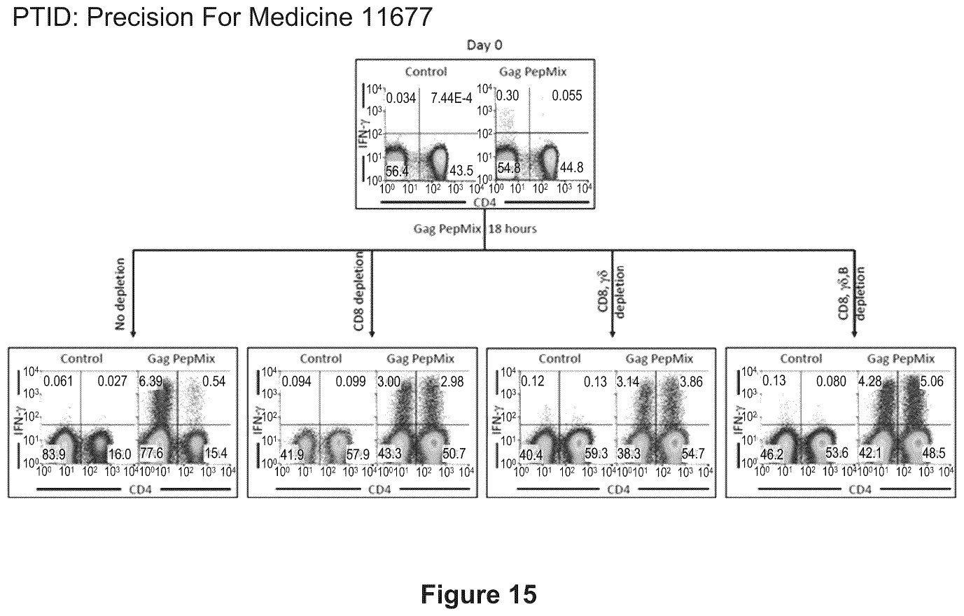

FIG. 15 shows comparisons of various cell depletion strategies, including “no depletion” control; CD8+ T cell depletion; CD8+ T cell and γδ T cell depletion; and CD8+ T cell, γδ T cell, and B cell depletion. Target cells were identified by their response to peptide re-stimulation with subsequent intracellular accumulation of interferon-gamma. The ex vivo sample contained 0.055% of total T cells that were CD4+ and specific for responding to HIV gag peptides. The protocol for cell depletion in Example 8 was used to deplete the cells of different factors. Briefly, cells were cultured for 18 hours with PBMC plus Gag peptides followed by cell depletion (or the “no-depletion” control). After cell depletion, cells were cultured for an additional 12 days during which the cells were regularly fed supplementation with IL-7 and IL-15 cytokines. Depletion of CD8+ T cells, γδ T cells, and CD19+ B cells produced the highest yield of target cells (CD4+ T cells and interferon-gamma+ cells) (FIG. 15).

Referring to FIG. 15, on day 0, the lower left quadrant, the lower right quadrant, the upper left quadrant, and the upper right quadrant of the control, had a fluorescence intensity of 56.4%, 43.5%, 0.034%, and 7.44E−4%, respectively. On day 0, the lower left quadrant, the lower right quadrant, the upper left quadrant, and the upper right quadrant of the GagPepMix, had a fluorescence intensity of 54.8%, 44.8%, 0.30%, and 0.055%, respectively. After 18 hours with no depletion, the lower left quadrant, the lower right quadrant, the upper left quadrant, and the upper right quadrant of the control, had a fluorescence intensity of 83.9%, 16.0%, 0.061%, and 0.027%, respectively. After 18 hours with no depletion, the lower left quadrant, the lower right quadrant, the upper left quadrant, and the upper right quadrant of the GagPepMix, had a fluorescence intensity of 77.6%, 15.4%, 6.39%, and 0.54%, respectively. After 18 hours with CD8 depletion, the lower left quadrant, the lower right quadrant, the upper left quadrant, and the upper right quadrant of the control, had a fluorescence intensity of 41.9%, 57.9%, 0.094%, and 0.099%, respectively. After 18 hours with CD8 depletion, the lower left quadrant, the lower right quadrant, the upper left quadrant, and the upper right quadrant of the GagPepMix, had a fluorescence intensity of 43.3%, 50.7%, 3.00%, and 2.98%, respectively. After 18 hours with CD8 and γδ depletion, the lower left quadrant, the lower right quadrant, the upper left quadrant, and the upper right quadrant of the control, had a fluorescence intensity of 40.4%, 59.3%, 0.12%, and 0.13%, respectively. After 18 hours with CD8 and γδ depletion, the lower left quadrant, the lower right quadrant, the upper left quadrant, and the upper right quadrant of the GagPepMix, had a fluorescence intensity of 38.3%, 54.7%, 3.14%, and 3.86%, respectively. After 18 hours with CD8, γδ, and B depletion, the lower left quadrant, the lower right quadrant, the upper left quadrant, and the upper right quadrant of the control, had a fluorescence intensity of 46.2%, 53.6%, 0.13%, and 0.080%, respectively. After 18 hours with CD8, γδ, and B depletion, the lower left quadrant, the lower right quadrant, the upper left quadrant, and the upper right quadrant of the GagPepMix, had a fluorescence intensity of 42.1%, 48.5%, 4.28%, and 5.06%, respectively.

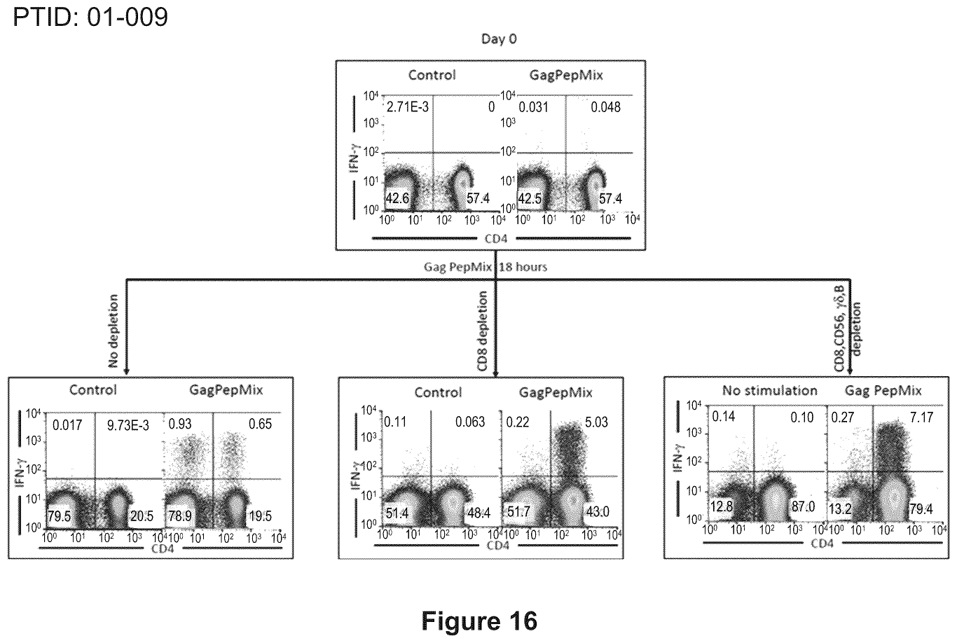

FIG. 16 shows comparison of various cell depletion strategies, include “no depletion control; CD8+ T cell depletion; and depletion of 4 cell types including CD8+ T cells, CD56+ NK cells, γδ T cells, and B cells. After depletion of the 4 cell types, a substantial increase in the yield of CD4+ T cells/interferon-gamma+ target cells was observed.

Referring to FIG. 16, on day 0, the lower left quadrant, the lower right quadrant, the upper left quadrant, and the upper right quadrant of the control, had a fluorescence intensity of 42.6%, 57.4%, 2.71E−3%, and 0.0%, respectively. On day 0, the lower left quadrant, the lower right quadrant, the upper left quadrant, and the upper right quadrant of the GagPepMix, had a fluorescence intensity of 42.5%, 57.4%, 0.031%, and 0.048%, respectively. After 18 hours with no depletion, the lower left quadrant, the lower right quadrant, the upper left quadrant, and the upper right quadrant of the control, had a fluorescence intensity of 79.5%, 20.5%, 0.017%, and 9.73E−3%, respectively. After 18 hours with no depletion, the lower left quadrant, the lower right quadrant, the upper left quadrant, and the upper right quadrant of the GagPepMix, had a fluorescence intensity of 78.9%, 19.5%, 0.93%, and 0.65%, respectively. After 18 hours with CD8 depletion, the lower left quadrant, the lower right quadrant, the upper left quadrant, and the upper right quadrant of the control, had a fluorescence intensity of 51.4%, 48.4%, 0.11%, and 0.063%, respectively. After 18 hours with CD8 depletion, the lower left quadrant, the lower right quadrant, the upper left quadrant, and the upper right quadrant of the GagPepMix, had a fluorescence intensity of 51.7%, 43.0%, 0.22%, and 5.03%, respectively. After 18 hours with CD8, CD56, γδ, and B depletion, the lower left quadrant, the lower right quadrant, the upper left quadrant, and the upper right quadrant of the cells that had no stimulation, had a fluorescence intensity of 12.8%, 87.0%, 0.14%, and 0.10%, respectively. After 18 hours with CD8, CD56, γδ, and B depletion, the lower left quadrant, the lower right quadrant, the upper left quadrant, and the upper right quadrant of the GagPepMix, had a fluorescence intensity of 13.2%, 79.4%, 0.27%, and 7.17%, respectively.

### Example 7: Generating a Population of CD4+ T Cells Using a 4-Way Cell Depletion Protocol

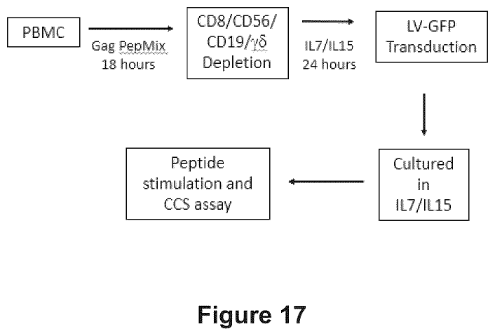

FIG. 17 depicts a flow diagram for an experiment using 4-way cell depletion, which depletes CD8+ T cells, CD56+ NK cells, CD19+ B cells, and γδ T cells. The LV-GFP surrogate viral vector is used to transduce the cells. This vector has identical cell tropism to AGT103 but it expresses the green fluorescence protein marker so that transduction efficiency can be easily assessed. T cells were stimulated with CD3/CD28 beads to increase transduction efficiency. An assay that measures interferon-gamma expression was used to measure transduction efficiency in multiple cell subsets.

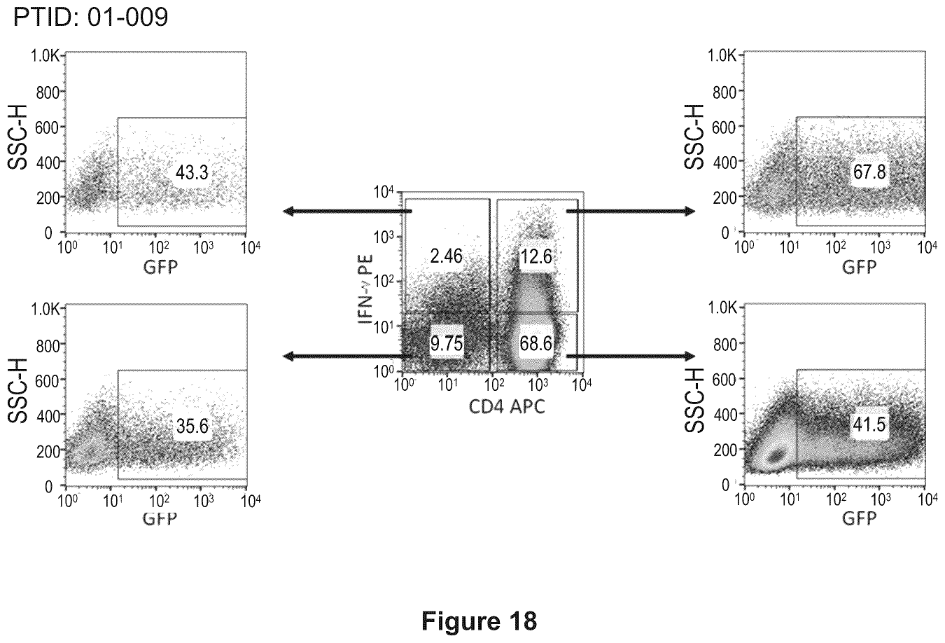

As shown in FIG. 18, peptide stimulation alone followed by depletion of competing cells, was sufficient for efficient lentivirus transduction and efficiency was highest in the CD4+/Gag-specific target cells. In this typical result, 68% of target cells were transduced with the LV-GFP vector. Referring to FIG. 18, on the middle graph the lower right quadrant (68.6% fluorescence) and upper right quadrant (12.6% fluorescence), had a GFP transduction efficiency of 41.5% (lower, right graph), and 67.8%, respectively (upper, right graph). This is in contrast to the lower left quadrant (9.75% fluorescence) and the upper left quadrant (2.46% fluorescence) of the middle graph, which had a GFP transduction efficiency of 35.6% (lower, left graph) and 43.3%, respectively (upper, left graph).

### Example 8: Laboratory Protocol for Cell Depletion

Day 0: Approximately 1×10⁷ viable peripheral blood mononuclear cells (PBMCs) from venous blood of HIV-positive participants were cultured in 1 mL of TexMACS GMP Medium (Miltenyi Biotec, Auburn, CA) containing 5% human serum (Gemini Bio Products, West Sacramento, CA), 10 ng/mL IL7/IL15 (Miltenyi Biotec, Auburn, CA) and 100 nM Saquinavir (NIH AIDS Reagent Program, Germantown, MD) in a 24-well plate. The cells were incubated with 1 μg/mL PepMix HIV-1 (GAG) Ultra (JPT Peptide Technologies, Berlin, Germany) for 16 to 18 hours at 37° C.; 1 μg/mL was the concentration for each individual peptide in the final stimulation condition.

Day 1: After 16 to 18 hours incubation, CD8, CD56, CD19 and/or γδ cells were depleted with PE anti-human CD8 Ab clone skl (BioLegend, San Diego, CA), PE anti-human CD56 Ab clone 5.1H11 (BioLegend, San Diego, CA), PE anti-human CD19 Ab clone HIB19 (BioLegend, San Diego, CA), and PE anti-human TCR γδ Ab clone B1 (BioLegend, San Diego, CA) and anti-PE microbeads (for example, Anti-PE MicroBeads UltroPure, Miltenyi Biotec, Auburn, CA) according to the manufacturer's instructions. Cells were stained with PE-conjugate antibodies for 10 minutes on ice. The labeled cells were washed and incubated with anti-PE MicroBeads for 15 minutes on ice, then separated in a magnetic field wherein cells expressing the target surface proteins were retained on the column and unlabeled (negatively-selected) cells passed through and were collected. Approximately 1×10⁶ viable negatively-selected PBMCs were seeded in 0.5 mL TexMACS GMP Medium containing 5% human serum, 10 ng/mL IL7/IL15, and 100 nM Saquinavir.

Day 2: Cells were transduced with lentivirus vector carrying GFP at an MOI of 5 to 10.

Day 4: Cells were fed with 0.5 mL TexMACS GMP Medium containing 5% human serum, 20 ng/mL IL7/IL15 (final 10 ng/mL) and 200 nM Saquinavir (final 100 nM).

Day 7: Cells were fed with 1 mL TexMACS GMP Medium containing 5% human serum, 20 ng/mL IL7/IL15 (final 10 ng/mL) and 200 nM Saquinavir (final 100 nM).

Day 10: Cells were mixed thoroughly including the clumps of cells on the bottom of the well. After mixing, the entire volume of cells was transferred into a 12-well plate. Cells were then fed with 2 mL TexMACS GMP Medium containing 5% human serum, 20 ng/mL IL7/IL15 (final long/mL) and 200 nM Saquinavir (final 100 nM).

Days 14 to 16: Cell counts were performed and cells were stimulated with peptide stimulation assay. 1×10⁶ expanded cells were stimulated without or with 1 μg/mL PepMix HIV-1 (GAG) Ultra for 4 hours in a 96-well plate.

To detect intracellular IFN-γ, Protein transport inhibitor GolgiPlug (Containing Brefeldin A) (BD Biosciences, San Jose, CA) was added. Cells were stained with FITC anti-human CD3 Ab clone OKT3, PE anti-human CD4 Ab clone OKT4 and PerCP anti-human CD8 Ab clone SK1. After staining, cells were fixed, permeabilized, and incubated for 45 min at 4° C. with APC anti-human IFN-γ clone B27. Intracellular staining solutions were obtained from the Cytofix/Cytoperm Kit (BD Biosciences, San Jose, CA).

GFP expression was analyzed to determine the transduction efficiency of HIV-specific CD4 T cells. An IFN-γ Secretion Assay Cell Enrich and Detect Kit (PE) (Miltenyi Biotec, Auburn, CA) was used to detect IFN-γ secreting cells according to the manufacturer's instructions. Cells were labeled with IFN-γ Catch Reagent for 5 minutes on ice. The labeled cells were incubated in a closed tube for 45 minutes at 37° C. under slow continuous rotation, then stained with PE anti-IFN-γ detection Ab, PerCP anti-human CD3 Ab clone OKT3 and APC anti-human CD4 Ab clone OKT4.

Data were acquired for at least 1×10⁵ lymphocytes (gated on the basis of forward- and side-scatter profiles) from each sample, using a FACSCalibur flow cytometer (BD Biosciences, San Jose, CA). All samples were analyzed using FlowJo software (FlowJo 10.1r1, Tree Star, San Carlos, CA).

### Example 9: Commercial Protocol for Cell Depletion

In a commercial version of the protocol for manufacturing AGT103-T, PBMC purification, peptide stimulation and cell depletion are performed as described above, and cultured for 2 days before transferring the total culture fluid (200 ml) to a GREX 500M static culture flask or similar container for cell culturing with no or minimal agitation, containing approximately 5 liters of culture medium including IL-7 and IL-15 cytokines. The filled flask is incubated at 37.0 with 5% CO2 for 7 days without agitation, then the entire volume is collected, cells are washed and resuspended in cryopreservation solution at approximately 1×10⁸ total nucleated cells per mL, then frozen and stored in the vapor phase of liquid N₂. Inclusion of the static phase culture further increases the target cell yield, as this cell subset is demonstrated in laboratory studies to be fragile and susceptible to depletion during mechanical collection and/or culture agitation in a non-static culture system.

### Sequences

The following sequences are referred to herein:

The following sequences are referred to herein:

| SEQ ID | | |
|---|---|---|
| NO: | Description | Sequence |
| 1 | miR30 CCR5 | AGGTATATTGCTGTTGACAGTGAGCGACTGTAAACTGAGCTTGCT |
| | coding | CTACTGTGAAGCCACAGATGGGTAGAGCAAGCACAGTTTACCGC |
| | sequence | TGCCTACTGCCTCGGACTTCAAGGGGCTT |
| 2 | miR21 Vif | CATCTCCATGGCTGTACCACCTTGTCGGGGGATGTGTACTTCTGA |
| | coding | ACTTGTGTTGAATCTCATGGAGTTCAGAAGAACACATCCGCACT |
| | sequence | GACATTTTGGTATCTTTCATCTGACCA |
| 3 | miR185 Tat | GGGCCTGGCTCGAGCAGGGGGCGAGGGATTCCGCTTCTTCCTGC |
| | coding | CATAGCGTGGTCCCCTCCCCTATGGCAGGCAGAAGCGGCAC |
| | sequence | CTTCCCTCCCAATGACCGCGTCTTCGTCG |
| 4 | Elongation | CCGGTGCCTAGAGAAGGTGGCGCGGGGTAAACTGGGAAAGTGA |
| | Factor-1 alpha | TGTCGTGTACTGGCTCCGCCTTTTTCCCGAGGGTGGGGGAGAACC |
| | (EF1-alpha) | GTATATAAGTGCAGTAGTCGCCGTGAACGTTCTTTTTCGCAACGG |
| | promoter | GTTTGCCGCCAGAACACAGGTAAGTGCCGTGTGTGGTTCCCGCG |
| | | GGCCTGGCCTCTTTACGGGTTATGGCCCTTGCGTGCCTTGAATTA |
| | | CTTCCACGCCCCTGGCTGCAGTACGTGATTCTTGATCCCGAGCTT |
| | | CGGGTTGGAAGTGGGTGGGAGAGTTCGAGGCCTTGCGCTTAAGG |
| | | AGCCCCTTCGCCTCGTGCTTGAGTTGAGGCCTGGCCTGGGCGCTG |
| | | GGGCCGCCGCGTGCGAATCTGGTGGCACCTTCGCGCCTGTCTCGC |
| | | TGCTTTCGATAAGTCTCTAGCCATTTAAAATTTTTGATGACCTGC |
| | | TGCGACGCTTTTTTTCTGGCAAGATAGTCTTGTAAATGCGGGCCA |
| | | AGATCTGCACACTGGTATTTCGGTTTTTGGGGCCGCGGGCGGCG |
| | | ACGGGGCCCGTGCGTCCCAGCGCACATGTTCGGCGAGGCGGGGC |
| | | CTGCGAGCGCGGCCACCGAGAATCGGACGGGGGTAGTCTCAAGC |
| | | TGGCCGGCCTGCTCTGGTGCCTGGCCTCGCGCCGCCGTGTATCGC |
| | | CCCGCCCTGGGCGGCAAGGCTGGCCCGGTCGGCACCAGTTGCGT |
| | | GAGCGGAAAGATGGCCGCTTCCCGGCCCTGCTGCAGGGAGCTCA |
| | | AAATGGAGGACGCGGCGCTCGGGAGAGCGGGCGGGTGAGTCAC |
| | | CCACACAAAGGAAAAGGGCCTTTCCGTCCTCAGCCGTCGCTTCA |
| | | TGTGACTCCACGGAGTACCGGGCGCCGTCCAGGCACCTCGATTA |
| | | GTTCTCGAGCTTTTGGAGTACGTCGTCTTTAGGTTGGGGGGAGGG |
| | | GTTTTATGCGATGGAGTTTCCCCACACTGAGTGGGTGGAGACTG |
| | | AAGTTAGGCCAGCTTGGCACTTGATGTAATTCTCCTTGGAATTTG |
| | | CCCTTTTTGAGTTTGGATCTTGGTTCATTCTCAAGCCTCAGACAG |
| | | TGGTTCAAAGTTTTTTTCTTCCATTTCAGGTGTCGTGA |
| 5 | CCR5 miRNA | GAGCAAGCTCAGTTTACA |
| | target | |
| | sequence | |
| 6 | Vif miRNA | GGGATGTGTACTTCTGAACTT |
| | coding | |
| | sequence | |
| 7 | Tat miRNA | TCCGCTTCTTCCTGCCATAG |
| | coding | |
| | sequence | |
| 8 | TAR decoy | CTTGCAATGATGTCGTAATTTGCGTCTTACCTCGTTCTCGACAGC |
| | sequence | GACCAGATCTGAGCCTGGGAGCTCTCTGGCTGTCAGTAAGCTGG |
| | | TACAGAAGGTTGACGAAAATTCTTACTGAGCAAGAAA |
| 9 | Rev/Tat | GCGGAGACAGCGACGAAGAGC |
| | shRNA target | |
| | sequence #1 | |
| 10 | Rev/Tat | GCGGAGACAGCGACGAAGAGCTTCAAGAGAGCTCTTCGTCGCTG |
| | shRNA coding | TCTCCGCTTTTT |
| | sequence #1 | |
| 11 | Gag shRNA | GAAGAAATGATGACAGCAT |
| | target sequence | |
| 12 | Gag shRNA | GAAGAAATGATGACAGCATTTCAAGAGAATGCTGTCATCATTTC |
| | coding | TTCTTTTT |
| | sequence | |
| 13 | Pol shRNA | CAGGAGCAGATGATACAG |
| | target sequence | |
| 14 | Pol shRNA | CAGGAGATGATACAGTTCAAGAGACTGTATCATCTGCTCCTGTTT |
| | coding | TT |
| | sequence | |
| 15 | CCR5 shRNA | GTGTCAAGTCCAATCTATG |
| | target sequence | |
| | #1 | |
| 16 | CCR5 shRNA | GTGTCAAGTCCAATCTATGTTCAAGAGACATAGATTGGACTTGA |
| | coding | CACTTTTT |
| | sequence #1 | |
| 17 | CCR5 shRNA | GAGCATGACTGACATCTAC |
| | target sequence | |
| | #2 | |
| 18 | CCR5 shRNA | GAGCATGACTGACATCTACTTCAAGAGAGTAGATGTCAGTCATG |
| | coding | CTCTTTTT |
| | sequence #2 | |
| 19 | CCR5 shRNA | GTAGCTCTAACAGGTTGGA |
| | target sequence | |
| | #3 | |
| 20 | CCR5 shRNA | GTAGCTCTAACAGGTTGGATTCAAGAGATCCAACCTGTTAGAGC |
| | coding | TACTTTTT |
| | sequence #3 | |
| 21 | CCR5 shRNA | GTTCAGAAACTACCTCTTA |
| | target sequence | |
| | #4 | |
| 22 | CCR5 shRNA | GTTCAGAAACTACCTCTTATTCAAGAGATAAGAGGTAGTTTCTGA |
| | coding | ACTTTTT |
| | sequence #4 | |
| 23 | CCR5 shRNA | GAGCAAGCTCAGTTTACACC |
| | target sequence | |
| | #5 | |
| 24 | CCR5 shRNA | GAGCAAGCTCAGTTTACACCTTCAAGAGAGGTGTAAACTGAGCT |
| | coding | TGCTCTTTTT |
| | sequence #5 | |
| 25 | Homo sapiens | ATGGATTATCAAGTGTCAAGTCCAATCTATGACATCAATTATTAT |
| | CCR5 mRNA | ACATCGGAGCCCTGCCAAAAAATCAATGTGAAGCAAATCGCAGC |
| | target sequence | CCGCCTCCTGCCTCCGCTCTACTCACTGGTGTTCATCTTTGGTTTT |
| | 1 | GTGGGC |
| 26 | Homo sapiens | AACATGCTGGTCATCCTCATCCTGATAAACTGCAAAAGGCTGAA |
| | CCR5 mRNA | GAGCATGACTGACATCTACCTGCTCAACCTGGCCATCTCTGACCT |
| | target sequence | GTTTTTCCTTCTTACTGTCCCCTTCTGGGCTCACTATGCTGCCGCC |
| | 2 | CAGTGGGACTTTGGAAATACAATGTGTCAACTCTTGACAGGGCT |
| | | CTATTTTATAGGCTTCTTCTCTGGAATCTTCTTCATCATCCTCCTG |
| | | ACAATCGATAGGTACCTGGCTGTCGTCCATGCTGTGTTTGCTTTA |
| | | AAAGCCAGGACGGTCACCTTTGGGGTGGTGACAAGTGTGATCAC |
| | | TTGGGTGGTGGCTGTGTTTGCGTCTCTCCCAGGAATCATCTTTAC |
| | | CAGATCTCAAAAAGAAGGTCTTCATTACACCTGCAGCTCTCATTT |
| | | TCCATACAGTCAGTATCAATTCTGGAAGAATTTCCAGACATTAAA |
| | | GATAGTCATCTTGGGGCTGGTCCTGCCGCTGCTTGTCATGGTCAT |
| | | CTGCTACTCGGGAATCCTAAAAACTCTGCTTCGGTGTCGAAATGA |
| | | GAAGAAGAGGCACAGGGCTGTGAGGCTTATCTTCACCATCATGA |
| | | TTGTTTATTTTCTCTTCTGGGCTCCCTACAACATTGTCCTTCTCCT |
| | | GAAC |
| 27 | Homo sapiens | ACCTTCCAGGAATTCTTTGGCCTGAATAATTGCAGTAGCTCTAAC |
| | CCR5 mRNA | AGGTTGGACCAAGCTATGCAGGTGA |
| | target sequence | |
| | 3 | |
| 28 | Homo sapiens | CAGAGACTCTTGGGATGACGCACTGCTGCATCAACCCCATCATCT |
| | CCR5 mRNA | ATGCCTTTGTCGGGGAGAAGTTCAGAAACTACCTCTTAGTCTTCT |
| | target sequence | TCCAAAAGCACATTGCCAAACGCTTCTGCAAATGCTGTTCTATTT |
| | 4 | TCCAG |
| 29 | Homo sapiens | CAAGAGGCTCCCGAGCGAGCAAGCTCAGTTTACACCCGATCCAC |
| | CCR5 mRNA | TGGGGAGCAGGAAATATCTGTGGGCTTGTGA |
| | target sequence | |
| | 5 | |
| 30 | miR30- | AGGTATATTGCTGTTGACAGTGAGCGACTGTAAACTGAGCTTGCT |
| | CCR5/miR21- | CTACTGTGAAGCCACAGATGGGTAGAGCAAGCACAGTTTACCGC |
| | Vif/miR185 | TGCCTACTGCCTCGGACTTCAAGGGGCTTCCCGGGCATCTCCATG |
| | Tat microRNA | GCTGTACCACCTTGTCGGGGGATGTGTACTTCTGAACTTGTGTTG |
| | cluster coding | AATCTCATGGAGTTCAGAAGAACACATCCGCACTGACATTTTGG |
| | sequence | TATCTTTCATCTGACCAGCTAGCGGGCCTGGCTCGAGCAGGGGG |
| | | CGAGGGATTCCGCTTCTTCCTGCCATAGCGTGGTCCCCTCCCCTA |
| | | TGGCAGGCAGAAGCGGCACCTTCCCTCCCAATGACCGCGTCTTC |
| | | GTC |
| 31 | Long WPRE | AATCAACCTCTGGATTACAAAATTTGTGAAAGATTGACTGGTATT |
| | sequence | CTTAACTATGTTGCTCCTTTTACGCTATGTGGATACGCTGCTTTAA |
| | | TGCCTTTGTATCATGCTATTGCTTCCCGTATGGCTTTCATTTTCTC |
| | | CTCCTTGTATAAATCCTGGTTGCTGTCTCTTTATGAGGAGTTGTG |
| | | GCCCGTTGTCAGGCAACGTGGCGTGGTGTGCACTGTGTTTGCTGA |
| | | CGCAACCCCCACTGGTTGGGGCATTGCCACCACCTGTCAGCTCCT |
| | | TTCCGGGACTTTCGCTTTCCCCCTCCCTATTGCCACGGCGGAACT |
| | | CATCGCCGCCTGCCTTGCCCGCTGCTGGACAGGGGCTCGGCTGTT |
| | | GGGCACTGACAATTCCGTGGTGTTGTCGGGGAAATCATCGTCCTT |
| | | TCCTTGGCTGCTCGCCTGTGTTGCCACCTGGATTCTGCGCGGGAC |
| | | GTCCTTCTGCTACGTCCCTTCGGCCCTCAATCCAGCGGACCTTCC |
| | | TTCCCGCGGCCTGCTGCCGGCTCTGCGGCCTCTTCCGCGTCTTCG |
| | | CCTTCGCCCTCAGACGAGTCGGATCTCCCTTTGGGCCGCCTCCCC |
| | | GCCT |
| 32 | Elongation | CCGGTGCCTAGAGAAGGTGGCGCGGGGTAAACTGGGAAAGTGA |
| | Factor-1 alpha | TGTCGTGTACTGGCTCCGCCTTTTTCCCGAGGGTGGGGGAGAACC |
| | (EF1-alpha) | GTATATAAGTGCAGTAGTCGCCGTGAACGTTCTTTTTCGCAACGG |
| | promoter; | GTTTGCCGCCAGAACACAGGTAAGTGCCGTGTGTGGTTCCCGCG |
| | miR30CCR5; | GGCCTGGCCTCTTTACGGGTTATGGCCCTTGCGTGCCTTGAATTA |
| | miR21Vif; | CTTCCACGCCCCTGGCTGCAGTACGTGATTCTTGATCCCGAGCTT |
| | miR185 Tat | CGGGTTGGAAGTGGGTGGGAGAGTTCGAGGCCTTGCGCTTAAGG |
| | cluster coding | AGCCCCTTCGCCTCGTGCTTGAGTTGAGGCCTGGCCTGGGCGCTG |
| | sequence | GGGCCGCCGCGTGCGAATCTGGTGGCACCTTCGCGCCTGTCTCGC |
| | | TGCTTTCGATAAGTCTCTAGCCATTTAAAATTTTTGATGACCTGC |
| | | TGCGACGCTTTTTTTCTGGCAAGATAGTCTTGTAAATGCGGGCCA |
| | | AGATCTGCACACTGGTATTTCGGTTTTTGGGGCCGCGGGCGGCG |
| | | ACGGGGCCCGTGCGTCCCAGCGCACATGTTCGGCGAGGCGGGGC |
| | | CTGCGAGCGCGGCCACCGAGAATCGGACGGGGGTAGTCTCAAGC |
| | | TGGCCGGCCTGCTCTGGTGCCTGGCCTCGCGCCGCCGTGTATCGC |
| | | CCCGCCCTGGGCGGCAAGGCTGGCCCGGTCGGCACCAGTTGCGT |
| | | GAGCGGAAAGATGGCCGCTTCCCGGCCCTGCTGCAGGGAGCTCA |
| | | AAATGGAGGACGCGGCGCTCGGGAGAGCGGGCGGGTGAGTCAC |
| | | CCACACAAAGGAAAAGGGCCTTTCCGTCCTCAGCCGTCGCTTCA |
| | | TGTGACTCCACGGAGTACCGGGCGCCGTCCAGGCACCTCGATTA |
| | | GTTCTCGAGCTTTTGGAGTACGTCGTCTTTAGGTTGGGGGGAGGG |
| | | GTTTTATGCGATGGAGTTTCCCCACACTGAGTGGGTGGAGACTG |
| | | AAGTTAGGCCAGCTTGGCACTTGATGTAATTCTCCTTGGAATTTG |
| | | CCCTTTTTGAGTTTGGATCTTGGTTCATTCTCAAGCCTCAGACAG |
| | | TGGTTCAAAGTTTTTTTCTTCCATTTCAGGTGTCGTGATGTACA |
| | | AGGTATATTGCTGTTGACAGTGAGCGACTGTAAACTGAGCTTGCT |
| | | CTACTGTGAAGCCACAGATGGGTAGAGCAAGCACAGTTTACCGC |
| | | TGCCTACTGCCTCGGACTTCAAGGGGCTTCCCGGGCATCTCCATG |
| | | GCTGTACCACCTTGTCGGGGGATGTGTACTTCTGAACTTGTGTTG |
| | | AATCTCATGGAGTTCAGAAGAACACATCCGCACTGACATTTTGG |
| | | TATCTTTCATCTGACCAGCTAGCGGGCCTGGCTCGAGCAGGGGG |
| | | CGAGGGATTCCGCTTCTTCCTGCCATAGCGTGGTCCCCTCCCCTA |
| | | TGGCAGGCAGAAGCGGCACCTTCCCTCCCAATGACCGCGTCTTC |
| | | GTC |
| 33 | Rous Sarcoma | GTAGTCTTATGCAATACTCTTGTAGTCTTGCAACATGGTAACGAT |
| | virus (RSV) | GAGTTAGCAACATGCCTTACAAGGAGAGAAAAAGCACCGTGCAT |
| | promoter | GCCGATTGGTGGAAGTAAGGTGGTACGATCGTGCCTTATTAGGA |
| | | AGGCAACAGACGGGTCTGACATGGATTGGACGAACCACTGAATT |
| | | GCCGCATTGCAGAGATATTGTATTTAAGTGCCTAGCTCGATACAA |
| | | TAAACG |
| 34 | 5′ Long | GGTCTCTCTGGTTAGACCAGATCTGAGCCTGGGAGCTCTCTGGCT |
| | terminal repeat | AACTAGGGAACCCACTGCTTAAGCCTCAATAAAGCTTGCCTTGA |
| | (LTR) | GTGCTTCAAGTAGTGTGTGCCCGTCTGTTGTGTGACTCTGGTAAC |
| | | TAGAGATCCCTCAGACCCTTTTAGTCAGTGTGGAAAATCTCTAGC |
| | | A |
| 35 | Psi Packaging | TACGCCAAAAATTTTGACTAGCGGAGGCTAGAAGGAGAGAG |
| | signal | |
| 36 | Rev response | AGGAGCTTTGTTCCTTGGGTTCTTGGGAGCAGCAGGAAGCACTA |
| | element (RRE) | TGGGCGCAGCCTCAATGACGCTGACGGTACAGGCCAGACAATTA |
| | | TTGTCTGGTATAGTGCAGCAGCAGAACAATTTGCTGAGGGCTATT |
| | | GAGGCGCAACAGCATCTGTTGCAACTCACAGTCTGGGGCATCAA |
| | | GCAGCTCCAGGCAAGAATCCTGGCTGTGGAAAGATACCTAAAGG |
| | | ATCAACAGCTCC |
| 37 | Central | TTTTAAAAGAAAAGGGGGGATTGGGGGGTACAGTGCAGGGGAA |
| | polypurine tract | AGAATAGTAGACATAATAGCAACAGACATACAAACTAAAGAATT |
| | (cPPT) | ACAAAAACAAATTACAAAATTCAAAATTTTA |
| 38 | 3′ delta LTR | TGGAAGGGCTAATTCACTCCCAACGAAGATAAGATCTGCTTTTTG |
| | | CTTGTACTGGGTCTCTCTGGTTAGACCAGATCTGAGCCTGGGAGC |
| | | TCTCTGGCTAACTAGGGAACCCACTGCTTAAGCCTCAATAAAGCT |
| | | TGCCTTGAGTGCTTCAAGTAGTGTGTGCCCGTCTGTTGTGTGACT |
| | | CTGGTAACTAGAGATCCCTCAGACCCTTTTAGTCAGTGTGGAAA |
| | | ATCTCTAGCAGTAGTAGTTCATGTCA |
| 39 | CMV early | TAGTTATTAATAGTAATCAATTACGGGGTCATTAGTTCATAGCCC |
| | (CAG) | ATATATGGAGTTCCGCGTTACATAACTTACGGTAAATGGCCCGCC |
| | enhancer; | TGGCTGACCGCCCAACGACCCCCGCCCATTGACGTCAATAATGA |
| | Enhance | CGTATGTTCCCATAGTAACGCCAATAGGGACTTTCCATTGACGTC |
| | Transcription | AATGGGTGGACTATTTACGGTAAACTGCCCACTTGGCAGTACAT |
| | | CAAGTGTATCATATGCCAAGTACGCCCCCTATTGACGTCAATGAC |
| | | GGTAAATGGCCCGCCTGGCATTATGCCCAGTACATGACCTTATG |
| | | GGACTTTCCTACTTGGCAGTACATCTACGTATTAGTCATC |
| 40 | Chicken beta | GCTATTACCATGGGTCGAGGTGAGCCCCACGTTCTGCTTCACTCT |
| | actin (CAG) | CCCCATCTCCCCCCCCTCCCCACCCCCAATTTTGTATTTATTTATT |
| | promoter; | TTTTAATTATTTTGTGCAGCGATGGGGGCGGGGGGGGGGGGGGC |
| | Transcription | GCGCGCCAGGCGGGGCGGGGCGGGGCGAGGGGCGGGGCGGGGC |
| | | GAGGCGGAGAGGTGCGGCGGCAGCCAATCAGAGCGGCGCGCTC |
| | | CGAAAGTTTCCTTTTATGGCGAGGCGGCGGCGGCGGCGGCCCTA |
| | | TAAAAAGCGAAGCGCGCGGCGGGCG |
| 41 | Chicken beta | GGAGTCGCTGCGTTGCCTTCGCCCCGTGCCCCGCTCCGCGCCGCC |
| | actin intron; | TCGCGCCGCCCGCCCCGGCTCTGACTGACCGCGTTACTCCCACAG |
| | Enhance gene | GTGAGCGGGCGGGACGGCCCTTCTCCTCCGGGCTGTAATTAGCG |
| | expression | CTTGGTTTAATGACGGCTCGTTTCTTTTCTGTGGCTGCGTGAAAG |
| | | CCTTAAAGGGCTCCGGGAGGGCCCTTTGTGCGGGGGGGAGCGGC |
| | | TCGGGGGGTGCGTGCGTGTGTGTGTGCGTGGGGAGCGCCGCGTG |
| | | CGGCCCGCGCTGCCCGGCGGCTGTGAGCGCTGCGGGCGCGGCGC |
| | | GGGGCTTTGTGCGCTCCGCGTGTGCGCGAGGGGAGCGCGGCCGG |
| | | GGGCGGTGCCCCGCGGTGCGGGGGGGCTGCGAGGGGAACAAAG |
| | | GCTGCGTGCGGGGTGTGTGCGTGGGGGGGTGAGCAGGGGGTGTG |
| | | GGCGCGGCGGTCGGGCTGTAACCCCCCCCTGCACCCCCCTCCCC |
| | | GAGTTGCTGAGCACGGCCCGGCTTCGGGTGCGGGGCTCCGTGCG |
| | | GGGCGTGGCGCGGGGCTCGCCGTGCCGGGCGGGGGGTGGCGGC |
| | | AGGTGGGGGTGCCGGGCGGGGCGGGGCCGCCTCGGGCCGGGGA |
| | | GGGCTCGGGGGAGGGGCGCGGCGGCCCCGGAGCGCCGGCGGCT |
| | | GTCGAGGCGCGGCGAGCCGCAGCCATTGCCTTTTATGGTAATCG |
| | | TGCGAGAGGGCGCAGGGACTTCCTTTGTCCCAAATCTGGCGGAG |
| | | CCGAAATCTGGGAGGCGCCGCCGCACCCCCTCTAGCGGGCGCGG |
| | | GCGAAGCGGTGCGGCGCCGGCAGGAAGGAAATGGGCGGGGAGG |
| | | GCCTTCGTGCGTCGCCGCGCCGCCGTCCCCTTCTCCATCTCCAGC |
| | | CTCGGGGCTGCCGCAGGGGGACGGCTGCCTTCGGGGGGGACGGG |
| | | GCAGGGCGGGGTTCGGCTTCTGGCGTGTGACCGGCGG |
| 42 | HIV Gag; Viral | ATGGGTGCGAGAGCGTCAGTATTAAGCGGGGGAGAATTAGATCG |
| | capsid | ATGGGAAAAAATTCGGTTAAGGCCAGGGGGAAAGAAAAAATAT |
| | | AAATTAAAACATATAGTATGGGCAAGCAGGGAGCTAGAACGATT |
| | | CGCAGTTAATCCTGGCCTGTTAGAAACATCAGAAGGCTGTAGAC |
| | | AAATACTGGGACAGCTACAACCATCCCTTCAGACAGGATCAGAA |
| | | GAACTTAGATCATTATATAATACAGTAGCAACCCTCTATTGTGTG |
| | | CATCAAAGGATAGAGATAAAAGACACCAAGGAAGCTTTAGACA |
| | | AGATAGAGGAAGAGCAAAACAAAAGTAAGAAAAAAGCACAGCA |
| | | AGCAGCAGCTGACACAGGACACAGCAATCAGGTCAGCCAAAATT |
| | | ACCCTATAGTGCAGAACATCCAGGGGCAAATGGTACATCAGGCC |
| | | ATATCACCTAGAACTTTAAATGCATGGGTAAAAGTAGTAGAAGA |
| | | GAAGGCTTTCAGCCCAGAAGTGATACCCATGTTTTCAGCATTATC |
| | | AGAAGGAGCCACCCCACAAGATTTAAACACCATGCTAAACACAG |
| | | TGGGGGGACATCAAGCAGCCATGCAAATGTTAAAAGAGACCATC |
| | | AATGAGGAAGCTGCAGAATGGGATAGAGTGCATCCAGTGCATGC |
| | | AGGGCCTATTGCACCAGGCCAGATGAGAGAACCAAGGGGAAGT |
| | | GACATAGCAGGAACTACTAGTACCCTTCAGGAACAAATAGGATG |
| | | GATGACACATAATCCACCTATCCCAGTAGGAGAAATCTATAAAA |
| | | GATGGATAATCCTGGGATTAAATAAAATAGTAAGAATGTATAGC |
| | | CCTACCAGCATTCTGGACATAAGACAAGGACCAAAGGAACCCTT |
| | | TAGAGACTATGTAGACCGATTCTATAAAACTCTAAGAGCCGAGC |
| | | AAGCTTCACAAGAGGTAAAAAATTGGATGACAGAAACCTTGTTG |
| | | GTCCAAAATGCGAACCCAGATTGTAAGACTATTTTAAAAGCATT |
| | | GGGACCAGGAGCGACACTAGAAGAAATGATGACAGCATGTCAG |
| | | GGAGTGGGGGGACCCGGCCATAAAGCAAGAGTTTTGGCTGAAGC |
| | | AATGAGCCAAGTAACAAATCCAGCTACCATAATGATACAGAAAG |
| | | GCAATTTTAGGAACCAAAGAAAGACTGTTAAGTGTTTCAATTGT |
| | | GGCAAAGAAGGGCACATAGCCAAAAATTGCAGGGCCCCTAGGA |
| | | AAAAGGGCTGTTGGAAATGTGGAAAGGAAGGACACCAAATGAA |
| | | AGATTGTACTGAGAGACAGGCTAATTTTTTAGGGAAGATCTGGC |
| | | CTTCCCACAAGGGAAGGCCAGGGAATTTTCTTCAGAGCAGACCA |
| | | GAGCCAACAGCCCCACCAGAAGAGAGCTTCAGGTTTGGGGAAG |
| | | AGACAACAACTCCCTCTCAGAAGCAGGAGCCGATAGACAAGGA |
| | | ACTGTATCCTTTAGCTTCCCTCAGATCACTCTTTGGCAGCGACCC |
| | | CTCGTCACAATAA |
| 43 | HIV Pol; | ATGAATTTGCCAGGAAGATGGAAACCAAAAATGATAGGGGGAA |
| | Protease and | TTGGAGGTTTTATCAAAGTAGGACAGTATGATCAGATACTCATA |
| | reverse | GAAATCTGCGGACATAAAGCTATAGGTACAGTATTAGTAGGACC |
| | transcriptase | TACACCTGTCAACATAATTGGAAGAAATCTGTTGACTCAGATTG |
| | | GCTGCACTTTAAATTTTCCCATTAGTCCTATTGAGACTGTACCAG |
| | | TAAAATTAAAGCCAGGAATGGATGGCCCAAAAGTTAAACAATGG |
| | | CCATTGACAGAAGAAAAAATAAAAGCATTAGTAGAAATTTGTAC |
| | | AGAAATGGAAAAGGAAGGAAAAATTTCAAAAATTGGGCCTGAA |
| | | AATCCATACAATACTCCAGTATTTGCCATAAAGAAAAAAGACAG |
| | | TACTAAATGGAGAAAATTAGTAGATTTCAGAGAACTTAATAAGA |
| | | GAACTCAAGATTTCTGGGAAGTTCAATTAGGAATACCACATCCT |
| | | GCAGGGTTAAAACAGAAAAAATCAGTAACAGTACTGGATGTGG |
| | | GCGATGCATATTTTTCAGTTCCCTTAGATAAAGACTTCAGGAAGT |
| | | ATACTGCATTTACCATACCTAGTATAAACAATGAGACACCAGGG |
| | | ATTAGATATCAGTACAATGTGCTTCCACAGGGATGGAAAGGATC |
| | | ACCAGCAATATTCCAGTGTAGCATGACAAAAATCTTAGAGCCTT |
| | | TTAGAAAACAAAATCCAGACATAGTCATCTATCAATACATGGAT |
| | | GATTTGTATGTAGGATCTGACTTAGAAATAGGGCAGCATAGAAC |
| | | AAAAATAGAGGAACTGAGACAACATCTGTTGAGGTGGGGATTTA |
| | | CCACACCAGACAAAAAACATCAGAAAGAACCTCCATTCCTTTGG |
| | | ATGGGTTATGAACTCCATCCTGATAAATGGACAGTACAGCCTAT |
| | | AGTGCTGCCAGAAAAGGACAGCTGGACTGTCAATGACATACAGA |
| | | AATTAGTGGGAAAATTGAATTGGGCAAGTCAGATTTATGCAGGG |
| | | ATTAAAGTAAGGCAATTATGTAAACTTCTTAGGGGAACCAAAGC |
| | | ACTAACAGAAGTAGTACCACTAACAGAAGAAGCAGAGCTAGAA |
| | | CTGGCAGAAAACAGGGAGATTCTAAAAGAACCGGTACATGGAG |
| | | TGTATTATGACCCATCAAAAGACTTAATAGCAGAAATACAGAAG |
| | | CAGGGGCAAGGCCAATGGACATATCAAATTTATCAAGAGCCATT |
| | | TAAAAATCTGAAAACAGGAAAATATGCAAGAATGAAGGGTGCC |
| | | CACACTAATGATGTGAAACAATTAACAGAGGCAGTACAAAAAAT |
| | | AGCCACAGAAAGCATAGTAATATGGGGAAAGACTCCTAAATTTA |
| | | AATTACCCATACAAAAGGAAACATGGGAAGCATGGTGGACAGA |
| | | GTATTGGCAAGCCACCTGGATTCCTGAGTGGGAGTTTGTCAATAC |
| | | CCCTCCCTTAGTGAAGTTATGGTACCAGTTAGAGAAAGAACCCA |
| | | TAATAGGAGCAGAAACTTTCTATGTAGATGGGGCAGCCAATAGG |
| | | GAAACTAAATTAGGAAAAGCAGGATATGTAACTGACAGAGGAA |
| | | GACAAAAAGTTGTCCCCCTAACGGACACAACAAATCAGAAGACT |
| | | GAGTTACAAGCAATTCATCTAGCTTTGCAGGATTCGGGATTAGA |
| | | AGTAAACATAGTGACAGACTCACAATATGCATTGGGAATCATTC |
| | | AAGCACAACCAGATAAGAGTGAATCAGAGTTAGTCAGTCAAATA |
| | | ATAGAGCAGTTAATAAAAAAGGAAAAAGTCTACCTGGCATGGGT |
| | | ACCAGCACACAAAGGAATTGGAGGAAATGAACAAGTAGATGGG |
| | | TTGGTCAGTGCTGGAATCAGGAAAGTACTA |
| 44 | HIV Integrase; | TTTTTAGATGGAATAGATAAGGCCCAAGAAGAACATGAGAAATA |
| | Integration of | TCACAGTAATTGGAGAGCAATGGCTAGTGATTTTAACCTACCAC |
| | viral RNA | CTGTAGTAGCAAAAGAAATAGTAGCCAGCTGTGATAAATGTCAG |
| | | CTAAAAGGGGAAGCCATGCATGGACAAGTAGACTGTAGCCCAG |
| | | GAATATGGCAGCTAGATTGTACACATTTAGAAGGAAAAGTTATC |
| | | TTGGTAGCAGTTCATGTAGCCAGTGGATATATAGAAGCAGAAGT |
| | | AATTCCAGCAGAGACAGGGCAAGAAACAGCATACTTCCTCTTAA |
| | | AATTAGCAGGAAGATGGCCAGTAAAAACAGTACATACAGACAA |
| | | TGGCAGCAATTTCACCAGTACTACAGTTAAGGCCGCCTGTTGGTG |
| | | GGCGGGGATCAAGCAGGAATTTGGCATTCCCTACAATCCCCAAA |
| | | GTCAAGGAGTAATAGAATCTATGAATAAAGAATTAAAGAAAATT |
| | | ATAGGACAGGTAAGAGATCAGGCTGAACATCTTAAGACAGCAGT |
| | | ACAAATGGCAGTATTCATCCACAATTTTAAAAGAAAAGGGGGGA |
| | | TTGGGGGGTACAGTGCAGGGGAAAGAATAGTAGACATAATAGC |
| | | AACAGACATACAAACTAAAGAATTACAAAAACAAATTACAAAA |
| | | ATTCAAAATTTTCGGGTTTATTACAGGGACAGCAGAGATCCAGTT |
| | | TGGAAAGGACCAGCAAAGCTCCTCTGGAAAGGTGAAGGGGCAG |
| | | TAGTAATACAAGATAATAGTGACATAAAAGTAGTGCCAAGAAGA |
| | | AAAGCAAAGATCATCAGGGATTATGGAAAACAGATGGCAGGTG |
| | | ATGATTGTGTGGCAAGTAGACAGGATGAGGATTAA |
| 45 | HIV RRE; | AGGAGCTTTGTTCCTTGGGTTCTTGGGAGCAGCAGGAAGCACTA |
| | Binds Rev | TGGGCGCAGCGTCAATGACGCTGACGGTACAGGCCAGACAATTA |
| | element | TTGTCTGGTATAGTGCAGCAGCAGAACAATTTGCTGAGGGCTATT |
| | | GAGGCGCAACAGCATCTGTTGCAACTCACAGTCTGGGGCATCAA |
| | | GCAGCTCCAGGCAAGAATCCTGGCTGTGGAAAGATACCTAAAGG |
| | | ATCAACAGCTCCT |
| 46 | HIV Rev; | ATGGCAGGAAGAAGCGGAGACAGCGACGAAGAACTCCTCAAGG |
| | Nuclear export | CAGTCAGACTCATCAAGTTTCTCTATCAAAGCAACCCACCTCCCA |
| | and stabilize | ATCCCGAGGGGACCCGACAGGCCCGAAGGAATAGAAGAAGAAG |
| | viral mRNA | GTGGAGAGAGAGACAGAGACAGATCCATTCGATTAGTGAACGG |
| | | ATCCTTAGCACTTATCTGGGACGATCTGCGGAGCCTGTGCCTCTT |
| | | CAGCTACCACCGCTTGAGAGACTTACTCTTGATTGTAACGAGGAT |
| | | TGTGGAACTTCTGGGACGCAGGGGGTGGGAAGCCCTCAAATATT |
| | | GGTGGAATCTCCTACAATATTGGAGTCAGGAGCTAAAGAATAG |
| 47 | Rabbit beta | AGATCTTTTTCCCTCTGCCAAAAATTATGGGGACATCATGAAGCC |
| | globin poly A; | CCTTGAGCATCTGACTTCTGGCTAATAAAGGAAATTTATTTTCAT |
| | RNA stability | TGCAATAGTGTGTTGGAATTTTTTGTGTCTCTCACTCGGAAGGAC |
| | | ATATGGGAGGGCAAATCATTTAAAACATCAGAATGAGTATTTGG |
| | | TTTAGAGTTTGGCAACATATGCCATATGCTGGCTGCCATGAACAA |
| | | AGGTGGCTATAAAGAGGTCATCAGTATATGAAACAGCCCCCTGC |
| | | TGTCCATTCCTTATTCCATAGAAAAGCCTTGACTTGAGGTTAGAT |
| | | TTTTTTTATATTTTGTTTTGTGTTATTTTTTTCTTTAACATCCCTAA |
| | | AATTTTCCTTACATGTTTTACTAGCCAGATTTTTCCTCCTCTCCTG |
| | | ACTACTCCCAGTCATAGCTGTCCCTCTTCTCTTATGAAGATC |
| 48 | Rabbit beta | AGATCTTTTTCCCTCTGCCAAAAATTATGGGGACATCATGAAGCC |
| | globin poly A; | CCTTGAGCATCTGACTTCTGGCTAATAAAGGAAATTTATTTTCAT |
| | RNA stability | TGCAATAGTGTGTTGGAATTTTTTGTGTCTCTCACTCGGAAGGAC |
| | | ATATGGGAGGGCAAATCATTTAAAACATCAGAATGAGTATTTGG |
| | | TTTAGAGTTTGGCAACATATGCCCATATGCTGGCTGCCATGAACA |
| | | AAGGTTGGCTATAAAGAGGTCATCAGTATATGAAACAGCCCCCT |
| | | GCTGTCCATTCCTTATTCCATAGAAAAGCCTTGACTTGAGGTTAG |
| | | ATTTTTTTTATATTTTGTTTTGTGTTATTTTTTTCTTTAACATCCCT |
| | | AAAATTTTCCTTACATGTTTTACTAGCCAGATTTTTCCTCCTCTCC |
| | | TGACTACTCCCAGTCATAGCTGTCCCTCTTCTCTTATGGAGATC |
| 49 | CMV | ACATTGATTATTGACTAGTTATTAATAGTAATCAATTACGGGGTC |
| | promoter; | ATTAGTTCATAGCCCATATATGGAGTTCCGCGTTACATAACTTAC |
| | Transcription | GGTAAATGGCCCGCCTGGCTGACCGCCCAACGACCCCCGCCCAT |
| | | TGACGTCAATAATGACGTATGTTCCCATAGTAACGCCAATAGGG |
| | | ACTTTCCATTGACGTCAATGGGTGGAGTATTTACGGTAAACTGCC |
| | | CACTTGGCAGTACATCAAGTGTATCATATGCCAAGTACGCCCCCT |
| | | ATTGACGTCAATGACGGTAAATGGCCCGCCTGGCATTATGCCCA |
| | | GTACATGACCTTATGGGACTTTCCTACTTGGCAGTACATCTACGT |
| | | ATTAGTCATCGCTATTACCATGGTGATGCGGTTTTGGCAGTACAT |
| | | CAATGGGCGTGGATAGCGGTTTGACTCACGGGGATTTCCAAGTC |
| | | TCCACCCCATTGACGTCAATGGGAGTTTGTTTTGGCACCAAAATC |
| | | AACGGGACTTTCCAAAATGTCGTAACAACTCCGCCCCATTGACG |
| | | CAAATGGGCGGTAGGCGTGTACGGTGGGAGGTCTATATAAGC |
| 50 | Beta globin | GTGAGTTTGGGGACCCTTGATTGTTCTTTCTTTTTCGCTATTGTAA |
| | intron; Enhance | AATTCATGTTATATGGAGGGGGCAAAGTTTTCAGGGTGTTGTTTA |
| | gene | GAATGGGAAGATGTCCCTTGTATCACCATGGACCCTCATGATAA |
| | expression | TTTTGTTTCTTTCACTTTCTACTCTGTTGACAACCATTGTCTCCTCT |
| | | TATTTTCTTTTCATTTTCTGTAACTTTTTCGTTAAACTTTAGCTTGC |
| | | ATTTGTAACGAATTTTTAAATTCACTTTTGTTTATTTGTCAGATTG |
| | | TAAGTACTTTCTCTAATCACTTTTTTTTCAAGGCAATCAGGGTAT |
| | | ATTATATTGTACTTCAGCACAGTTTTAGAGAACAATTGTTATAAT |
| | | TAAATGATAAGGTAGAATATTTCTGCATATAAATTCTGGCTGGCG |
| | | TGGAAATATTCTTATTGGTAGAAACAACTACACCCTGGTCATCAT |
| | | CCTGCCTTTCTCTTTATGGTTACAATGATATACACTGTTTGAGAT |
| | | GAGGATAAAATACTCTGAGTCCAAACCGGGCCCCTCTGCTAACC |
| | | ATGTTCATGCCTTCTTCTCTTTCCTACAG |
| 51 | VSV-G; | ATGAAGTGCCTTTTGTACTTAGCCTTTTTATTCATTGGGGTGAATT |
| | Glycoprotein | GCAAGTTCACCATAGTTTTTCCACACAACCAAAAAGGAAACTGG |
| | envelope-cell | AAAAATGTTCCTTCTAATTACCATTATTGCCCGTCAAGCTCAGAT |
| | entry | TTAAATTGGCATAATGACTTAATAGGCACAGCCTTACAAGTCAA |
| | | AATGCCCAAGAGTCACAAGGCTATTCAAGCAGACGGTTGGATGT |
| | | GTCATGCTTCCAAATGGGTCACTACTTGTGATTTCCGCTGGTATG |
| | | GACCGAAGTATATAACACATTCCATCCGATCCTTCACTCCATCTG |
| | | TAGAACAATGCAAGGAAAGCATTGAACAAACGAAACAAGGAAC |
| | | TTGGCTGAATCCAGGCTTCCCTCCTCAAAGTTGTGGATATGCAAC |
| | | TGTGACGGATGCCGAAGCAGTGATTGTCCAGGTGACTCCTCACC |
| | | ATGTGCTGGTTGATGAATACACAGGAGAATGGGTTGATTCACAG |
| | | TTCATCAACGGAAAATGCAGCAATTACATATGCCCCACTGTCCAT |
| | | AACTCTACAACCTGGCATTCTGACTATAAGGTCAAAGGGCTATG |
| | | TGATTCTAACCTCATTTCCATGGACATCACCTTCTTCTCAGAGGA |
| | | CGGAGAGCTATCATCCCTGGGAAAGGAGGGCACAGGGTTCAGA |
| | | AGTAACTACTTTGCTTATGAAACTGGAGGCAAGGCCTGCAAAAT |
| | | GCAATACTGCAAGCATTGGGGAGTCAGACTCCCATCAGGTGTCT |
| | | GGTTCGAGATGGCTGATAAGGATCTCTTTGCTGCAGCCAGATTCC |
| | | CTGAATGCCCAGAAGGGTCAAGTATCTCTGCTCCATCTCAGACCT |
| | | CAGTGGATGTAAGTCTAATTCAGGACGTTGAGAGGATCTTGGAT |
| | | TATTCCCTCTGCCAAGAAACCTGGAGCAAAATCAGAGCGGGTCT |
| | | TCCAATCTCTCCAGTGGATCTCAGCTATCTTGCTCCTAAAAACCC |
| | | AGGAACCGGTCCTGCTTTCACCATAATCAATGGTACCCTAAAAT |
| | | ACTTTGAGACCAGATACATCAGAGTCGATATTGCTGCTCCAATCC |
| | | TCTCAAGAATGGTCGGAATGATCAGTGGAACTACCACAGAAAGG |
| | | GAACTGTGGGATGACTGGGCACCATATGAAGACGTGGAAATTGG |
| | | ACCCAATGGAGTTCTGAGGACCAGTTCAGGATATAAGTTTCCTTT |
| | | ATACATGATTGGACATGGTATGTTGGACTCCGATCTTCATCTTAG |
| | | CTCAAAGGCTCAGGTGTTCGAACATCCTCACATTCAAGACGCTG |
| | | CTTCGCAACTTCCTGATGATGAGAGTTTATTTTTTGGTGATACTG |
| | | GGCTATCCAAAAATCCAATCGAGCTTGTAGAAGGTTGGTTCAGT |
| | | AGTTGGAAAAGCTCTATTGCCTCTTTTTTCTTTATCATAGGGTTA |
| | | ATCATTGGACTATTCTTGGTTCTCCGAGTTGGTATCCATCTTTGCA |
| | | TTAAATTAAAGCACACCAAGAAAAGACAGATTTATACAGACATA |
| | | GAGATGA |
| 52 | Promoter; PGK | GGGGTTGGGGTTGCGCCTTTTCCAAGGCAGCCCTGGGTTTGCGCA |
| | | GGGACGCGGCTGCTCTGGGCGTGGTTCCGGGAAACGCAGCGGCG |
| | | CCGACCCTGGGTCTCGCACATTCTTCACGTCCGTTCGCAGCGTCA |
| | | CCCGGATCTTCGCCGCTACCCTTGTGGGCCCCCCGGCGACGCTTC |
| | | CTGCTCCGCCCCTAAGTCGGGAAGGTTCCTTGCGGTTCGCGGCGT |
| | | GCCGGACGTGACAAACGGAAGCCGCACGTCTCACTAGTACCCTC |
| | | GCAGACGGACAGCGCCAGGGAGCAATGGCAGCGCGCCGACCGC |
| | | GATGGGCTGTGGCCAATAGCGGCTGCTCAGCAGGGCGCGCCGAG |
| | | AGCAGCGGCCGGGAAGGGGCGGTGCGGGAGGCGGGGTGTGGGG |
| | | CGGTAGTGTGGGCCCTGTTCCTGCCCGCGCGGTGTTCCGCATTCT |
| | | GCAAGCCTCCGGAGCGCACGTCGGCAGTCGGCTCCCTCGTTGAC |
| | | CGAATCACCGACCTCTCTCCCCAG |
| 53 | Promoter; UbC | GCGCCGGGTTTTGGCGCCTCCCGCGGGCGCCCCCCTCCTCACGGC |
| | | GAGCGCTGCCACGTCAGACGAAGGGCGCAGGAGCGTTCCTGATC |
| | | CTTCCGCCCGGACGCTCAGGACAGCGGCCCGCTGCTCATAAGAC |
| | | TCGGCCTTAGAACCCCAGTATCAGCAGAAGGACATTTTAGGACG |
| | | GGACTTGGGTGACTCTAGGGCACTGGTTTTCTTTCCAGAGAGCGG |
| | | AACAGGCGAGGAAAAGTAGTCCCTTCTCGGCGATTCTGCGGAGG |
| | | GATCTCCGTGGGGCGGTGAACGCCGATGATTATATAAGGACGCG |
| | | CCGGGTGTGGCACAGCTAGTTCCGTCGCAGCCGGGATTTGGGTC |
| | | GCGGTTCTTGTTTGTGGATCGCTGTGATCGTCACTTGGTGAGTTG |
| | | CGGGCTGCTGGGCTGGCCGGGGCTTTCGTGGCCGCCGGGCCGCT |
| | | CGGTGGGACGGAAGCGTGTGGAGAGACCGCCAAGGGCTGTAGT |
| | | CTGGGTCCGCGAGCAAGGTTGCCCTGAACTGGGGGTTGGGGGGA |
| | | GCGCACAAAATGGCGGCTGTTCCCGAGTCTTGAATGGAAGACGC |
| | | TTGTAAGGCGGGCTGTGAGGTCGTTGAAACAAGGTGGGGGGCAT |
| | | GGTGGGCGGCAAGAACCCAAGGTCTTGAGGCCTTCGCTAATGCG |
| | | GGAAAGCTCTTATTCGGGTGAGATGGGCTGGGGCACCATCTGGG |
| | | GACCCTGACGTGAAGTTTGTCACTGACTGGAGAACTCGGGTTTGT |
| | | CGTCTGGTTGCGGGGGCGGCAGTTATGCGGTGCCGTTGGGCAGT |
| | | GCACCCGTACCTTTGGGAGCGCGCGCCTCGTCGTGTCGTGACGTC |
| | | ACCCGTTCTGTTGGCTTATAATGCAGGGTGGGGCCACCTGCCGGT |
| | | AGGTGTGCGGTAGGCTTTTCTCCGTCGCAGGACGCAGGGTTCGG |
| | | GCCTAGGGTAGGCTCTCCTGAATCGACAGGCGCCGGACCTCTGG |
| | | TGAGGGGAGGGATAAGTGAGGCGTCAGTTTCTTTGGTCGGTTTT |
| | | ATGTACCTATCTTCTTAAGTAGCTGAAGCTCCGGTTTTGAACTAT |
| | | GCGCTCGGGGTTGGCGAGTGTGTTTTGTGAAGTTTTTTAGGCACC |
| | | TTTTGAAATGTAATCATTTGGGTCAATATGTAATTTTCAGTGTTA |
| | | GACTAGTAAA |
| 54 | Poly A; SV40 | GTTTATTGCAGCTTATAATGGTTACAAATAAAGCAATAGCATCAC |
| | | AAATTTCACAAATAAAGCATTTTTTTCACTGCATTCTAGTTGTGG |
| | | TTTGTCCAAACTCATCAATGTATCTTATCA |
| 55 | Poly A; bGH | GACTGTGCCTTCTAGTTGCCAGCCATCTGTTGTTTGCCCCTCCCCC |
| | | GTGCCTTCCTTGACCCTGGAAGGTGCCACTCCCACTGTCCTTTCC |
| | | TAATAAAATGAGGAAATTGCATCGCATTGTCTGAGTAGGTGTCA |
| | | TTCTATTCTGGGGGGTGGGGTGGGGCAGGACAGCAAGGGGGAG |
| | | GATTGGGAAGACAATAGCAGGCATGCTGGGGATGCGGTGGGCTC |
| | | TATGG |
| 56 | HIV Gag; Bal | ATGGGTGCGAGAGCGTCAGTATTAAGCGGGGGAGAATTAGATAG |
| | | GTGGGAAAAAATTCGGTTAAGGCCAGGGGGAAAGAAAAAATAT |
| | | AGATTAAAACATATAGTATGGGCAAGCAGGGAACTAGAAAGATT |
| | | CGCAGTCAATCCTGGCCTGTTAGAAACATCAGAAGGCTGCAGAC |
| | | AAATACTGGGACAGCTACAACCATCCCTTCAGACAGGATCAGAA |
| | | GAACTTAGATCATTATATAATACAGTAGCAACCCTCTATTGTGTA |
| | | CATCAAAAGATAGAGGTAAAAGACACCAAGGAAGCTTTAGACA |
| | | AAATAGAGGAAGAGCAAAACAAATGTAAGAAAAAGGCACAGCA |
| | | AGCAGCAGCTGACACAGGAAACAGCGGTCAGGTCAGCCAAAAT |
| | | TTCCCTATAGTGCAGAACCTCCAGGGGCAAATGGTACATCAGGC |
| | | CATATCACCTAGAACTTTAAATGCATGGGTAAAAGTAATAGAAG |
| | | AGAAAGCTTTCAGCCCAGAAGTAATACCCATGTTTTCAGCATTAT |
| | | CAGAAGGAGCCACCCCACAAGATTTAAACACCATGCTAAACACA |
| | | GTGGGGGGACATCAAGCAGCCATGCAAATGTTAAAAGAACCCAT |
| | | CAATGAGGAAGCTGCAAGATGGGATAGATTGCATCCCGTGCAGG |
| | | CAGGGCCTGTTGCACCAGGCCAGATAAGAGATCCAAGGGGAAGT |
| | | GACATAGCAGGAACTACCAGTACCCTTCAGGAACAAATAGGATG |
| | | GATGACAAGTAATCCACCTATCCCAGTAGGAGAAATCTATAAAA |
| | | GATGGATAATCCTGGGATTAAATAAAATAGTAAGGATGTATAGC |
| | | CCTACCAGCATTTTGGACATAAGACAAGGACCAAAGGAACCCTT |
| | | TAGAGACTATGTAGACCGGTTCTATAAAACTCTAAGAGCCGAGC |
| | | AAGCTTCACAGGAGGTAAAAAATTGGATGACAGAAACCTTGTTG |
| | | GTCCAAAATGCGAACCCAGATTGTAAGACTATTTTAAAAGCATT |
| | | GGGACCAGCAGCTACACTAGAAGAAATGATGACAGCATGTCAG |
| | | GGAGTGGGAGGACCCAGCCATAAAGCAAGAATTTTGGCAGAAG |
| | | CAATGAGCCAAGTAACAAATTCAGCTACCATAATGATGCAGAAA |
| | | GGCAATTTTAGGAACCAAAGAAAGATTGTTAAATGTTTCAATTG |
| | | TGGCAAAGAAGGGCACATAGCCAGAAACTGCAGGGCCCCTAGG |
| | | AAAAGGGGCTGTTGGAAATGTGGAAAGGAAGGACACCAAATGA |
| | | AAGACTGTACTGAGAGACAGGCTAATTTTTTAGGGAAAATCTGG |
| | | CCTTCCCACAAAGGAAGGCCAGGGAATTTCCTTCAGAGCAGACC |
| | | AGAGCCAACAGCCCCACCAGCCCCACCAGAAGAGAGCTTCAGGT |
| | | TTGGGGAAGAGACAACAACTCCCTCTCAGAAGCAGGAGCTGATA |
| | | GACAAGGAACTGTATCCTTTAGCTTCCCTCAGATCACTCTTTGGC |
| | | AACGACCCCTCGTCACAATAA |
| 57 | HIV Pol; Bal | ATGAATTTGCCAGGAAGATGGAAACCAAAAATGATAGGGGGAA |
| | | TTGGAGGTTTTATCAAAGTAAGACAGTATGATCAGATACTCATA |
| | | GAAATCTGTGGACATAAAGCTATAGGTACAGTATTAATAGGACC |
| | | TACACCTGTCAACATAATTGGAAGAAATCTGTTGACTCAGATTG |
| | | GTTGCACTTTAAATTTTCCCATTAGTCCTATTGAAACTGTACCAG |
| | | TAAAATTAAAACCAGGAATGGATGGCCCAAAAGTTAAACAATGG |
| | | CCACTGACAGAAGAAAAAATAAAAGCATTAATGGAAATCTGTAC |
| | | AGAAATGGAAAAGGAAGGGAAAATTTCAAAAATTGGGCCTGAA |
| | | AATCCATACAATACTCCAGTATTTGCCATAAAGAAAAAAGACAG |
| | | TACTAAATGGAGAAAATTAGTAGATTTCAGAGAACTTAATAAGA |
| | | AAACTCAAGACTTCTGGGAAGTACAATTAGGAATACACATCCCG |
| | | CAGGGGTTAAAAAAGAAAAAATCAGTAACAGTACTGGATGTGG |
| | | GTGATGCATATTTTTCAGTTCCCTTAGATAAAGAATTCAGGAAGT |
| | | ATACTGCATTTACCATACCTAGTATAAACAATGAAACACCAGGG |
| | | ATCAGATATCAGTACAATGTACTTCCACAGGGATGGAAAGGATC |
| | | ACCAGCAATATTTCAAAGTAGCATGACAAGAATCTTAGAGCCTT |
| | | TTAGAAAACAAAATCCAGAAATAGTGATCTATCAATACATGGAT |
| | | GATTTGTATGTAGGATCTGACTTAGAAATAGGGCAGCATAGAAC |
| | | AAAAATAGAGGAACTGAGACAACATCTGTTGAGGTGGGGATTTA |
| | | CCACACCAGACAAAAAACATCAGAAAGAACCTCCATTCCTTTGG |
| | | ATGGGTTATGAACTCCATCCTGATAAATGGACAGTACAGCCTAT |
| | | AGTGCTGCCAGAAAAAGACAGCTGGACTGTCAATGACATACAGA |
| | | AGTTAGTGGGAAAATTGAATTGGGCAAGTCAGATTTACCCAGGA |
| | | ATTAAAGTAAAGCAATTATGTAGGCTCCTTAGGGGAACCAAGGC |
| | | ATTAACAGAAGTAATACCACTAACAAAAGAAACAGAGCTAGAA |
| | | CTGGCAGAGAACAGGGAAATTCTAAAAGAACCAGTACATGGGG |
| | | TGTATTATGACCCATCAAAAGACTTAATAGCAGAAATACAGAAG |
| | | CAGGGGCAAGGCCAATGGACATATCAAATTTATCAAGAGCCATT |
| | | TAAAAATCTGAAAACAGGAAAATATGCAAGAATGAGGGGTGCC |
| | | CACACTAATGATGTAAAACAATTAACAGAGGCAGTGCAAAAAAT |
| | | AACCACAGAAAGCATAGTAATATGGGGAAAGACTCCTAAATTTA |
| | | AACTACCCATACAAAAAGAAACATGGGAAACATGGTGGACAGA |
| | | GTATTGGCAAGCCACCTGGATTCCTGAGTGGGAGTTTGTCAATAC |
| | | CCCTCCCTTAGTGAAATTATGGTACCAGTTAGAGAAAGAACCCA |
| | | TAATAGGAGCAGAAACATTCTATGTAGATGGAGCAGCTAACCGG |
| | | GAGACTAAATTAGGAAAAGCAGGATATGTTACTAACAGAGGAA |
| | | GACAAAAAGTTGTCTCCCTAACTGACACAACAAATCAGAAGACT |
| | | GAGTTACAAGCAATTCATCTAGCTTTACAAGATTCAGGATTAGA |
| | | AGTAAACATAGTAACAGACTCACAATATGCATTAGGAATCATTC |
| | | AAGCACAACCAGATAAAAGTGAATCAGAGTTAGTCAGTCAAATA |
| | | ATAGAACAGTTAATAAAAAAGGAAAAGGTCTACCTGGCATGGGT |
| | | ACCAGCGCACAAAGGAATTGGAGGAAATGAACAAGTAGATAAA |
| | | TTAGTCAGTACTGGAATCAGGAAAGTACTA |
| 58 | HIV Integrase; | TTTTTAGATGGAATAGATATAGCCCAAGAAGAACATGAGAAATA |
| | Bal | TCACAGTAATTGGAGAGCAATGGCTAGTGATTTTAACCTGCCAC |
| | | CTGTGGTAGCAAAAGAAATAGTAGCCAGCTGTGATAAATGTCAG |
| | | CTAAAAGGAGAAGCCATGCATGGACAAGTAGACTGTAGTCCAGG |
| | | AATATGGCAACTAGATTGTACACATTTAGAAGGAAAAATTATCC |
| | | TGGTAGCAGTTCATGTAGCCAGTGGATATATAGAAGCAGAAGTT |
| | | ATTCCAGCAGAGACAGGGCAGGAAACAGCATACTTTCTCTTAAA |
| | | ATTAGCAGGAAGATGGCCAGTAAAAACAATACATACAGACAAT |
| | | GGCAGCAATTTCACTAGTACTACAGTCAAGGCCGCCTGTTGGTG |
| | | GGCGGGGATCAAGCAGGAATTTGGCATTCCCTACAATCCCCAAA |
| | | GTCAGGGAGTAGTAGAATCTATAAATAAAGAATTAAAGAAAATT |
| | | ATAGGACAGGTAAGAGATCAGGCTGAACATCTTAAAACAGCAGT |
| | | ACAAATGGCAGTATTCATCCACAATTTTAAAAGAAAAGGGGGGA |
| | | TTGGGGGGTATAGTGCAGGGGAAAGAATAGTAGACATAATAGC |
| | | AACAGACATACAAACTAAAGAATTACAAAAACAAATTACAAAA |
| | | ATTCAAAATTTTCGGGTTTATTACAGGGACAGCAGAGATCCACTT |
| | | TGGAAAGGACCAGCAAAGCTTCTCTGGAAAGGTGAAGGGGCAG |
| | | TAGTAATACAAGATAATAGTGACATAAAAGTAGTACCAAGAAGA |
| | | AAAGCAAAGATCATTAGGGATTATGGAAAACAGATGGCAGGTG |
| | | ATGATTGTGTGGCAAGTAGACAGGATGAGGATTAG |
| 59 | Envelope; | ATGAAACTCCCAACAGGAATGGTCATTTTATGTAGCCTAATAAT |
| | RD114 | AGTTCGGGCAGGGTTTGACGACCCCCGCAAGGCTATCGCATTAG |
| | | TACAAAAACAACATGGTAAACCATGCGAATGCAGCGGAGGGCA |
| | | GGTATCCGAGGCCCCACCGAACTCCATCCAACAGGTAACTTGCC |
| | | CAGGCAAGACGGCCTACTTAATGACCAACCAAAAATGGAAATGC |
| | | AGAGTCACTCCAAAAAATCTCACCCCTAGCGGGGGAGAACTCCA |
| | | GAACTGCCCCTGTAACACTTTCCAGGACTCGATGCACAGTTCTTG |
| | | TTATACTGAATACCGGCAATGCAGGGCGAATAATAAGACATACT |
| | | ACACGGCCACCTTGCTTAAAATACGGTCTGGGAGCCTCAACGAG |
| | | GTACAGATATTACAAAACCCCAATCAGCTCCTACAGTCCCCTTGT |
| | | AGGGGCTCTATAAATCAGCCCGTTTGCTGGAGTGCCACAGCCCC |
| | | CATCCATATCTCCGATGGTGGAGGACCCCTCGATACTAAGAGAG |
| | | TGTGGACAGTCCAAAAAAGGCTAGAACAAATTCATAAGGCTATG |
| | | CATCCTGAACTTCAATACCACCCCTTAGCCCTGCCCAAAGTCAGA |
| | | GATGACCTTAGCCTTGATGCACGGACTTTTGATATCCTGAATACC |
| | | ACTTTTAGGTTACTCCAGATGTCCAATTTTAGCCTTGCCCAAGAT |
| | | TGTTGGCTCTGTTTAAAACTAGGTACCCCTACCCCTCTTGCGATA |
| | | CCCACTCCCTCTTTAACCTACTCCCTAGCAGACTCCCTAGCGAAT |
| | | GCCTCCTGTCAGATTATACCTCCCCTCTTGGTTCAACCGATGCAG |
| | | TTCTCCAACTCGTCCTGTTTATCTTCCCCTTTCATTAACGATACGG |
| | | AACAAATAGACTTAGGTGCAGTCACCTTTACTAACTGCACCTCTG |
| | | TAGCCAATGTCAGTAGTCCTTTATGTGCCCTAAACGGGTCAGTCT |
| | | TCCTCTGTGGAAATAACATGGCATACACCTATTTACCCCAAAACT |
| | | GGACAGGACTTTGCGTCCAAGCCTCCCTCCTCCCCGACATTGACA |
| | | TCATCCCGGGGGATGAGCCAGTCCCCATTCCTGCCATTGATCATT |
| | | ATATACATAGACCTAAACGAGCTGTACAGTTCATCCCTTTACTAG |
| | | CTGGACTGGGAATCACCGCAGCATTCACCACCGGAGCTACAGGC |
| | | CTAGGTGTCTCCGTCACCCAGTATACAAAATTATCCCATCAGTTA |
| | | ATATCTGATGTCCAAGTCTTATCCGGTACCATACAAGATTTACAA |
| | | GACCAGGTAGACTCGTTAGCTGAAGTAGTTCTCCAAAATAGGAG |
| | | GGGACTGGACCTACTAACGGCAGAACAAGGAGGAATTTGTTTAG |
| | | CCTTACAAGAAAAATGCTGTTTTTATGCTAACAAGTCAGGAATTG |
| | | TGAGAAACAAAATAAGAACCCTACAAGAAGAATTACAAAAACG |
| | | CAGGGAAAGCCTGGCATCCAACCCTCTCTGGACCGGGCTGCAGG |
| | | GCTTTCTTCCGTACCTCCTACCTCTCCTGGGACCCCTACTCACCCT |
| | | CCTACTCATACTAACCATTGGGCCATGCGTTTTCAATCGATTGGT |
| | | CCAATTTGTTAAAGACAGGATCTCAGTGGTCCAGGCTCTGGTTTT |
| | | GACTCAGCAATATCACCAGCTAAAACCCATAGAGTACGAGCCAT |
| | | GA |
| 60 | Envelope; | ATGCTTCTCACCTCAAGCCCGCACCACCTTCGGCACCAGATGAGT |
| | GALV | CCTGGGAGCTGGAAAAGACTGATCATCCTCTTAAGCTGCGTATTC |
| | | GGAGACGGCAAAACGAGTCTGCAGAATAAGAACCCCCACCAGC |
| | | CTGTGACCCTCACCTGGCAGGTACTGTCCCAAACTGGGGACGTT |
| | | GTCTGGGACAAAAAGGCAGTCCAGCCCCTTTGGACTTGGTGGCC |
| | | CTCTCTTACACCTGATGTATGTGCCCTGGCGGCCGGTCTTGAGTC |
| | | CTGGGATATCCCGGGATCCGATGTATCGTCCTCTAAAAGAGTTA |
| | | GACCTCCTGATTCAGACTATACTGCCGCTTATAAGCAAATCACCT |
| | | GGGGAGCCATAGGGTGCAGCTACCCTCGGGCTAGGACCAGGATG |
| | | GCAAATTCCCCCTTCTACGTGTGTCCCCGAGCTGGCCGAACCCAT |
| | | TCAGAAGCTAGGAGGTGTGGGGGGCTAGAATCCCTATACTGTAA |
| | | AGAATGGAGTTGTGAGACCACGGGTACCGTTTATTGGCAACCCA |
| | | AGTCCTCATGGGACCTCATAACTGTAAAATGGGACCAAAATGTG |
| | | AAATGGGAGCAAAAATTTCAAAAGTGTGAACAAACCGGCTGGTG |
| | | TAACCCCCTCAAGATAGACTTCACAGAAAAAGGAAAACTCTCCA |
| | | GAGATTGGATAACGGAAAAAACCTGGGAATTAAGGTTCTATGTA |
| | | TATGGACACCCAGGCATACAGTTGACTATCCGCTTAGAGGTCAC |
| | | TAACATGCCGGTTGTGGCAGTGGGCCCAGACCCTGTCCTTGCGG |
| | | AACAGGGACCTCCTAGCAAGCCCCTCACTCTCCCTCTCTCCCCAC |
| | | GGAAAGCGCCGCCCACCCCTCTACCCCCGGCGGCTAGTGAGCAA |
| | | ACCCCTGCGGTGCATGGAGAAACTGTTACCCTAAACTCTCCGCCT |
| | | CCCACCAGTGGCGACCGACTCTTTGGCCTTGTGCAGGGGGCCTTC |
| | | CTAACCTTGAATGCTACCAACCCAGGGGCCACTAAGTCTTGCTG |
| | | GCTCTGTTTGGGCATGAGCCCCCCTTATTATGAAGGGATAGCCTC |
| | | TTCAGGAGAGGTCGCTTATACCTCCAACCATACCCGATGCCACTG |
| | | GGGGGCCCAAGGAAAGCTTACCCTCACTGAGGTCTCCGGACTCG |
| | | GGTCATGCATAGGGAAGGTGCCTCTTACCCATCAACATCTTTGCA |
| | | ACCAGACCTTACCCATCAATTCCTCTAAAAACCATCAGTATCTGC |
| | | TCCCCTCAAACCATAGCTGGTGGGCCTGCAGCACTGGCCTCACCC |
| | | CCTGCCTCTCCACCTCAGTTTTTAATCAGTCTAAAGACTTCTGTGT |
| | | CCAGGTCCAGCTGATCCCCCGCATCTATTACCATTCTGAAGAAAC |
| | | CTTGTTACAAGCCTATGACAAATCACCCCCCAGGTTTAAAAGAG |
| | | AGCCTGCCTCACTTACCCTAGCTGTCTTCCTGGGGTTAGGGATTG |
| | | CGGCAGGTATAGGTACTGGCTCAACCGCCCTAATTAAAGGGCCC |
| | | ATAGACCTCCAGCAAGGCCTAACCAGCCTCCAAATCGCCATTGA |
| | | CGCTGACCTCCGGGCCCTTCAGGACTCAATCAGCAAGCTAGAGG |
| | | ACTCACTGACTTCCCTATCTGAGGTAGTACTCCAAAATAGGAGA |
| | | GGCCTTGACTTACTATTCCTTAAAGAAGGAGGCCTCTGCGCGGCC |
| | | CTAAAAGAAGAGTGCTGTTTTTATGTAGACCACTCAGGTGCAGT |
| | | ACGAGACTCCATGAAAAAACTTAAAGAAAGACTAGATAAAAGA |
| | | CAGTTAGAGCGCCAGAAAAACCAAAACTGGTATGAAGGGTGGTT |
| | | CAATAACTCCCCTTGGTTTACTACCCTACTATCAACCATCGCTGG |
| | | GCCCCTATTGCTCCTCCTTTTGTTACTCACTCTTGGGCCCTGCATC |
| | | ATCAATAAATTAATCCAATTCATCAATGATAGGATAAGTGCAGT |
| | | CAAAATTTTAGTCCTTAGACAGAAATATCAGACCCTAGATAACG |
| | | AGGAAAACCTTTAA |
| 61 | Envelope; FUG | ATGGTTCCGCAGGTTCTTTTGTTTGTACTCCTTCTGGGTTTTTCGT |
| | | TGTGTTTCGGGAAGTTCCCCATTTACACGATACCAGACGAACTTG |
| | | GTCCCTGGAGCCCTATTGACATACACCATCTCAGCTGTCCAAATA |
| | | ACCTGGTTGTGGAGGATGAAGGATGTACCAACCTGTCCGAGTTC |
| | | TCCTACATGGAACTCAAAGTGGGATACATCTCAGCCATCAAAGT |
| | | GAACGGGTTCACTTGCACAGGTGTTGTGACAGAGGCAGAGACCT |
| | | ACACCAACTTTGTTGGTTATGTCACAACCACATTCAAGAGAAAG |
| | | CATTTCCGCCCCACCCCAGACGCATGTAGAGCCGCGTATAACTG |
| | | GAAGATGGCCGGTGACCCCAGATATGAAGAGTCCCTACACAATC |
| | | CATACCCCGACTACCACTGGCTTCGAACTGTAAGAACCACCAAA |
| | | GAGTCCCTCATTATCATATCCCCAAGTGTGACAGATTTGGACCCA |
| | | TATGACAAATCCCTTCACTCAAGGGTCTTCCCTGGCGGAAAGTGC |
| | | TCAGGAATAACGGTGTCCTCTACCTACTGCTCAACTAACCATGAT |
| | | TACACCATTTGGATGCCCGAGAATCCGAGACCAAGGACACCTTG |
| | | TGACATTTTTACCAATAGCAGAGGGAAGAGAGCATCCAACGGGA |
| | | ACAAGACTTGCGGCTTTGTGGATGAAAGAGGCCTGTATAAGTCT |
| | | CTAAAAGGAGCATGCAGGCTCAAGTTATGTGGAGTTCTTGGACT |
| | | TAGACTTATGGATGGAACATGGGTCGCGATGCAAACATCAGATG |
| | | AGACCAAATGGTGCCCTCCAGATCAGTTGGTGAATTTGCACGAC |
| | | TTTCGCTCAGACGAGATCGAGCATCTCGTTGTGGAGGAGTTAGTT |
| | | AAGAAAAGAGAGGAATGTCTGGATGCATTAGAGTCCATCATGAC |
| | | CACCAAGTCAGTAAGTTTCAGACGTCTCAGTCACCTGAGAAAAC |
| | | TTGTCCCAGGGTTTGGAAAAGCATATACCATATTCAACAAAACC |
| | | TTGATGGAGGCTGATGCTCACTACAAGTCAGTCCGGACCTGGAA |
| | | TGAGATCATCCCCTCAAAAGGGTGTTTGAAAGTTGGAGGAAGGT |
| | | GCCATCCTCATGTGAACGGGGTGTTTTTCAATGGTATAATATTAG |
| | | GGCCTGACGACCATGTCCTAATCCCAGAGATGCAATCATCCCTCC |
| | | TCCAGCAACATATGGAGTTGTTGGAATCTTCAGTTATCCCCCTGA |
| | | TGCACCCCCTGGCAGACCCTTCTACAGTTTTCAAAGAAGGTGATG |
| | | AGGCTGAGGATTTTGTTGAAGTTCACCTCCCCGATGTGTACAAAC |
| | | AGATCTCAGGGGTTGACCTGGGTCTCCCGAACTGGGGAAAGTAT |
| | | GTATTGATGACTGCAGGGGCCATGATTGGCCTGGTGTTGATATTT |
| | | TCCCTAATGACATGGTGCAGAGTTGGTATCCATCTTTGCATTAAA |
| | | TTAAAGCACACCAAGAAAAGACAGATTTATACAGACATAGAGAT |
| | | GAACCGACTTGGAAAGTAA |
| 62 | Envelope; | ATGGGTCAGATTGTGACAATGTTTGAGGCTCTGCCTCACATCATC |
| | LCMV | GATGAGGTGATCAACATTGTCATTATTGTGCTTATCGTGATCACG |
| | | GGTATCAAGGCTGTCTACAATTTTGCCACCTGTGGGATATTCGCA |
| | | TTGATCAGTTTCCTACTTCTGGCTGGCAGGTCCTGTGGCATGTAC |
| | | GGTCTTAAGGGACCCGACATTTACAAAGGAGTTTACCAATTTAA |
| | | GTCAGTGGAGTTTGATATGTCACATCTGAACCTGACCATGCCCAA |
| | | CGCATGTTCAGCCAACAACTCCCACCATTACATCAGTATGGGGA |
| | | CTTCTGGACTAGAATTGACCTTCACCAATGATTCCATCATCAGTC |
| | | ACAACTTTTGCAATCTGACCTCTGCCTTCAACAAAAAGACCTTTG |
| | | ACCACACACTCATGAGTATAGTTTCGAGCCTACACCTCAGTATCA |
| | | GAGGGAACTCCAACTATAAGGCAGTATCCTGCGACTTCAACAAT |
| | | GGCATAACCATCCAATACAACTTGACATTCTCAGATCGACAAAG |
| | | TGCTCAGAGCCAGTGTAGAACCTTCAGAGGTAGAGTCCTAGATA |
| | | TGTTTAGAACTGCCTTCGGGGGGAAATACATGAGGAGTGGCTGG |
| | | GGCTGGACAGGCTCAGATGGCAAGACCACCTGGTGTAGCCAGAC |
| | | GAGTTACCAATACCTGATTATACAAAATAGAACCTGGGAAAACC |
| | | ACTGCACATATGCAGGTCCTTTTGGGATGTCCAGGATTCTCCTTT |
| | | CCCAAGAGAAGACTAAGTTCTTCACTAGGAGACTAGCGGGCACA |
| | | TTCACCTGGACTTTGTCAGACTCTTCAGGGGTGGAGAATCCAGGT |
| | | GGTTATTGCCTGACCAAATGGATGATTCTTGCTGCAGAGCTTAAG |
| | | TGTTTCGGGAACACAGCAGTTGCGAAATGCAATGTAAATCATGA |
| | | TGCCGAATTCTGTGACATGCTGCGACTAATTGACTACAACAAGG |
| | | CTGCTTTGAGTAAGTTCAAAGAGGACGTAGAATCTGCCTTGCACT |
| | | TATTCAAAACAACAGTGAATTCTTTGATTTCAGATCAACTACTGA |
| | | TGAGGAACCACTTGAGAGATCTGATGGGGGTGCCATATTGCAAT |
| | | TACTCAAAGTTTTGGTACCTAGAACATGCAAAGACCGGCGAAAC |
| | | TAGTGTCCCCAAGTGCTGGCTTGTCACCAATGGTTCTTACTTAAA |
| | | TGAGACCCACTTCAGTGATCAAATCGAACAGGAAGCCGATAACA |
| | | TGATTACAGAGATGTTGAGGAAGGATTACATAAAGAGGCAGGG |
| | | GAGTACCCCCCTAGCATTGATGGACCTTCTGATGTTTTCCACATC |
| | | TGCATATCTAGTCAGCATCTTCCTGCACCTTGTCAAAATACCAAC |
| | | ACACAGGCACATAAAAGGTGGCTCATGTCCAAAGCCACACCGAT |
| | | TAACCAACAAAGGAATTTGTAGTTGTGGTGCATTTAAGGTGCCT |
| | | GGTGTAAAAACCGTCTGGAAAAGACGCTGA |
| 63 | Envelope; FPV | ATGAACACTCAAATCCTGGTTTTCGCCCTTGTGGCAGTCATCCCC |
| | | ACAAATGCAGACAAAATTTGTCTTGGACATCATGCTGTATCAAA |
| | | TGGCACCAAAGTAAACACACTCACTGAGAGAGGAGTAGAAGTTG |
| | | TCAATGCAACGGAAACAGTGGAGCGGACAAACATCCCCAAAATT |
| | | TGCTCAAAAGGGAAAAGAACCACTGATCTTGGCCAATGCGGACT |
| | | GTTAGGGACCATTACCGGACCACCTCAATGCGACCAATTTCTAG |
| | | AATTTTCAGCTGATCTAATAATCGAGAGACGAGAAGGAAATGAT |
| | | GTTTGTTACCCGGGGAAGTTTGTTAATGAAGAGGCATTGCGACA |
| | | AATCCTCAGAGGATCAGGTGGGATTGACAAAGAAACAATGGGAT |
| | | TCACATATAGTGGAATAAGGACCAACGGAACAACTAGTGCATGT |
| | | AGAAGATCAGGGTCTTCATTCTATGCAGAAATGGAGTGGCTCCT |
| | | GTCAAATACAGACAATGCTGCTTTCCCACAAATGACAAAATCAT |
| | | ACAAAAACACAAGGAGAGAATCAGCTCTGATAGTCTGGGGAATC |
| | | CACCATTCAGGATCAACCACCGAACAGACCAAACTATATGGGAG |
| | | TGGAAATAAACTGATAACAGTCGGGAGTTCCAAATATCATCAAT |
| | | CTTTTGTGCCGAGTCCAGGAACACGACCGCAGATAAATGGCCAG |
| | | TCCGGACGGATTGATTTTCATTGGTTGATCTTGGATCCCAATGAT |
| | | ACAGTTACTTTTAGTTTCAATGGGGCTTTCATAGCTCCAAATCGT |
| | | GCCAGCTTCTTGAGGGGAAAGTCCATGGGGATCCAGAGCGATGT |
| | | GCAGGTTGATGCCAATTGCGAAGGGGAATGCTACCACAGTGGAG |
| | | GGACTATAACAAGCAGATTGCCTTTTCAAAACATCAATAGCAGA |
| | | GCAGTTGGCAAATGCCCAAGATATGTAAAACAGGAAAGTTTATT |
| | | ATTGGCAACTGGGATGAAGAACGTTCCCGAACCTTCCAAAAAAA |
| | | GGAAAAAAAGAGGCCTGTTTGGCGCTATAGCAGGGTTTATTGAA |
| | | AATGGTTGGGAAGGTCTGGTCGACGGGTGGTACGGTTTCAGGCA |
| | | TCAGAATGCACAAGGAGAAGGAACTGCAGCAGACTACAAAAGC |
| | | ACCCAATCGGCAATTGATCAGATAACCGGAAAGTTAAATAGACT |
| | | CATTGAGAAAACCAACCAGCAATTTGAGCTAATAGATAATGAAT |
| | | TCACTGAGGTGGAAAAGCAGATTGGCAATTTAATTAACTGGACC |
| | | AAAGACTCCATCACAGAAGTATGGTCTTACAATGCTGAACTTCTT |
| | | GTGGCAATGGAAAACCAGCACACTATTGATTTGGCTGATTCAGA |
| | | GATGAACAAGCTGTATGAGCGAGTGAGGAAACAATTAAGGGAA |
| | | AATGCTGAAGAGGATGGCACTGGTTGCTTTGAAATTTTTCATAAA |
| | | TGTGACGATGATTGTATGGCTAGTATAAGGAACAATACTTATGA |
| | | TCACAGCAAATACAGAGAAGAAGCGATGCAAAATAGAATACAA |
| | | ATTGACCCAGTCAAATTGAGTAGTGGCTACAAAGATGTGATACT |
| | | TTGGTTTAGCTTCGGGGCATCATGCTTTTTGCTTCTTGCCATTGCA |
| | | ATGGGCCTTGTTTTCATATGTGTGAAGAACGGAAACATGCGGTG |
| | | CACTATTTGTATATAA |
| 64 | Envelope; RRV | AGTGTAACAGAGCACTTTAATGTGTATAAGGCTACTAGACCATA |
| | | CCTAGCACATTGCGCCGATTGCGGGGACGGGTACTTCTGCTATA |
| | | GCCCAGTTGCTATCGAGGAGATCCGAGATGAGGCGTCTGATGGC |
| | | ATGCTTAAGATCCAAGTCTCCGCCCAAATAGGTCTGGACAAGGC |
| | | AGGCACCCACGCCCACACGAAGCTCCGATATATGGCTGGTCATG |
| | | ATGTTCAGGAATCTAAGAGAGATTCCTTGAGGGTGTACACGTCC |
| | | GCAGCGTGCTCCATACATGGGACGATGGGACACTTCATCGTCGC |
| | | ACACTGTCCACCAGGCGACTACCTCAAGGTTTCGTTCGAGGACG |
| | | CAGATTCGCACGTGAAGGCATGTAAGGTCCAATACAAGCACAAT |
| | | CCATTGCCGGTGGGTAGAGAGAAGTTCGTGGTTAGACCACACTT |
| | | TGGCGTAGAGCTGCCATGCACCTCATACCAGCTGACAACGGCTC |
| | | CCACCGACGAGGAGATTGACATGCATACACCGCCAGATATACCG |
| | | GATCGCACCCTGCTATCACAGACGGCGGGCAACGTCAAAATAAC |
| | | AGCAGGCGGCAGGACTATCAGGTACAACTGTACCTGCGGCCGTG |
| | | ACAACGTAGGCACTACCAGTACTGACAAGACCATCAACACATGC |
| | | AAGATTGACCAATGCCATGCTGCCGTCACCAGCCATGACAAATG |
| | | GCAATTTACCTCTCCATTTGTTCCCAGGGCTGATCAGACAGCTAG |
| | | GAAAGGCAAGGTACACGTTCCGTTCCCTCTGACTAACGTCACCT |
| | | GCCGAGTGCCGTTGGCTCGAGCGCCGGATGCCACCTATGGTAAG |
| | | AAGGAGGTGACCCTGAGATTACACCCAGATCATCCGACGCTCTT |
| | | CTCCTATAGGAGTTTAGGAGCCGAACCGCACCCGTACGAGGAAT |
| | | GGGTTGACAAGTTCTCTGAGCGCATCATCCCAGTGACGGAAGAA |
| | | GGGATTGAGTACCAGTGGGGCAACAACCCGCCGGTCTGCCTGTG |
| | | GGCGCAACTGACGACCGAGGGCAAACCCCATGGCTGGCCACATG |
| | | AAATCATTCAGTACTATTATGGACTATACCCCGCCGCCACTATTG |
| | | CCGCAGTATCCGGGGCGAGTCTGATGGCCCTCCTAACTCTGGCG |
| | | GCCACATGCTGCATGCTGGCCACCGCGAGGAGAAAGTGCCTAAC |
| | | ACCGTACGCCCTGACGCCAGGAGCGGTGGTACCGTTGACACTGG |
| | | GGCTGCTTTGCTGCGCACCGAGGGCGAATGCA |
| 65 | Envelope; | ATGGAAGGTCCAGCGTTCTCAAAACCCCTTAAAGATAAGATTAA |
| | MLV 10A1 | CCCGTGGAAGTCCTTAATGGTCATGGGGGTCTATTTAAGAGTAG |
| | | GGATGGCAGAGAGCCCCCATCAGGTCTTTAATGTAACCTGGAGA |
| | | GTCACCAACCTGATGACTGGGCGTACCGCCAATGCCACCTCCCTT |
| | | TTAGGAACTGTACAAGATGCCTTCCCAAGATTATATTTTGATCTA |
| | | TGTGATCTGGTCGGAGAAGAGTGGGACCCTTCAGACCAGGAACC |
| | | ATATGTCGGGTATGGCTGCAAATACCCCGGAGGGAGAAAGCGGA |
| | | CCCGGACTTTTGACTTTTACGTGTGCCCTGGGCATACCGTAAAAT |
| | | CGGGGTGTGGGGGGCCAAGAGAGGGCTACTGTGGTGAATGGGG |
| | | TTGTGAAACCACCGGACAGGCTTACTGGAAGCCCACATCATCAT |
| | | GGGACCTAATCTCCCTTAAGCGCGGTAACACCCCCTGGGACACG |
| | | GGATGCTCCAAAATGGCTTGTGGCCCCTGCTACGACCTCTCCAAA |
| | | GTATCCAATTCCTTCCAAGGGGCTACTCGAGGGGGCAGATGCAA |
| | | CCCTCTAGTCCTAGAATTCACTGATGCAGGAAAAAAGGCTAATT |
| | | GGGACGGGCCCAAATCGTGGGGACTGAGACTGTACCGGACAGG |
| | | AACAGATCCTATTACCATGTTCTCCCTGACCCGCCAGGTCCTCAA |
| | | TATAGGGCCCCGCATCCCCATTGGGCCTAATCCCGTGATCACTGG |
| | | TCAACTACCCCCCTCCCGACCCGTGCAGATCAGGCTCCCCAGGCC |
| | | TCCTCAGCCTCCTCCTACAGGCGCAGCCTCTATAGTCCCTGAGAC |
| | | TGCCCCACCTTCTCAACAACCTGGGACGGGAGACAGGCTGCTAA |
| | | ACCTGGTAGAAGGAGCCTATCAGGCGCTTAACCTCACCAATCCC |
| | | GACAAGACCCAAGAATGTTGGCTGTGCTTAGTGTCGGGACCTCC |
| | | TTATTACGAAGGAGTAGCGGTCGTGGGCACTTATACCAATCATTC |
| | | TACCGCCCCGGCCAGCTGTACGGCCACTTCCCAACATAAGCTTAC |
| | | CCTATCTGAAGTGACAGGACAGGGCCTATGCATGGGAGCACTAC |
| | | CTAAAACTCACCAGGCCTTATGTAACACCACCCAAAGTGCCGGC |
| | | TCAGGATCCTACTACCTTGCAGCACCCGCTGGAACAATGTGGGC |
| | | TTGTAGCACTGGATTGACTCCCTGCTTGTCCACCACGATGCTCAA |
| | | TCTAACCACAGACTATTGTGTATTAGTTGAGCTCTGGCCCAGAAT |
| | | AATTTACCACTCCCCCGATTATATGTATGGTCAGCTTGAACAGCG |
| | | TACCAAATATAAGAGGGAGCCAGTATCGTTGACCCTGGCCCTTC |
| | | TGCTAGGAGGATTAACCATGGGAGGGATTGCAGCTGGAATAGGG |
| | | ACGGGGACCACTGCCCTAATCAAAACCCAGCAGTTTGAGCAGCT |
| | | TCACGCCGCTATCCAGACAGACCTCAACGAAGTCGAAAAATCAA |
| | | TTACCAACCTAGAAAAGTCACTGACCTCGTTGTCTGAAGTAGTCC |
| | | TACAGAACCGAAGAGGCCTAGATTTGCTCTTCCTAAAAGAGGGA |
| | | GGTCTCTGCGCAGCCCTAAAAGAAGAATGTTGTTTTTATGCAGAC |
| | | CACACGGGACTAGTGAGAGACAGCATGGCCAAACTAAGGGAAA |
| | | GGCTTAATCAGAGACAAAAACTATTTGAGTCAGGCCAAGGTTGG |
| | | TTCGAAGGGCAGTTTAATAGATCCCCCTGGTTTACCACCTTAATC |
| | | TCCACCATCATGGGACCTCTAATAGTACTCTTACTGATCTTACTC |
| | | TTTGGACCCTGCATTCTCAATCGATTGGTCCAATTTGTTAAAGAC |
| | | AGGATCTCAGTGGTCCAGGCTCTGGTTTTGACTCAACAATATCAC |
| | | CAGCTAAAACCTATAGAGTACGAGCCATGA |
| 66 | Envelope; | ATGGGTGTTACAGGAATATTGCAGTTACCTCGTGATCGATTCAAG |
| | Ebola | AGGACATCATTCTTTCTTTGGGTAATTATCCTTTTCCAAAGAACA |
| | | TTTTCCATCCCACTTGGAGTCATCCACAATAGCACATTACAGGTT |
| | | AGTGATGTCGACAAACTGGTTTGCCGTGACAAACTGTCATCCAC |
| | | AAATCAATTGAGATCAGTTGGACTGAATCTCGAAGGGAATGGAG |
| | | TGGCAACTGACGTGCCATCTGCAACTAAAAGATGGGGCTTCAGG |
| | | TCCGGTGTCCCACCAAAGGTGGTCAATTATGAAGCTGGTGAATG |
| | | GGCTGAAAACTGCTACAATCTTGAAATCAAAAAACCTGACGGGA |
| | | GTGAGTGTCTACCAGCAGCGCCAGACGGGATTCGGGGCTTCCCC |
| | | CGGTGCCGGTATGTGCACAAAGTATCAGGAACGGGACCGTGTGC |
| | | CGGAGACTTTGCCTTCCACAAAGAGGGTGCTTTCTTCCTGTATGA |
| | | CCGACTTGCTTCCACAGTTATCTACCGAGGAACGACTTTCGCTGA |
| | | AGGTGTCGTTGCATTTCTGATACTGCCCCAAGCTAAGAAGGACTT |
| | | CTTCAGCTCACACCCCTTGAGAGAGCCGGTCAATGCAACGGAGG |
| | | ACCCGTCTAGTGGCTACTATTCTACCACAATTAGATATCAAGCTA |
| | | CCGGTTTTGGAACCAATGAGACAGAGTATTTGTTCGAGGTTGAC |
| | | AATTTGACCTACGTCCAACTTGAATCAAGATTCACACCACAGTTT |
| | | CTGCTCCAGCTGAATGAGACAATATATACAAGTGGGAAAAGGAG |
| | | CAATACCACGGGAAAACTAATTTGGAAGGTCAACCCCGAAATTG |
| | | ATACAACAATCGGGGAGTGGGCCTTCTGGGAAACTAAAAAAACC |
| | | TCACTAGAAAAATTCGCAGTGAAGAGTTGTCTTTCACAGCTGTAT |
| | | CAAACAGAGCCAAAAACATCAGTGGTCAGAGTCCGGCGCGAACT |
| | | TCTTCCGACCCAGGGACCAACACAACAACTGAAGACCACAAAAT |
| | | CATGGCTTCAGAAAATTCCTCTGCAATGGTTCAAGTGCACAGTCA |
| | | AGGAAGGGAAGCTGCAGTGTCGCATCTGACAACCCTTGCCACAA |
| | | TCTCCACGAGTCCTCAACCCCCCACAACCAAACCAGGTCCGGAC |
| | | AACAGCACCCACAATACACCCGTGTATAAACTTGACATCTCTGA |
| | | GGCAACTCAAGTTGAACAACATCACCGCAGAACAGACAACGAC |
| | | AGCACAGCCTCCGACACTCCCCCCGCCACGACCGCAGCCGGACC |
| | | CCTAAAAGCAGAGAACACCAACACGAGCAAGGGTACCGACCTC |
| | | CTGGACCCCGCCACCACAACAAGTCCCCAAAACCACAGCGAGAC |
| | | CGCTGGCAACAACAACACTCATCACCAAGATACCGGAGAAGAG |
| | | AGTGCCAGCAGCGGGAAGCTAGGCTTAATTACCAATACTATTGC |
| | | TGGAGTCGCAGGACTGATCACAGGCGGGAGGAGAGCTCGAAGA |
| | | GAAGCAATTGTCAATGCTCAACCCAAATGCAACCCTAATTTACA |
| | | TTACTGGACTACTCAGGATGAAGGTGCTGCAATCGGACTGGCCT |
| | | GGATACCATATTTCGGGCCAGCAGCCGAGGGAATTTACATAGAG |
| | | GGGCTGATGCACAATCAAGATGGTTTAATCTGTGGGTTGAGACA |
| | | GCTGGCCAACGAGACGACTCAAGCTCTTCAACTGTTCCTGAGAG |
| | | CCACAACCGAGCTACGCACCTTTTCAATCCTCAACCGTAAGGCA |
| | | ATTGATTTCTTGCTGCAGCGATGGGGCGGCACATGCCACATTTTG |
| | | GGACCGGACTGCTGTATCGAACCACATGATTGGACCAAGAACAT |
| | | AACAGACAAAATTGATCAGATTATTCATGATTTTGTTGATAAAAC |
| | | CCTTCCGGACCAGGGGGACAATGACAATTGGTGGACAGGATGGA |
| | | GACAATGGATACCGGCAGGTATTGGAGTTACAGGCGTTATAATT |
| | | GCAGTTATCGCTTTATTCTGTATATGCAAATTTGTCTTTTAG |
| 67 | Short WPRE | AATCAACCTCTGGATTACAAAATTTGTGAAAGATTGACTGATATT |
| | sequence | CTTAACTATGTTGCTCCTTTTACGCTGTGTGGATATGCTGCTTTAA |
| | | TGCCTCTGTATCATGCTATTGCTTCCCGTACGGCTTTCGTTTTCTC |
| | | CTCCTTGTATAAATCCTGGTTGCTGTCTCTTTATGAGGAGTTGTG |
| | | GCCCGTTGTCCGTCAACGTGGCGTGGTGTGCTCTGTGTTTGCTGA |
| | | CGCAACCCCCACTGGCTGGGGCATTGCCACCACCTGTCAACTCCT |
| | | TTCTGGGACTTTCGCTTTCCCCCTCCCGATCGCCACGGCAGAACT |
| | | CATCGCCGCCTGCCTTGCCCGCTGCTGGACAGGGGCTAGGTTGCT |
| | | GGGCACTGATAATTCCGTGGTGTTGTC |
| 68 | Helper Plasmid | TAAGCAGAATTCATGAATTTGCCAGGAAGAT |
| | Forward Primer | |
| 69 | Helper Plasmid | CCATACAATGAATGGACACTAGGCGGCCGCACGAAT |
| | Reverse Primer | |
| 70 | Gag, Pol, | GAATTCATGAATTTGCCAGGAAGATGGAAACCAAAAATGATAGG |
| | Integrase | GGGAATTGGAGGTTTTATCAAAGTAAGACAGTATGATCAGATAC |
| | fragment | TCATAGAAATCTGCGGACATAAAGCTATAGGTACAGTATTAGTA |
| | | GGACCTACACCTGTCAACATAATTGGAAGAAATCTGTTGACTCA |
| | | GATTGGCTGCACTTTAAATTTTCCCATTAGTCCTATTGAGACTGT |
| | | ACCAGTAAAATTAAAGCCAGGAATGGATGGCCCAAAAGTTAAAC |
| | | AATGGCCATTGACAGAAGAAAAAATAAAAGCATTAGTAGAAATT |
| | | TGTACAGAAATGGAAAAGGAAGGAAAAATTTCAAAAATTGGGC |
| | | CTGAAAATCCATACAATACTCCAGTATTTGCCATAAAGAAAAAA |
| | | GACAGTACTAAATGGAGAAAATTAGTAGATTTCAGAGAACTTAA |
| | | TAAGAGAACTCAAGATTTCTGGGAAGTTCAATTAGGAATACCAC |
| | | ATCCTGCAGGGTTAAAACAGAAAAAATCAGTAACAGTACTGGAT |
| | | GTGGGCGATGCATATTTTTCAGTTCCCTTAGATAAAGACTTCAGG |
| | | AAGTATACTGCATTTACCATACCTAGTATAAACAATGAGACACC |
| | | AGGGATTAGATATCAGTACAATGTGCTTCCACAGGGATGGAAAG |
| | | GATCACCAGCAATATTCCAGTGTAGCATGACAAAAATCTTAGAG |
| | | CCTTTTAGAAAACAAAATCCAGACATAGTCATCTATCAATACAT |
| | | GGATGATTTGTATGTAGGATCTGACTTAGAAATAGGGCAGCATA |
| | | GAACAAAAATAGAGGAACTGAGACAACATCTGTTGAGGTGGGG |
| | | ATTTACCACACCAGACAAAAAACATCAGAAAGAACCTCCATTCC |
| | | TTTGGATGGGTTATGAACTCCATCCTGATAAATGGACAGTACAG |
| | | CCTATAGTGCTGCCAGAAAAGGACAGCTGGACTGTCAATGACAT |
| | | ACAGAAATTAGTGGGAAAATTGAATTGGGCAAGTCAGATTTATG |
| | | CAGGGATTAAAGTAAGGCAATTATGTAAACTTCTTAGGGGAACC |
| | | AAAGCACTAACAGAAGTAGTACCACTAACAGAAGAAGCAGAGC |
| | | TAGAACTGGCAGAAAACAGGGAGATTCTAAAAGAACCGGTACA |
| | | TGGAGTGTATTATGACCCATCAAAAGACTTAATAGCAGAAATAC |
| | | AGAAGCAGGGGCAAGGCCAATGGACATATCAAATTTATCAAGA |
| | | GCCATTTAAAAATCTGAAAACAGGAAAGTATGCAAGAATGAAG |
| | | GGTGCCCACACTAATGATGTGAAACAATTAACAGAGGCAGTACA |
| | | AAAAATAGCCACAGAAAGCATAGTAATATGGGGAAAGACTCCT |
| | | AAATTTAAATTACCCATACAAAAGGAAACATGGGAAGCATGGTG |
| | | GACAGAGTATTGGCAAGCCACCTGGATTCCTGAGTGGGAGTTTG |
| | | TCAATACCCCTCCCTTAGTGAAGTTATGGTACCAGTTAGAGAAA |
| | | GAACCCATAATAGGAGCAGAAACTTTCTATGTAGATGGGGCAGC |
| | | CAATAGGGAAACTAAATTAGGAAAAGCAGGATATGTAACTGAC |
| | | AGAGGAAGACAAAAAGTTGTCCCCCTAACGGACACAACAAATC |
| | | AGAAGACTGAGTTACAAGCAATTCATCTAGCTTTGCAGGATTCG |
| | | GGATTAGAAGTAAACATAGTGACAGACTCACAATATGCATTGGG |
| | | AATCATTCAAGCACAACCAGATAAGAGTGAATCAGAGTTAGTCA |
| | | GTCAAATAATAGAGCAGTTAATAAAAAAGGAAAAAGTCTACCTG |
| | | GCATGGGTACCAGCACACAAAGGAATTGGAGGAAATGAACAAG |
| | | TAGATAAATTGGTCAGTGCTGGAATCAGGAAAGTACTATTTTTA |
| | | GATGGAATAGATAAGGCCCAAGAAGAACATGAGAAATATCACA |
| | | GTAATTGGAGAGCAATGGCTAGTGATTTTAACCTACCACCTGTA |
| | | GTAGCAAAAGAAATAGTAGCCAGCTGTGATAAATGTCAGCTAAA |
| | | AGGGGAAGCCATGCATGGACAAGTAGACTGTAGCCCAGGAATAT |
| | | GGCAGCTAGATTGTACACATTTAGAAGGAAAAGTTATCTTGGTA |
| | | GCAGTTCATGTAGCCAGTGGATATATAGAAGCAGAAGTAATTCC |
| | | AGCAGAGACAGGGCAAGAAACAGCATACTTCCTCTTAAAATTAG |
| | | CAGGAAGATGGCCAGTAAAAACAGTACATACAGACAATGGCAG |
| | | CAATTTCACCAGTACTACAGTTAAGGCCGCCTGTTGGTGGGCGG |
| | | GGATCAAGCAGGAATTTGGCATTCCCTACAATCCCCAAAGTCAA |
| | | GGAGTAATAGAATCTATGAATAAAGAATTAAAGAAAATTATAGG |
| | | ACAGGTAAGAGATCAGGCTGAACATCTTAAGACAGCAGTACAAA |
| | | TGGCAGTATTCATCCACAATTTTAAAAGAAAAGGGGGGATTGGG |
| | | GGGTACAGTGCAGGGGAAAGAATAGTAGACATAATAGCAACAG |
| | | ACATACAAACTAAAGAATTACAAAAACAAATTACAAAAATTCAA |
| | | AATTTTCGGGTTTATTACAGGGACAGCAGAGATCCAGTTTGGAA |
| | | AGGACCAGCAAAGCTCCTCTGGAAAGGTGAAGGGGCAGTAGTA |
| | | ATACAAGATAATAGTGACATAAAAGTAGTGCCAAGAAGAAAAG |
| | | CAAAGATCATCAGGGATTATGGAAAACAGATGGCAGGTGATGAT |
| | | TGTGTGGCAAGTAGACAGGATGAGGATTAA |
| 71 | DNA Fragment | TCTAGAATGGCAGGAAGAAGCGGAGACAGCGACGAAGAGCTCA |
| | containing Rev, | TCAGAACAGTCAGACTCATCAAGCTTCTCTATCAAAGCAACCCA |
| | RRE and rabbit | CCTCCCAATCCCGAGGGGACCCGACAGGCCCGAAGGAATAGAA |
| | beta globin | GAAGAAGGTGGAGAGAGAGACAGAGACAGATCCATTCGATTAG |
| | poly A | TGAACGGATCCTTGGCACTTATCTGGGACGATCTGCGGAGCCTGT |
| | | GCCTCTTCAGCTACCACCGCTTGAGAGACTTACTCTTGATTGTAA |
| | | CGAGGATTGTGGAACTTCTGGGACGCAGGGGGTGGGAAGCCCTC |
| | | AAATATTGGTGGAATCTCCTACAATATTGGAGTCAGGAGCTAAA |
| | | GAATAGAGGAGCTTTGTTCCTTGGGTTCTTGGGAGCAGCAGGAA |
| | | GCACTATGGGCGCAGCGTCAATGACGCTGACGGTACAGGCCAGA |
| | | CAATTATTGTCTGGTATAGTGCAGCAGCAGAACAATTTGCTGAG |
| | | GGCTATTGAGGCGCAACAGCATCTGTTGCAACTCACAGTCTGGG |
| | | GCATCAAGCAGCTCCAGGCAAGAATCCTGGCTGTGGAAAGATAC |
| | | CTAAAGGATCAACAGCTCCTAGATCTTTTTCCCTCTGCCAAAAAT |
| | | TATGGGGACATCATGAAGCCCCTTGAGCATCTGACTTCTGGCTAA |
| | | TAAAGGAAATTTATTTTCATTGCAATAGTGTGTTGGAATTTTTTG |
| | | TGTCTCTCACTCGGAAGGACATATGGGAGGGCAAATCATTTAAA |
| | | ACATCAGAATGAGTATTTGGTTTAGAGTTTGGCAACATATGCCAT |
| | | ATGCTGGCTGCCATGAACAAAGGTGGCTATAAAGAGGTCATCAG |
| | | TATATGAAACAGCCCCCTGCTGTCCATTCCTTATTCCATAGAAAA |
| | | GCCTTGACTTGAGGTTAGATTTTTTTTATATTTTGTTTTGTGTTAT |
| | | TTTTTTCTTTAACATCCCTAAAATTTTCCTTACATGTTTTACTAGC |
| | | CAGATTTTTCCTCCTCTCCTGACTACTCCCAGTCATAGCTGTCCCT |
| | | CTTCTCTTATGAAGATCCCTCGACCTGCAGCCCAAGCTTGGCGTA |
| | | ATCATGGTCATAGCTGTTTCCTGTGTGAAATTGTTATCCGCTCAC |
| | | AATTCCACACAACATACGAGCCGGAAGCATAAAGTGTAAAGCCT |
| | | GGGGTGCCTAATGAGTGAGCTAACTCACATTAATTGCGTTGCGCT |
| | | CACTGCCCGCTTTCCAGTCGGGAAACCTGTCGTGCCAGCGGATCC |
| | | GCATCTCAATTAGTCAGCAACCATAGTCCCGCCCCTAACTCCGCC |
| | | CATCCCGCCCCTAACTCCGCCCAGTTCCGCCCATTCTCCGCCCCA |
| | | TGGCTGACTAATTTTTTTTATTTATGCAGAGGCCGAGGCCGCCTC |
| | | GGCCTCTGAGCTATTCCAGAAGTAGTGAGGAGGCTTTTTTGGAG |
| | | GCCTAGGCTTTTGCAAAAAGCTAACTTGTTTATTGCAGCTTATAA |
| | | TGGTTACAAATAAAGCAATAGCATCACAAATTTCACAAATAAAG |
| | | CATTTTTTTCACTGCATTCTAGTTGTGGTTTGTCCAAACTCATCAA |
| | | TGTATCTTATCAGCGGCCGCCCCGGG |
| 72 | DNA fragment | ACGCGTTAGTTATTAATAGTAATCAATTACGGGGTCATTAGTTCA |
| | containing the | TAGCCCATATATGGAGTTCCGCGTTACATAACTTACGGTAAATGG |
| | CAG | CCCGCCTGGCTGACCGCCCAACGACCCCCGCCCATTGACGTCAA |
| | enhancer/prom | TAATGACGTATGTTCCCATAGTAACGCCAATAGGGACTTTCCATT |
| | oter/intron | GACGTCAATGGGTGGACTATTTACGGTAAACTGCCCACTTGGCA |
| | sequence | GTACATCAAGTGTATCATATGCCAAGTACGCCCCCTATTGACGTC |
| | | AATGACGGTAAATGGCCCGCCTGGCATTATGCCCAGTACATGAC |
| | | CTTATGGGACTTTCCTACTTGGCAGTACATCTACGTATTAGTCAT |
| | | CGCTATTACCATGGGTCGAGGTGAGCCCCACGTTCTGCTTCACTC |
| | | TCCCCATCTCCCCCCCCTCCCCACCCCCAATTTTGTATTTATTTAT |
| | | TTTTTAATTATTTTGTGCAGCGATGGGGGCGGGGGGGGGGGGGG |
| | | CGCGCGCCAGGCGGGGCGGGGCGGGGCGAGGGGCGGGGCGGGG |
| | | CGAGGCGGAGAGGTGCGGCGGCAGCCAATCAGAGCGGCGCGCT |
| | | CCGAAAGTTTCCTTTTATGGCGAGGCGGCGGCGGCGGCGGCCCT |
| | | ATAAAAAGCGAAGCGCGCGGCGGGCGGGAGTCGCTGCGTTGCCT |
| | | TCGCCCCGTGCCCCGCTCCGCGCCGCCTCGCGCCGCCCGCCCCGG |
| | | CTCTGACTGACCGCGTTACTCCCACAGGTGAGCGGGCGGGACGG |
| | | CCCTTCTCCTCCGGGCTGTAATTAGCGCTTGGTTTAATGACGGCT |
| | | CGTTTCTTTTCTGTGGCTGCGTGAAAGCCTTAAAGGGCTCCGGGA |
| | | GGGCCCTTTGTGCGGGGGGGAGCGGCTCGGGGGGTGCGTGCGTG |
| | | TGTGTGTGCGTGGGGAGCGCCGCGTGCGGCCCGCGCTGCCCGGC |
| | | GGCTGTGAGCGCTGCGGGCGCGGCGCGGGGCTTTGTGCGCTCCG |
| | | CGTGTGCGCGAGGGGAGCGCGGCCGGGGGCGGTGCCCCGCGGT |
| | | GCGGGGGGGCTGCGAGGGGAACAAAGGCTGCGTGCGGGGTGTG |
| | | TGCGTGGGGGGGTGAGCAGGGGGTGTGGGCGCGGCGGTCGGGC |
| | | TGTAACCCCCCCCTGCACCCCCCTCCCCGAGTTGCTGAGCACGGC |
| | | CCGGCTTCGGGTGCGGGGCTCCGTGCGGGGCGTGGCGCGGGGCT |
| | | CGCCGTGCCGGGCGGGGGGTGGCGGCAGGTGGGGGTGCCGGGC |
| | | GGGGCGGGGCCGCCTCGGGCCGGGGAGGGCTCGGGGGAGGGGC |
| | | GCGGCGGCCCCGGAGCGCCGGCGGCTGTCGAGGCGCGGCGAGC |
| | | CGCAGCCATTGCCTTTTATGGTAATCGTGCGAGAGGGCGCAGGG |
| | | ACTTCCTTTGTCCCAAATCTGGCGGAGCCGAAATCTGGGAGGCG |
| | | CCGCCGCACCCCCTCTAGCGGGCGCGGGCGAAGCGGTGCGGCGC |
| | | CGGCAGGAAGGAAATGGGCGGGGAGGGCCTTCGTGCGTCGCCG |
| | | CGCCGCCGTCCCCTTCTCCATCTCCAGCCTCGGGGCTGCCGCAGG |
| | | GGGACGGCTGCCTTCGGGGGGGACGGGGCAGGGCGGGGTTCGG |
| | | CTTCTGGCGTGTGACCGGCGGGAATTC |
| 73 | DNA fragment | GAATTCATGAAGTGCCTTTTGTACTTAGCCTTTTTATTCATTGGG |
| | containing | GTGAATTGCAAGTTCACCATAGTTTTTCCACACAACCAAAAAGG |
| | VSV-G | AAACTGGAAAAATGTTCCTTCTAATTACCATTATTGCCCGTCAAG |
| | | CTCAGATTTAAATTGGCATAATGACTTAATAGGCACAGCCTTACA |
| | | AGTCAAAATGCCCAAGAGTCACAAGGCTATTCAAGCAGACGGTT |
| | | GGATGTGTCATGCTTCCAAATGGGTCACTACTTGTGATTTCCGCT |
| | | GGTATGGACCGAAGTATATAACACATTCCATCCGATCCTTCACTC |
| | | CATCTGTAGAACAATGCAAGGAAAGCATTGAACAAACGAAACA |
| | | AGGAACTTGGCTGAATCCAGGCTTCCCTCCTCAAAGTTGTGGATA |
| | | TGCAACTGTGACGGATGCCGAAGCAGTGATTGTCCAGGTGACTC |
| | | CTCACCATGTGCTGGTTGATGAATACACAGGAGAATGGGTTGAT |
| | | TCACAGTTCATCAACGGAAAATGCAGCAATTACATATGCCCCAC |
| | | TGTCCATAACTCTACAACCTGGCATTCTGACTATAAGGTCAAAGG |
| | | GCTATGTGATTCTAACCTCATTTCCATGGACATCACCTTCTTCTCA |
| | | GAGGACGGAGAGCTATCATCCCTGGGAAAGGAGGGCACAGGGT |
| | | TCAGAAGTAACTACTTTGCTTATGAAACTGGAGGCAAGGCCTGC |
| | | AAAATGCAATACTGCAAGCATTGGGGAGTCAGACTCCCATCAGG |
| | | TGTCTGGTTCGAGATGGCTGATAAGGATCTCTTTGCTGCAGCCAG |
| | | ATTCCCTGAATGCCCAGAAGGGTCAAGTATCTCTGCTCCATCTCA |
| | | GACCTCAGTGGATGTAAGTCTAATTCAGGACGTTGAGAGGATCT |
| | | TGGATTATTCCCTCTGCCAAGAAACCTGGAGCAAAATCAGAGCG |
| | | GGTCTTCCAATCTCTCCAGTGGATCTCAGCTATCTTGCTCCTAAA |
| | | AACCCAGGAACCGGTCCTGCTTTCACCATAATCAATGGTACCCTA |
| | | AAATACTTTGAGACCAGATACATCAGAGTCGATATTGCTGCTCC |
| | | AATCCTCTCAAGAATGGTCGGAATGATCAGTGGAACTACCACAG |
| | | AAAGGGAACTGTGGGATGACTGGGCACCATATGAAGACGTGGA |
| | | AATTGGACCCAATGGAGTTCTGAGGACCAGTTCAGGATATAAGT |
| | | TTCCTTTATACATGATTGGACATGGTATGTTGGACTCCGATCTTC |
| | | ATCTTAGCTCAAAGGCTCAGGTGTTCGAACATCCTCACATTCAAG |
| | | ACGCTGCTTCGCAACTTCCTGATGATGAGAGTTTATTTTTTGGTG |
| | | ATACTGGGCTATCCAAAAATCCAATCGAGCTTGTAGAAGGTTGG |
| | | TTCAGTAGTTGGAAAAGCTCTATTGCCTCTTTTTTCTTTATCATAG |
| | | GGTTAATCATTGGACTATTCTTGGTTCTCCGAGTTGGTATCCATC |
| | | TTTGCATTAAATTAAAGCACACCAAGAAAAGACAGATTTATACA |
| | | GACATAGAGATGAGAATTC |
| 74 | Helper plasmid | TCTAGAAGGAGCTTTGTTCCTTGGGTTCTTGGGAGCAGCAGGAA |
| | containing | GCACTATGGGCGCAGCGTCAATGACGCTGACGGTACAGGCCAGA |
| | RRE and rabbit | CAATTATTGTCTGGTATAGTGCAGCAGCAGAACAATTTGCTGAG |
| | beta globin | GGCTATTGAGGCGCAACAGCATCTGTTGCAACTCACAGTCTGGG |
| | poly A | GCATCAAGCAGCTCCAGGCAAGAATCCTGGCTGTGGAAAGATAC |
| | | CTAAAGGATCAACAGCTCCTAGATCTTTTTCCCTCTGCCAAAAAT |
| | | TATGGGGACATCATGAAGCCCCTTGAGCATCTGACTTCTGGCTAA |
| | | TAAAGGAAATTTATTTTCATTGCAATAGTGTGTTGGAATTTTTTG |
| | | TGTCTCTCACTCGGAAGGACATATGGGAGGGCAAATCATTTAAA |
| | | ACATCAGAATGAGTATTTGGTTTAGAGTTTGGCAACATATGCCAT |
| | | ATGCTGGCTGCCATGAACAAAGGTGGCTATAAAGAGGTCATCAG |
| | | TATATGAAACAGCCCCCTGCTGTCCATTCCTTATTCCATAGAAAA |
| | | GCCTTGACTTGAGGTTAGATTTTTTTTATATTTTGTTTTGTGTTAT |
| | | TTTTTTCTTTAACATCCCTAAAATTTTCCTTACATGTTTTACTAGC |
| | | CAGATTTTTCCTCCTCTCCTGACTACTCCCAGTCATAGCTGTCCCT |
| | | CTTCTCTTATGAAGATCCCTCGACCTGCAGCCCAAGCTTGGCGTA |
| | | ATCATGGTCATAGCTGTTTCCTGTGTGAAATTGTTATCCGCTCAC |
| | | AATTCCACACAACATACGAGCCGGAAGCATAAAGTGTAAAGCCT |
| | | GGGGTGCCTAATGAGTGAGCTAACTCACATTAATTGCGTTGCGCT |
| | | CACTGCCCGCTTTCCAGTCGGGAAACCTGTCGTGCCAGCGGATCC |
| | | GCATCTCAATTAGTCAGCAACCATAGTCCCGCCCCTAACTCCGCC |
| | | CATCCCGCCCCTAACTCCGCCCAGTTCCGCCCATTCTCCGCCCCA |
| | | TGGCTGACTAATTTTTTTTATTTATGCAGAGGCCGAGGCCGCCTC |
| | | GGCCTCTGAGCTATTCCAGAAGTAGTGAGGAGGCTTTTTTGGAG |
| | | GCCTAGGCTTTTGCAAAAAGCTAACTTGTTTATTGCAGCTTATAA |
| | | TGGTTACAAATAAAGCAATAGCATCACAAATTTCACAAATAAAG |
| | | CATTTTTTTCACTGCATTCTAGTTGTGGTTTGTCCAAACTCATCAA |
| | | TGTATCTTATCACCCGGG |
| 75 | RSV promoter | CAATTGCGATGTACGGGCCAGATATACGCGTATCTGAGGGGACT |
| | and HIV Rev | AGGGTGTGTTTAGGCGAAAAGCGGGGCTTCGGTTGTACGCGGTT |
| | | AGGAGTCCCCTCAGGATATAGTAGTTTCGCTTTTGCATAGGGAG |
| | | GGGGAAATGTAGTCTTATGCAATACACTTGTAGTCTTGCAACATG |
| | | GTAACGATGAGTTAGCAACATGCCTTACAAGGAGAGAAAAAGC |
| | | ACCGTGCATGCCGATTGGTGGAAGTAAGGTGGTACGATCGTGCC |
| | | TTATTAGGAAGGCAACAGACAGGTCTGACATGGATTGGACGAAC |
| | | CACTGAATTCCGCATTGCAGAGATAATTGTATTTAAGTGCCTAGC |
| | | TCGATACAATAAACGCCATTTGACCATTCACCACATTGGTGTGCA |
| | | CCTCCAAGCTCGAGCTCGTTTAGTGAACCGTCAGATCGCCTGGA |
| | | GACGCCATCCACGCTGTTTTGACCTCCATAGAAGACACCGGGAC |
| | | CGATCCAGCCTCCCCTCGAAGCTAGCGATTAGGCATCTCCTATGG |
| | | CAGGAAGAAGCGGAGACAGCGACGAAGAACTCCTCAAGGCAGT |
| | | CAGACTCATCAAGTTTCTCTATCAAAGCAACCCACCTCCCAATCC |
| | | CGAGGGGACCCGACAGGCCCGAAGGAATAGAAGAAGAAGGTGG |
| | | AGAGAGAGACAGAGACAGATCCATTCGATTAGTGAACGGATCCT |
| | | TAGCACTTATCTGGGACGATCTGCGGAGCCTGTGCCTCTTCAGCT |
| | | ACCACCGCTTGAGAGACTTACTCTTGATTGTAACGAGGATTGTGG |
| | | AACTTCTGGGACGCAGGGGGTGGGAAGCCCTCAAATATTGGTGG |
| | | AATCTCCTACAATATTGGAGTCAGGAGCTAAAGAATAGTCTAGA |
| 76 | Rev/Tat | ATGGCAGGAAGAAGCGGAG |
| | shRNA target | |
| | sequence #2 | |
| 77 | Rev/Tat | ATGGCAGGAAGAAGCGGAGTTCAAGAGACTCCGCTTCTTCCTGC |
| | shRNA coding | CATTTTTT |
| | sequence #2 | |
| 78 | H1 promoter | GAACGCTGACGTCATCAACCCGCTCCAAGGAATCGCGGGCCCAG |
| | and shRT | TGTCACTAGGCGGGAACACCCAGCGCGCGTGCGCCCTGGCAGGA |
| | sequence | AGATGGCTGTGAGGGACAGGGGAGTGGCGCCCTGCAATATTTGC |
| | | ATGTCGCTATGTGTTCTGGGAAATCACCATAAACGTGAAATGTCT |
| | | TTGGATTTGGGAATCTTATAAGTTCTGTATGAGACCACTTGGATC |
| | | CGCGGAGACAGCGACGAAGAGCTTCAAGAGAGCTCTTCGTCGCT |
| | | GTCTCCGCTTTTT |
| 79 | H1 CCR5 | GAACGCTGACGTCATCAACCCGCTCCAAGGAATCGCGGGCCCAG |
| | sequence | TGTCACTAGGCGGGAACACCCAGCGCGCGTGCGCCCTGGCAGGA |
| | | AGATGGCTGTGAGGGACAGGGGAGTGGCGCCCTGCAATATTTGC |
| | | ATGTCGCTATGTGTTCTGGGAAATCACCATAAACGTGAAATGTCT |
| | | TTGGATTTGGGAATCTTATAAGTTCTGTATGAGACCACTTGGATC |
| | | CGTGTCAAGTCCAATCTATGTTCAAGAGACATAGATTGGACTTG |
| | | ACACTTTTT |
| 80 | CAG promoter | TAGTTATTAATAGTAATCAATTACGGGGTCATTAGTTCATAGCCC |
| | | ATATATGGAGTTCCGCGTTACATAACTTACGGTAAATGGCCCGCC |
| | | TGGCTGACCGCCCAACGACCCCCGCCCATTGACGTCAATAATGA |
| | | CGTATGTTCCCATAGTAACGCCAATAGGGACTTTCCATTGACGTC |
| | | AATGGGTGGACTATTTACGGTAAACTGCCCACTTGGCAGTACAT |
| | | CAAGTGTATCATATGCCAAGTACGCCCCCTATTGACGTCAATGAC |
| | | GGTAAATGGCCCGCCTGGCATTATGCCCAGTACATGACCTTATG |
| | | GGACTTTCCTACTTGGCAGTACATCTACGTATTAGTCATCGCTAT |
| | | TACCATGGGTCGAGGTGAGCCCCACGTTCTGCTTCACTCTCCCCA |
| | | TCTCCCCCCCCTCCCCACCCCCAATTTTGTATTTATTTATTTTTTA |
| | | ATTATTTTGTGCAGCGATGGGGGCGGGGGGGGGGGGGGCGCGCG |
| | | CCAGGCGGGGCGGGGCGGGGCGAGGGGCGGGGCGGGGCGAGGC |
| | | GGAGAGGTGCGGCGGCAGCCAATCAGAGCGGCGCGCTCCGAAA |
| | | GTTTCCTTTTATGGCGAGGCGGCGGCGGCGGCGGCCCTATAAAA |
| | | AGCGAAGCGCGCGGCGGGCG |
| 81 | pRSV Rev | AGCGCCCAATACGCAAACCGCCTCTCCCCGCGCGTTGGCCGATT |
| | | CATTAATGCAGCTGGCACGACAGGTTTCCCGACTGGAAAGCGGG |
| | | CAGTGAGCGCAACGCAATTAATGTGAGTTAGCTCACTCATTAGG |
| | | CACCCCAGGCTTTACACTTTATGCTTCCGGCTCGTATGTTGTGTG |
| | | GAATTGTGAGCGGATAACAATTTCACACAGGAAACAGCTATGAC |
| | | CATGATTACGAATTCGATGTACGGGCCAGATATACGCGTATCTG |
| | | AGGGGACTAGGGTGTGTTTAGGCGAAAAGCGGGGCTTCGGTTGT |
| | | ACGCGGTTAGGAGTCCCCTCAGGATATAGTAGTTTCGCTTTTGCA |
| | | TAGGGAGGGGGAAATGTAGTCTTATGCAATACACTTGTAGTCTT |
| | | GCAACATGGTAACGATGAGTTAGCAACATGCCTTACAAGGAGAG |
| | | AAAAAGCACCGTGCATGCCGATTGGTGGAAGTAAGGTGGTACGA |
| | | TCGTGCCTTATTAGGAAGGCAACAGACAGGTCTGACATGGATTG |
| | | GACGAACCACTGAATTCCGCATTGCAGAGATAATTGTATTTAAG |
| | | TGCCTAGCTCGATACAATAAACGCCATTTGACCATTCACCACATT |
| | | GGTGTGCACCTCCAAGCTCGAGCTCGTTTAGTGAACCGTCAGATC |
| | | GCCTGGAGACGCCATCCACGCTGTTTTGACCTCCATAGAAGACA |
| | | CCGGGACCGATCCAGCCTCCCCTCGAAGCTAGTCGATTAGGCAT |
| | | CTCCTATGGCAGGAAGAAGCGGAGACAGCGACGAAGACCTCCTC |
| | | AAGGCAGTCAGACTCATCAAGTTTCTCTATCAAAGCAACCCACC |
| | | TCCCAATCCCGAGGGGACCCGACAGGCCCGAAGGAATAGAAGA |
| | | AGAAGGTGGAGAGAGAGACAGAGACAGATCCATTCGATTAGTG |
| | | AACGGATCCTTAGCACTTATCTGGGACGATCTGCGGAGCCTGTG |
| | | CCTCTTCAGCTACCACCGCTTGAGAGACTTACTCTTGATTGTAAC |
| | | GAGGATTGTGGAACTTCTGGGACGCAGGGGGTGGGAAGCCCTCA |
| | | AATATTGGTGGAATCTCCTACAATATTGGAGTCAGGAGCTAAAG |
| | | AATAGTGCTGTTAGCTTGCTCAATGCCACAGCTATAGCAGTAGCT |
| | | GAGGGGACAGATAGGGTTATAGAAGTAGTACAAGAAGCTTGGC |
| | | ACTGGCCGTCGTTTTACATGATCTGAGCCTGGGAGATCTCTGGCT |
| | | AACTAGGGAACCCACTGCTTAAGCCTCAATAAAGCTTGCCTTGA |
| | | GTGCTTCAAGTAGTGTGTGCCCGTCTGTTGTGTGACTCTGGTAAC |
| | | TAGAGATCACAAAGCAACCATAGTACGCGCCCTGTAGCGGCGCA |
| | | TTAAGCGCGGCGGGTGTGGTGGTTACGCGCAGCGTGACCGCTAC |
| | | ACTTGCCAGCGCCCTAGCGCCCGCTCCTTTCGCTTTCTTCCCTTCC |
| | | TTTCTCGCCACGTTCGCCGGCTTTCCCCGTCAAGCTCTAAATCGG |
| | | GGGCTCCCTTTAGGGTTCCGATTTAGTGCTTTACGGCACCTCGAC |
| | | CCCAAAAAACTTGATTTGGGTGATGGTTCACGTAGTGGGCCATC |
| | | GCCCTGATAGACGGTTTTTCGCCCTTTGACGTTGGAGTCCACGTT |
| | | CTTTAATAGTGGACTCTTGTTCCAAACTGGAACAACACTCAACCC |
| | | TATCTCGGGCTATTCTTTTGATTTATAAGGGATTTTGCCGATTTCG |
| | | GCCTATTGGTTAAAAAATGAGCTGATTTAACAAAAATTTAACGC |
| | | GAATTTTAACAAAATATTAACGTTTACAATTTTATGGTGCACTCT |
| | | CAGTACAATCTGCTCTGATGCCGCATAGTTAAGCCAGCCCCGAC |
| | | ACCCGCCAACACCCGCTGACGCGCCCTGACGGGCTTGTCTGCTCC |
| | | CGGCATCCGCTTACAGACAAGCTGTGACCGTCTCCGGGAGCTGC |
| | | ATGTGTCAGAGGTTTTCACCGTCATCACCGAAACGCGCGAGACG |
| | | AAAGGGCCTCGTGATACGCCTATTTTTATAGGTTAATGTCATGAT |
| | | AATAATGGTTTCTTAGACGTCAGGTGGCACTTTTCGGGGAAATGT |
| | | GCGCGGAACCCCTATTTGTTTATTTTTCTAAATACATTCAAATAT |
| | | GTATCCGCTCATGAGACAATAACCCTGATAAATGCTTCAATAAT |
| | | ATTGAAAAAGGAAGAGTATGAGTATTCAACATTTCCGTGTCGCC |
| | | CTTATTCCCTTTTTTGCGGCATTTTGCCTTCCTGTTTTTGCTCACCC |
| | | AGAAACGCTGGTGAAAGTAAAAGATGCTGAAGATCAGTTGGGTG |
| | | CACGAGTGGGTTACATCGAACTGGATCTCAACAGCGGTAAGATC |
| | | CTTGAGAGTTTTCGCCCCGAAGAACGTTTTCCAATGATGAGCACT |
| | | TTTAAAGTTCTGCTATGTGGCGCGGTATTATCCCGTATTGACGCC |
| | | GGGCAAGAGCAACTCGGTCGCCGCATACACTATTCTCAGAATGA |
| | | CTTGGTTGAGTACTCACCAGTCACAGAAAAGCATCTTACGGATG |
| | | GCATGACAGTAAGAGAATTATGCAGTGCTGCCATAACCATGAGT |
| | | GATAACACTGCGGCCAACTTACTTCTGACAACGATCGGAGGACC |
| | | GAAGGAGCTAACCGCTTTTTTGCACAACATGGGGGATCATGTAA |
| | | CTCGCCTTGATCGTTGGGAACCGGAGCTGAATGAAGCCATACCA |
| | | AACGACGAGCGTGACACCACGATGCCTGTAGCAATGGCAACAAC |
| | | GTTGCGCAAACTATTAACTGGCGAACTACTTACTCTAGCTTCCCG |
| | | GCAACAATTAATAGACTGGATGGAGGCGGATAAAGTTGCAGGAC |
| | | CACTTCTGCGCTCGGCCCTTCCGGCTGGCTGGTTTATTGCTGATA |
| | | AATCTGGAGCCGGTGAGCGTGGGTCTCGCGGTATCATTGCAGCA |
| | | CTGGGGCCAGATGGTAAGCCCTCCCGTATCGTAGTTATCTACACG |
| | | ACGGGGAGTCAGGCAACTATGGATGAACGAAATAGACAGATCG |
| | | CTGAGATAGGTGCCTCACTGATTAAGCATTGGTAACTGTCAGAC |
| | | CAAGTTTACTCATATATACTTTAGATTGATTTAAAACTTCATTTTT |
| | | AATTTAAAAGGATCTAGGTGAAGATCCTTTTTGATAATCTCATGA |
| | | CCAAAATCCCTTAACGTGAGTTTTCGTTCCACTGAGCGTCAGACC |
| | | CCGTAGAAAAGATCAAAGGATCTTCTTGAGATCCTTTTTTTCTGC |
| | | GCGTAATCTGCTGCTTGCAAACAAAAAAACCACCGCTACCAGCG |
| | | GTGGTTTGTTTGCCGGATCAAGAGCTACCAACTCTTTTTCCGAAG |
| | | GTAACTGGCTTCAGCAGAGCGCAGATACCAAATACTGTCCTTCT |
| | | AGTGTAGCCGTAGTTAGGCCACCACTTCAAGAACTCTGTAGCAC |
| | | CGCCTACATACCTCGCTCTGCTAATCCTGTTACCAGTGGCTGCTG |
| | | CCAGTGGCGATAAGTCGTGTCTTACCGGGTTGGACTCAAGACGA |
| | | TAGTTACCGGATAAGGCGCAGCGGTCGGGCTGAACGGGGGGTTC |
| | | GTGCACACAGCCCAGCTTGGAGCGAACGACCTACACCGAACTGA |
| | | GATACCTACAGCGTGAGCTATGAGAAAGCGCCACGCTTCCCGAA |
| | | GGGAGAAAGGCGGACAGGTATCCGGTAAGCGGCAGGGTCGGAA |
| | | CAGGAGAGCGCACGAGGGAGCTTCCAGGGGGAAACGCCTGGTA |
| | | TCTTTATAGTCCTGTCGGGTTTCGCCACCTCTGACTTGAGCGTCG |
| | | ATTTTTGTGATGCTCGTCAGGGGGGCGGAGCCTATGGAAAAACG |
| | | CCAGCAACGCGGCCTTTTTACGGTTCCTGGCCTTTTGCTGGCCTT |
| | | TTGCTCACATGTTCTTTCCTGCGTTATCCCCTGATTCTGTGGATAA |
| | | CCGTATTACCGCCTTTGAGTGAGCTGATACCGCTCGCCGCAGCCG |
| | | AACGACCGAGCGCAGCGAGTCAGTGAGCGAGGAAGCGGAAG |
| 82 | pCMV-VSV-G | GAGCTTGGCCCATTGCATACGTTGTATCCATATCATAATATGTAC |
| | | ATTTATATTGGCTCATGTCCAACATTACCGCCATGTTGACATTGA |
| | | TTATTGACTAGTTATTAATAGTAATCAATTACGGGGTCATTAGTT |
| | | CATAGCCCATATATGGAGTTCCGCGTTACATAACTTACGGTAAAT |
| | | GGCCCGCCTGGCTGACCGCCCAACGACCCCCGCCCATTGACGTC |
| | | AATAATGACGTATGTTCCCATAGTAACGCCAATAGGGACTTTCC |
| | | ATTGACGTCAATGGGTGGAGTATTTACGGTAAACTGCCCACTTG |
| | | GCAGTACATCAAGTGTATCATATGCCAAGTACGCCCCCTATTGAC |
| | | GTCAATGACGGTAAATGGCCCGCCTGGCATTATGCCCAGTACAT |
| | | GACCTTATGGGACTTTCCTACTTGGCAGTACATCTACGTATTAGT |
| | | CATCGCTATTACCATGGTGATGCGGTTTTGGCAGTACATCAATGG |
| | | GCGTGGATAGCGGTTTGACTCACGGGGATTTCCAAGTCTCCACCC |
| | | CATTGACGTCAATGGGAGTTTGTTTTGGCACCAAAATCAACGGG |
| | | ACTTTCCAAAATGTCGTAACAACTCCGCCCCATTGACGCAAATG |
| | | GGCGGTAGGCGTGTACGGTGGGAGGTCTATATAAGCAGAGCTCG |
| | | TTTAGTGAACCGTCAGATCGCCTGGAGACGCCATCCACGCTGTTT |
| | | TGACCTCCATAGAAGACACCGGGACCGATCCAGCCTCCGGTCGA |
| | | CCGATCCTGAGAACTTCAGGGTGAGTTTGGGGACCCTTGATTGTT |
| | | CTTTCTTTTTCGCTATTGTAAAATTCATGTTATATGGAGGGGGCA |
| | | AAGTTTTCAGGGTGTTGTTTAGAATGGGAAGATGTCCCTTGTATC |
| | | ACCATGGACCCTCATGATAATTTTGTTTCTTTCACTTTCTACTCTG |
| | | TTGACAACCATTGTCTCCTCTTATTTTCTTTTCATTTTCTGTAACTT |
| | | TTTCGTTAAACTTTAGCTTGCATTTGTAACGAATTTTTAAATTCAC |
| | | TTTTGTTTATTTGTCAGATTGTAAGTACTTTCTCTAATCACTTTTT |
| | | TTTCAAGGCAATCAGGGTATATTATATTGTACTTCAGCACAGTTT |
| | | TAGAGAACAATTGTTATAATTAAATGATAAGGTAGAATATTTCT |
| | | GCATATAAATTCTGGCTGGCGTGGAAATATTCTTATTGGTAGAAA |
| | | CAACTACACCCTGGTCATCATCCTGCCTTTCTCTTTATGGTTACA |
| | | ATGATATACACTGTTTGAGATGAGGATAAAATACTCTGAGTCCA |
| | | AACCGGGCCCCTCTGCTAACCATGTTCATGCCTTCTTCTCTTTCCT |
| | | ACAGCTCCTGGGCAACGTGCTGGTTGTTGTGCTGTCTCATCATTT |
| | | TGGCAAAGAATTCCTCGACGGATCCGCCATGAAGTGCCTTTTGTA |
| | | CTTAGCCTTTTTATTCATTGGGGTGAATTGCAAGTTCACCATAGT |
| | | TTTTCCACACAACCAAAAAGGAAACTGGAAAAATGTTCCTTCTA |
| | | ATTACCATTATTGCCCGTCAAGCTCAGATTTAAATTGGCATAATG |
| | | ACTTAATAGGCACAGCCTTACAAGTCAAAATGCCCAAGAGTCAC |
| | | AAGGCTATTCAAGCAGACGGTTGGATGTGTCATGCTTCCAAATG |
| | | GGTCACTACTTGTGATTTCCGCTGGTATGGACCGAAGTATATAAC |
| | | ACATTCCATCCGATCCTTCACTCCATCTGTAGAACAATGCAAGGA |
| | | AAGCATTGAACAAACGAAACAAGGAACTTGGCTGAATCCAGGCT |
| | | TCCCTCCTCAAAGTTGTGGATATGCAACTGTGACGGATGCCGAA |
| | | GCAGTGATTGTCCAGGTGACTCCTCACCATGTGCTGGTTGATGAA |
| | | TACACAGGAGAATGGGTTGATTCACAGTTCATCAACGGAAAATG |
| | | CAGCAATTACATATGCCCCACTGTCCATAACTCTACAACCTGGCA |
| | | TTCTGACTATAAGGTCAAAGGGCTATGTGATTCTAACCTCATTTC |
| | | CATGGACATCACCTTCTTCTCAGAGGACGGAGAGCTATCATCCCT |
| | | GGGAAAGGAGGGCACAGGGTTCAGAAGTAACTACTTTGCTTATG |
| | | AAACTGGAGGCAAGGCCTGCAAAATGCAATACTGCAAGCATTGG |
| | | GGAGTCAGACTCCCATCAGGTGTCTGGTTCGAGATGGCTGATAA |
| | | GGATCTCTTTGCTGCAGCCAGATTCCCTGAATGCCCAGAAGGGTC |
| | | AAGTATCTCTGCTCCATCTCAGACCTCAGTGGATGTAAGTCTAAT |
| | | TCAGGACGTTGAGAGGATCTTGGATTATTCCCTCTGCCAAGAAA |
| | | CCTGGAGCAAAATCAGAGCGGGTCTTCCAATCTCTCCAGTGGAT |
| | | CTCAGCTATCTTGCTCCTAAAAACCCAGGAACCGGTCCTGCTTTC |
| | | ACCATAATCAATGGTACCCTAAAATACTTTGAGACCAGATACAT |
| | | CAGAGTCGATATTGCTGCTCCAATCCTCTCAAGAATGGTCGGAAT |
| | | GATCAGTGGAACTACCACAGAAAGGGAACTGTGGGATGACTGG |
| | | GCACCATATGAAGACGTGGAAATTGGACCCAATGGAGTTCTGAG |
| | | GACCAGTTCAGGATATAAGTTTCCTTTATACATGATTGGACATGG |
| | | TATGTTGGACTCCGATCTTCATCTTAGCTCAAAGGCTCAGGTGTT |
| | | CGAACATCCTCACATTCAAGACGCTGCTTCGCAACTTCCTGATGA |
| | | TGAGAGTTTATTTTTTGGTGATACTGGGCTATCCAAAAATCCAAT |
| | | CGAGCTTGTAGAAGGTTGGTTCAGTAGTTGGAAAAGCTCTATTG |
| | | CCTCTTTTTTCTTTATCATAGGGTTAATCATTGGACTATTCTTGGT |
| | | TCTCCGAGTTGGTATCCATCTTTGCATTAAATTAAAGCACACCAA |
| | | GAAAAGACAGATTTATACAGACATAGAGATGAACCGACTTGGAA |
| | | AGTGATAAGGATCCGTCGAGGAATTCACTCCTCAGGTGCAGGCT |
| | | GCCTATCAGAAGGTGGTGGCTGGTGTGGCCAATGCCCTGGCTCA |
| | | CAAATACCACTGAGATCTTTTTCCCTCTGCCAAAAATTATGGGGA |
| | | CATCATGAAGCCCCTTGAGCATCTGACTTCTGGCTAATAAAGGA |
| | | AATTTATTTTCATTGCAATAGTGTGTTGGAATTTTTTGTGTCTCTC |
| | | ACTCGGAAGGACATATGGGAGGGCAAATCATTTAAAACATCAGA |
| | | ATGAGTATTTGGTTTAGAGTTTGGCAACATATGCCCATATGCTGG |
| | | CTGCCATGAACAAAGGTTGGCTATAAAGAGGTCATCAGTATATG |
| | | AAACAGCCCCCTGCTGTCCATTCCTTATTCCATAGAAAAGCCTTG |
| | | ACTTGAGGTTAGATTTTTTTTATATTTTGTTTTGTGTTATTTTTTTC |
| | | TTTAACATCCCTAAAATTTTCCTTACATGTTTTACTAGCCAGATTT |
| | | TTCCTCCTCTCCTGACTACTCCCAGTCATAGCTGTCCCTCTTCTCT |
| | | TATGGAGATCCCTCGACGGATCGGCCGCAATTCGTAATCATGTC |
| | | ATAGCTGTTTCCTGTGTGAAATTGTTATCCGCTCACAATTCCACA |
| | | CAACATACGAGCCGGAAGCATAAAGTGTAAAGCCTGGGGTGCCT |
| | | AATGAGTGAGCTAACTCACATTAATTGCGTTGCGCTCACTGCCCG |
| | | CTTTCCAGTCGGGAAACCTGTCGTGCCAGCTGCATTAATGAATCG |
| | | GCCAACGCGCGGGGAGAGGCGGTTTGCGTATTGGGCGCTCTTCC |
| | | GCTTCCTCGCTCACTGACTCGCTGCGCTCGGTCGTTCGGCTGCGG |
| | | CGAGCGGTATCAGCTCACTCAAAGGCGGTAATACGGTTATCCAC |
| | | AGAATCAGGGGATAACGCAGGAAAGAACATGTGAGCAAAAGGC |
| | | CAGCAAAAGGCCAGGAACCGTAAAAAGGCCGCGTTGCTGGCGTT |
| | | TTTCCATAGGCTCCGCCCCCCTGACGAGCATCACAAAAATCGAC |
| | | GCTCAAGTCAGAGGTGGCGAAACCCGACAGGACTATAAAGATAC |
| | | CAGGCGTTTCCCCCTGGAAGCTCCCTCGTGCGCTCTCCTGTTCCG |
| | | ACCCTGCCGCTTACCGGATACCTGTCCGCCTTTCTCCCTTCGGGA |
| | | AGCGTGGCGCTTTCTCATAGCTCACGCTGTAGGTATCTCAGTTCG |
| | | GTGTAGGTCGTTCGCTCCAAGCTGGGCTGTGTGCACGAACCCCCC |
| | | GTTCAGCCCGACCGCTGCGCCTTATCCGGTAACTATCGTCTTGAG |
| | | TCCAACCCGGTAAGACACGACTTATCGCCACTGGCAGCAGCCAC |
| | | TGGTAACAGGATTAGCAGAGCGAGGTATGTAGGCGGTGCTACAG |
| | | AGTTCTTGAAGTGGTGGCCTAACTACGGCTACACTAGAAGAACA |
| | | GTATTTGGTATCTGCGCTCTGCTGAAGCCAGTTACCTTCGGAAAA |
| | | AGAGTTGGTAGCTCTTGATCCGGCAAACAAACCACCGCTGGTAG |
| | | CGGTGGTTTTTTTGTTTGCAAGCAGCAGATTACGCGCAGAAAAA |
| | | AAGGATCTCAAGAAGATCCTTTGATCTTTTCTACGGGGTCTGACG |
| | | CTCAGTGGAACGAAAACTCACGTTAAGGGATTTTGGTCATGAGA |
| | | TTATCAAAAAGGATCTTCACCTAGATCCTTTTAAATTAAAAATGA |
| | | AGTTTTAAATCAATCTAAAGTATATATGAGTAAACTTGGTCTGAC |
| | | AGTTACCAATGCTTAATCAGTGAGGCACCTATCTCAGCGATCTGT |
| | | CTATTTCGTTCATCCATAGTTGCCTGACTCCCCGTCGTGTAGATA |
| | | ACTACGATACGGGAGGGCTTACCATCTGGCCCCAGTGCTGCAAT |
| | | GATACCGCGAGACCCACGCTCACCGGCTCCAGATTTATCAGCAA |
| | | TAAACCAGCCAGCCGGAAGGGCCGAGCGCAGAAGTGGTCCTGC |
| | | AACTTTATCCGCCTCCATCCAGTCTATTAATTGTTGCCGGGAAGC |
| | | TAGAGTAAGTAGTTCGCCAGTTAATAGTTTGCGCAACGTTGTTGC |
| | | CATTGCTACAGGCATCGTGGTGTCACGCTCGTCGTTTGGTATGGC |
| | | TTCATTCAGCTCCGGTTCCCAACGATCAAGGCGAGTTACATGATC |
| | | CCCCATGTTGTGCAAAAAAGCGGTTAGCTCCTTCGGTCCTCCGAT |
| | | CGTTGTCAGAAGTAAGTTGGCCGCAGTGTTATCACTCATGGTTAT |
| | | GGCAGCACTGCATAATTCTCTTACTGTCATGCCATCCGTAAGATG |
| | | CTTTTCTGTGACTGGTGAGTACTCAACCAAGTCATTCTGAGAATA |
| | | GTGTATGCGGCGACCGAGTTGCTCTTGCCCGGCGTCAATACGGG |
| | | ATAATACCGCGCCACATAGCAGAACTTTAAAAGTGCTCATCATT |
| | | GGAAAACGTTCTTCGGGGCGAAAACTCTCAAGGATCTTACCGCT |
| | | GTTGAGATCCAGTTCGATGTAACCCACTCGTGCACCCAACTGATC |
| | | TTCAGCATCTTTTACTTTCACCAGCGTTTCTGGGTGAGCAAAAAC |
| | | AGGAAGGCAAAATGCCGCAAAAAAGGGAATAAGGGCGACACGG |
| | | AAATGTTGAATACTCATACTCTTCCTTTTTCAATATTATTGAAGC |
| | | ATTTATCAGGGTTATTGTCTCATGAGCGGATACATATTTGAATGT |
| | | ATTTAGAAAAATAAACAAATAGGGGTTCCGCGCACATTTCCCCG |
| | | AAAAGTGCCACCTAAATTGTAAGCGTTAATATTTTGTTAAAATTC |
| | | GCGTTAAATTTTTGTTAAATCAGCTCATTTTTTAACCAATAGGCC |
| | | GAAATCGGCAAAATCCCTTATAAATCAAAAGAATAGACCGAGAT |
| | | AGGGTTGAGTGTTGTTCCAGTTTGGAACAAGAGTCCACTATTAA |
| | | AGAACGTGGACTCCAACGTCAAAGGGCGAAAAACCGTCTATCAG |
| | | GGCGATGGCCCACTACGTGAACCATCACCCTAATCAAGTTTTTTG |
| | | GGGTCGAGGTGCCGTAAAGCACTAAATCGGAACCCTAAAGGGA |
| | | GCCCCCGATTTAGAGCTTGACGGGGAAAGCCGGCGAACGTGGCG |
| | | AGAAAGGAAGGGAAGAAAGCGAAAGGAGCGGGCGCTAGGGCG |
| | | CTGGCAAGTGTAGCGGTCACGCTGCGCGTAACCACCACACCCGC |
| | | CGCGCTTAATGCGCCGCTACAGGGCGCGTCCCATTCGCCATTCAG |
| | | GCTGCGCAACTGTTGGGAAGGGCGATCGGTGCGGGCCTCTTCGC |
| | | TATTACGCCAGCTGGCGAAAGGGGGATGTGCTGCAAGGCGATTA |
| | | AGTTGGGTAACGCCAGGGTTTTCCCAGTCACGACGTTGTAAAAC |
| | | GACGGCCAGTGAGCGCGCGTAATACGACTCACTATAGGGCGAAT |
| | | TGGAGCTCCACCGCGGTGGCGGCCGCTCTAGA |
| 83 | PSPAX2 delta | GTCGACATTGATTATTGACTAGTTATTAATAGTAATCAATTACGG |
| | Rev | GGTCATTAGTTCATAGCCCATATATGGAGTTCCGCGTTACATAAC |
| | | TTACGGTAAATGGCCCGCCTGGCTGACCGCCCAACGACCCCCGC |
| | | CCATTGACGTCAATAATGACGTATGTTCCCATAGTAACGCCAATA |
| | | GGGACTTTCCATTGACGTCAATGGGTGGACTATTTACGGTAAACT |
| | | GCCCACTTGGCAGTACATCAAGTGTATCATATGCCAAGTACGCC |
| | | CCCTATTGACGTCAATGACGGTAAATGGCCCGCCTGGCATTATGC |
| | | CCAGTACATGACCTTATGGGACTTTCCTACTTGGCAGTACATCTA |
| | | CGTATTAGTCATCGCTATTACCATGGGTCGAGGTGAGCCCCACGT |
| | | TCTGCTTCACTCTCCCCATCTCCCCCCCCTCCCCACCCCCAATTTT |
| | | GTATTTATTTATTTTTTAATTATTTTGTGCAGCGATGGGGGCGGG |
| | | GGGGGGGGGGGCGCGCGCCAGGCGGGGCGGGGCGGGGCGAGGG |
| | | GCGGGGCGGGGCGAGGCGGAGAGGTGCGGCGGCAGCCAATCAG |
| | | AGCGGCGCGCTCCGAAAGTTTCCTTTTATGGCGAGGCGGCGGCG |
| | | GCGGCGGCCCTATAAAAAGCGAAGCGCGCGGCGGGCGGGAGTC |
| | | GCTGCGTTGCCTTCGCCCCGTGCCCCGCTCCGCGCCGCCTCGCGC |
| | | CGCCCGCCCCGGCTCTGACTGACCGCGTTACTCCCACAGGTGAG |
| | | CGGGCGGGACGGCCCTTCTCCTCCGGGCTGTAATTAGCGCTTGGT |
| | | TTAATGACGGCTCGTTTCTTTTCTGTGGCTGCGTGAAAGCCTTAA |
| | | AGGGCTCCGGGAGGGCCCTTTGTGCGGGGGGGAGCGGCTCGGGG |
| | | GGTGCGTGCGTGTGTGTGTGCGTGGGGAGCGCCGCGTGCGGCCC |
| | | GCGCTGCCCGGCGGCTGTGAGCGCTGCGGGCGCGGCGCGGGGCT |
| | | TTGTGCGCTCCGCGTGTGCGCGAGGGGAGCGCGGCCGGGGGCGG |
| | | TGCCCCGCGGTGCGGGGGGGCTGCGAGGGGAACAAAGGCTGCG |
| | | TGCGGGGTGTGTGCGTGGGGGGGTGAGCAGGGGGTGTGGGCGC |
| | | GGCGGTCGGGCTGTAACCCCCCCCTGCACCCCCCTCCCCGAGTTG |
| | | CTGAGCACGGCCCGGCTTCGGGTGCGGGGCTCCGTGCGGGGCGT |
| | | GGCGCGGGGCTCGCCGTGCCGGGCGGGGGGTGGCGGCAGGTGG |
| | | GGGTGCCGGGCGGGGCGGGGCCGCCTCGGGCCGGGGAGGGCTC |
| | | GGGGGAGGGGCGCGGCGGCCCCGGAGCGCCGGCGGCTGTCGAG |
| | | GCGCGGCGAGCCGCAGCCATTGCCTTTTATGGTAATCGTGCGAG |
| | | AGGGCGCAGGGACTTCCTTTGTCCCAAATCTGGCGGAGCCGAAA |
| | | TCTGGGAGGCGCCGCCGCACCCCCTCTAGCGGGCGCGGGCGAAG |
| | | CGGTGCGGCGCCGGCAGGAAGGAAATGGGCGGGGAGGGCCTTC |
| | | GTGCGTCGCCGCGCCGCCGTCCCCTTCTCCATCTCCAGCCTCGGG |
| | | GCTGCCGCAGGGGGACGGCTGCCTTCGGGGGGGACGGGGCAGG |
| | | GCGGGGTTCGGCTTCTGGCGTGTGACCGGCGGCTCTAGAGCCTCT |
| | | GCTAACCATGTTCATGCCTTCTTCTTTTTCCTACAGCTCCTGGGCA |
| | | ACGTGCTGGTTATTGTGCTGTCTCATCATTTTGGCAAAGAATTCG |
| | | GGCCGGCCGCGTTGACGCGCACGGCAAGAGGCGAGGGGCGGCG |
| | | ACTGGTGAGAGATGGGTGCGAGAGCGTCAGTATTAAGCGGGGG |
| | | AGAATTAGATCGATGGGAAAAAATTCGGTTAAGGCCAGGGGGA |
| | | AAGAAAAAATATAAATTAAAACATATAGTATGGGCAAGCAGGG |
| | | AGCTAGAACGATTCGCAGTTAATCCTGGCCTGTTAGAAACATCA |
| | | GAAGGCTGTAGACAAATACTGGGACAGCTACAACCATCCCTTCA |
| | | GACAGGATCAGAAGAACTTAGATCATTATATAATACAGTAGCAA |
| | | CCCTCTATTGTGTGCATCAAAGGATAGAGATAAAAGACACCAAG |
| | | GAAGCTTTAGACAAGATAGAGGAAGAGCAAAACAAAAGTAAGA |
| | | AAAAAGCACAGCAAGCAGCAGCTGACACAGGACACAGCAATCA |
| | | GGTCAGCCAAAATTACCCTATAGTGCAGAACATCCAGGGGCAAA |
| | | TGGTACATCAGGCCATATCACCTAGAACTTTAAATGCATGGGTA |
| | | AAAGTAGTAGAAGAGAAGGCTTTCAGCCCAGAAGTGATACCCAT |
| | | GTTTTCAGCATTATCAGAAGGAGCCACCCCACAAGATTTAAACA |
| | | CCATGCTAAACACAGTGGGGGGACATCAAGCAGCCATGCAAATG |
| | | TTAAAAGAGACCATCAATGAGGAAGCTGCAGAATGGGATAGAG |
| | | TGCATCCAGTGCATGCAGGGCCTATTGCACCAGGCCAGATGAGA |
| | | GAACCAAGGGGAAGTGACATAGCAGGAACTACTAGTACCCTTCA |
| | | GGAACAAATAGGATGGATGACACATAATCCACCTATCCCAGTAG |
| | | GAGAAATCTATAAAAGATGGATAATCCTGGGATTAAATAAAATA |
| | | GTAAGAATGTATAGCCCTACCAGCATTCTGGACATAAGACAAGG |
| | | ACCAAAGGAACCCTTTAGAGACTATGTAGACCGATTCTATAAAA |
| | | CTCTAAGAGCCGAGCAAGCTTCACAAGAGGTAAAAAATTGGATG |
| | | ACAGAAACCTTGTTGGTCCAAAATGCGAACCCAGATTGTAAGAC |
| | | TATTTTAAAAGCATTGGGACCAGGAGCGACACTAGAAGAAATGA |
| | | TGACAGCATGTCAGGGAGTGGGGGGACCCGGCCATAAAGCAAG |
| | | AGTTTTGGCTGAAGCAATGAGCCAAGTAACAAATCCAGCTACCA |
| | | TAATGATACAGAAAGGCAATTTTAGGAACCAAAGAAAGACTGTT |
| | | AAGTGTTTCAATTGTGGCAAAGAAGGGCACATAGCCAAAAATTG |
| | | CAGGGCCCCTAGGAAAAAGGGCTGTTGGAAATGTGGAAAGGAA |
| | | GGACACCAAATGAAAGATTGTACTGAGAGACAGGCTAATTTTTT |
| | | AGGGAAGATCTGGCCTTCCCACAAGGGAAGGCCAGGGAATTTTC |
| | | TTCAGAGCAGACCAGAGCCAACAGCCCCACCAGAAGAGAGCTTC |
| | | AGGTTTGGGGAAGAGACAACAACTCCCTCTCAGAAGCAGGAGCC |
| | | GATAGACAAGGAACTGTATCCTTTAGCTTCCCTCAGATCACTCTT |
| | | TGGCAGCGACCCCTCGTCACAATAAAGATAGGGGGGCAATTAAA |
| | | GGAAGCTCTATTAGATACAGGAGCAGATGATACAGTATTAGAAG |
| | | AAATGAATTTGCCAGGAAGATGGAAACCAAAAATGATAGGGGG |
| | | AATTGGAGGTTTTATCAAAGTAGGACAGTATGATCAGATACTCA |
| | | TAGAAATCTGCGGACATAAAGCTATAGGTACAGTATTAGTAGGA |
| | | CCTACACCTGTCAACATAATTGGAAGAAATCTGTTGACTCAGATT |
| | | GGCTGCACTTTAAATTTTCCCATTAGTCCTATTGAGACTGTACCA |
| | | GTAAAATTAAAGCCAGGAATGGATGGCCCAAAAGTTAAACAATG |
| | | GCCATTGACAGAAGAAAAAATAAAAGCATTAGTAGAAATTTGTA |
| | | CAGAAATGGAAAAGGAAGGAAAAATTTCAAAAATTGGGCCTGA |
| | | AAATCCATACAATACTCCAGTATTTGCCATAAAGAAAAAAGACA |
| | | GTACTAAATGGAGAAAATTAGTAGATTTCAGAGAACTTAATAAG |
| | | AGAACTCAAGATTTCTGGGAAGTTCAATTAGGAATACCACATCC |
| | | TGCAGGGTTAAAACAGAAAAAATCAGTAACAGTACTGGATGTGG |
| | | GCGATGCATATTTTTCAGTTCCCTTAGATAAAGACTTCAGGAAGT |
| | | ATACTGCATTTACCATACCTAGTATAAACAATGAGACACCAGGG |
| | | ATTAGATATCAGTACAATGTGCTTCCACAGGGATGGAAAGGATC |
| | | ACCAGCAATATTCCAGTGTAGCATGACAAAAATCTTAGAGCCTT |
| | | TTAGAAAACAAAATCCAGACATAGTCATCTATCAATACATGGAT |
| | | GATTTGTATGTAGGATCTGACTTAGAAATAGGGCAGCATAGAAC |
| | | AAAAATAGAGGAACTGAGACAACATCTGTTGAGGTGGGGATTTA |
| | | CCACACCAGACAAAAAACATCAGAAAGAACCTCCATTCCTTTGG |
| | | ATGGGTTATGAACTCCATCCTGATAAATGGACAGTACAGCCTAT |
| | | AGTGCTGCCAGAAAAGGACAGCTGGACTGTCAATGACATACAGA |
| | | AATTAGTGGGAAAATTGAATTGGGCAAGTCAGATTTATGCAGGG |
| | | ATTAAAGTAAGGCAATTATGTAAACTTCTTAGGGGAACCAAAGC |
| | | ACTAACAGAAGTAGTACCACTAACAGAAGAAGCAGAGCTAGAA |
| | | CTGGCAGAAAACAGGGAGATTCTAAAAGAACCGGTACATGGAG |
| | | TGTATTATGACCCATCAAAAGACTTAATAGCAGAAATACAGAAG |
| | | CAGGGGCAAGGCCAATGGACATATCAAATTTATCAAGAGCCATT |
| | | TAAAAATCTGAAAACAGGAAAATATGCAAGAATGAAGGGTGCC |
| | | CACACTAATGATGTGAAACAATTAACAGAGGCAGTACAAAAAAT |
| | | AGCCACAGAAAGCATAGTAATATGGGGAAAGACTCCTAAATTTA |
| | | AATTACCCATACAAAAGGAAACATGGGAAGCATGGTGGACAGA |
| | | GTATTGGCAAGCCACCTGGATTCCTGAGTGGGAGTTTGTCAATAC |
| | | CCCTCCCTTAGTGAAGTTATGGTACCAGTTAGAGAAAGAACCCA |
| | | TAATAGGAGCAGAAACTTTCTATGTAGATGGGGCAGCCAATAGG |
| | | GAAACTAAATTAGGAAAAGCAGGATATGTAACTGACAGAGGAA |
| | | GACAAAAAGTTGTCCCCCTAACGGACACAACAAATCAGAAGACT |
| | | GAGTTACAAGCAATTCATCTAGCTTTGCAGGATTCGGGATTAGA |
| | | AGTAAACATAGTGACAGACTCACAATATGCATTGGGAATCATTC |
| | | AAGCACAACCAGATAAGAGTGAATCAGAGTTAGTCAGTCAAATA |
| | | ATAGAGCAGTTAATAAAAAAGGAAAAAGTCTACCTGGCATGGGT |
| | | ACCAGCACACAAAGGAATTGGAGGAAATGAACAAGTAGATGGG |
| | | TTGGTCAGTGCTGGAATCAGGAAAGTACTATTTTTAGATGGAAT |
| | | AGATAAGGCCCAAGAAGAACATGAGAAATATCACAGTAATTGG |
| | | AGAGCAATGGCTAGTGATTTTAACCTACCACCTGTAGTAGCAAA |
| | | AGAAATAGTAGCCAGCTGTGATAAATGTCAGCTAAAAGGGGAA |
| | | GCCATGCATGGACAAGTAGACTGTAGCCCAGGAATATGGCAGCT |
| | | AGATTGTACACATTTAGAAGGAAAAGTTATCTTGGTAGCAGTTC |
| | | ATGTAGCCAGTGGATATATAGAAGCAGAAGTAATTCCAGCAGAG |
| | | ACAGGGCAAGAAACAGCATACTTCCTCTTAAAATTAGCAGGAAG |
| | | ATGGCCAGTAAAAACAGTACATACAGACAATGGCAGCAATTTCA |
| | | CCAGTACTACAGTTAAGGCCGCCTGTTGGTGGGCGGGGATCAAG |
| | | CAGGAATTTGGCATTCCCTACAATCCCCAAAGTCAAGGAGTAAT |
| | | AGAATCTATGAATAAAGAATTAAAGAAAATTATAGGACAGGTAA |
| | | GAGATCAGGCTGAACATCTTAAGACAGCAGTACAAATGGCAGTA |
| | | TTCATCCACAATTTTAAAAGAAAAGGGGGGATTGGGGGGTACAG |
| | | TGCAGGGGAAAGAATAGTAGACATAATAGCAACAGACATACAA |
| | | ACTAAAGAATTACAAAAACAAATTACAAAAATTCAAAATTTTCG |
| | | GGTTTATTACAGGGACAGCAGAGATCCAGTTTGGAAAGGACCAG |
| | | CAAAGCTCCTCTGGAAAGGTGAAGGGGCAGTAGTAATACAAGAT |
| | | AATAGTGACATAAAAGTAGTGCCAAGAAGAAAAGCAAAGATCA |
| | | TCAGGGATTATGGAAAACAGATGGCAGGTGATGATTGTGTGGCA |
| | | AGTAGACAGGATGAGGATTAACACATGGAATTCTGCAACAACTG |
| | | CTGTTTATCCATTTCAGAATTGGAGGAGCTTTGTTCCTTGGGTTCT |
| | | TGGGAGCAGCAGGAAGCACTATGGGCGCAGCGTCAATGACGCTG |
| | | ACGGTACAGGCCAGACAATTATTGTCTGGTATAGTGCAGCAGCA |
| | | GAACAATTTGCTGAGGGCTATTGAGGCGCAACAGCATCTGTTGC |
| | | AACTCACAGTCTGGGGCATCAAGCAGCTCCAGGCAAGAATCCTG |
| | | GCTGTGGAAAGATACCTAAAGGATCAACAGCTCCTGGGGATTTG |
| | | GGGTTGCTCTGGAAAACTCATTTGCACCACTGCTGTGCCTTGGAA |
| | | TGCTAGTTGGAGTAATAAATCTCTGGAACAGATTTGGAATCACA |
| | | CGACCTGGATGGAGTGGGACAGAGAAATTAACAATTACACAAGC |
| | | TTGCTAGCAGATCTTTTTCCCTCTGCCAAAAATTATGGGGACATC |
| | | ATGAAGCCCCTTGAGCATCTGACTTCTGGCTAATAAAGGAAATTT |
| | | ATTTTCATTGCAATAGTGTGTTGGAATTTTTTGTGTCTCTCACTCG |
| | | GAAGGACATATGGGAGGGCAAATCATTTAAAACATCAGAATGA |
| | | GTATTTGGTTTAGAGTTTGGCAACATATGCCATATGCTGGCTGCC |
| | | ATGAACAAAGGTGGCTATAAAGAGGTCATCAGTATATGAAACAG |
| | | CCCCCTGCTGTCCATTCCTTATTCCATAGAAAAGCCTTGACTTGA |
| | | GGTTAGATTTTTTTTATATTTTGTTTTGTGTTATTTTTTTCTTTAAC |
| | | ATCCCTAAAATTTTCCTTACATGTTTTACTAGCCAGATTTTTCCTC |
| | | CTCTCCTGACTACTCCCAGTCATAGCTGTCCCTCTTCTCTTATGAA |
| | | GATCCCTCGACCTGCAGCCCAAGCTTGGCGTAATCATGGTCATA |
| | | GCTGTTTCCTGTGTGAAATTGTTATCCGCTCACAATTCCACACAA |
| | | CATACGAGCCGGAAGCATAAAGTGTAAAGCCTGGGGTGCCTAAT |
| | | GAGTGAGCTAACTCACATTAATTGCGTTGCGCTCACTGCCCGCTT |
| | | TCCAGTCGGGAAACCTGTCGTGCCAGCGGATCCGCATCTCAATT |
| | | AGTCAGCAACCATAGTCCCGCCCCTAACTCCGCCCATCCCGCCCC |
| | | TAACTCCGCCCAGTTCCGCCCATTCTCCGCCCCATGGCTGACTAA |
| | | TTTTTTTTATTTATGCAGAGGCCGAGGCCGCCTCGGCCTCTGAGC |
| | | TATTCCAGAAGTAGTGAGGAGGCTTTTTTGGAGGCCTAGGCTTTT |
| | | GCAAAAAGCTAACTTGTTTATTGCAGCTTATAATGGTTACAAATA |
| | | AAGCAATAGCATCACAAATTTCACAAATAAAGCATTTTTTTCACT |
| | | GCATTCTAGTTGTGGTTTGTCCAAACTCATCAATGTATCTTATCA |
| | | TGTCTGGATCCGCTGCATTAATGAATCGGCCAACGCGCGGGGAG |
| | | AGGCGGTTTGCGTATTGGGCGCTCTTCCGCTTCCTCGCTCACTGA |
| | | CTCGCTGCGCTCGGTCGTTCGGCTGCGGCGAGCGGTATCAGCTCA |
| | | CTCAAAGGCGGTAATACGGTTATCCACAGAATCAGGGGATAACG |
| | | CAGGAAAGAACATGTGAGCAAAAGGCCAGCAAAAGGCCAGGAA |
| | | CCGTAAAAAGGCCGCGTTGCTGGCGTTTTTCCATAGGCTCCGCCC |
| | | CCCTGACGAGCATCACAAAAATCGACGCTCAAGTCAGAGGTGGC |
| | | GAAACCCGACAGGACTATAAAGATACCAGGCGTTTCCCCCTGGA |
| | | AGCTCCCTCGTGCGCTCTCCTGTTCCGACCCTGCCGCTTACCGGA |
| | | TACCTGTCCGCCTTTCTCCCTTCGGGAAGCGTGGCGCTTTCTCAA |
| | | TGCTCACGCTGTAGGTATCTCAGTTCGGTGTAGGTCGTTCGCTCC |
| | | AAGCTGGGCTGTGTGCACGAACCCCCCGTTCAGCCCGACCGCTG |
| | | CGCCTTATCCGGTAACTATCGTCTTGAGTCCAACCCGGTAAGACA |
| | | CGACTTATCGCCACTGGCAGCAGCCACTGGTAACAGGATTAGCA |
| | | GAGCGAGGTATGTAGGCGGTGCTACAGAGTTCTTGAAGTGGTGG |
| | | CCTAACTACGGCTACACTAGAAGGACAGTATTTGGTATCTGCGCT |
| | | CTGCTGAAGCCAGTTACCTTCGGAAAAAGAGTTGGTAGCTCTTG |
| | | ATCCGGCAAACAAACCACCGCTGGTAGCGGTGGTTTTTTTGTTTG |
| | | CAAGCAGCAGATTACGCGCAGAAAAAAAGGATCTCAAGAAGAT |
| | | CCTTTGATCTTTTCTACGGGGTCTGACGCTCAGTGGAACGAAAAC |
| | | TCACGTTAAGGGATTTTGGTCATGAGATTATCAAAAAGGATCTTC |
| | | ACCTAGATCCTTTTAAATTAAAAATGAAGTTTTAAATCAATCTAA |
| | | AGTATATATGAGTAAACTTGGTCTGACAGTTACCAATGCTTAATC |
| | | AGTGAGGCACCTATCTCAGCGATCTGTCTATTTCGTTCATCCATA |
| | | GTTGCCTGACTCCCCGTCGTGTAGATAACTACGATACGGGAGGG |
| | | CTTACCATCTGGCCCCAGTGCTGCAATGATACCGCGAGACCCAC |
| | | GCTCACCGGCTCCAGATTTATCAGCAATAAACCAGCCAGCCGGA |
| | | AGGGCCGAGCGCAGAAGTGGTCCTGCAACTTTATCCGCCTCCAT |
| | | CCAGTCTATTAATTGTTGCCGGGAAGCTAGAGTAAGTAGTTCGCC |
| | | AGTTAATAGTTTGCGCAACGTTGTTGCCATTGCTACAGGCATCGT |
| | | GGTGTCACGCTCGTCGTTTGGTATGGCTTCATTCAGCTCCGGTTC |
| | | CCAACGATCAAGGCGAGTTACATGATCCCCCATGTTGTGCAAAA |
| | | AAGCGGTTAGCTCCTTCGGTCCTCCGATCGTTGTCAGAAGTAAGT |
| | | TGGCCGCAGTGTTATCACTCATGGTTATGGCAGCACTGCATAATT |
| | | CTCTTACTGTCATGCCATCCGTAAGATGCTTTTCTGTGACTGGTG |
| | | AGTACTCAACCAAGTCATTCTGAGAATAGTGTATGCGGCGACCG |
| | | AGTTGCTCTTGCCCGGCGTCAATACGGGATAATACCGCGCCACA |
| | | TAGCAGAACTTTAAAAGTGCTCATCATTGGAAAACGTTCTTCGG |
| | | GGCGAAAACTCTCAAGGATCTTACCGCTGTTGAGATCCAGTTCG |
| | | ATGTAACCCACTCGTGCACCCAACTGATCTTCAGCATCTTTTACT |
| | | TTCACCAGCGTTTCTGGGTGAGCAAAAACAGGAAGGCAAAATGC |
| | | CGCAAAAAAGGGAATAAGGGCGACACGGAAATGTTGAATACTC |
| | | ATACTCTTCCTTTTTCAATATTATTGAAGCATTTATCAGGGTTATT |
| | | GTCTCATGAGCGGATACATATTTGAATGTATTTAGAAAAATAAA |
| | | CAAATAGGGGTTCCGCGCACATTTCCCCGAAAAGTGCCACCTG |
| 84 | Vif miRNA | AAGTTCAGAAGTACACATCCC |
| | target sequence | |
| 85 | Tat miRNA | CTATGGCAGGAAGAAGCGGA |
| | target sequence | |
| 86 | Elongation | CCGGTGCCTAGAGAAGGTGGCGCGGGGTAAACTGGGAAAGTGA |
| | Factor-1 alpha | TGTCGTGTACTGGCTCCGCCTTTTTCCCGAGGGTGGGGGAGAACC |
| | (EF1-alpha) | GTATATAAGTGCAGTAGTCGCCGTGAACGTTCTTTTTCGCAACGG |
| | promoter with | GTTTGCCGCCAGAACACAGGTAAGTGCCGTGTGTGGTTCCCGCG |
| | 3′ restriction | GGCCTGGCCTCTTTACGGGTTATGGCCCTTGCGTGCCTTGAATTA |
| | recognition site | CTTCCACGCCCCTGGCTGCAGTACGTGATTCTTGATCCCGAGCTT |
| | | CGGGTTGGAAGTGGGTGGGAGAGTTCGAGGCCTTGCGCTTAAGG |
| | | AGCCCCTTCGCCTCGTGCTTGAGTTGAGGCCTGGCCTGGGCGCTG |
| | | GGGCCGCCGCGTGCGAATCTGGTGGCACCTTCGCGCCTGTCTCGC |
| | | TGCTTTCGATAAGTCTCTAGCCATTTAAAATTTTTGATGACCTGC |
| | | TGCGACGCTTTTTTTCTGGCAAGATAGTCTTGTAAATGCGGGCCA |
| | | AGATCTGCACACTGGTATTTCGGTTTTTGGGGCCGCGGGCGGCG |
| | | ACGGGGCCCGTGCGTCCCAGCGCACATGTTCGGCGAGGCGGGGC |
| | | CTGCGAGCGCGGCCACCGAGAATCGGACGGGGGTAGTCTCAAGC |
| | | TGGCCGGCCTGCTCTGGTGCCTGGCCTCGCGCCGCCGTGTATCGC |
| | | CCCGCCCTGGGCGGCAAGGCTGGCCCGGTCGGCACCAGTTGCGT |
| | | GAGCGGAAAGATGGCCGCTTCCCGGCCCTGCTGCAGGGAGCTCA |
| | | AAATGGAGGACGCGGCGCTCGGGAGAGCGGGCGGGTGAGTCAC |
| | | CCACACAAAGGAAAAGGGCCTTTCCGTCCTCAGCCGTCGCTTCA |
| | | TGTGACTCCACGGAGTACCGGGCGCCGTCCAGGCACCTCGATTA |
| | | GTTCTCGAGCTTTTGGAGTACGTCGTCTTTAGGTTGGGGGGAGGG |
| | | GTTTTATGCGATGGAGTTTCCCCACACTGAGTGGGTGGAGACTG |
| | | AAGTTAGGCCAGCTTGGCACTTGATGTAATTCTCCTTGGAATTTG |
| | | CCCTTTTTGAGTTTGGATCTTGGTTCATTCTCAAGCCTCAGACAG |
| | | TGGTTCAAAGTTTTTTTCTTCCATTTCAGGTGTCGTGATGTACA |
| 87 | miR30 CCR5 | TGTACAAGGTATATTGCTGTTGACAGTGAGCGACTGTAAACTGA |
| | coding | GCTTGCTCTACTGTGAAGCCACAGATGGGTAGAGCAAGCACAGT |
| | sequence with | TTACCGCTGCCTACTGCCTCGGACTTCAAGGGGCTTGCTAGC |
| | 5′ and 3′ | |
| | restriction | |
| | recognition | |
| | sites | |
| 88 | miR21 Vif | CCCGGGCATCTCCATGGCTGTACCACCTTGTCGGGGGATGTGTAC |
| | coding | TTCTGAACTTGTGTTGAATCTCATGGAGTTCAGAAGAACACATCC |
| | sequence with | GCACTGACATTTTGGTATCTTTCATCTGACCA |
| | 5′ restriction | |
| | recognition site | |
| 89 | miR185 Tat | GCTAGCGGGCCTGGCTCGAGCAGGGGGCGAGGGATTCCGCTTCT |
| | coding | TCCTGCCATAGCGTGGTCCCCTCCCCTATGGCAGGCAGAAGCGG |
| | sequence with | CACCTTCCCTCCCAATGACCGCGTCTTCGTCCCCGGG |
| | 5′ and 3′ | |
| | restriction | |
| | recognition | |
| | sites | |

While certain of the preferred embodiments of the present disclosure have been described and specifically exemplified above, it is not intended that the disclosure be limited to such embodiments. Various modifications may be made thereto without departing from the scope and spirit of the present disclosure.

What is claimed is:

1. A method comprising:
(a) ex vivo contacting PBMC isolated from a subject with a stimulatory agent;
(b) depleting at least one subset of cells from the PBMC, wherein the at least one subset of cells comprises any one or more of CD8+ T cells, CD4+ T cells, γδ cells, NK cells, B cells, neutrophils, basophils, eosinophils, mast cells, dendritic cells, T regulatory cells, NKT cells, and erythrocytes;
(c) transducing the depleted PBMC ex vivo with a viral delivery system encoding at least one genetic element; and
(d) culturing the transduced PBMC for at least one day.
